SEQUENCE LISTING (152 sequences)

SEQ ID NO: 1 - DNA, 4154 bp, Rattus norvegicus
  misc_feature (1)..(4154): where n may be a or g or c or t/u, unknown, or other
cttcaggagtacgaagaccctgcctacgaaggaactcagctctgtgttcctgccagctcccccgccagcttcacttgccaccaacgctgccacaactgctgccaccaccgctgccacctctgcgatgctcttccgagctgtgctgctctgcgcttgccctggcctcagccatgcagcaaatccttgctgttccaacccatgtcaaaaccgtggtgaatgtatgagcataggatttgaccaatataaatgtgactgtacccggactggattctacggtgaaaactgtactacgccgagattcctgacaagaatcaaattaccgctgaagcccaccccaaacacagtacactacatcctgacccacttcaagggagtctggaacattgtgaacaacattcccttccttcgaattcaatccatgagatacgtgttgacgtccagatcacatttgattgacagcccaccaacttacaatgtgcactacggttacaaaagttgggaagctttctccaacctctcctactacaccagggcccttcctcctgtggctgatgactgcccaactcccatgggtgtgaaaggaaataaggaacttcctgattcaaaagaagttctggaaaaggttcttctgaggagagagttcatcccggatccccaaggcacaaatatgatgttcgcattctttgcccagcacttcactcatcagtttttcaagacagatcagaagcgaggacctgggttcacccgaggactgggccatggagtggacttaaatcatgtttacggtgaaactctagacagacaacataaacttcgacttttccaggatggaaaattgaaatatcaggtcatcggtggagaggtgtatcctcccacagtcaaagacactcaggtagacatgatctaccctccccacgtccctgagcacctgcggttcgctgtggggcaggaagtctttggtctggtgccgggtctgatgatgtatgctaccatctggcttcgggagcacaacagagtgtgtgatattctcaaacaggagcatcctgagtgggatgacgagcgactgttccaaaccagcaggctcatactgataggagagacgatcaagatagtgatcgaagactacgtgcaacacctgaggggttaccacttccaactcaagttcgacccagacctgcttttcaaccagcagttccagtatcagaaccgcattgcctctgaattcaagacactctatcactggcatccgctgctgccggacaccttcaacattgaagaccaggagtacactttcaaacagtttctctacaacaactccatcctccttgaacacggacttgctcactttgttgagtcattcaccagacagattgctggccgggttgctgggggaaggaatgttccaatcgctgtacaagcagtggcaaaggcctccattgaccagagcagagagatgaaataccagtctctcaatgagtaccgcaaacgcttctccctgaaaccttacacatcgtttgaagaacttacaggagagaaagaaatggctgcagagttgaaagccctctaccatgacatcgatgccatggaactgtatcccgccctgctggtggaaaagcctcgnccagatgctatctttggggagaccatggtagaacttggagctccattctccttgaaaggccttatgggtaatcccatctgttctcctcaatactggaaacctagcaccttcggaggagaagtggggtttaggatcatcaacactgcctcaattcagtctctcatctgcaataatgtgaaagggtgtcccttcgcctctttcaatgtgcaagacccgcaggctaccaagacagccaccatcaacgcaagtgcctcccactccagactagatgacattaaccctacagtactaatcaaaaggcgttcaactgagctgtaagagtctactgaccatatttatttatttatatgaacaatttaatttaattatttaatattatacagaatgcttttttttcacttaacatcttctataacagaaggcaatattcttgaacaatgttccatttgtgaagattcctgtgttgtacttttaaatatggttatccgaaagtgaaagggaaaaaaaagaacactttcatttttcggcataagccagtgagaagggaaatgaattttgatatctttatacttgaatgtcagctcatgactagccttaattaagaacaaatgaaaggtgtatgaatatttaaatgctgttacaaggagggaaatgtgaatatttaaatgctgttacaaggagggaaatgctgcattgttggttatgactgtgtcatccttactatgttaggagcaaccgatgtggaattgtttttaaatcttgcatatctttatctcatcaaagcaaaggggtacaagtccagttttaaatgaacatgaaggcagataccggcaactgtctttttattttttaaaagcaatctttgaaacaaatgatttgcaatgtctaaatcgggagttggaatcactttcgaaagcccttactttcttgagctgtcaaatttgtacccacacagattaagcagctaccataaacacaaatctaaaactggggaaaactattatgactgatggttaagataccatgtcagggatctttcttttctcaggagtagtgaaaagctactatgacaatcagaccttccttgtacgtcagattgctggcgtaggaaggtggcggagcccgtgatgttctgtcctaaacgatggaaaagctttaaagcttgtgtgtgagtggtagccagcaaaacctatcatagcaacaaaagagtccacaaacaaaataaccaagaacaaagaagggttcccaagcttaaagaccgcatcgagggttaaactttttggaagggagacttttcagatcctcctgtgggtgcttggcttgtgactttggcaggctggattttaaagagtttttctgttgcacagtatgacacaacagcccatctctcaatgcaaaaggtatcagtgggcttcttcaaaactttgaaatgtcttccagctcacggtaccagaagtgcagtaggccccatgcaatgtgtgagttcagcctggatgccagcatgatgctctccttactctgtttcttgtagtcattttgctctgagaaactgctgattgatctgtttttgtagctgtgttccaggctcttagtattctttctttaacctccattaatattttctctacttgaagttttacattcaggaaaaacctcagctcaggactactgtgtagctccccttcggaggaagaagttactttagacaaaagggaaaaaaaattaaatgtatttttcatttgtaattaaatggaagggccctaccaagattctagaattggagggggttctgacaagaaagttacattcttgtcctgaagaattgctttcttatttaaaaacagagtcagttagtgggtagttctgggcaatagaaataaatataaaacaataatgacaatcattctctacatctcattatcagctgaggtactgtatattactgaatttactgaagatagttatgtctttcagacattgttgttataaactatgtttaagcctactacaagtgtttcttttttgcattatgtcagaattgatgtaccttttttttaatgattacctccctgtactactgtgcgaacaatcaaacaaaatgatgagattaatggtcatggataaatttcaagaaaactagtgtatttgattgaaaagtttaaagttagaacttaggccattggaatttactcatatagcaaactgcgtagagccaatattgactcacctacacacgttatacagattgacattttagacatttggaaggccccgtaggtattttattagttagaacttaattttttgaaaaaacatatccaaagcacaataggcattagaatttgtgcatcgagaactgattacaaataatattgatatgtatgtaaataactgagaagtatgtcttatgaagaaatatattttattacaaaaaattataaaacattttcaagattatatgctttaaaagtttaagatcagaaaataatcaactttagaaaaacacgtttaaaaattgttaacatcattgatt

SEQ ID NO: 2 - DNA, 1582 bp, Rattus norvegicus
gaaagggagcggcggcgggaggcgcggagcgcgggcgccgggaagatgctgcagtccctggccggcagctcgtgcgtgcgcctggtggagcggcaccgctcggcctggtgcttcggcttcctggtgctgggctacctgctctacctggtgttcggcgccgtggtcttctcgtccgtggagctgccttatgaggacctgctgcgccaggagctgcgcaagctgaagcggcgcttcctggaggagcacgagtgcctgtcggagccccagctggagcagttcctgggccgcgtgctggaggccagcaattatggagtgtcggtgctcagcaacgcctcggggaattggaattgggacttcacctcggcgctcttcttcgccagcacggtgctctccaccacaggctatggccacacggtgcccttgtcagatgggggcaaggccttctgcatcatctactctgtcattggcatcccgttcaccctcctcttcctgacggccgtggtccagcgtgtcaccgtgcatgtcacccgcagaccggtcctctacttccacatacgctggggcttctccaagcaggtggtggccatcgtccatgccgttctgctgggattcgtcaccgtgtcctgcttcttcttcatcccggccgccgtgttctccgtgctggaggatgactggaacttcctcgaatctttttacttctgtttcatctccctgagcaccatcggcctgggggactacgttccaggggaaggctacaaccagaagttccgggagctgtacaagatcgggatcacgtgttacctgctcctgggactcatagccatgctggttgtcctggaaaccttctgtgagctccacgagctgaagaaattcaggaaaatgttctatgtgaagaaagacaaggatgaagaccaagttcacatcatggagcatgaccaactgtccttttcctccatcactgagcaggcggccggcctgaaggaggagcagaagcaaaacgagccttttgtggcctcccagtcaccaccctatgaggatggctctgcaaaccattgagcatgggtcaccagaccccggacatgaggcagagcctagactgtgttcatttttacgagaaagtcaaagctaagatgatgttattttaagaaatatctactgttaacaatattttaaaaacacggaactttggatcctgggggagtgggttttaatctctgggcaaatgaggtgtgcacccataactcacaggcaatgtgatcacctgacatcatgcagctgtaactcacaggcgatgtcatcacctggcatcctgcacctgtaactcacaggtgatgtcatcgcctggcatcctgcacctgtaactcacaggtgatattatcacctggcatcctgcacctgtaactcacaggtgatgtcatcacctggcatcctgcacttgtaactcacaggttgatgtcatcgcctggcatcctgcacctgtaactccacagttgatgtcatcacctgggcattacacactggggaagaaatacttgaaacatgttactgcaccggaaatcccattctaaaaccaaacttacta

SEQ ID NO: 3 - DNA, 645 bp, Rattus sp.
atgaactttctgctctcttgggtgcactggaccctggctttactgctgtacctccaccatgccaagtggtcccaggctgcacccacgacagaaggggagcagaaagcccatgaagtggtgaagttcatggacgtctaccagcgcagctattgccgtccaattgagaccctggtggacatcttccaggagtaccccgatgagatagagtatatcttcaagccgtcctgtgtgcccctaatgcggtgtgcgggctgctgcaatgatgaagccctggagtgcgtgcccacgtcggagagcaacgtcactatgcagatcatgcggatcaaacctcaccaaagccagcacataggagagatgagcttcctgcagcatagcagatgtgaatgcagaccaaagaaagatagaacaaagccagaaaaaaaatcagttcgaggaaagggaaagggtcaaaaacgaaagcgcaagaaatcccggtttaaatcctggagcgttcactgtgagccttgttcagagcggagaaagcatttgtttgtccaagatccgcagacgtgtaaatgttcctgcaaaaacacagactcgcgttgcaaggcgaggcagcttgagttaaacgaacgtacttgcagatgtgacaagccaaggcggtga

SEQ ID NO: 4 - DNA, 367 bp, Rattus norvegicus
tttttttttttttttgtctgttttctgaaagagggacagtttattatcaattcacaattaaagcagcatgcaatttattattttttttaactttttgttttattcctggcaacggcaacaaaccacaacattatcgaggaatgttatgcagacttttaaagttgtgcgcaaatgactgtttcattctggtcatggatatgtccaataaatagattgtagaaccactgtactgtataaacttcatttatacatgcagttcataaaattatctttttcttaactgaataatttaccctgttatgtctatatacaaatagataatttttgtctcaatataatctatacaacataaatccctcgtgccgaa

SEQ ID NO: 5 - DNA, 405 bp, Rattus sp.
ggcttttattattcacatgctcggtagaaaacggggtttagtaaactgggtggaggtgtacggcaagactctgagttggtccggaaattatttacacctgagggcagcagcactgttcgtcacttcaggcacagcacgtgcacttgtccgaggcacctttgcaaacacagccctgggcacatttggagcagcccacggggcagcaggagcagcagctcttcttgcaggaggtgcatttgcagtttttgcagccgcaggagctggaccaggtgcaggagccgccggtggagcaggaccagttggggtccattccgagatctggtgaatctggagcaacggggtaagctacaagaaggcagtccctcgtgccgaattcttggcctctagggccaaattccctatagg

SEQ ID NO: 6 - DNA, 1080 bp, Rattus norvegicus
ctcatgacccagataagtggagtgaagaaactgatgcacagctcaagcctgaacaacacaagcatctcacgctttggagtcaacacggaaaatgaggatcatctagccaaggagctggaagacctgaacaaatggggccttaacatcttcaacgtggctgggtactcccataatcggcccctcacatgcatcatgtacgccattttccaggaaagagaccttctaaagacgtttaaaatctcctccgacaccttcgtaacctacatgatgactttagaagaccattaccattctgatgtggcgtatcacaacagcctgcacgctgctgacgtggcccagtcaacgcacgttctcctctctacgccagcactggatgctgtcttcacagacctggaaatcctggctgccatttttgcagctgccatccatgatgttgatcatcctggagtctccaatcagtttctcatcaatacaaattccgaacttgctttgatgtataatgacgaatctgtgctggaaaaccatcacctcgctgtgggattcaagctccttcaagaggaacattgcgacatctttcagaatcttaccaagaagcaacgccagacactcaggaaaatggtgattgacatggtgttagcaactgatatgtccaagcacatgagcctcctggctgaccttaaaacgatggtagaaaccaaaaaggtgacgagctccggtgttctcctcctggacaactatactgaccggatacaggttcttcgcaacatggtacattgtgcagacctgagcaaccctaccaagtccttggagttgtatcggcaatggactgatcgcatcatggaggagtttttccaacagggagacaaagaacgggagaggggaatggagattagcccaatgtgtgataaacacacagcttctgtggaaaagtcccaggttggtttcattgactacattgtccatccattgtgggagacctgggcagacctggttcagcctgatgctcaagacattttggacacactagaagataacaggaactggtaccagagtatgattccccagagcccctctccacca

SEQ ID NO: 7 - DNA, 580 bp, Rattus norvegicus
tttttttttttttttttctgatcttaattcattttattctacaaaatgctactcagtggaaagtaggaaagccaacaagacaacaagaacataaaacgagaacaaaccccgagggaaaataagttttaatatgttcttccctccatagcagcaagctctaaacagctttccttagtgcaaatactgtaggcttgtgtcacacacagtacacagaacaacgcaacacacaccaccacagatgcttctgagcagagatactcctcaaaaatttaaaactatacaaagattttttgagcacgtggtcctgcctggagaattcgactagagagaccctcctaggaccatttcaccattactgtaaaaacgggacaaaaggtccccagaaaggaaattagaattccccatggagccataaaaccttgtacaactcgtttgcctccagggtctaatagcaaatttcactgcacgtcattgacatatcccaaatacggatgcataaagcttgagtttctacgatataccaaaatacgatatatatacaactcccactgcaaaagaaaccctgatacctagtctttat

SEQ ID NO: 8 - DNA, 1159 bp, Rattus norvegicus
  misc_feature (1)..(1159): where n may be a or g or c or t/u, unknown, or other
ccgattaggtccccaaaacgggggacgtccatggggatgaacatgatttccaagggtacggagaaagcacttctgaagcttcaagagttctttcctgagctgcagattctggcggtcagtggtaactattgcaccgacaagaaacctgctgccataaactggatcgaagggagaggaaagactgtggtttgtgaagctgtcattccagccaaggtggtgagagaagtattaaagagcactacggaagctatggttgacgtaaacattaataagaatcttgtgggctctgccatggctggtagcataggaggctacaacgcccatgctgccaacatcgtcactgccatctacattgcatgtgcccaggatgcagcacagaatgtggggagttcaaactgtattactttaatggaagcaagtggtcccncnnntgaagacttgnnnnncagctgnnncatgccgtctatagagatcggaaccgtgggtggtgggaccaaccttctacctcagcaagcctgcctgcagatgctaggtgttcaaggggcgtgcaaagacaatcctggagaaaatgcacggcagcttgcccgaattgtgtgtggcactgtaatggctggtgagttgtccttgatggcagcattggcagcaggacatcttgtcagaagtcacatggttcacaacagatcaaagataaatttacaagatctgcagggaacatgcaccaagaaggcagcttgagcatcctgacatacttgaactgaaacacgggcattgggttctcaaggtctaacatgaaatctgtgaattaaaaatgtcagtgcagtgtcttgtggaagatgaacgtgatcagtgagcctgcttggtttctggctctttcagagacgtctgcggtcctttgcaccagactcctcagacgtgggaactatggttctttccgtgccgtattctagaaagatctcatgtggatgtcatggtgctctgagcaccacagatgtgactgcagctcgtttctaaaagctgccacaagctggaagctggtgttttgacgaaatgatggatcttggtgatcagtgtggggctcacctccaatgggttaaaatggagttttaaatgacactgtagctgacagaactctcgatttttatttattcagtctgggcgg

SEQ ID NO: 9 - DNA, 539 bp, Rattus norvegicus
caagctcattcctcgcagaggcgcccagagcagagcacccgctgcgcagagaccacagcccgcccgccatgatgctaggtaacaaacgaatggggctgtgtggactgaccctcgctctatccctgctcgtgtgtttgggcattctggctgaggggtacccctccaagccggacaatccgggcgaggacgcgccagcagaggacatggccagatactactccgctctgcgacactacatcaatctcatcaccagacagagatatggcaagagatccagccctgagacactgatttcagatctcttaatgagagaaagcacagaaaatgcccccagaacaaggcttgaagacccttccatgtggtgatgggaaatgaaacttgctctcctgacttttcctagtttccccccacatctcatctcatcctgtgaaaccagtctgcctgtcccacccaatgcatgccaccaccaggctggattccgacccatttcccttgttgtcgttgtatatatgtgtgtttaaataaagtatcatgcattc

SEQ ID NO: 10 - DNA, 465 bp, Rattus norvegicus
tttttttttttttttttgaaagtttaggcattttaatctacacaaaaaactgcaagcaaactaatatctaaggtagagtaaaatgcctaaactttcaaaacaaaaggccaaaaaaaagaactttagctgcacttccaagcaattaaattaatgagagttccaatccttgggctccccttagcaatgtacagctgttcaacctcaaataccaacaacagtggaagaaatgatagttttctatacttagccacgtccacggagttgactgtaaagactaggaataataagcaagataactctaggagaagatgacacaaactcactttctagctgcatttctgtaccgtaatttcagcctctatattctcgaaaccaacgctttaaaagaatcacactgcaaagctggtctcaagtataaatggcaaaaacaaagtacctgatgtgtatgcctcgtgccgaattctt

SEQ ID NO: 11 - DNA, 2002 bp, Rattus norvegicus
ccctttaatttcctcgaaaactccaatcactcggctgaagccatgccttgtgttcaggcgcagtatgggtcctcgcctcaaggagccagccccgcttctcagagctacagttaccactcttcgggagaatacagctccgatttcttaactccagagtttgtcaagtttagcatggacctcaccaacactgaaattactgccaccacttctctccccagcttcagtacctttatggacaactacagcacaggctacgacgtcaagccaccttgcttgtaccaaatgcccctgtccggacagcagtcctccattaaggtagaagacattcagatgcacaactaccagcaacacagccacctgccccctcagtccgaggagatgatgccacacagcgggtcggtttactacaagccctcttcgcccccgacacccagcaccccgggcttccaggtgcagcatagcccgatgtgggacgatccgggctcccttcacaacttccaccagaactacgtggccactacgcatatgatcgagcagaggaagacacctgtctcccgcctttcactcttctcctttaagcagtcccgcccgggcactcctgtgtctagctgccagatgcgctttgacgggcctctgcacgtccccatgaacccggagcccgcgggcagccaccacgtagtggatgggcagaccttcgccgtgcccaatcccattcgcaagccggcatccatgggcttcccgggcctgcagatcggccacgcgtcgcagttgcttgacacgcaggtgccctcgccggcgtcccggggctctccctccaatgagggtctgtgcgctgtttgcggtgacaacgcggcctgtcagcattacggtgttcgcacttgtgagggctgcaaaggtttctttaagcgcacggtgcaaaaaaacgcgaaatatgtgtgtttagcaaataaaaattgcccagtggacaagcgccgccgaaatcgttgtcagtactgtcggtttcagaagtgcctggctgttgggatggttaaagaagtggttcgcacggacagtttaaaaggccggagaggtcgtctaccctcaaaaccgaagagcccacaggatccctctcccccctcacctccggtgagtctgatcagtgccctcgtcagagcccacgtcgactccaatccggcaatgaccagcctggactattccaggttccaggcaaaccctgactatcagatgagtggagatgatactcaacatatccagcagttctacgatctcctgactggctctatggagatcatcagagggtgggcagagaagattcctggctttgctgacctgcccaaagccgatcaggacctgctttttgaatcagctttcttagaattatttgttctacgcttagcatacaggtccaacccagtggagggtaaactcatcttttgcaatggggtggtcttgcacaggttgcaatgcgtgcgtggctttggggaatggattgattccattgttgaattctcctccaacttgcagaatatgaacatcgacatttctgccttctcctgcattgctgccctggctatggtcacagagagacacgggctcaaggaacccaagagagtggaagagctacaaaacaaaattgtaaattgtcttaaagaccatgtgactttcaataatgggggattgaaccgacccaactacctgtccaaactgttggggaagctcccagaacttcgcaccctttgcacacaggggctccagcgcattttctacctgaaattggaagacttggtaccaccaccagcaataattgacaaacttttcctggacaccttacctttctaagactttctcccatgcacgtcaaagaactggaaagaaaaaaaaaatccagagggggctggtcaagatgggtagagagctggctgaagtgtccggttcatgtctcccttctgtagacccctagccctcacccctaaagtaaacaaacaaacaagcaaacaaacgg

SEQ ID NO: 12 - DNA, 2469 bp, Rattus norvegicus
atgtggggctggaggggcctcctcttctgggctgtgctggtcacagccactctctgcactgccagaccagccccaaccttgcccgaacaagctcagccctggggagtccctgtggaagtggagtctctcctggtccaccctggtgacctgctacagcttcgctgccggctgcgcgatgatgtgcagagcatcaactggctgcgggatggggtgcagctggcggaaagcaaccgtacacgcatcacaggggaggaggtggaggtgcgggattccatccccgctgactctggcctctacgcttgtgtgaccaacagcccctctggcagcgataccacctacttctccgtcaatgtctcagatgcactgccatcctcggaggacgatgacgatgatgatgactcctcctcagaggagaaagagacagacaacaccaaaccaaaccgtaggcctgtggcgccatactggacatccccagagaaaatggagaagaaactgcacgcagtgccagctgccaagacggtgaaattcaaatgcccgtcgagtgggacacccagccccactttgcgctggttgaaaaacggcaaggaattcaaacctgaccaccggatcggaggctacaaggttcgttacgccacttggagcatcataatggactctgtggtgccttctgacaagggcaactacacctgcatcgtggagaacgagtatgggagcattaaccacacctaccagctagacgttgtggagcgatcccctcaccggcccatccttcaggcagggctaccagccaacaagaccgtggccctgggcagcaacgtggagttcatgtgcaaggtgtacagtgacccccagcctcacatccagtggctgaagcacatcgaggtgaatgggagtaagatcggtccagacaacttgccgtatgaccagatcctgaagactgctggagttaataccaccgacaaggaaatggaggtgcttcatctacggaatgtctcctttgaggatgcgggggagtatacgtgcttggcgggtaactctatcggactctcccatcactctgcatggttgaccgttctggaagccctggaagagagaccagccgtgatgacctcacctctgtacctggaaatcattatctactgcaccggggccttcctgatctcctgtatggtgggctccgtcatcatctacaagatgaagagcggcaccaagaagagcgacttccatagccagatggctgtgcataagctggctaagagcatccctctccgcagacaggtaacagtgtcagctgactccagcgcatccatgaactccggggttctcctggttcggccttcgcgactgtcctccagcggaacccccatgctagctggcgtctctgaatatgagctccctgaagatccccgctgggagctgccccgggacagactggtcttaggaaaaccgcttggcgagggctgcttcgggcaggtggtgttggccgaagccatcggtctggataaggacaaacccaaccgcgtgaccaaagtggccgtgaagatgttgaagtctgatgcgacggagaaggacctgtcggacctgatctcggagatggagatgatgaaaatgatcgggaagcacaagaatatcatcaacctgctgggggcgtgcacacaggatggtcctctctatgtcattgtggagtatgcctccaaaggcaatcttcgggagtatctgcaggcccggaggcctcctgggctggagtattgctacaaccccagccacaaccctgaggaacagctgtcttccaaagatctggtgtcctgtgcctatcaggtggcccggggcatggagtatcttgcctcgaagaagtgtatacaccgagacctggctgctaggaatgtcctggtgacagaggataatgtcatgaagatcgcagactttggcctagctcgagacattcaccatatcgactactataagaaaaccaccaatggccggctgcctgtgaagtggatggcacctgaggcattgtttgaccggatctacacccaccagagtgatgtgtggtcttttggggtgctcttatgggagatattcactctgggtggctcaccaaaccccggcgtgcctgtggaagaacttttcaagctgttgaaggagggtcatcgaatggacaagcccagtaactgtaccaatgagctgtacatgatgatgcgggactgctggaacgcagtgccctctcagagaccaactttcaagcagttggtggaagacctggaccggattgtggccttgacctccaaccaggagtatctggacctgtccatgccactggaccaggactcgccaagctttccggacacacggagctctacctgctcttcaggggaggactctgtcttctctcatgagccatttcctgaggagccctgtctgccccgacaccccacccagcttgcaaatggcggactcaaccggcgctga

SEQ ID NO: 13 - DNA, 3165 bp, Rattus norvegicus
  misc_feature (1)..(3165): where n may be a or g or c or t/u, unknown, or other
gcagaatttggcaggcccaggctagggtgcaccaacccttaggctcagaatgacgagacagggccgggctctttccctccggctattgccacacttcctgcctcggctctttttccctagcctgtttctaaggaagggagtggggttgggcgaccgcaccccagctatcccgcctcttcggccctccaaaagctgacaggatatcgggcgagcccagagtgactaaggggaggctttggactcaggtacagggtatgtcagtgcctagagaccacttacgaggtacagtctcattcttacaagccccttgtctctgggatcctccaagtcgtcctctcgggtagatctcagataccctcctggcatcgcctttacagctcagaaggaagccatgtcctgaccattttaactttccatcagagttctgcaaataatcacagtgaccccaatctttgctagatatgttcccatcttctactcctattgcctcagcgtttccaggacccttgttcactttctgcatatctaaattgacctccccaaaattactccccttttccacttatgggagaaccctcagactcatctgtgggctttagagcccctactcatttctatacaataggtacctgaccctgcttcctgaaccttctctcccacctgttcttaagcacttgcactcggggcctctagttaattcctaggtcaaatgttaacaattgctcccgttttgtaccttccctgttttctggggtcttctagtatattgtcagttcacaagtcacagctattgtgggtcgccgcgagactattccgggaatatcctgaggcaccccatctaccttgcagtgtacagagatctcatagccaccgggttgtactcccgcagtcctaactcctatgtgcctcggtccctcaacatccctttgcttgagtgtctctgtctctacagcccctccccctgcagccgcgcagagccaccgggctgctggccgctgtttacaaggacacgcgcttcctgacagtgacgcgagccgcctcctccccttccccacgctctaggagggggccgcgggggcctggctcccgcgtcggccaatcggagtgcacttccgcagctgacaaattcagtataaaatgcttggggctggggccgaacactggggaccttgagggtggccaggccagctttggatcctgcagggagcggggagctgagagaagagacgctgagaaagcgggcgcaccacggagggagagaaaagctccagaagccgggcagcgcttttacgcacagctaccaactggccgctgccgaccgtctccagctcccgaggacgcgcgaccggacgccgggtcccgccacagccgaggacagctcgccggtcgccgcaggcaggcccggagcggccttcagggggacctttcccagatcgcccaggccgcccggatgtgcacgaaaatggaacaggctttctatcacgacgactcttacgcagcggcaggatacggtcggagccctggcagtctttctcttcacgactacaaactcctgaaacccaccttagcgctcaacctggcagatccttatcggggtctcaagggtcctggggcgcggggtccaggcccagagggcagtggggcaggcagctacttttcgggtcagggatcagacacaggcgcatctctgaagctagcctccacggaactggagcgcttgatcgtccccaacagcaacggcgtgatcacgacgacgcccacgcctccgggacagtacttttacccccgtgggggcggcagcggcggaggtacagggggcggcgtcaccgaggagcaggagggctttgcggacggttttgtcaaagccctggacgacctgcagaagatgaaccacgtgacgccccccaacgtgtctctgggcgccagcgggggtccccaggccgggccagggggcgtctatgctggtccggagccgcctccggtctacaccaacctcagcagttactccccagcctctgcaccctctggaggttccgggaccgccgtcgggactgggagctcatacccgacggccaccatcagctacctcccacatgcaccaccctttgcgggcggccacccggcacagctgggcttgagccgtggcgcttccgcctttaaagaggaaccgcagaccgtaccggaggcacgcagccgcgacgccacgccgcctgtgtcccccatcaacatggaagaccaggagcgcatcaaagtggagcgaaagcggctgcggaacaggctggcggccaccaaatgccggaagcggaagctggagcgcatcgcgcgcctggaggacaaggtgaagacactcaaggctgagaacgcggggctgtcaagtgctgccggcctcctacgggagcaagtggcgcagctcaagcagaaggtcatgacccacgtcagcaacggctgccagttgctgctaggggtcaagggacacgccttctgagagcctcccttgctccatacggacacccccagccttgaaggctgggcgcccgccccccactggggtgaggggggcaggcgatgggcactcgcccagaggtctggggcgcagctcacacactggactctggcctgcccgcctgcgcccagtccttccacctcgaggtttacatggcccccttccagcgtattttgtatgttttttttttctggaaagagactgaattcatattgaatataatatatttgtgtatttaacagggaggggagaagggggttgtcgcggcggagtggccccgccgcctggtactcagcctgtggggatactagggaggaacctccgccccctgccctccccctctgcacagtactgtggagaagaaacacgcacttcgtgtctaaagtctattttaagatgtgtttgtgtgtgtgtttgactttttattgaatctatttaagtaaaaaaaaagtctttattaatttctgtggtctctttcttccaagctgnncgnatggagggagaagattgggctgnnccaagcccggggcagtttgtagttctctcctttcgggtatcttaaggtccagtacaagcgctcaaacctcccatcccctgaggtcctggcaccagagctgcgcaggcaggcagtggccgagttacaagctatccaggccgaattc

SEQ ID NO: 14 - DNA, 421 bp, Rattus sp.
atctcttatttacaaacactgggtaggacacccaaacaaacaaacatggaataacttacaaaggcaggaagctgtttattatagacagtaatcagctttcatcaaattaaaaaaaaatatatgtacatacacagttgagagaggcaggccaggggagttcatccgcaatctagcctggtactcacaagcctccctccccttcccagccctccctgctttgtgttcttacggagcactacagaagcaatctacagtctctattgcagtttgtaagcccccacccaccccccttaatactgaatgagatcgaatgttaggtccatgcagttcttggtcaatgttaacgaaaagtccatcgttctggtcgcgcgggcacagcccgttcgcaaagcgtggcggtcaacaggccgctgctctctgg

SEQ ID NO: 15 - DNA, 2957 bp, Rattus norvegicus
ctcggacagcatccgccgcgctcgcccggggctcctagagaacccgggggcgcttgaccgcgcgcggcggcccggcgtcgtacatcgcgaggtcgtcgcactcgcgcaacccagagccaggcccgctgtgcccggagctcatgagcaccatgcacctgctgacattcgccctgcttttttcctgctccttcgcccgcgccgcctgcgaccccaagatcgtcaacatcggcgcggtgctgagcacgcgcaagcatgaacagatgttccgcgaggcagtaaaccaggccaataagcgacacggctcttggaagatacagctcaacgccacttctgtcacccacaagcccaacgccatacagatggccctgtcagtgtgtgaggacctcatctctagccaggtctacgctatcctagttagccacccgcctactcccaacgaccacttcactcccacccctgtctcctacacagctggcttctacagaatccctgtcctgggactgactacccgaatgtccatctactctgacaagagtatccacctgagtttccttcgcacggtgccgccctactcccaccagtccagcgtctggtttgagatgatgcgagtctacaactggaaccacatcatcctgctggtcagcgacgaccacgagggacgggcagcgcagaagcgcttggagacgttgctggaggaacgggagtccaaggcagagaaggtgctgcagtttgacccaggaaccaagaatgtgacggctctgctgatggaggcccgggaactggaggcccgggtcatcatcctttctgcaagcgaggacgacgctgccacagtgtaccgcgcagccgcaatgctgaacatgacgggctctgggtacgtgtggctggtcggggaacgcgagatctctgggaacgccctgcgctacgctcctgatggcatcatcggacttcagctcatcaatggcaagaatgagtcagcccacatcagtgacgccgtgggcgtggtggcacaggcagttcacgaactcctagagaaggagaatatcactgacccaccgcggggttgcgtgggcaacaccaacatctggaagacaggaccattgttcaagagggtgctgatgtcttctaagtatgcggacggagtgactggccgtgtggaattcaatgaggatggggaccggaagtttgccaactatagtatcatgaacctgcagaaccgcaagctggtgcaagtgggcatctacaatggtacccatgtcatcccaaatgacaggaagatcatctggccaggaggagagacagagaaacctcgaggataccagatgtccaccagactaaagatagtgacaatccaccaagagcccttcgtgtacgtcaagcccacaatgagtgatgggacatgcaaagaggagttcacagtcaatggtgacccagtgaagaaggtcatctgtacggggcctaatgacacgttcccaggcagcccacgccacacagtgccccagtgctgctatggcttctgcatagacctgctcatcaagctggcgcggaccatgaattttacctatgaggtgcacctggtggcagatggcaagtttggcacacaggagcgggtaaacaacagcaacaaaaaggagtggaacggaatgatgggcgagctactcagtggccaagcggacatgattgtggcaccactgaccatcaacaatgagcgtgcgcagtacatagagttctccaagcccttcaagtaccagggcctgaccattttggtcaagaaggagattcccaggagcacactggactcatttatgcagccttttcagagcacactgtggttgctagtaggactgtcagttcatgtggtggctgtgatgctgtacctgctggaccgcttcagtccctttggccgattcaaggtgaacagtgaggaggaggaggaagatgcactgaccctgtcctctgccatgtggttttcctggggcgtcctgctcaactccggcattggggaaggtgccccccggagtttctctgcacgtatcctaggcatggtgtgggctggtttcgccatgatcatagtggcttcctacactgccaacttggcagctttcctggtgctggatcggcctgaggagcgcatcacgggcatcaatgaccccaggctcagaaacccctcagacaagttcatctacgcaactgtaaagcagagctccgtggacatctacttccggaggcaggtggagttgagtaccatgtaccggcacatggaaaaacacaattacgagagcgcagctgaggccatccaggctgtgcgggacaacaagctgcacgcctttatctgggactcggccgtgctggagtttgaggcttcacagaagtgcgatctggtgaccacgggtgagctgttcttccgctcaggctttggcatcggcatgcgcaaggacagcccctggaagcagaacgtttccctgtccatactcaagtcccatgagaatggcttcatggaagatctggataagacatgggttcggtatcaggaatgcgactcccgcagcaatgctcctgcaaccctcacttttgagaacatggcaggggtcttcatgctggtggctggaggcatcgtagctgggattttcctcattttcattgagatcgcctacaagcgacacaaggatgcccgtaggaagcagatgcagctggcttttgcagccgtgaacgtgtggaggaagaacctgcaggatagaaagagtggtagagcagagcccgaccctaaaaagaaagccacatttagggctatcacctccaccctggcctccagcttcaagagacgtaggtcctccaaagacacgagcaccgggggtggacgcggcgctttgcaaaaccaaaaagacacagtgctgccgcgacgcgctattgagagggaggagggccagctgcagctgtgttcccgtcatagggagagctga

SEQ ID NO: 16 - DNA, 644 bp, Rattus sp.
gctgtgtgatagttctttatttcaccatttaagagaaagaaagatggaggaaaggtaaacagtgttcaggcttcagcttttgccaggggaaggcttcgggtcatcgagaccccaaggtattgccaggtgcacaaatctggattccgtggcaggcaggcaaagtgatcgctctggtagcccttctcagagcccatgaggatctgatctgtccacaagcaatgactgtcactctccagtttgcaagggatggctgaacagggaaacactgtgcacaccccacagccagcactataggtctttacgaaggccttttgctgagcagggctcagattatgccagggaaccaggaagctgcaggcagtgatgtgcaaatttccgttccttaaacggcccgcgatgagaaactcctcgctgcggttctgggacttgtggacatatccacagaggctctccatggctggggtgtaggcgaaccggaaacctgtggcatttcccacagcgtcgaatcctttgagcatcttagtcatcttgatctcataacgctggtataaggtggtctcgatgatttctggggaacccatgaatttagcccttataaccaggtccgagttgcagaaagctgtctgtgggtgggttggggcaca

SEQ ID NO: 17 - DNA, 2562 bp, Rattus sp.
tggtgctggcgtttccctgcttgcacgcggttccctcgagcgccgctccgaccgacgtagccggccgcgaaggcgcccagacggcaagccagcgacccatgctgaagtgagcgcccaggtcagcgagatgctggcgctgctgaccgccggcgtggcgctcgccgtggccgcgggacaagcccaggataacccgatacctggcagtcgcttcgtgtgcaccgcgctgccccccgaagcggcgcgcgccggctgcccgctgcccgcgatgcccatgcagggaggcgcgctgagccctgaggaggagctgcgagccgctgtgctgcactggcgcgagaccgtcgtgcagcagaaggagacgctgggcgctcagcgagaagccatccgagaactcaccagcaagctggcccgctgtgagggactagccggcggtaaggcgcgcggcacgggggccacgggcaaggacaccatgggcgacctgccgcgggacccgggccacgtcgtggagcagcttagccgctcgctgcagaccctcaaggaccgcttggagagcctcgagctccaactccacaccaacgcgtctaatgccgggctgccgagcgacttccgagaggtgctccagcggaggctgggggagctggagaggcagttgctacgcaaggtggccgagctggaagacgagaagtccctgctccacaatgagacctcggctcaccggcagaagacagagaacacactgaatgcactgctgcagagggtgactgagctggagagaggcaacagtgcattcaagtcaccagatgcattcaaagtgtccctccctctccgtacaaactacctatacggcaagatcaagaagacgttgcccgagctgtatgccttcaccatctgcctgtggctgcggtccagcgcctcgccaggcatcggcacgccattctcctacgctgtgcctgggcaagccaatgagattgtgctgatagagtggggtaacaatcccatagagctgcttatcaacgacaaggtcgcacagctgcccctgtttgtcagcgatggcaagtggcaccatatctgcatcacctggaccactcgagacggcatgtgggaagcattccaggacggggagaagctgggcaccggggagaacctggcaccctggcatcccatcaagccagggggtgtgctcatcctggggcaggagcaggacactgtgggaggcagatttgatgccacacaggccttcgttggagagcttagccagttcaacatatgggaccgtgtcctccgggcacaagagatcatcaacatcgccaactgctccacgaacatgcctggaaacatcatcccatgggtggacaacaatgtcgatgtgtttggaggggcttccaagtggcctgtggagacgtgcgaagagcgtctcctggacttgtagctaccttctccctgtcccagaggccaagagcgggctgttctggggagttcaaggcatctattcccgagttcaactaaaatctctggcctgagtaggaaagaaccagagcccctaaggcaggctgtgtggcctcctttgtcttaggctcctatgttcttactgctttgttctttggtgggaagtgaccgaagccctgggaagagtcctgagccacttcctgctggggtttctagtaaagtctgtgagcctctccacccctcctgtaaatgctagtgcaacccagccctgcctgtcattttggatccttagtgtctcgtgtgtgcttcccgtctgtcccctttgatggctgtgtggtcatcctaccggggtggcctgggtcccttgtgtgtgtagcacatccctgcttttgactgaacacagtgcacagaagctacccgcccctgaaacagggtctctccctcagtgtcatgtgcactctggtctctccctctgaggggactgcagctgctggagggccacgtgcccagacagtccccagcatccccaaagcagaccctccgccatggagaaagtcccccacagcttccccaccctctgtccacctctcagaccccacgcttctaaggaccattgctgggttggctttcaaaagctgctgctctcatctggtgccaaaagttcatttgcagcttctacaccgttctgtgtggtttggggattgactttattcccccacaaaagaggaacagccattagaagccagcctcccctccttttgatgctcagcccactgtgaagagtgagcttgcttgtaagccacattggtttctgtgagcatctgactctcccccgtccagtattttccccggaactggagattcgagtgccattcggctgctacctgcttagtgactccaggctgcatcatgtatcataatttattttaaagacaaagtgattcagtggggaaatttataaagctataaatattatatattttatttttcatacatgtttaaagtgcggatccatggatgttccatttgtaggaccagcttgacgtgcccatcctgacattgtatgccacaagagctcttgtgatgatggaattttgattaaagtgcactggaagatga

SEQ ID NO: 18 - DNA, 2858 bp, Rattus norvegicus
gaattcggcacgagcggaactgtgaagggctcccatcaggctcccgactgacagagagctagaaggcacagagaaacctgaggattctcatttaactctgggaactgcttcaagaagctatagtaccagagaacacctgggaagtgtgagaattcctgcagctgggaccaaaatgtctttcatagatccttatcagcacataatagtggaacaccagtattcccataagtttacagtagtggttctacgtgccaccaaagtaaccaaggggacctttggcgatatgctggacactcctgatccttatgtggaacttttcatctctacaacccctgacagcaggaagcgaacaagacacttcaataatgatataaaccctgtgtggaatgagacctttgagttcattttggatcctaatcaggaaaatgttttggagatcacattgatggatgccaattacgtcatggatgaaaccctaggcacagctacatttcctgtatcttctatgaaggtgggagagaagaaagaagtcccttttattttcaaccaagtcacagaaatgattctggaaatgtctcttgaagtttcgtcatgcccagacctacggttcagcatggcactgtgtgatcaggagaaaacattcaggcagcagaggaaagagaacataaaagagaacatgaagaaacttttgggtccaaaaaagagcgaggggctttattccacacgtgatgtgcctgtggtggccattttgggttccggcgggggtttccgggccatggtgggattctccggtgtgatgaaggcgctctatgaatcagggattttggattgtgcgacctacgttgctggtctgtccggctccacatggtacatgtcaaccttgtactcccaccctgattttccagagaaaggtcctgaggagattaatgaagagctaatgaaaaatgttagccacaaccctctcttacttcttacgccacagaaagttaaaagatatgttgagtctttatggaagaagaaaagttctggccttcctgtcacctttactgacatctttggaatgttaataggagaaacactaattcaaaatagaatagtaccgaccttgagtagcttgaaggaaaaggtcagcgccgcccggtgtcctctgcctctcttcacctgtctccatgtcaaaccggacgtgtcagagctgatgtttgccgattgggtagaatttagtccatacgaaattggcatggcaaaatatggtacctttatgactcctgacctgtttggaagcaaattttttatgggaacagttgtaaaaaaatatgaagaaaaccccttgcatttcttaatgggtgtctggggcagtgccttttctatactgttcaacagagttttgggagtttctggcttacagaataaaggttctacaatggaggaggaattagaaaatattacagcaaagcacattgtgagtaacgacagctctgacagcgatgacgaggcccaaggacccaaaggcaccgagaatgaagatgcggaaagagagtaccaaaatgacaaccaagcaagttgggtccatcggatgctaatggccttggtgagtgactcagctttattcaatacccgagaaggacgtgctgggaaggtgcataacttcatgttgggcttgaatctcaacacatcgtatccactgtctcccctgagagacttcagcccccaagattccttcgatgatgatgaactcgacgcagcggtagcagatccagatgaatttgaacgaatatatgaaccactggatgtcaaaagtaaaaagattcatgttgtagacagtgggctcacgtttaacctgccgtatcccttgattctgcgacctcagagaggtgtggatctcatcatttcctttgacttttctgcaaggccaagtgacaccagccctccattcaaggaacttctgcttgcagagaagtgggctaaaatgaacaagctcccttttccaaagattgatccttacgtgtttgatcgggaaggattgaaggaatgctatgtgtttaaacctaagaatcctgatgtggagaaggattgcacaaccattatccactttgttctggccaacatcaacttcagaaagtacaaggccccaggtgttctgagggaaaccaaggaagagaaagaaatagctgactttgacattttcgatgaccccgaatcgccattttcaaccttcaacttccagtatccaaatcaagcattcaaaaggctacatgatctgatgtacttcaacacactgaacaacattgatgtgataaaggatgccattgttgagagcattgaatacagaagacagaacccatctcgttgctctgtttccctcagtaatgttgaggcaagaaaattcttcaacaaggagttcctaagtaaacccacagcagagtccatttgaattccatgactactggagttcagagcacatgagagatcatcttactatgcacaagagactgactgctactcagagttgtggggacggaggcgtgtgttaggtgaaaacggtgttgattatgcaatacttggcaacagtttctgacagtatgaattttttgacataagcatagggctatatactgtattttaaacattcctcacatttttacctgagcatttttatatatatataaaaatatcctttccttttataaatattaatagtttaactcagtaaaaaaaagcttcccattgtgtgtgaatgttattctgaactagatttgttcatgccatgttacaacactatttttatttaaatgttcatatctacacatgcgaaataaatactttgatatacaaattgccaaaaaaaaaaaaaaaaaaaa

SEQ ID NO: 19 - DNA, 1194 bp, Rattus norvegicus
ttcgccgcgcccgcgcccgcgcaccacgacttcctttccgacctcttcgccgacgactacggcgccaagccgagcaagaagccgtccgactacggttacgtgagcctcggccgcgcgggcgccaaggccgcaccgcccgcctgcttcccgccgccgcctcccgccgcactcaaggccgagccgggcttcgaacccgcggactgcaagcgcgcggacgacgcgcccgccatggcggccggcttcccgttcgccctgcgcgcctacctgggctaccaggcgacgccgagcggcagcagcggcagcctgtccacgtcgtcgtcgtccagcccgcccgggacgccgagccccgccgacgccaaggccgcgcccgccgcctgcttcgcggggccgccggccgcgcccgccaaggccaaggccaagaaggcggtggacaagctgagcgacgagtacaagatgcggcgcgagcgcaacaacatcgcggtgcgcaagagccgcgacaaggccaagatgcgcaacctggagacgcagcacaaggtgctggagctgacggcggagaacgagcggctgcagaagaaggtggagcagctgtcgcgagagctcagcacgctgcggaacttgttcaagcagctgcccgagccgctgctggcctcggcgggtcactgctagcccggcgggggtggcgtgggggcgccgcggccaccctgggcaccgtgcgccctgccccgcgcgctccgtccccgcgcgcgcccggggcaccgtgcgtgcaccgcgcgcacctgcacctgcaccgaggggacaccgtgggcaccgcgcgcacgcacctgcaccgcgcaccgggtttcgggacttgatgcaatccggatcaaacgtggctgagcgcgtgtggacacgggactgacgcaacacacgtgtaactgtcagccgggccctgagtaatcacttaaagatgttcctgcggggttgttgctgttgatgtttttgtttttgttttttgttttttgttttttttttggtcttattatttttttgtattatataaaaaagttctatttctatgagaaaagaggcgtatgtatattttgagaaccttttccgtttcgagcattaaagtgaagacattttaataaacttttttggagaatgtttaaaaaaaaaaaaaaaaaaaaaaaaaaaaaaaaaaaaaaaaaaaaaaaa

SEQ ID NO: 20 - DNA, 1227 bp, Rattus norvegicus
ggaattcgcaacgcctacacaaagatccctatcactgagcacccggaggcaaggctcagaaccccaggatcagcaacatggagttctcccgtccaccgctagtgcatgtgaagggtatcccactcatcaaatactttgcagagacaattgggccattgcagaacttcacagcctggcctgatgacttgctgatcagcacatacccaaagtctggtactacctggatgagtgagatcctggatatgatctatcagggtggcaagctagagaagtgtggccgcgcccccatctatgcccgggtacccttccttgagttcaaatgtccaggggttccctcaggtcttgaaactttggaagagacaccagccccacggctccttaagacacatctgcccctgtccttgctccctcagagtctgctggatcagaaggtcaaggtgatctacattgcccgaaatgcaaaggatgtggttgtctcctattataacttctacaacatggccaagctgcaccctgatccaggcacctgggacagcttcttggagaacttcatggatggggaagtgtcctatgggtcgtggtaccagcacgtgaaggagtggtgggagctgagacacactcaccctgttctctatctcttctatgaagacataaaggagaaccccaaaagggagatcaagaagattctagagtttttggggcgctctctacccgaggagactgtggattccattgttcaccacacatctttcaagaaaatgaaagagaactgcatgactaactacacaaccatccccactgagattatggaccacaatgtttctcccttcatgaggaaaggtactactggggactggaaaaataccttcactgtagcccagaatgagcgctttgatgcccactatgctaagacaatgacagattgtgacttcaagtttcgttgtgaactatgagtggattatggctatactgggaaccaaggcaaactgacacagcccatcatgatctcaagtaaaatgtgatgtgttcaatctacttgttgtatgcctagaggaaatctgagctaagagaataggattggggatgtggctgaggcagagggttttatcaacgcatgtcaggaaagcaatcagtcccaacacctaaaaagaacctaaagtacaaacatgcaaaaaatagtaagataaactatattttacctgaaagaataaatgccactgggaaatg

SEQ ID NO: 21 - DNA, 2507 bp, Rattus norvegicus
gagcgagagcgctgttgctgacccagctgagcccagctcctaggacgccagccctcgaccatctttcatactccagccacggaacggagccagggcagacgggtccggattttccccctgccccgaccctcctctccacctcccgccgtcgtgacaccggctgtctctggcagcccgttggtcatgaaaaccttcacgttgccagcatccgtcctcttctgcttccttctactcatccgggggttgggagcagcaccccccgggcgctccgatgtttatcctcctcccctcggctctgagcataatgggcaggtagctgaggacgcagtgtcccggccaaaggatgacagcgtcccagaggtccgagcggctcggaattccgagcctcaggaccagggagagctcttccagggcgtggatccccgggcgctggccgcggtactgttgcaggcactggaccgtccggcctcgcccccggctgtcccggcaggttcccagcagggaacacccgaagaagcagcagaagctctgctgaccgagtccgtgcgcagtcagacccatagcctcccggcatcagaaatccaagcgtccgctgtggcgccccctcgccctcagactcaggacaacgatcccgaggcagacgaccgctcagaagagctggaggcactagcatccttgctccaagaacttcgagatttcagtccgagtaatgctaagcgccagcaagagacggcggcagcagagactgaaacccgcacgcacacgctgacccgagtcaatctggagagccccgggccagagcgcgtatggcgcgcttcctggggagagttccaggcgcgcgtcccggagcgtgctcctctgccgccctcggtcccttctcaattccaggctcgaatgtccgaaaacgttccccttcccgaaacccatcagttcggggaaggagtgtcctcccctaaaacacatcttggtgagactttgacacccttatccaaggcgtaccaaagtctaagtgcccccttccccaaggtgcgtcggctcgagggctcattcctgggcggttccgaggcaggagagcgcctgcttcaacaagggttagctcaggtagaggcagggaggaggcaggcggaggccacccggcaggccgcagcgcaagaagagcggctggccgatctcgcctccgacctgctgctccagtatttgctgcagggcggcgcccggcagcgcgatctcgggggtcgcgggctgcaggagacgcagcaagagcgggagaacgagagggaggaggaggcggagcaggagagacgcggtggtggggaggacgaggtgggggaagaggatgaggaggcggcagaggcggaggcggaggcagaggaggcggagagggcgcggcagaacgcgctcctgttcgccgaggaggaggacggggaagccggagccgaggacaagcgctcccaggaggaggcgccaggccatcggcggaaggatgctgaggggacagaggagggcggggaggaggatgacgacgacgaagagatggatccgcagacgatcgatagtctcattgaactgtccaccaaactccacctgccagcagacgatgtggtcagcatcatcgaagaggtggaggagaaacggaagcggaagaagaacgcccctcccgagccggtgccaccccccagggctgccccagccccgacccatgtccgctccccgcagcccccacctcccgccccggcccgggatgagttgccggactggaacgaagtactcccaccctgggatcgggaggaggatgaggtgtttcccccggggccctatcaccccttcccaaactacattcggccgcggacactgcagccgcccgcatcctcccgccgccgtcacttccatcacgcgttgccacctgcgcgccaccatcccgatctggaggcccaggccaggcgcgcgcaggaggaagcggacgcggaggagcgccggctgcaggagcaggaggagctggagaattacattgagcacgtgctgctgcaccgcccgtgacccgcccctgcgcgcccgctcccaactgcgcgcgccgccacgcccccctccgtgtcgctcctcctccctctcggtgtttgcatgcgccccggctccgcccctcggctgccgcccggccccgccccacaaggccccgccccgggttctgtcaggaccagacctgtcagacttctttggggtctgatcctggggccagcccaggcgggtgtgtggtttgtgcgagtccccttacacccccacttcctccaggggcctcgtccccatctagtttctctagcgacttcctggtcccaaacggggaaaagctgttctatttaatcgtgtgaagtgtctgtctcccagccttggggcccccggagcctcccttctccaaattgctgtgaacttacccacatcttgcccttctgttgtaaatacccctcacggaggaaatagttttgctaagaaataaaagtgactattttattagg

SEQ ID NO: 22 - DNA, 2175 bp, Rattus norvegicus
  misc_feature (1)..(2175): where n may be a or g or c or t/u, unknown, or other
atgagcgcagaggggtaccagtacagagcgctgtatgactacaaaaaggaacgagaggaagacatcgacctacacttgggggacatcctgactgtgaataaaggctccttagtggcacttggattcagtgatggccaggaagcccggccagaagatattggctggttaaatggttacaatgaaactactggggagaggggagactttccaggaacttatgttgagtacattggaaggaaaagaatttcaccccccactcctaagccacggccccctcgacctcttcctgtagcacccggttcttcaaaaactgaagcagacactgagcaaccagtgttgacccttcctgatctggccgagcagtttgcccctcctgatgttgccccgcctctccttataaagctcctggaagccattgagaagaaaggactggaatgttcaactctatacagaacacagagctccagcaaccctgcagagttacgacagcttcttgattgtgatcccccctcagtggacttggatgtgttcgatgaacacgttttagcagatgctttcaaacgctatctcgccgacttaccaaatcctgtcattccagtagctgtttacaatgagatgatgtctttagcccaagaagtaccaagctccgaagactacatccagctgttgaagaagctcattaggtcgcctaatatacctcatcagtattggcttacgctccagtatttgctcaagcacttcttcaagctctctcaagcctccagcaagaaccttctgaatgcaagagccctctctgaaattttcagccacgtgcttttcagattcccagcagccagctctgataatactgaacacctcataaaagcggtagagcttttaatctcagcggagtggagtgagcgacagccagcaccagcactgccccctaaaccacccaagcccacttctatagccaacaacagcatgaacaacaatatgtccttacaggatgctgaatggtactggggagatatctcaagggaagaagtaaatgaaaaactccgagacactgctgatgggacctttctggtacgagatgcatccactaaaatgcacggngactacactctcacactaaggaaaggaggaaataacaaattaatcaaaatctttcaccgagatgggaaatacggtttctctgatccattaaccttcaactctgtggttgaattaataaaccactaccggaatgagtccttagctcagtacaatcccaagctggatgtgaagttactctacccagtgtctaaataccagcaggatcaagttgtcaaagaagataatattgaagctgtggggaaaaaattacatgaatataatactcaatttcaagaaaaaagtcgggaatatgatagattatatgaggaatacactcgtacttcccaggaaatacagatgaaaagaacagctatcgaagcatttaatgacaccataaaaatattcgaagagcagtgccacccccaggagcggtacagcaaagactacatagagaagtttaaacgcgaaggcaacgagaaggaaattcaaaggataatgcataatcacgataagctgaagtctcggatcagcgagatcatcgacagcaggaggagactggaggaagacttgaagaagcaggcagccgagtatcgcgagatcgacaagcgcatgaatagcattaagccggacctcattcagctgagaaagacaagagatcaatacttgatgtggctgacgcagaaaggggtccggcagaagaagctgaacgaatggttgggaaacgaaaacacagaagaccaatactcactggtagacgatgacgaggatttgccccaccatgatgagaagacgtggaatgtggggagcagcaaccgaaacaaagccgagaacctattgcgagggaaacgagatggcactttcctggtccgggagagcagtaagcagggctgctatgcctgctctgtagtggtagatggcgaagtcaaacattgcgtcatcaacaagactgccaccggctatggcttcgccgagccctacaacctgtacagctccctgaaagagctggtgctacattaccaacacacctccctggtgcagcacaatgactccctcaatgtcacactagcatacccagtatatgcacaacagaggcgatga

SEQ ID NO: 23 - DNA, 2116 bp, Rattus rattus
cgcaactgagaagactggatagagccggcggagccgcgaacgagcagtgaccgcgctcccacccagctctgctctgcagctcccaccagtgtctacccctggacccctcgccgagctttgcccaaaccacgaccatgatgttctcgggtttcaacgcggactacgaggcgtcatcctcccgctgcagtagcgcctccccggccggggacagcctttcctactaccattccccagccgactccttctccagcatgggctcccctgtcaacacacaggacttttgcgcagatctgtccgtctctagtgccaactttatccccacggtgacagccatctccaccagcccagacctgcagtggctggtgcagcccactctggtctcctccgtggccccatcgcagaccagagcgccccatccttacggactccccaccccgtcgaccggggcttacgccagagcgggagtggtgaagaccatgtcaggcggcagagcgcagagcatcggcagaaggggcaaagtagagcagctatctcctgaagaggaagagaaacggagaatccgaagggaaaggaataagatggctgcagccaagtgccggaatcggaggagggagctgacagatacgctccaagcggagacagatcaacttgaagacgagaagtctgcgttgcagaccgagattgccaatctactgaaagagaaggaaaaactggagtttattttggcagcccaccgacctgcctgcaagatccccaatgacctgggcttcccagaggagatgtctgtgacctccctggacttgactgggggtctgcctgaggctaccaccccagagtctgaggaggccttcaccctgcctcttctcaatgaccctgagcccaagccatccttggagccggtcaagaacattagcaacatggagctgaaggctgaaccctttgatgacttcttgtttccggcatcatctaggcccagtggctcggagactgcccgctctgtgccagatgtggacctgtctggttccttctatgcagcagactgggagcctctgcacagcagttccctggggatggggcccatggtcacagagctggagcccctgtgcactcccgttgtcacctgcactcccagctgcactacctatacgtcttcctttgtcttcacctaccccgaggctgactccttccctagctgcgcagctgcccaccgaaagggcagcagcagcaacgagccctcctctgactcactgagctcgcccacactgctagccctgtgagcagtcagagaaggcagggcagccggcactgactgagctggtgcattacagagaggagaaacacgtcttccctcgaggggttcccgtagacctagggaggaccttatctgtgcgtgaaacacaccaagctgtggacctcaaggacttgaaagcatccacatctggactccagtcctcacctcttccggagatgtagcaaaaaaacaaaaaaacaaaacaaaaaaaaaacaaaacaaaaaatcaaaagcaaccgcatggagtgtattgtttgtagtgacacctgagagctggtagttagtagcatgtgagccaggcctgggtctgtgtctcttttctctttctccttagtcttctcatagcattaactaatctgttgggttcattattggaattaacctggtgctggatatttttcggattgtatctagtgcagctgattttaacaatacctactgtgttcctggcaatagtgtgttccaatttagaaatgaccaatattaaactaagaaaagatagaactttattttccggtagatagaaataaatcgctatatccacgtactgtagctcttcagcgtccatgttcattgtcatgtaactgatcatgcattgttgaggtggtctgaatgttctgacattaacagttttccatgaaaacgttttattgtgttttcaatttatttattaagatggattctcagatatttatatttttattttatttttttctatcctgaggtctttcgacatgtggaaagtgaatttgaatgaaaaaattttaagcattgtttgcttattgttccaagacattgtcaataaaagcatttaagttgaatgcg

SEQ ID NO: 24 - DNA, 617 bp, Rattus sp.
gatataaataagtttattgctggatttcctcattaacattatagaaatattttaaaatcactaaaagtcacaaattgagagcccaacaggtaaccatactttataaaaaagaaagtataaaagcggcttataaaattttctgaaaagtatatcagctgacagcaaagaaataggaaccagtcagtaaatggcacaaatacatcactgaagttctcagtcgtcacctacaagtcagtgtctgagctatgaagtagacctgttttataagttacacactgagtagtgttccgaactgtccttcctcacttccataagagaagccaaatcttttactgctatggggacaaagtactatccataaactggagaagaaggctcaccttcctctcccagtacctgaacaacagaacagtgtgcacagaaacggctttggcacttgaaccctcagacactgtcttaaacgttgttgcttgaatattgtagttttacctggtgctctgacctacattgttgtctccatgctctgtggatatcacacacacacacacacacacacacacacacagatataaacagccaaggcactcatacacataaaagtaacaataataattttt

SEQ ID NO: 25 - DNA, 7204 bp, Rattus norvegicus
cgcaaagttgggcagggagctgggggaggaggaccgcgagcgagggtggcggagcaggggcgggaggagcggagggaggaggggaccggagcgtgtcactcgcgcgctccctctgtgcacagaggatgtgctgaatggtgcgcttccaggcggcggccgagcaggatcaggcggcgggcggctcgcactgccgggctctgctccttcttgtctcccgaggctctacaatcaccgcgggctccagaccctgcgtcccgcccggggcatggcaggctgctgtgccttgcggtgtagtcccgcttactaagcggcgcgggccagaggtgcggaggccaagaggccgggaggtcggcgggcagcggaggcaagagcctgctgaaccgagagccaagcccgcttgcgcccggagctccgtgtcccgtctccactgcgctcgcccgggccgcccggagccccgatgagcccagatggctggggctcagcccggagtgcacgccttgcaactcaagcccgtgtgcgtgtccgacagcctcaagaagggcaccaaattcgtcaagtgggatgatgactccactatagttactccaattattttgaggaccgatcctcagggatttttcttttactggacagatcagaataaggagacggagctgttagatctcagcctcgtcaaggatgccaggtgtgggaagcacgccaaagctcccaaggaccccaagttacgtgaacttctggatgttgggaacatcggacacttggaacagcgcatgataactgtggtgtatgggccagacctggtgaatatctcccacctgaatcttgtggcttttcaagaggaagtggccaaggaatggacaaatgaggttttcagtttggcaacaaacctgctggctcagaacatgtccagggacgcatttctggagaaagcatatactaagctcaagcttcaggtgaccccagaagggcgcattcctcttaaaaacatctatcgactgttttcggcagaccggaagcgagtggaaactgcgctagaggcttgtagtcttccatcgtcaaggaacgattccatccctcaagaggactttactccagatgtatacagagttttcctgaacaatctttgtccccgacctgaaattgataacatcttctctgaatttggtgccaaaagcaaaccgtaccttactgttgatcagatgatggattttatcaaccttaagcagagagatccccggctgaatgaaatactttacccacctctgaagcaagagcaggtccaagtgttgattgagaagtacgagcccaacagcagcctcgccaagaaagggcagatgtcagtggatggattcatgcgctacctgagcggagaagaaaatggagtcgtttcacctgagaaactggatttgaacgaagacatgtctcagcccctgtctcactatttcatcaattcctcacacaacacctacctcacagctggccagttggctgggaactcgtctgtagagatgtatcgccaggtgcttctgtctggatgtcgctgtgtggagctggactgctggaagggcaggaccgctgaggaagagcctgtcatcacccatggattcaccatgacaacagaaatatccttcaaggaagtcatagaagccatcgcagagtgtgcgttcaagacttctccttttcccatcctcctttcctttgagaaccatgtggattccccgaagcaacaagccaagatggccgagtattgccgattaatctttggtgatgccctccttatggagccactggaaaaatacccactggaatctggggtacctcttccaagccctatggatttaatgtataaaatcttggtgaaaaacaagaagaagtcacacaagtcgtcagagggaagtggtaaaaagaagctctctgagcaagcttccaacacgtacagcgactcttccagcgtgttcgagccttcgtctccgggagctggggaagcagatacggagagtgatgacgatgacgacgatgatgactgtaaaaagtcttccatggatgaggggactgctggcagcgaggccatggccacagaagagatgtctaacctggtgaactatattcagcctgtcaagtttgagtcctttgaaacttcaaaaaaaagaaataaaagctttgaaatgtcttccttcgtggaaaccaaaggactcgaacaactcacgaagtctccagttgaatttgtcgaatacaacaagatgcagcttagcaggatatatcccaaaggaacacgcgtggactcatccaactacatgcctcagctcttctggaatgctggctgtcagatggtggcgctcaacttccagacagtggatctagctatgcagataaacatgggcatgtacgaatacaatgggaagagtggctacaggctgaagccagagttcatgaggaggccagacaagcattttgatccatttactgaaggaatcgtagatgggatagtggccaacactttatctgttaagattatttcaggtcagtttctctctgataagaaagttgggacttatgtggaagtggatatgtttggtttgcctgtggacacaagaaggaaggcatttaaaaccaagacatcccaaggaaatgctgtaaatcctgtctgggaagaagagccaattgtattcaaaaaggtagttctgccttctctggcctgtttaaggatagcagcatatgaggaaggaggcaaatttattggccaccggatcttgcctgtgcaagcaattcggccaggctatcactacatctgcctgcggaatgagaggaaccagcccctgatgctgccagctgtctttgtctacatagaagtcaaagactatgtcccagacacgtatgcagatgtaattgaagcattatcaaacccaatccgatatgtcaatctgatggaacagagagctaagcagttggctgcattgacactggaggatgaagaggaagtcaagaaggaggctgaccccggggaaacgtcatccgaggctccaagtgaaaccaggacaactccagcagagaatggggtgaatcacaccgcaacccttgcacccaagccaccttcccaggctccacacagccagcctgctccagggtctgtgaaggcacccgccaaaacagaggatctgattcagagcgtgttaacagaagtagaggcgcagaccattgaagagctcaagcaacagaaatcgttcgtgaaacttcaaaagaagcactacaaagaaatgaaagacctggtcaagagacaccacaagaaaaccaccgagctcatcaaggagcataccaccaagtacaatgagattcagaatgactacttgagaaggagggcagccttggaaaagtccgccaaaaaggatagcaagaagaaatctgaacccagcagcccagatcatggctcatccgccattgagcaagacctcgcggccctggatgcagaaatgactcagaagttgatagacttgaaagacaagcaacaacagcagctgcttaatcttcggcaagagcagtattacagtgagaagtaccagaagcgggagcacattaaattgctcattcagaagttgacagatgttgctgaagagtgtcagaacaatcagttgaagaagctgaaggaaatctgcgagaaagagaagaaggaattaaagaagaaaatggataagaagaggcaggagaagataacagaagccaagtccaaagacaaaagccagatggaagaggagaagacagagatgatccgatcatacatccaggaggtggtgcagtacatcaagaggttagaggaagcacaaagtaaaagacaagaaaaacttgtggaaaaacacaaggagatccgccagcagatcctggatgagaagcccaagggggaaggctcctcctcagtcttgtcggaaagttgccatgaggatccctctgttccccccaactttactccccccaaccctcaagctctcaagtggtgagcaccgtccttctagccagctgcagatggagctggagcaagaataccaagacaagttcaaaagactgcccctggagattctggagtttgtacaggaagccatgaaagggaaggttagtgaggacagcaatcacggctctgcccctccctcgctggcctcagaccctgctaaggtgaacctcaagtctccctccagtgaggaggtacaaggagagaacgcgggaagagagtttgatactcctctgtgatgtccctgccgggcctaccagacatgcacggctgcttgaactccatcggactctaaagacaaagatcactgcccgggccatcttcctgagaaacatcccttagcctgaaatccacaccaaagggagagttccagaaggatccgtgtgaaggtcccatacccttgtcccttgtgtcatgtggaaactattgtggtcttagagagaagggtgcatgtatgcaggatttttcttttcgttccaatagtaaattaaaagcaggcagctccaggctccatggactatgtaatgaaggacaatgtcttctttgaagaaaactagagctcgtgtcttcgtttgaagccctggtgtacagtatttccaagtaaaagagagagtttgagaagtgcgcggcaccatttaacactctggaacattccactctgagcattgtttccctgactgccccgcaaacccatgttttcaagttaacgtgtatagtgcattgtttcaccctttgctttgcaggcactggtggcttgccatttgctaatttatttatcccagaggcatctgtatttgctattgacatggctttattagataccatagtgactcatataagctggtttcttttataaaaaaaaatcacatgactgtatcatttcccagtgaagccattttacgattagcaatatgggttgtatatttggctgcacgtctttggatatctatcggttgttgacctgaggctatcaaacagctgcaatgggtttgttctgttgagtcagggtttccgccagactgaacaggatccagtgggctattttattgtcagatattttttcgtggttcatttatttttactgtagaaaggaagatagaatatttatctaaatgcacatgtataaatgatatacattatctccatgtatatagtacatgtatgcacccagccatataagtgcacacacatacatacgcatgagtgtacatgtgtgtttattaattggcagtgacccaaatctcttccataagatttaaaagcaagttcagggatacatggacagagaagaaatgggtcgaatatatgttatgtggataattaaattatacggaaatgctaagaatcagtttatggagtcaatgatgcaacttgaggtaatcttagaatttagaaaatgagtttactaaaaaaattggaggatcaaaaattggattttaagtatcagattttgagcttgttttaatggtcagtgtatgcaaaaatgcataggaaatagagtgcaaatgtattactactatcttcaataaattttaactgaataatttcaattttatactgatagaaataaagctctggttgtattttagtttgtatatttacttcacttagttatgtcttttttaattcttattttattctaaagtgaataagagaaacaaattatatcatgctaacgctaataaaatcataattaaaaggtcttaagaaataatattatatagaaaaaatatggtttagagagtgggacaaagaacattaaaacccagcaatgacaatgcccatggcttcattttattttaaagtgaacctgtctgcttataatttagcaaagcaaaaaacagttgtgtcctgaagtttgtgtttcaagaatttagtatttttctggaaataattttatttaactttaagcaataactaggaccataattaagtgttaatcaaaatgaaggcttgtttcaaacaccatgaaagtgtttgatcacacacatacaccacacacacacacacacacacacacacacacacacacacacacacacacctattgagacacaaggggaaaaaaatcacatccccttcagagatgattgtattttatctgaaggatcaagagtgttaattagtcacttactaaagcttaccttccactgaagagagatctcttgtgaaccatacaacttttgcactttgaaggaaaggcattactcagaaggaagtgaggacagtccaaatggaatgctgcattttacacacacagttacagactgttgaaaattggggaagagtgaatggataacaggattcgaaaccaaacttagtggtggttgttgcggaacattggatttcaacatactccatacagcgactgaggttgaataaccttgactttcaaatcctgttttggtagttggacttcattatcttggtgattctagtcattttacaatgttttgtatttggtcatttactgtaatcacatttttatatctgtacagtgacactttttgcagttgtggggtagtgtgtaacactgtgcatcttgcatcatcgaaactactaccgtgatactatccattgataatattaatattacttgaaaaatgacaaaggtaaagaaaaggggtctgtatgatgtgcagttttgtgcctttatgtatttgccttgttcttcgttgaatgtgtgaaattctgtactgtggtttttccaatagtagaaagtagagtcatgcattaaattagactgtatccctgacacctttaaactactgagaataacgtggttggccgtgtaattcagtgttcaaagttctaatgacatgccatgtgctttggtttttaacatttcatgaccaccatacattaattaatactcctgtaatagataagcagtcattaattaagttccaaaagaaagggccattgcttgcattcctttgaatttaatgttgcccttgtacactgtgttaatactgtatgtaatggattgaacattgtgattctcgccttttaagaagagaaagagagagaaggaaaagtatttgatgctcttaaaatgtacatatttgggttcttctatctcaaattatttaaaatgcataattcacatttttttgtaatcattctatgcaattttgtggcatgacgtttcttccacttgtaattttctgtgctttcatcacaagtccaaaggaaacaataaaaatt

SEQ ID NO: 26 - DNA, 3700 bp, Rattus norvegicus
ccaagaggaagaaacatgaagtttttgctgcttagtgcacttttatttttgcatagttccttagcttggacaagagaaaagcattattacatcggaattactgaagcagtttgggactatgcttctggcagtgaagaaaaggaacttatttcagttgacacggaacagtccaatttctatcttcgaaatggtccagatcgtattggaagaaagtataagaaggccctttattctgagtacacagatggcacctttacgaagactatagacaaaccagcctggctagggtttttaggccctgtcatcaaagctgaagttggagacaaagtttctgttcacgtaaagaactttgcctctaggccctacacttttcatgctcatggggtaacttacaccaaggcgaacgagggggccatctaccctgacaacaccactgattttcaaagagccgatgacaaactgtttcctggacagcagtatttgtacgtgctgcgtgccaatgagccaagtcctggcgagggagacagcaattgtgtgaccaggatttaccactctcatgtggatgctccaaaagatattgcatcaggactcataggaccgttgatactctgtaaaaaaggttctctgcataaggaaaaagaggaaaatattgaccaagaattcgtactgatgttctctgtggtggatgaaaatctcagctggtacctagaagataacatcaaaaccttctgctctgaaccagagaaagtcgataaagacaatgaagacttccaggaaagcaacaggatgtactctataaatggatatacatttggaagcctcccagggctctcgatgtgtgcagaagacagagtgaagtggtacctttttgggatggggaatgaagttgacgtgcattcagagctctttcatggtcaagccctgaccagcaagaactatcatactgatataatcaacctgttccctgccactctaattgatgtttctatggtggcccagaatcctggagtctggatgctcagttgccagaacctgaaccatctgaaagctggtttgcaggcctttttccaggttcgtgactgcaacaagccctcaccggacgacgatatccaagacagacatgtgagacattattacatcgctgccgaggagaccatttgggactatgctccgtctgggacagacaccttcactggagagaacttcaccagtctgggaagtgattcaagggtcttttttgagcaaggtgctacaagaattggtggctcttataaaaaattggtttatcgtgagtacacagatgattccttcacaaaccggaaggaaagaggccctgatgaggaacatcttggaatccttggtcctgtcatttgggcagaagtaggagacatcattagagtcacctttcataacaaaggacaatttcctctcagcattcagccaatgggggtaagattcaccaaggaaaatgagggaacatactatggcccagatggccgttcctcaaagcaagcctcccatgtggctcccaaagaaacctttacgtatgaatggactgtccccaaagaaatgggacccacttatgcagatcctgtgtgcctatctaagatgtattattctggagttgacctcaccaaagatatatttactgggcttattgggccaatgaaaatatgcaagaaaggcagcttacttgcagatgggagacagaaagatgtagacaaggagttctacttgtttgcaacagtgtttgatgagaatgagagtttactcttggatgataatatcagaatgttcacaactgcacctgagaatgtggacaaggaagatgaagactttcaggagtccaacaagatgcactccatgaatggattcatgtatggcaatctgcctggcctcaatatgtgcctaggagaatccatcgtgtggtatttgttcagcgctggaaatgaggcagacgtgcatgggatatacttttcaggaaatacctatctgtccaaaggagaaagaagagacactgcaaatctgtttcctcataaaagtctcacccttctcatgacacctgacacagaagggtcttttgatgttgagtgtcttacaacagatcactacaccggcggcatgaagcaaaagtacactgtgaaccagtgcaaggggcagtttgaagatgtcactctctaccagggagaaaggacctactatattgcagcagtggaggtggaatgggattattcaccaagcagggactgggaaatggagctgcaccatttgcaagagcaaaatgtttcaaatgcatttttggataaggaagagtttttcataggctcaaagtacaagaaggttgtgtatcgagagtttactgacagcacattcagagaacaggtgaagagaagagctgaagaggagcacttgggcatgctcggtccactgattcatgcagatgttggagccaaagttaaagttgtctttaaaaatatggcaacaaggccatattcaatacatgcccacggagtgaaaacaaagagttctacagttgctccaacgttgccaggtgaagttcgcacttatatatggcaaattccagaaagatcaggtgctggaacggaggattcaccttgtatcccatgggcttattactcaaccgtggatcgagttaaggatctctacagtgggctaataggcccattgattgtttgtcggaaatcttatgtgaaagtattcaatcctaaaaagaaaatggagttttcccttttgtttctagtttttgatgagaatgaatcttggtacttagatgataacatcaatacataccccgatcaccctgagaaagataacaaagacaacgaggaattcatagaaagcaataaaatgcatgctatcaatgggaaaatgttcggaaacctccaaggtctcacgatgcacgtgggagatgaggtcaactggtatgtgatggctatgggcaatgaaatagacctgcacactgtacacttccacggccacagcttccaatacaagcacaggggaattcatagttctgatgtctttgactttttccctggaacataccaaaccctagaaatgtttccccaaacgcctggaacctggttactccactgccatgtgactgaccatattcatgcggggatggtaactacctacactgttttaccaaatcaagagactaagtctggctgaaagaaataaattggtgataagtggaatacgagcacaatgacgttgttttaaacatttaaaaaaatcaaagccacacaaatgttcatttgtgagggaattggtaatgccgatggacagatgaacagactgtatcatgacatgtatttgtttgctgggtaacagaatcgctttacatagtccacttacacctgcactgaaaggactctgaaaagtggaaaaaaataagcaaaaccgtatgatcagatgctgtccttgactgtcctcacaggatcactataaagtccactaaactgtctccaactcttctcatcaagtcctctaacaaaccatggggtaagagggtatagaaaagaaggaaagatgaagataccaagatgcactttgtaaaaatctgaaaaacagttgaaggatgctctcggaaaatagagaaagtcaggatccaattatgttacattttgaaaaaatgaaatggagataataaagtaataaattttaaaatgccaat

SEQ ID NO: 27 - DNA, 4011 bp, Rattus norvegicus
ggatccagcatgtcttggctctgtgggcatctacactcgcatgcacatatccacacagagacataaaatttacaataataaaaataaatctaggggttggggatttagctcagcggtagagagcttgcctagcaagcgcaaggccctgggttcggtccccagatccaaaaaaaaagaaccaaaaaaaaaaaaaatcttaaaatgctctttcaggctgccataatggcctttaatcccagaagcatccggtcccctgcaggtggcagaacagggactgccacttctttggaggacaatttgtcagctgtctgtcaaaacttgaatatcctgtagcttagacttacatgtgtgagaatttatcccggatgagttcatattcatagttaagggacagcaacaccaacatttttttaaaaagcagaacgttgtgttcgtaaataggaaccaatccgaggctaattcagggtcttcctgagaactattcagccttagaaagtaacaaactaggtctctgtcttcagacacgatgcaatctttcagctaaagtaaaaagaaaatttccagaacagtttgatgtatggacaaaaatattttcccaaaaactacttaagaaatgtcaacatggctacctcaagaaaaaaaaatgagaaacctttaattttcagaatgtactttcagcttggcatggtgacacccacctttactctcagggctcaggaggcagagacagaggctggtggatctctataagtacagggccatcctggtctacactgtgagttccaggtcatccagaactccatgtgagaccatgtctcaaaaaagaaaggaatgctcttgtatactagttgattccatccacctccattatcaaatctttcattatgacgattttttgtttttccaaagcacagagtaaaatgttaaaacaggaacacacacacacacacacccctaaataaaggtgatctttaaggacctgtttatctcagacccccaatgcaatcacatctagccttttctgtggatggtttcaaactcagatcctcatgcaagtcaatcctgcttgcacctcatgtgctcttaaccacttcagggttcctaagtgtgccggggacttcatgttatctggttatgtctctccttagcaagcgcacaggtgaagaaactggtgttaggtggaaacgaacgttttaaatgggaaacagaggactcgaactagtgattctacggactggagggcgccgcgcatcccgggctctcacatggccctcagcccctgcggggcgcacccgtggcccgacgcgcacgaggctccacgctctggggactcgcgcgcacgtagcggcgcgagcccggcggcggcagcgacgtcacaggccgagctttccttttcgggagtccccggcatacatcctgtgtccatgtttggtcatttacgtcacggcgccagggccggggcctcccgaaatggcagtagcccggggagtcggaagcccggagccagcgccgccgcagctatataagtgggggaactgtaggttgggggagcccggttgcactttggagaaaccaggagccgccgcccgaggcaggtgcgggcgagccagggcgccgcggccccccaaccctcgtccccgagccaagcgccgggagcctggagctggggcgcccggattccctgccagccacgcgcgccccagcccagcgaggccccgggcgccccgccgccaccacgccatgctccacctgagcgacttctccggccccgacgcgctcctctccaagcccaccgaaggctgcgcccacaccagccccgagctgccccggctgcctgctagggacgctccctcggccgccgcgtatcctggaggtaaggagcggggctaggagctcgaagaagccgaatgcgacgaggggtgatgggagaggcgtgcgggggtggggtgggggtccccaagcgcgcggtaccattcccactaaggccacaggcgtctgtgtgggagcccgtgcgtatgcgcgccctggggcgtccgtgggggtcccctgcgctgcaggatccgaagtgcacgaggcgctctgcgggtggcttgcgccaggagggctcgggtcgagggactcgcggctgcaggctccgaggcacgcccttggatgtgtgcggggctgatgcgtggtccctcctttggcaggcgacttcttgagctgggctctgagcacctgcggcgccgggggggacttaacagactcctgcttcctggagggccctgcacccacgcccccttcgggcctcagctacagcggcagcttcttcatccaggcggttcccgaacacccgcacgacccggaggccctcttcaacctcatgtctggcatcttgggcttggcacccttccctagccccgaggcggcagcgtctcggtcccccctggatgtccctttccccgcgggtcccgatgccttgttgccggacctttactccccggatctgagttcggccgccttcccggaggcgttttgggaggccgcgccttcggcgggcgctccctcgcagtgcctgttcgagccccagctctccccgcccgacgtcaagcccgggctgagggcgcctcccgcttcgccagcgctggacgctgctgcttcggccttcaaagggccctacgccccctgggagctgctgtcggccggggctccggggaactgtgggtcgcagggaagcttccagaccaccccggaggcacgcttttccgccgtggggaccaaggtcgaggacctgctgtccatcagctgccccgccgagctgcccggtccggctagcagactctacccgccaggggcctacgatgccttctcgctggccccaggtgacttaggggaggggaccgagggcctcccggcgctgctgacccctccgggcggggagggagggagcggcggcgaaggcggagagttcctggccgtccctcaagcgcagctgtccccgctgggcctgcgcggcgccgccacggcagacttctccaaagccctggtggccgacctcccggggggcagcggagtggcggcgccttcatcccccgccacctccttccccgcggccaaagcccggcgcaagggacgccggggcggcaagtgcagcgcgcgctgcttctgcccgcggccgcacgtcaaggccttcgcctgccccgtggagagctgcgtgcggacgttcgcgcgctccgacgagctcaaccgccacctgcgcatccacacgggccacaagcccttccagtgccgcatctgcctgcgcaacttcagccgcagcgaccacctcaccacgcacgtgcgcacccacaccggcgagaagcccttcgcctgcgacgtgtgcggccgccgcttcgcgcgcagcgacgagaagaagcgacacagcaaggtgcacctcaagcagaaggcgcgcgccgaggagcgcctcaagggcctgggcttctactcgctgggcctctctttcgccgcgctgtagccggagctggctccgtaggtcggcgccgccggccgtcggcgcacgcgacacggtcctgccgctccctcgtccctgctgccttccctgcctcttccacgcacgtccggggccacccgcagcccagctccagttcccccgaagcgcccgccgctcacgcccttcagcacgggctccgcggacagcgcccgctgttttcggagccgccttcctctagccacccgctctggggactgtcctctcggtccacccacagagcaggcgataccttaggactgaagagagtttttgtaactggcgtacgccccacgccttcctctttatcccttcccagagtcaagctggggatgtaccgagccggtctctcaagaactttgtacagcaagtccagcaagcctttggatgtgatgtctttgctttggggttatttcctttttgttgtcgttcattttttgtaaagcagacgctactctcaagcatttgacaaaactgtttatttttgcaattaaaattattgtgct

SEQ ID NO: 28 - DNA, 256 bp, Rattus norvegicus
ctgcccttgactcctagactccaggatgccgggaccttggttgctgctggctctggctttgatcttcaccctaactggtatccctgaatcctgcgccttgccggaggcagcccaggaggaaggtgcagtgactcctgaccttcctggcctggagaatgttcaggtccggccagaacgtcgattcttgtggaaagacctccagcgggtgagaggggacctcggtgctgccttaggtaagcaccaggggaggggagcc

SEQ ID NO: 29 - DNA, 8797 bp, Rattus norvegicus
  misc_feature (1)..(8797): where n may be a or g or c or t/u, unknown, or other
agaagcagggcaagatggagcggagacgtatcacctctgcacgccgctcctatgcctcctccgagacgatggtcaggggccatggtcctaccagacacctgggtaccattccgcgcctctccctgtctcgaatgacgcctccactccctgccagggtagacttctccctggccggggcgctcaatgccggcttcaaagagactcgggccagcgagcgcgcggagatgatggagctcaatgaccgctttgctagctacatcgagaaggtccgcttcctggaacagcaaaacaaggcgctggcagctgagctgaaccagcttcgagccaaggagcccaccaaactggctgacgtttaccaggcagaacttcgggagctgcggctgcgtctggaccagcttactaccaacagtgcccggctggaggtggagagggacaatctcacacaggacctcggcaccctgaggcagaagtgagaaggggaataggaaaatggctagtgagcagagagactgaattgagagaggagcctggaagaagggtactgcctccccctaaggagagcctcttagctgctgtgtttcaaaagggagagctgtctttgttccctaatatgatgcagaatagccaggaagagactgaaaacttcagggatcagattcccagagagagctgagcattctgggtacttaccaccaagctgagggaggactgtgtgtgggtagccagggtacaagcaggcaggtgcttcagtcactgggcactatgaatggccagagaggtttgcgtccacaaggagggctcttggtctctgaaatataagaaatgcaagaaagagcaggcaggcctggatgccgggcccagatggctgtctacgacctgcttttgaccctgctcaagtcattttgattggttgtagctgtaaaggagaggtcatgatacaatctgtcctaaaagatggtaaccgaggtcaaactctggggcgtggtgctttgctgggtggtggatattcacgtgtccctctggtgcaggaggggcactggtgtcagtgacagtgggcgcacggtggtggatgcgcctctgggaacaagagcaggtggcaccaaccccgataaggcacctcagtaatgagttaaggaggaaggctcttatctgttcacgcgtcaccaggcctctgtgtaggaacctgccattgcttactcttcctgccactctaccgatcctgtgggatccgtttgtaacartcccctccctctggaggttcggatagggacccaagagtcctcaggaaaacctaaagcctgtactgtgttccctgtcccaggctccaagatgaaaccaacctgaggctggaggcggagaacaacctggctgtgtacagacaggtcagagagatgagaggaagggtggggagggaaggcggaggcccgccaccaaacaccgcagttgtctgcttccaagatccccacaaggagggggaagacaaagttggaaattttagtagctaggaccctggggtcaaagagattctctccctgctccaggaggcggatgaagccaccttggctcgtgtggatctggagaggaaggttgagtcgctggaggaggagatccagttcttgaggaagatccatgaggaggtgaggccagggagggggagggaggggccttacgagaaactgcaggtaatagacagagacgctcacagaggnaaacngagaaaccaaatttcaggaagaaaacaaaaacaagagaaagagtnaagacagagacttagggagatgtgtccagtatttactacccgtgnaaacacacgcaggccttccgccgccatgttctaagaagcacagaaccgagtctgagtttctccttcacatcagctctcagccttatcactacctcttccacaactgtagggaccttgaggcctagagagtgaaacacacccaaagtcacacagctagaacccctctagacctgtcacagtggtatgcatctcagcctgtgagtggtagacgcggaacgtttgaactgttcaaggccaatttcwgcnacannaghaaactgaaaccagccacgcnccgcncgcgcacatctattctcttacctcagaacctngctgcaagacatattyattcatgcctggttcctgactatgcagtcctgagaggaaggcacctcaaattctacttccatgcccattagacgctttacttttactttttgacaggatatcactgtgtagctggcctggaacatgcacttcctccagtaacatctgcctcatttccgtgagccattgctataaatggcagacagaggcgtgagtctgggagagtgcagcctaggccgtagcttgagccttaataccccttcttttaccttattaggaagttcgagaactccaggagcagctggcccagcagcaggtccacgtggagatggatgtggccaagccagacctcacagcggctctgagagagattcgcactcagtacgaggcagtggccaccagtaacatgcaagaaacagaagagtggtatcggtccaaggtacctgtggggcagcttgcctcttccaggaagtcttcctgctccctcctgaagaataaaggaaggaaggaagctagggaagggtaacctatctaggtgtccagtcttgcccagagagaggagacttgcccagaaccacgcaagcattattaacttcccaaactactcttggttactgctattataagtaatatcagtaatagttaataatagctaaaggctcaccagtttgctcagtgtcacacagctcctaagtgacaaagggatagcgtttcctatcctaacccatatcatcagagagcctgctttttgatgcctctgccagcctatgccctgcattaccgaacatctactgtgtgtcacgttctttttttctttttttggagctggggaccgaacccagggccttgcacttgctaagcaagcgctctaccactgagctaagtcccaaccccgtgtgccacgttctttagtccagatgaggccagcgtttatacagactgtaggaggcagggacgatgggggatggactaggggtgggacttgtgctagacaggcagcctagagactcaggagaagagggaagggcatctcaggtggagaacaggggctgtgaggaggcctgagacagaaaagcacagagaccagaaatgtcgctgcccagggcgccaagcccagtgtgagttaatgagcctcacatgcagggacagtctgggaggatgaaggcctaagaagttcttgggctacagttgttcctcctaagggagccctgaggttcctggtttttcagtgatcatgtatatctcccagctcaggcagctctctgggctgtgccttgcgcactttccctcaatttttggtcctctctgggtttagtttgcagacctcacagacgttgcttcccgcaacgcagagctgctccgccaggccaagcacgaggctaatgactatcgccgccaactgcaggccttgacctgcgaccttgagtccttgcgcggcacggtgagcacggatcactggttggatggggcagcaagaggttgctggagggggacagggatggagggaccggggtggcataggcgaacacggaagagatcaggggttgcagcctcgaagtttctcaaaatctcccggccaagcgatcagttccatcccgcagaacgagtccttggagaggcaaatgcgcgaacaggaggagcgccacgcgcgggagtcggcgagttaccaggaggcactcgctcggctggaggaggagggccaaagcctcaaggaggagatggcccgccacctgcaggagtaccaggatctactcaacgttaagctagccctggacatcgagatcgccacctacaggaaattgctggagggcgaagaaaaccggtgagctctacctcagcccgagaattcctcttgttcctttacagtactccggagttagccttgtatccttcccacccagagtgttcagcctacgaacaatagatattcaagaaatgtctgaagaatagatgggtccaactactggtaattctcgggactgaggacttgagttattccccagattaagtggcttaccacaagcatggccaacaaaaatggaatttcccatggtctgaagggcttgttctgcctctatgaagttattgtgacttaaaaaaaaaattccatctccaaagcctccttcctggaagtattctgtgggggcagataacacagtcctgcagacacttccggctcctggagagtgttggctgctgggttgaaagttggtggtggaggctggagagatagttagggctttgggttccaacatccatcttatggcttgcaatggcttgtagtaacactccagttccaggggaacccacatctgccattgtatgtgtgtgtgtgtgtgtgtgtgtgtgtgtgtgtgtgtgtgtgtgtgtatgtatgcatgcatgtatgtatgtatgtatgtatgtatgtatcagtcattatgttgctgctttagtcccagcacttgagaggaagaggccagcagatctctgtgagtttgaggctaacctggtatatatattgaatcagcgccatatagagaggctttgtaagaaaaagttggcgaagagaaagagaggagaggagacagagaggagcagaaaggagagagagcaggagagagacagacagacagacagacacacacacgcatgcacacaggacagagagaaaatgtgtgtgtgtgtgtgaaagagagagagagtgtgtgtgtgtgtgtgtatgtgagtgtgtgtgtgtatgtgtgtgtgtgtgtgtgtgtgtgtgtgtgtgtgtgtgtgtcctggtaaagggctttgagattttcatgctggccctggaccagccctggcctggggatgtcctagggcagcctcctcatccctatccctccttttccagcatcaccattcctgtacagactttctccaacctccagatccgaggtcagtatagtggggccttgtgggaaagggtaatgaatcccctccctcctcccccaggcctgtgaactgctctcatggactgggaggcaaagagacccacgctctccttgagagcttcgtgaccctccacggagaccctagatggagtgcagaatcttagggcttatagctggtccctgcctccctctgccctgtgtccccatcccctggaaaacccaacaggtctcaactgatgccaggtctgcaagccatgaacaagctccgccttctaagtgttttgtgttcttgttttttttttttttttttctcctctctctcttaactgcttgtcactacagggggcaaaagcaccaaagaaggggaaggccacaaagtcacaagacatctcaaaaggctcacaatacaagttataccaatacaggctctcgccagattgtaaacggagtcctgccagcactccgttagacacgtgcacttcagttgtgtacttttcccggcttgccttcttcctcccaggcctcttctaagggactgtaccgtgtcctttgtccagaaccttcagtacccacgctaggtcctggctctgtgtaattaggttatactgatagagctagcctacgttaaaggttaggtcatgctaacagagctagcctatgctaacagagctagcctatgctcaaggtgaggtcatgctaacagagctagcctgtgttaaaggcagcatgtctctgggagctcaaaggagatgctctgacccctctgagcaaatgcctctgcctcaccacttttgtgtcgctaggtggtccccttggattgcagtgcctgtgggcaggctctgtgtttgattcatgtgtccccagagttctattgcttcattcagtgctgactcagcccagaggttccccctggatggctgctcttgtagtgaataaactctaggttccctgctcttcgttttacatattagtgagtggcgacacgtatattttcctctctaagggggttggatcaaattacgttttactggtatgtggcccttaaatgattcttagatatgggggaagattccatttaggaacattggccgtgcatattgcagggtgtaattgagctttagaaagttaaccatgctggccaggcaaaggtgccaaataccaggcagtcaaaggtcactagagccagataagtgacagggaaataagaatggggaaaggtccgtgcgtattaaccactaatctgcaccacgtggtatacgtcagtgagttgacactcgcatgtccaggagtagtcactatgagcccattttgtaagagaggaaactgaggctccttccttggtgcagcccttggctgtctctcagccaggtgccagaacagaaggcagagcgggctacaggacagcagcagggtgtgcccagcgctgggggacagatgggagtctccagtcacttcggaaaagtcatgcttttcgctgcctagctagttagccccgtccgtattcacgatcctgggtgcatgatggggaacttgggtgcagggcaggatgtctagtgtcccaagagcccacggagacctcctctcctcgtacctgcagaaaccagcctggacaccaaatctgtgtcagaaggccacctcaagaggaacatcgtggtaaagacggtggagatgcgggatggcgaggtgaggaaacgttccagtggccccggggagttcttgaggctgtactggagaaagcctggtactagctcactgaaggtccttattaactaccaggagagctctggtagaagggatgggccttgaatgtaattctccttttccagctctttcatgggtggcgtgctaagggcaacctgtatatggaggccaccatctttctggaacacttggtggagggaggctttcaaattttactcagaagaactttccataggggaattggaagaggggctagacatagaatggccgtcttcatttgttgggatgttagcagcaatggttgcccttacgtcatagtcctctacaagtggcatttcatatctcaatgtgtgatgcaatcaagccgatagtcctggaagacacatccagggcagtcaggggctcgcctcggggatgctcagcctagcaaactggatctgggattctggagcccctaactctgtgctgtgactcttccctctgcctgcaggtcattaaggagtcgaagcaggagcacaaggatgtgatgtgaggtgtgcccagctggcggcccttgccatacagtgtgagggcctaaagctccctcctcagatagtcttgtttgctaggcccaattcccatccacaccagtgctccccttccttctgtttttatgcccacggctcggtcagtgcggagtctcatggacggcacagaccaccctgcatctccaactaacaggatactcaccccaaaggggcaatcaggaggggaggacccccctccccccagctgggttagaactggaagaaagaggaaagacaggggcagggagacttaacaaatcccttccttcatccttgttgttatggaaaccgttgccagagctggaggtctctgggaactggactttgagttttcataggctgctggagcaagacaaacattcagacagaaaggaaaagttcccgaggcaaagaatctctagccagaggcctaggcatctggaagaaccctgacgatgtaggagtgggtagggcagacttgctacctggaatggccactaaggcagtcctgaagggcccccctccggagggatgaccctcgtgtatcggccccactgagcagccctgcaggttgatgccccacgagcctgtgaaaacttggttcttggcatgtggcaggctctatagcataagtggagagggaaggtgtactggagggtatagaggagggctctctggcccctaagtatggatgcggagaggggggagcccaggaaggctaccccgctcaggctgcaggggtgccatggcggaggaaccggtggagataacttggacaatggagttggaagttgtaggcaactagttacacttggctctgaatccttggaatcaaggaaatgacctgttctctcaaagacactgaaacaggagagagggacttccatccactgggcagggtacaggcgcgtctcagttgtgaaggtctattcctggttgctcagtccccaactgcgcatcaccctgggcttctcaacctggaagagtccacaaccatccttctgaggccctccatccccacaaccactagctgttgttctccaagccaagggccccattccctttcttatgcatgtacggagtatcgcctagactttaagcgtccatcctgtttgaaagtttgggaaactgacacacgttgtgttcaagcagcctggtgtggagtgccttcgtattagtgtaccctctcggaagctggttggtgggcaggtgaggaagaaatggagctgaaagtgtcccctcagttgtcctttcctccccctctaaggtccctcacttttcccaggacatcgtacactcccccccttgtcacctctgctaaccttcagagcagtactgtcacctttactcactgggcagaaataaagacagtgtcagaggcttccacagagkatctgatttgttcagaaggtggggtgaggacagacaagacccaaccttgctcgttatcaccaccgaatgtctagcgagcctaaacttccacaacaaaccctccacagcagcagctggcgttaaatccgcctccaacgacgaggtgccct

SEQ ID NO: 30 - DNA, 1453 bp, Rattus norvegicus
gacggtatcgatcgaaccccttcgataacagcggaatcccccgtctacctctctccttggtcctggaatagcgctaccgatcacaaagtagccctaagacataataaaccctcaactgctccgtagtttttcttatgaaagccaagtaaaggggacgtaagcaaaaaaatatttttttttgcgtgaaggattccaaaaataaaattctctggggattgagaagaaagaaaaaaaggaaaatgccagctgatataatggagaaaaattcctcgtccccggtggctgctaccccagccagtgtcaacacgacaccggacaaaccaaagacagcctctgagcacagaaagtcatcaaagcctatcatggagaagaggcgccgggcaagaataaatgaaagtttgagccaactgaaaacactgattttggatgcacttaagaaagatagctcccggcattccaagctggagaaggcagacattctggaaatgacagtgaagcacctccggaacctgcagcgggcgcagatgaccgccgctctcagcacagacccgagcgtgttggggaagtaccgcgccggcttcagcgagtgcatgaacgaggtgacccgcttcctgtccacgtgtgagggcgttaacaccgaggtgcgcactcggctgctgggccacctggccaactgcatgacccagatcaacgccatgacctaccccgggcaggcgcaccccgccttgcaggcgccgccgccgccgcccccgtcaggacctggcggtccccagcacgcgccattcgcgccgccgccgccgcttgtgcccatccccgggggcgcggcgccccctcccggcagcgcaccctgcaagttgggcagccaggctggagaggctgccaaggtttttggcggcttccaagtggtgccggctcctgacggccaatttgctttcctcatccccaatggggccttcgcccacagcggcccggtcatcccggtctacaccagcaacagcgggacctcggtgggtcctaacgcagtgtcgccttccagcggctcctcgctcactgcggactccatgtggaggccgtggcggaactgaggggctcaggccactgctaatcataaactccctagcccacctctctcttctgacggacactaaatacgaacttggactttaggagagacttttataagtcggtggttactttgttgcttttttaaattctaaaaagttactttttgtagagagctgtattaagtgactgaccatgcactgcatttgtatatattttatatgttcatattggattgcgcctttgtattataaaagttgagatgacatttcgttttttacacgagatttctttttttatgtgatgccaaagatgtttgaaaaatgctcttaaaatatcttcctttggggaagtttatttgagaaaatataataaaagagtgaaggctttt

SEQ ID NO: 31 - DNA, 4831 bp, Rattus norvegicus
agcccgctccccgagcagccttagccatccctaactaatccaactgcttcccagtctgcgggtgctgggctcggtgctgagaagaggctctggctcttggctattgtcaggtcaaacactaagactgtgtccatgttagaactcatagaagttaatggaacccctggcagtcagctctccactccgcgctccggcaagtcaccaagtccatcgcccaccagcccaggaagcctgcggaagcagagggacctgtaccgccctctctcgtcggatgatttggactcagtaggagactcagtgtaaaagacacagtggagcgatgtgtatggtttgtgatgggatcaaggtttcccatttcctaagcaagcatctaacagcaaaatacacaaaagagttttgcataagcaattaaattatacaaaagcaagagtgcagacattttctactcggcggtattctttgaaagaaaaaaaaacttttttaaagcatctcctccatttgatgtaaccgaggagagtttgaatacaatatttgttcatcactgaggattttatgactaaggtctggttccagataaatttcatatatgactccaatattaagtacaagaatcaccctaacgatccagcattatggagtcaccatttctataaatgagcctgctttttaatcagtaagtagatatataaatacaatttttctgtacttagtatcttttacctttttagtttaaatttttttaaaaaaatcagacagctaattagttatttcattgcaaaataacataaaagaatctcttcaggattttttatttgagtgaatacgtctgaaggggagtggaggaactccgtaagagacgtctcccagagaccaacattaggcagcgagaacgtttctggttttttttttttttttttttttgtttttgttttttgccacatggattttcttttcccagatttttataaaaatagtttcaactacatggaaacatgaataacttggcattattattaattttataatggttccctacagcaacaacagtatatatatctttttcaagtgtgtaaagttttttgacctaaaacaaatgggacttgtactgtttttcaaaaaataaactcagtttgagaattggaattcttctacacagagcttgtacgttgggcctccaggtagctaaactccattctgatcatcctgcatcccgttgtttgggatatcttcacatgcaaaactccttttgtgtcaaaccgagtgtgtctatgattataacccactgttaaaattaacctactaattcttctcctttagtcctctactttcaaaagcgtgagtcagctttgcttaaatgtaaaacagttattttttaaatctagatgttagaaatcaatgtgtacggtgtattgtatttagaccatacccgtgaccgtttctttttactagctcaaacactaacaatttaagacttacaaagcatgggttttatattcgccaggcttgacatatctcagtagggtgacagatttaaaatgtatggtgccatttggagccttttacttgagatttctttcaaatttcacttgaaaacattttgaaaatgcctgattcaagatggagaggagttgccttgtcatacggtctcaaatgttattgatttatttctctcagtagactgttcgactagcaatttttcccatctggtttatcgttacctgtgctagcatctcacttgacaagtgaataaaatattccaactgaaaaaagaaaaataagacctccaggcaacctaccagcatttgaaaagttagccgagtgataattagtgcctcattaccctgcctgttagagaggagagggcacttctttggcgtgttatttgagaactgcagactgatccctccatcttgaaagcacagagaatgatcatacctgttccagacttcggattttaaaactgtttccaatagacacgaacagagactcctgctgagatgaaagggccgctttgtctttagctgaaacagtaaacatctgggaggatgctgtgctgcctgttggagtgttacgtctttgtaagatgcattgcttaatatagtccaacccgtaaaagattagtacagatttgactaacctgtcaaaaaatgcaatgaaagcagtgggtcagatggtccagaacaaaacataaattccagtaatttcaactcccctatgaagtcaatcttatggacgactactgctaggagaaacctaataaatgttatctgctataaaaatcgtggctttgtttagacacagcatagagtagacttggactaattgccagtaggtattgtggccaactttccattcaagcagttccagagagatgcaatcccatggcatttcatcctggaacatacgatttttattaaaggattgttcaagggccatatatctcatttacttttgaaactaaagtccctctgagattgaaacaacacaccctgagatctctttctatgcacggaataattctattatccaaaagcagagtttttgaacatccaagcagcccaccaggtcattagtaaagacccaatgggcaattagcagtcactcaagacgtggctgcttttagaatccttctaacatcattagtcgctgaactctaaaaatgaccttctgaggagagagctccagccaacaaaagccacatgcttttctctcactgataaagcctctgctgtgttgacctttcaggcctttgcatgtatggttacatttttttagacatacctagaaagaaagatgctataaagagtcccctgctttcagcattttatgagctaaaaccctagtgatgcacaaaatgtgttgctctgcgtgaactccgaagccatgtgggaaagtcattgtcatttcagcagaaggtctgtactcttgtaggtttctaaaccggaagtttgtgctccacctatatgtacgcatgcctgcaatgcacacatgaggtcaccttaacatgtcaagatctgatcccgttgtctgccaaccccagagcagctctgacacctatgctcccctgccaagcagcgagggcactcgccctccccttccttgcagccgcaccgagctgtacttttgtttccatttaaaagaactgaattccaaaagcctcgttttaaaaaagcaattgggaatgaaaatacataagaataatcctaaaagataaatccatatatttaacctgttgaactactaggttttttttttcttttcctggtggccatatatctatacaggtgtgtaggtgtgcctacataaatatctgtgtttgtatatagaacatgaagaataaaggatgatatttgtgatatggtcttataagttactggctcctgtagaattaactgtgaagctttatgtgataaagttgtgacgtatgtattagttggcacatgtttaagctcaaaacaagttggctaaattatcatgaagcagtaccaactgttcacgggtacgaatttatccaagaccgattggaatccagacaaacaaaaaaattgaagtccaagcaaatccaatcttacagtgattgataaatgtaaatactcattgatgttttcactgtaaattcttgatgctcagccagtaatctgacagaatgcagtgacactttcctgttgtgtgtgtactgatgactacagcgattatcaatgggaaattcaccaaacaaactgatttctagacttgtggacagttgagtgttgtgctgaaataaaagtagaaaaggtcaaagtaaattcggtttgaagccttgttatgaagccagacaacatgtaaggccatgctgtgtgtcctttttataaactgttttggagacacttgctgtgtgtgtgcatgtgtgtctacatgagtgtatacacacttgaaaatgcttattgactcaagttcactgtctgtatacatctgtgcctatctgcattggtctggccagcgcctctaactgtgcatggtagtgacagccctgtgtgattggatgaacccacagaaaaatagtgtggaaatcagagctgcgtcaaactcattataacaattgcctgatatagcatattaaattgtcagctgaaaaacgtttgtaatgttgtgcctgtaacatgtgtagggaaaactggaaaatctgaggccctcactataattttaagggtgcatgacatgtggtgtctttaaagatgtatcttattgtaataaaaggtgatggataaattctgaggtagcgtaagtgaagcatgaaacgctaggtgtttgtattatatatggccacacaatatttttgaaggcagggtagtaaatgtttgaaggttaataggcaaagaatatggaagttaattttactgtgatgtgtttttgaggtgtttttacacattgccagacaaggaacttagttcctgtttcccccctgtggactctttgaaacactatgaatgttacaaagttggattttctttataaaggtcgcaaaccatttgcagttggagttgcttggatgtatagtctcctctttggggaaaaaaaccactttggtattaaaatggtttaagcactaatacattgggaaatacgtcgtctgaggccatctgcagacatctgtagagatgagaactaatgaaatgtgttactgaactacccagttttgatagcttccatgcctctctgctgtagtctcctcgaaaatgctgtaccttgtgtttcacatgcaaccatgttactctctcccgaacttacctttgtgacacttagcagcttagccaaaccacgtaataaacaggaagaactgcaaaaaaaaaaa

SEQ ID NO: 32 - DNA, 666 bp, Rattus sp.
  misc_feature (1)..(666): where n may be a or g or c or t/u, unknown, or other
atgacagtggtctttattgtttaggtgacactgtttcatgatagacagtgaagacagagcatgttaaatgttggtatatcacattagttaattctgtgtgcagaggatctgtgtgcactgtatcagctatatacagattaagaacatacatgacaaagtacaaaagcacaagaagaggttaaatagggaattatttctgggttataatcacctggtaacatacaccttctgtcacacaactgtgtcactgctaaaaacagtaattagcatacatggtgatgatctctatttaatctagcacagccctggtgtgaatacataagggcatgataatatttcacaggtacaggctaagttctggggcatgaactcttttgagtaattcataaaacacacctgaggcatattttttgtttttgaaaaaaaaaagcaacgttctgacatagtccaagctttactgtgcaagggagacatttaataaaaacatacttttgctgccctgacaggaggtggatactctgatgaagatggtgaccagcaaaagaaatccctctgtagcagaataaggcaggctttccaactaagccagtaatctattagcttccgaatatgacaatngntactctttcctgacttgaatcctccttcctttcactgggttctaaa

SEQ ID NO: 33 - DNA, 602 bp, Rattus norvegicus
gctgcacgcagtgcccacctatgctcgccatgatgctcaacactacgctctctgcttgcttcctgagcctgctggccctcacctctgcctgctacttccagaactgcccaagaggaggcaagagggccacatccgacatggagctgagacagtgtctcccctgcggccctggcggcaaagggcgctgcttcgggccgagcatctgctgcgcggacgagctgggctgcttcctgggcaccgccgaggcgctgcgctgccaggaggagaactacctgccctcgccctgccagtctggccagaagccttgcggaagcggaggccgctgcgctgccgcgggcatctgctgcagcgatgagagctgcgtggccgagcccgagtgtcgagagggttttttccgcctcacccgcgctcgggagcagagcaacgccacgcagctggacgggccagcccgggagctgctgcttaggctggtacagctggctgggacacaagagtccgtggattctgccaagccccgggtctactgagccatcgccccccacgcctcccccctacagcatggaaaataaacttttaaaaaatgcaaaaaaaaaaaaaaaaaa

SEQ ID NO: 34 - DNA, 3101 bp, Rattus norvegicus
tgtctacaccataattcctttgtctttgagccagctcacaaatgtcactgtggttctgagtgtgggggtcttggtgcagtccctcccctcccagtcccttccgtcgaggagcatggtgctagtgctgccacagcctggagacgcacacaaccccccaaaatctctccagacgaccgtcccacgatcacaggacagaaccctccaaatcgaaacggaggaaacggacagccattgaacatggacgaaggaatccctcatttgcaagagagacagttactggaacatagggattttataggactggactattcctctttgtatatgtgtaaacccaaaaggagcttgaagcgagacgacaccaaggatacctacaaattaccgcacagattaatagaaaagaagagacgagaccgaattaatgaatgtattgctcagctgaaagacttactgcctgaacatctgaaattgacaacactggggcatctggagaaagcagtagtcttggaattaactttgaagcacttaaaagctttaacagccttaacggagcagcagcatcagaagataattgctttacagaatggggagcgctctctgaaatcgccggtccaggccgacttggatgcgttccactcggggtttcaaacctgcgccaaagaagtcttgcaatacctcgcgcgctttgagagctggacgcccagggaaccgcgctgcgcacagctcgtcagccacctgcacgccgtggctacccagcttctgacgccacaggtgaccccaggcaggggccctgggcgcgcgccctgcagcgctggggctgcagccgcctccggttccgagcgcgtcgcccgctgcgtgccggtcatccagcggactcagcccggcacggagcccgagcacgacacggacaccgacagcggctatggaggcgaggcggagcagggccgcgccgccgtcaagcaggagccacccggggacccgtcgctgcgcccaagaggctgaagctggaggcgcgcggcgcgctcctgggcccggagcccgcgctgctcggctctctcgtggcgttgggcgggggtgcgcccttcgcgcagcccgccgccgcgcccttctgcctgcccttctacctgctgtcgccgtccgccgccgcctacgtacagccctggctagacaagagtggcctggacaagtatctgtaccccgcggcggccgcgcccttcccgctgctgtatcccggcatccccgcagcagccgccgctgccgccgccgccgctttcccttgcttgtcgtccgtgctatcgccacccccggagaaggcaggttcggccgctggtgccccattcctggcgcacgaggtggcgcccccggggtcgctgcgcccccagcacgcgcatagccgcacccacctgccgcacgccgtgaacccagagagctctcaggaagatgccacgcagccggccaaggacgccccctgaacccagcattccttccagaacagggcagggggctcctgaggagtcgccaggtttccaagttcaaacatcccctaaggcgtaccagggaggaagagtaagagatgctctgctcgacaggcttaggacaaaaacaggtgttttgtgtatgtttggagttcctgttttgcccctttctcacccttctgccaccccaccctctaccctttgacactcccttccccatccctgctgtcacagagcctccctgagaaatactggttatcttaaattaccctcccttacatttagttcacgtcctctgtttccaaacatagaccctggttcaggagtctgttgggtgggagagccacacggaaccagttagagtgcctggtatcagggctccttgacccaggcctggaacagtagctgtgtcccctgtctgtccccttaggaggtgacccataactgagggtctctgaaagttacattgacgtgtcagtattttgtattcttcagctttttggaaggtacctctttttcaaagaagtgaggatgccattgccctgttgtgaggtggctggagtggtgtctttataccttgcacctgttgggagaaactgagagttggggccatcttcaggcactgtgtcagtgtgggagctggaagagggagtttggagcccgtggcgcctttctcgcactttattgacaaattgacctcaacccctttgtcccatgtctcaactcacagatatatgtcataggttatatatttgtgtttctgatccctcgttattttatccatcatggtcccaaatttttgtaatgttactggggtttggggtggggtggggtgttaaagtgctctgggctggaaaaagacaagcccaaacctattgattgtcgaattcttagatgacagaagtggagagaggggcttgtggtcccttgtgatgggaagtgctgtgaacatgtagaaggccctgccagcctcgctctctcaagtctgtatgtatttttcgggagaccaaaccagacaccagataatcaggaagaaagctttttaaaataaggcaaaaaccgagaccttgtctagatatttttagtttgttgccaaggtagcactgagaaatctcacttgaatgttacataaggagtgattcacaatagtctagagtgaagaaagttatctgggtctgtgagtgttcgggtccgtttgctgctgctgttgctactgtttgcctcaaacgctgtgtttaaacaacgttaaacttcttagcctaccaaggcggccgtatgtacatagctgttaatacccccaactaatgtctgacatgctatttttgtagggagaagatacctgctagtgatattttgagttaaaatatcttttggggcggacttggtgaaatgtttgcactttggtcacaatgcttctactgcttggtgcaacgttacgctgtcttaaattattaaacaaataaaaaatactatctgcaagaaaaaccagctggtttagacaagtttagtatgtaaagataagctagaaactatctttatattctagtattttcagcactccatattacctaaatattgccacactattttgtgatttaaaagttcttactaaggaataaaatctttata

SEQ ID NO: 35 - DNA, 343 bp, Rattus norvegicus
  misc_feature (1)..(343): where n may be a or g or c or t/u, unknown, or other
tttttttttttttttctggctccttttctggctctgggaggagctttgctcaaaagggacaccacctatccttagcatgcttctcttgaggtacagtatgcacaaccaataggagacccaagtcaataatatataaaaggtgcttaaaaaaaaaaaagcaaacagtaacacacacgaagaaatcaaccaaaaattggtggacatctgttttttattataatatagattctgaatattttaaggaataaagagttattgttttattacattgccctctaatctgtatggaataaaattatgactttgtnaaacaaaaaaaaaaaaaaaaaaaaacctcgtgccg

SEQ ID NO: 36 - DNA, 619 bp, Rattus norvegicus
gcggccatggagcccgacaacagtccacggaagatccagtttacggttccgctgctggagcctcacctggacccggaggcagccgagcagattcggaggcgccgccccacccctgccacacttgtgctgaccagcgaccagtcatccccagaagtagatgaagaccggatccccaacccacttctcaagtccacactgtcaatgtctccacggcaacggaagaagatgacaaggactacacccaccatgaaagagctccagacaatggttgaacatcacctagggcaacagaaacaaggggaagaacctgagggagccactgagagcacagggaaccaggagtcctgcccacctgggatcccagacacaggctcagcgtcaaggccagatacctcggggacagcacaaaagcctgcagaatccaaacccaagactcaggagcagcgtggtgtggagcccagcacagaggacctttcagcccacatgctaccactggattcccaaggagccagcttggtctgacagaagttgacatccggggatcgccagtgagtgtggaagttcatggacactggatgtttcttaatctcttgtttttaaacgtgataaatttggtgtt

SEQ ID NO: 37 - DNA, 4614 bp, Rattus norvegicus
aattccgtgggcggcgggcggcggagaagctccgagcggggccgggcggcggcagattggagtcgcgacacagccgccgtccgcttcgggctccacgtagaaggaaccatgctggccgtcggctgcaccctgctggtcgccctgctggccgcgcccgcagtcgcgctggtccttgggagctgccgcgcgctggaggtggcaaatggtacggtgacgagcctgccaggggccactgttaccctgatctgccctgggaaggaagcagcaggcaatgctaccattcactgggtgtactcaggctcacagagcagagaatggactaccacgggaaacacactggttctgagggccgtgcaggtcaatgacactgggcactatttgtgcttcctggatgatcatctggttgggactgtgcccttgctggtggatgttcccccagaggagcccaagctctcctgcttccggaagaacccccttgtaaatgccttttgtgagtggcatccaagcagcactccctctccaaccacgaaggctgtgatgtttgcaaagaaaatcaacaccaccaatgggaagagtgacttccaggtgccttgccagtattctcagcagctgaaaagcttctcctgcgaggtggagatcctggagggtgacaaagtgtaccacatagtgtcactgtgcgttgcaaacagtgtcggaagcaggtccagccacaatgtagtatttcagagtttaaaaatggtgcagccggatccacctgccaaccttgtggtatcagccatacctggaagcctcgttggctcaaagtcagttggcaagaccctgagtcctgggacccaagttactacttgttgcaattcgagcttcgataccgacctgtatggtcaaagaacgttcacggtgtggccgctccaggtggcccagcatcaatgtgtcatccatgatgccttgcgaggagtaaagcatgtggtgcaggtccgagggaaggaggagtttgacattggccagtggagcaaatggtccccggaggtcacaggcactccttggctagcagagcccaggaccactccggcagggatcccggggaaccccacacaggtctctgttgaagactatgacaaccacgaggatcagtacggaagttctacagaagcaacgagtgtcctcgccccagtgcaaggatcctcgcctatacccctgcccacattcctggtagctggaggaagcctggcgtttggattgcttctctgtgtcttcatcatcttgagactcaagaagaaatggaagtcacaggctgagaaggaaagcaagacgacttctcccccaccgtatcccttgggaccgctgaagccgaccttcctcctggttcctctcctcaccccatcagggtcccataacagctctgggactgacaacaccggaagccacagctgcctgggtgtcagggacccacagtgccctaatgacaacagcaacagagactacttattccccagataattgtctggagggtacctggcagctggcacgcaagtttctcactgccggccccgtccaccagggctgggggcggggtgggcggggctgcagcttcacgatcccacaggagccttgcaaaggttctgagtgggagaagactggtgtgctgcacgggcttcgaaagaaggggctgtgaggagcacgagccatcatgaagagagcctgtgatgactctgaatagagacgcccgcccatcagctacacacctgatggtggctctcaagctatcctctcaggaagcctctgggaggggcgacaaaggctgccccagttgcctagctctggctcactggcccaagctgccttttagcttgaactcctaaaatccaagcaccttggccattctcttcctaggccaccgaggccgcggggaagcttggttctactttccttctcaacacctggagaagcagctgcccggtggtggtgactaacgtatcagggcctgatggcttatgaggaatgacaattaattcctcataagcagtttttaaatgtgaatagtaatcctaggcactgctgacttgaggttttattttcttcgatctcaggacttcaggagagaagcagagcagaagtagagagaggatgggtgtccattgtccgtgtggtacttgagggggatacagcctggaaaacacgtttcctgtccccctactctcccagaagaggtagggggtggcgcctcttccagggcagagagtataactactttacctggccttgcccatactggtttcaactggacttgagctactaggaaaaatgacattcatgcaaaaagaaaactttaactagcaagaatgcacttccactttggtttctagaggactgttgctcctcttgagacgctggaagaggccgctcactgtaccctggtgtatgagccctcaccccccaccccagggtaagtgcagtaactttagtctaaacaccgagtcaggtaaaaatcgaggaaaaaacaaccctgtttcctgtaacagaaaagcctttggtttcgttttgtattttgattttttttttgtcttaaaaagtgtaaaaatagtctgtccatactctgcttcagggaatgacctgtgaatactccccaggcgtgggcaggaagggtgtctgcttcctgctacacctcactgccacctcggccttccttgctttacattcaactgagttgcctcagctgctttcccctggggcgctgaaaaagccagtgatgttggtggtcaccgagaagaccacagagccacagagtaatgctgtgattgaagcgagttacgcaaccacagcaccccacatttgctgtattatagaactatgctaggagcttgccttttcacaaaataccaccaccacgagacgtggcagagctcggaagctgtcaccttgtgccatctgcttgccagctccaaggggccactgacttaagcagttattttctttgtgggctttgttcatttcagggcctgttgctgtcttagaaaaagctctgtcggttgacaaaaacatcagacaggtagtcatgtttatttattttttttccttctttgctaagtctttgggactcaagggtagtaaaaaatgctgtgaaaagggaaacattagaaacagcgatcttcggggaataggtgactgtgcccacgcactgttcttcagtccctcacgtggctctgcccgagtgctgttccaagccaggcagagcaggctggcggaagattgaaatccagatagctcgttatctctgagagctaaatagctttgatctccaagctgttattgctttcactattgtaacaggatagcctccccccccatgtcaaaaggatgcttttcccttttgactttttataagctaagtcagtgaagtctgtttcatctgagctccagcttcgttcagttcgcacaggtgtatgccctcagctgcttcgggcctcagatctgtgctagttgaatggttgtcccatccttgggtcatccttaccagagtttctgcagcccacaggtctgccttgtcaacagtaccacttaacaccagcattcagtgcccaggcagccagatgtggagggtttacccagagatgatttaaacatgaccttaaacgtgtatggtagaacgaggggaacccataccagctcaggttctaaagagatctttgattcttctggcattagtgaaatagctttaaactatttcaaggaagaagccttggccacacccacgacatttggtgacaatcctttctctccatgagccttgtctttacaccttctcacctggctgaaagctcacactgaatctttcctatgtccctggtgtcttgggagaaaggaaactggtatgggcttcactgctggaattggcttggagccagcgtgtggcgcagccctggcagggtgggccaggcttagttatggtgtgctggtttaaggaatgcctggcttgcctggttgcttgggttctgagctgcagagtttcctagcagttctttatggctgacctagttggggaagattcccacactcaactgcaggtggaggtggtgagaaagctgttttcatttggagaggcaggatcagcccaagaagctttcagtgggagagcctacagtgaggctgtacctcactgtgggaggaggcaggccagctggctcaggtcctgggactggcactggggagggtctgccaaaggtccctccagcctgtagtcctagcatagtcgggtgccagttccaggaagtttctatggcaaccttagtgctcattaaggaacattgtcagttttgtgaacatatgctcagatggagatcttgttttcagagaaaggactggtacagtgtgtaacaagctggagcagacagagagactttttggcaagagatcacatccgttaagcagaatacctcagtgctacatgtttttgtctttgagacaatgtttttaaggtttttatgctctgttacctgtaagctgatacctaaaactttctgcaaagtcagggtttttcaatgccttttttttttttttgccattgtttgctttaaagtgaagattgtaactgtttgaaataaataatttctaaaactg

SEQ ID NO: 38 - DNA, 2952 bp, Rattus norvegicus
tgctgggcgaggggacacactgactgttataataacactacatcagcaactcctggctccccaacagccggatcccaggcaggagagagtcagtggcagatagccatttttttttcttccttaagaagccaacaacttggttgctagttttatttctgttagaattttttttttgtgtgtgtgtggatgtgtggtggtggtggtcttttctaagtgtggagggcaaaaggagataccatcccaggctcagtccaacccctctccaaaaacggcttctctggcactccaggtagcgagggagttgggtctccaggttgtgcgaggagcaaatgatgaccgccaaggccgtagacaaaatcccagtaactctcagtggttttatgcaccagctgcctgacagcctctacccggtggaagacctcgccgccccgtcggtgaccatcttccccaatggtgaactgggaggcccctttgatcagatgaacggagtggctggagatggcatgatcaacattgacatgaccggagagaagaggcccttggatctcccatatccgagtagcttcgctcccatctctgcgcctagaaaccagaccttcacttacatgggcaaattctccattgaccctcagtaccctggtgccagctgctacccagaaggcatcatcaatattgtgagtgcgggcatcttgcaaggggtcacccctccagcttcaaccacagcctcttccagcgtcacctctgcctcccccaacccactggccacgggacccctgggtgtgtgtaccatgtcccagactcagcctgaactggaccacctctactctccaccaccacctcctcctccttattcgggctgtacaggagacctctaccaggatccttcagcattcttatcgccgccacccaccacttccacctcctctctggcctaccagccacctccttcctacccatcccccaagccggctatggacccaggtctcattcctatgatcccagactatcctggattttttccatctccgtgccagagagatccacatggcgcggctggccctgatcgaaagccgttcccctgccctctggactccctgcgggtcccccctccgctcacgccactctccaccatccgtaattttactcttggggggcccagtgctggcgtcacgggaccaggggcaagtggaggcggtgagggtccccgactgcctggcagtgggtctgcagcagtgactgctaccccctacaatccgcaccacctgcctttaaggcccatcctgagacctcgaaagtaccccaacaggcccagcaagacgccagtgcacgaaaggccgtacccctgcccagcagagggttgcgacaggaggttctcacgctctgatgagctgaccaggcacatccgaatccacaccggtcacaagcccttccagtgtcggatctgcatgcgaaacttcagccgaagtgaccacctcaccactcacatccgcacccacaccggggagaagccctttgcctgtgactattgtggccgtaagtttgcccggagtgacgaaaggaagcgccacaccaagatccaccttcgccagaaggagaggaagagcagtgccccctcgtcatctgcatccgcccagtcttcagcctctggtcctgggggctcgcaggccgggggcagcctgtgcggtaacagcgccattggaggaccactggcctcctgcacctctcggaccaggaccccgtgagatgaagctcccgctgacacaccagtttcttcaggccccagaggccctctatccactcgagctgcaaacactaccgcccttctgtgttcttccccgtgatcccgtgaacctgtgatcctgggcaaaggaccctaatggagcccagctctgtcccaccttctcacagacggccttctgaaaacttaggccattttaaaggagttgactgtcactccaagaaatggggagccagaagagggctgggcgagggcccctggcctacagggctgtgctctaaccctgacagagagatgtttgactatggtctgcgagcccttccctttgaccctcgatgccagttgctctgagactttttctacaataggttgggagttgttgattcctttgagcaaggacagcgaaaaagactaaattaaagcaaaaccgatgtggcactttaatggcttgggacggacttggggtaggggtggggggttgtacagtgagcacagtttagccctggcctggccgctgcactctgtggccctagaacagtgaatggaagtttctcgagccatctcaacccttaagcaatatgtcctataaactcaagagaacgaacggaagtgcaatgtcggggaggacaaagccaatattggctccttttttttgagaaacaaagattattttccagtgtatatccatttagatttttgtgtattttttctttctggatgtgcactgtttctccgagttctgaacctttgggaaaaaagtgtaaaacatttatgatctcttgaatcgagtcaaaagttaacttatttaaaggggggtgtacataggatgcatgcagtggtgttgcaagtgtcctctgtgccttgtgtgatgtgggcagtgttacagggtctgcatgtgtacaggatgccttactatgaaaaaaaaatcactccctgggtttaagtatggctgtatatttctgcctattaatatttggaatttttttagaaagtatatttttgtatgctctgttttgtgacttgaaagtgttaccttcgcagtcaaatttcagatgagagtgtgcttaacgtcactgcagctgacttgtttggttattagctcttaatagttgtggaaagattaaacaatctattctaacacagaaccactaactggagttcagatatcggacggcttatggcaatggtgtaaaataatacttttcaacaataaa

SEQ ID NO: 39 - DNA, 2032 bp, Rattus norvegicus
gaattcccccggttcttctctctaggtcccctatctccgccccgggcctgaggggcgcaccgaccgccaccatgagttcgttcagctacgagccgtacttttcgacctcctacaagcggcgctacgtggagacgccccgggtgcacatctccagcgtgcgcagcggctacagcacggcgcgctctgcgtactccagctactccgcgcccgtctcctcctctctgtcggtgcgccgcagctactcatccagctccggctctttgatgcccagcctggagaacctcgatctgagccaggtagccgccatcagcaacgacctcaagtctatccgcacacaggagaaggcacagctgcaggacctcaacgatcgcttcgccagcttcatcgagcgcgtgcacgagctggagcagcagaacaaggtcctggaagccgagctgttggtgctgcgccagaagcactcagagccttcccgcttccgcgccctgtatgagcaggagatccgtgatctgcgactggcggccgaagacgccactaacgagaagcaggcgctgcagggcgagcgcgaggggctggaggagactctgcgcaacctgcaggctcgctacgaggaggaggtgctgagccgcgaggacgccgagggccggctgatggaagcccgcaaaggcgcggatgaggctgcgctcgcccgcgccgagctggagaagcgcatcgacagcctgatggacgagatagccttcctgaaaaaggtgcacgaggaagagatcgccgagctgcaggctcagatccagtatgctcagatctccgtggagatggacgtgtcctccaagcccgacctctccgccgctctcaaggacatccgcgctcagtacgagaagctggccgccaagaatatgcagaatgccgaagagtggttcaagagccgcttcacggtgctaaccgagagcgccgccaagaacaccgacgcagtgcgcgctgccaaggacgaggtgtcggaaagccgccgcctacttaaggctaagaccctggagatcgaagcctgccggggtatgaacgaagctctagagaagcagctgcaggagctggaggacaagcagaatgcagacatcagcgccatgcaggacacaatcaacaaactggagaatgagctgcgaagcacgaagagcgagatggccaggtacctgaaggagtaccaggacctcctcaatgtcaagatggcattggacattgagattgcagcttacaggaaactcttggaaggcgaagaaaccaggctcagtttcaccagcgtgggtagcataaccagcggctactctcagagctcgcaggtctttggccgttctgcttacagtggcttgcagagcagctcctacttgatgtctgctcgagcattcccagcctactataccagccacgtccaggaagagcagtcagaggtggaggagaccattgaggctacgaaagctgaggaggccaaggatgagcccccctctgaaggagaagcagaagaggaggagaaggagaaagaggagggggaggaagaggaaggtgctgaggaggaagaagctgccaaggatgagtctgaagatgccaaagaagaagaaggtggtgagggtgaagaggaagacaccaaagagtcagaagaggaagagaagaaagaggagagtgctggggaggagcaagctgctaagaagaaagattgagccccattcccaactatcccaggaaaaaagtctccccaaatcaggtcaacctcatcaccaaaccaaccagttgagttccagatcctatacagattaagaagtcaatatatgtataattctgagatgacttaggttggacattcaatgttgtgctatgactttcctccttatgcagagtatctgtttgcttgcagagtggctttctggcttgctgccaacctgtgcatggtccatgcttatgagttcaggatctacggcaatgtgaatcacacagatgtttacaataataataataaaaaaaccacacatacacaacatgaataaatgaattc

SEQ ID NO: 40 - DNA, 3070 bp, Rattus norvegicus
cggacgcgtgggcagccacacaccccaaggcctccaagatgagctacacgctggactcgctgggcaacccgtccgcctaccggcgcgtcaccgagaccccgtccagcttcagtcgtgtgagcggttccccgtccagcggcttccgctcgcagtcctggtcccgcggctcgcccagcaccgtgtcctcctcctacaagcgcagcgcgctcgccccgcgcctcgcctacagctcggctatgctcagctcggccgagagcagcctcgacttcagccagtcctcttcgctgcttaacggcggctccggcggcgactacaagctgtcccgctcaaacgagaaagagcagctgcaggggctgaacgaccgtttcgccggctacatcgagaaagtgcactacttggaacaacagaacaaggagatcgaggcagagatccacgcgctgcggcagaagcaggcctcgcacgcccagctgggtgacgcttacgaccaggagatccgagagctgcgcgccaccctggagatggtgaatcacgagaaggctcaagtgcagctggactctgatcacttggaggaagacatccaccggctcaaggagcgcttcgaggaggaggcgcggctgcgggacgacaccgaggctgccatccgggcgctgcgcaaagacatagaggagtcgtcgatggttaaggtggagctggacaagaaggtgcagtcgctgcaggatgaggtggccttcctgcggagcaatcacgaagaggaggtggccgacctgctggcccagatccaggcgtcgcacatcaccgtagagcgcaaagactacctgaagacagacatctccacggcgctgaaagagatccgctcccagctcgagtgtcactccgaccagaacatgcaccaggccgaagagtggttcaaatgccgctacgccaagctcaccgaggcggccgagcagaacaaggaggccatccgctccgctaaagaagagatcgccgagtaccggcgccagctgcagtccaagagcattgagctcgagtcggtgcgaggcactaaggagtccctggaacggcagctcagcgacatcgaggagcgccacaaccacgacctcagcagctaccaggacaccatccagcagctggaaaatgagcttcggggaacaaagtgggaaatggctcgtcatttgcgagaataccaggatctccttaacgtcaagatggctctggacatcgagatcgccgcatataggaaactactggagggtgaagagaccagatttagcacattttcaggaagcatcactgggcctctgtacacacaccgacagccctcagtcacaatatccagtaagattcagaagaccaaagtcgaggcccccaagctcaaggtccaacacaaatttgtggaggagatcattgaggagactaaagtggaagatgagaagtcagaaatggaagacgccctcacagtcattgcagaggaattggcagcctctgccaaagaggagaaagaagaggcagaagaaaaggaagaggaaccggaagttgaaaagtctcccgtgaagtctcctgaggctaaggaagaggaggaaggggaaaaggaggaagaagaggaaggccaagaggaagaagaggaggaagatgaaggtgtcaagtcagaccaggcagaagagggaggatctgagaaggaaggctcgagtgaaaaggatgaaggtgagcaagaagaagaaggggaaactgaggcagaaggtgaaggagaggaagcagaagctaaggaggaaaagaaaacagagggaaaggtcgaggaaatggctatcaaggaggaaatcaaggtcgagaagcccgagaaagccaagtcccctgtgccaaaatcacccgtggaagaagtaaagccaaaaccagaagccaaagccggaaaggatgagcagaaggaggaagagaaagttgaggagaagaaggaggtagccaaggaatcacccaaggaagagaaggtggagaaaaaggaggagaagccaaaagatgtcccagataaaaagaaggctgagtccccagtgaaagaaaaggccgtagaggaaatgatcaccattactaagtcggtaaaggtgagcctggagaaagacaccaaagaggagaagcctcagcagcaggagaaggtgaaggagaaggcagaggaggaggggggtagtgaggaggaagtgggtgacaaaagcccgcaagaatccaagaaggaagacatagctatcaatggggaggtggaaggaaaagaggaggaggagcaggaaactcaggagaagggcagtgggcaagaggaggagaaaggggtggtcactaatggcttagatgtgagccctgcggaggaaaagaaaggggaggatagaagtgatgacaaagtggtggtgaccaagaaggtagaaaaaatcaccagcgagggaggcgatggtgctaccaaatacatcaccaaatctgttactgtcactcaaaaggttgaagagcatgaggagacctttgaggagaagctggtgtcaactaaaaaggtagaaaaggtcacttcacatgccatagtcaaggaagtcacccagggtgactaagatcggagtccattgcaaaaggttaagccatacaacaatttcaaaatgcatgtgattgacagcttcaaaacagaatgggttctcccatgagggctccagacattgtattttcctttgtgcaatatgagggaactgcatgcaagctcagggtgcccccctcctcagtccttgggggaattcaaatgcatgttcatgtatgtacctagggaatttgccagtttcctaagctgttgaaagaggggcactcggggggatgtcttgagatgtattacgcaaagtaccaactgagccaaaaataataagtgaaacagaactctcttagccttaagaaagctatatatgaatacttatgtttacctcactggtgcatttaaaatggacttcagttcatgggagaaccttgctgacctgcacagttcgcaaccttatgttgattgatgttaaatgtcacagcagttcttgctcaataaaggtcatactggaaccataaaaaa

SEQ ID NO: 41 - DNA, 496 bp, Rattus sp.
agcaaaactggtaaaaacaaaattgtaatcgttgaacatagcgctctggcaatcaagacgtttgaaaccgtcaatcttctggggcgaagaaagcactgtgcgacacttagaactctgattaacagacaaggtggtcacaaattttcctggcttgaagacttccacaactttcctgatcaggtcatcataggaggtctgacttaggtttgtttcaaagctaacataagaaaattctggttctggagtgatgtgaatattccaatatgttccatccgatttcattccattcattgagtagccacaaggattgaacagtgtggcatcaatgacagaacctggtatcaggtcacgaattccactctcacgagtgacatcctttgcagtaacaccatctttcatgtagaactggtccataactgctgggtcaagctcactcatcagaatttccacggtttgatctggctgattgattactcggctctctgggagcctcgtg

SEQ ID NO: 42 - DNA, 458 bp, Rattus norvegicus
tttttttttttttttctgaagtaaatagagacatgtagagtcttccctccatgtcaggctgcacttcattagccccagctcagtaatgcagggaaccctagtgacccatcggccaagaaactcccagaagcattaaaaaaaaagttatattccgctgccaagtggatagtcatttagctgtttgtcccttgttttttatttattccataattatgtttgtgctttttcttgtgtgaacagtagtgaggcgtatgtttttatgtggctttagagaaaacttcagtcttcaaagaactgttctaattagttccttctcggaaaaagttatgcgttaatttgtttcaaaatatttaggcattctttgaattataaacttgtgatgcagggatttgtgaacgagacgttcacaagtgaagatgacttcacttagcatctgtgtaaacagaataagatgtgta

SEQ ID NO: 43 - DNA, 4757 bp, Rattus norvegicus
atctgtgtgcgagtgcgtgtgcgtgcgtgtgtgtgtgtgtgtgtgtgtgtgtgtgtgtgtgtgtgagcgtgtgtgtttttggatttcatactaattttctggagtttctgcccctgctctgcgtcagccctcacgtcacttcgccagcagtagcagaggcggcggcggccgccggttagagcccagtcgctgcttcagctgctgttgctgcttctgcggcgctctgctccctgcgctggctacgggaggccgggggagccgcgccgacagtcctctgtggccagggccggcactgtcctgctaccgcagttgctccccagccctgaggtgcgcaccgatatcgatatccgtgccggtttagcggttctgcgacccaaagagtccagggagagccaccgagtggcgcctggcgtataggaccatgcagccgccttgtggcttggagcagcggcccgtgatgttccagccactgtgaaccatttggtcagcgccaacctgctcagccccagcaccgacaggctcagcctctggtacgctcctctcggcgggaggccatcagcaccaagcagcaagagggctcagggaaggcctcccccctccggcgggggacgcctggctcagcgtagggacacgcactctgactgactggcactggcagctcgggatgtcgccctggccgaggtggcatggacccgccatggcgcggctctggggcttatgcttgctggtcttgggcttctggagggcttctcttgcctgccccatgtcctgcaaatgcagcaccactaggatttggtgtaccgagccttctcctggcatcgtggcatttccgaggttggaacctaacagcattgacccagagaacatcaccgaaattctcattgcaaaccagaaaaggttagaaatcatcaatgaagatgatgtcgaagcttacgtggggctgaaaaaccttacaattgtggattccggcttaaagtttgtggcttacaaggcgtttctgaagaacggcaacctgcggcacatcaatttcactcgaaacaagctgacgagtttgtccaggagacatttccgccaccttgacttgtctgacctgatcctgacgggtaatccgttcacgtgttcctgtgacatcatgtggctcaagactctccaggagacgaaatccagccccgacactcaggatttgtattgcctcaatgagagcagcaagaatacccctctggcgaacctgcagattcccaattgtggtctgccgtctgcacgtctggccgctcctaacctcacggtggaggaagggaagtctgtgaccatttcctgcagcgtcgggggtgacccgctccccaccttgtactgggacgttgggaatttggtttccaaacacatgaatgaaacaagccacacacagggctccttaaggataacaaacatttcatcggatgacagtgggaaacaaatctcttgtgtggcagaaaacctcgtcggagaagatcaagactctgtgaacctcactgtgcattttgcaccaaccatcacatttctcgaatctccaacctcagaccaccactggtgcatcccattcactgtgagaggcaaccccaagccagcacttcagtggttctacaacggagccatactgaatgaatccaagtacatctgtaccaaaatacacgtcaccaatcacacggagtaccacggctgcctccagctggataaccccactcatatgaataatggagactacaccctaatggccaagaatgaatatgggaaggacgagagacagatttctgctcacttcatgggccggcctggagttgactatgagacaaacccaaattaccctgaagtcctctatgaagactggaccacgccaactgacatcggggatactacaaacaaaagtaatgagatcccctccacggatgttgctgaccaaaccaatcgggagcatctctcggtctatgctgtggtggtgattgcctctgtggtaggattctgcctgctggtgatgctgcttctgctcaagttggcgagacattccaagtttggcatgaaaggcccagcttccgtcatcagcaacgacgatgactctgccagccctctccaccacatctccaacgggagcaacactccgtcttcttcggagggcgggcccgatgctgtcatcattgggatgaccaagatccctgtcattgaaaacccccagtacttcggtatcaccaacagccagctcaagccggacacatttgttcagcacatcaagagacacaacatcgttctgaagagggagcttggagaaggagcctttgggaaagttttcctagcggagtgctataacctctgccccgagcaggataagatcctggtggccgtgaagacgctgaaggacgccagcgacaatgctcgcaaggactttcatcgcgaagccgagctgctgaccaacctccagcacgagcacattgtcaagttctacggtgtctgtgtggagggcgacccactcatcatggtctttgagtacatgaagcacggggacctcaacaagttccttagggcacacgggccagatgcagtgctgatggcagagggtaacccgcccaccgagctgacgcagtcgcagatgctgcacatcgctcagcaaatcgcagcaggcatggtctacctggcatcccaacacttcgtgcaccgagacctggccacccggaactgcttggtaggagagaacctgctggtgaaaattggggacttcgggatgtcccgggatgtatacagcaccgactactaccgggttggtggccacacaatgttgcccatccgatggatgcctccagagagcatcatgtacaggaaattcaccaccgagagtgacgtctggagcctgggagttgtgttgtgggagatcttcacctacggcaagcagccctggtatcagctatcaaacaacgaggtgatagaatgcatcacccagggcagagtccttcagcggcctcgcacgtgtccccaggaggtgtacgagctgatgctgggatgctggcagcgggaaccacacacaaggaagaacatcaagaacatccacacactccttcagaacttggcgaaggcgtcgcccgtctacctggacatcctaggctagactccctcttctcccagacggcccttcccaaggcacccctcagacctcttaactgccgctgatgtcaccaccttgctgtccttcgctctgacagtgttaacaagacaaggagcggctctccggggtgaggcagtgcgcacttccccatccacagacagtatcgactcgcttctggctttgtcgctttctctccctttggtttgtttctttcttttgcccattctccatttatttatttatttatttatttatttatttatttatttatttatttatctatctatctatctatttatttatttatttattggtcttcactgcttcatggtcctcggcctctctccttgaccgatctggcttctgtactcctattcactgtacatagacaaaggccttaacaaacctgatttgttatatcagcagacactccagtttgcccaccacaactaacaatgccttgttgtattcctgcctttgatgtggatgaaaaaaagggaaaaaaaataatcaaacatctgacttaaaccgtcacttccgatgtacagacacggggcgtttctatggattcacttctatctatctatttatttatttatctatttatttatttctcttctttgttgttttccggtggttttagcctgtgtatgagaagggaaagtcatgtacagtctgggaaaactttatctgtgggaaatggaaaccagaaggggaaagaagctttaccataaagcacagcaggagtgagacacagaaaagccattggatcagccagagtccgtcctgcataggaaaacccagcagccatcaggctggaggatcatgttcggcactgacccccgaggacctttctgaggaggacacagaatgttaaactctgcatcatggacacagtttccgatcacagatactggccttcaatggaaaaaaaaaaaaaaaaaacccagatagttcttgtgagacctggacagcacgtccaacatccagacattgtggtcgggcacagtgacagagttgatgcatttctcacgggttattctacagagcttttgtcaagtccaatggaaggaggtagattcttgttcagatatgatttcgggaaaaaccgagtccttgacaaagacaggagacaccctcagttgggaggcaagtttctcttaccttggactttctcacacagcaattctcacccccaccccctccactctcacctgtcttgtaactgtgcaaacaaaagtgtgcatggtctttgtcagttgatacctttgtgcacctctgtgcagaaactgctgtctgtcccggctgtggtacccgatcagtggggtagatccacgaaaggtctcattttaggccgctttgggaaggtaaccagatcggtagctggaagcactctccagtaggtggcgaagggtgagtgggtctgctgaagcctgcatatcttcacccacctcaaacccaccgggctgcacaggggacaggcacaggccacccctgagggacagggaagctctcttgggataccacctgagtttacattcagtgtgctcaggtcaagtctctcgctcggggctctgtttcggggagaatggtttcattccaacgcactcattatcaggattctgttttc

SEQ ID NO: 44 - DNA, 861 bp, Rattus norvegicus
aaggcgcggatgagctcgctcgcccgcgccgagctggagaagcgcatcgacagcctgatggacgagatagccttcctgaaaaaggtgcacgaggaagagatcgccgagctgcaggctcagatccagtatgctcagatctccgtggagatggacgtgtcctccaagcccgacctctccgccgctctcaaggacatccgcgctcagtacgagaagctggccgccaagaatatgcagaatgccgaagagtggttcaagagccgcttcacggtgctaaccgagagcgccgccaagaacaccgacgcagtgcgcgctgccaaggacgaggtgtcggaaagccgccgcctactcaaggctaagaccctagagatcgaagcctgccggggtatgaacgaagctctagagaagcagctgcaggagctggaggacaagcagaatgcagacatcagcgccatgcaggacacaatcaacaaactggagaatgagctgcgaagcacgaagagcgagatggctaggtacctgaaggagtaccaggacctcctcaatgtcaagatggcattggacattgagattgcagcttacaggaaactcttggaaggcgaagaaaccaagctcagtttcaccagcgtgggtagcataaccagcggctactctcagagctcgcaggtctttggccgttctgcttacagtggcttgcagagcagctcctacttgatgtctgctcgagcattcccagcctactataccagccacgtccaggaggagcagtcagaggtggaggagaccattgaggctacgaaagctgaggaggccaaggatgagcccccctctgaaggagaagaagaagaggagaagaaggatgaa

SEQ ID NO: 45 - DNA, 5865 bp, Rattus norvegicus
  misc_feature (1)..(5865): where n may be a or g or c or t/u, unknown, or other
ctcgtgagaacgaatcgatccttcccagccttctctgcctgctctccacctcctctctgctccgagtcttaggagaacgaacattcaaaggacagattccaatgtggtgtgctgtgcacatcgcgagcggctggggtttgcacttcgagatttctttataatttttttttttaatgtaagggagacagtggaattgctacccgtagaatttttattcaagtgcacgtcgcgttgggttgcacgctccacccccagggacctggtgtggtgaaatttgaacccaccgccttagcccaaaggccgagtaacctggctgcttgagtgtcgtggaagacgtgagcgaaatgatcagcgaactcattttttatcagactcgctgaagctggcttttgcgtttttctacacgtacactaattttatggaatagttaaagtgctatattctccgcgcaaccttttcaaattccaaatgtttgaacgttttggtgtcagcgcgagtgaaatcattttaccgacaagaactaactgaattgtctgcctcgttgagttgcctccggaaaagatctcgggggtggaaaagcaactgcaaaataacagacggagaaaattccttggaagttatttctgtagcataagagcagaaacttaagagcaagttttcattgggcaaaatgggggaacaacctatcttcagcactcgagctcatgtcttccagatcgacccaaacacaaagaagaactgggtacccaccagcaagcatgcagttactgtgtcttatttctatgacagcacaaggaatgtgtataggataatcagtctagacggctcaaaggcaataataaatagcaccatcactccaaacatgacatttactaaaacatctcaaaagtttggccaatgggctgatagccgggcaaacactgtttatggactgggattctcctctgagcatcatctctcaaaatttgcagaaaagtttcaggaatttaaagaagctgctcggctggcaaaggagaagtcgcaggagaagatggaactgaccagtaccccttcacaggaatcagcaggaggagatcttcagtctcctttaacaccagaaagtatcaatgggacagatgatgagagaacacccgatgtgacacagaactcagagccaagggctgagccagctcagaatgcattgccattttcacataggtacacattcaattcagcaatcatgattaaatgagatggataaatatgaagttcatttggtttcagaaactcttgagtgaaaaatcccaggtcagacttctttaattaattaattgtttgctgttgctcagattgactgaatatttccattatctgtgtagaaaaaggaacgttaattataggagaaactttttcaatggacaaaacattccattctatctatattttaaagatcccttttgctaaccagttttctgattttctacatgttacgtaagactaataacttgtgattaggatcaatggactcctgctccaaaggaaagccttgccacaggcccacagaggtgccacagaggacggggccaggcaggaacccgtcagcattgaaggttgtttttgtatgccaacaggaggaaagcttgagttgctgctgattcttaaaagaattctgtattctaaaagatacacatcatgttctaaatgcattttaaactagtgacattagttattgggcatactgtggtattactagactacaaagagggatatgaagtggcaccattganagtatttttttaaaagcctgtctaccttaacactaatttttacccttatttaaatgctttttactaaacagttttaggtaaaattaagaaaacagttttgttgactgcacatcttttagaaggaccaacttttagagaattacattctttgacagattaaaaattgcaaagtgagatatttcaaactcttaagtgagttttattgccgttggactgcattaatacggacatacgattaaacttagtagaccaacactgagggatctccttaccaggctgcagaacaaggaaattaagcaataaatgggacttgtgaatggaaggacactctactgctagtgctagtaattctgcataagatggtatacattttgaagaaagctgcttttaattacttttaataatgattttaattactctagtgcaagtgcttcctcgagctataaaggtagctgagcacagcagacctttattccctcagtctgactcctgtactcatattcatttagtgaacatagtcttttaacagaagaccacagttctttgatagcattacaanacttacgttatttaaacgttataaagaacgttattgtaggataaaatgttaaaaactgtatcaaggacaggaagaattcctatctattaagtagtggtttccacctccacttaagactgaactgcactgaacaggtaactgtatacttggtctgacacctagattgaggccatccgcactgaatactgtgacatttaggagtaagaacttttaaatttaacatttaaagaagctacttccagtttatgcacctaaatttgtctaaatgttttccattttgctgaccccattgtattcatactgctccccagagcctagagttgtcctcatcctgacttcctgtccctgagtgtctgagaggagtcactttcactgtaaagacactgcttctgcgccttgtagggaggacttgacagtgctcccatagaaatcctacattatttcaacctcatagttacagtaaaggcaggttataaccagtctttcttattattttaagaatttccagccctagtgttttatgaaagtattcctgtgaatttgatatcttatgatcctatattcatctaattccttaactgaaataaaaatgtccatgtgaggtaggttatttacagcgattgcaggagacatggtgttcttcagagttcccaaaccaggatagtttcaaataggtttttcatggcttctgacgaagaagaccgtaaagttccctgcagtgtgtcagtgatgtgcaagctgaattagtgcaaagtgtcacactgtgaaagcacgtgcttttggcttattatgagaaaacgaaatctttaaattcagtttatgtgtcttaggtccagtttactttgatttgacaactcagttcttctgaccccaccctagtatgtatgtatatgtgtgtgtatgtgtgtgtatgtctgtatgtatatacatacatatacacacacattgtatacatatgctatatatacagtatgtgtatatatatactatatatgaatatatgaatatatatattcaattagttaatagtacatttaagccaaatatccaacataagcacactatgtaagtatctatctggaaagacctatatagaattgagatcaacatttcatgagttagaaacaaaggattttataattaatattacttaagtctaaagtacccatatatttaaattagatatgcaatttttccctcttggcaaagaaagacaaaaatcttgtgtttagagatgatgtagattgtcatttttgcctttccttcctgagtacttgttttaacaacaacaaaaaaagactagtttaagaaaagggattgtccagtatttttctgctttgttaagtctaattttactgttaaacagagagcagaatcactggagtactgggggggttttttgttgtttttttttttttttcttttctgtttttttcggagctggggagcgaacccagggccttgcgctcactaggcaagcgctctaccgctgagctaaatccccaacccctggagtatctgttttaaaagaaagccaggaccgttatgatggccatacccngggtacatagtgaaaacaacagagaccaagcaatgagagtgtgagagtaccaatccaccagtactgctgccggacatggcagctgcctgtgcttttctgaagagtcatagtgtatgctaagtctagaaccattacttagtaaagaggctatgacttttatttgggcctgaaaattttagtggtgtggtcatagtctattctgtatttgtaagctttatttttaaattactgtgttgatttaggaacacaagaaatgtttttatttttaattatgagtgtatataaggttttcagatatgcacagactacaataatagactcccatggagataccacttcagccttaacagtcagggagaaggagcctcactttatcaccgcactcaccctgctctccactgatctgttgttactgcggtgtggaggttcacacgcatgcaggtcttcacacatgatgggtaggcccgcaccaagtgagcctctcccagccttgccgtttcgtttttttattttaatcttacatgtatgggtgttttgcatccaggcatgtcatgcctgtgtccacagaagccagagagggtatcagattccctaaaactggagttctcgatgatcgtgagcgngccattgtgggtgctgggaactgaagctgggtcctctacaagagcagccagcgctcttaaccattgagccactatctgccctgtgttttatttatttatttatttatttatttatttatttatttatttatttattggttctttttttttggactggggaccgaagccagggccttgcacttcctaggcaagcgctctaccactgagctaaatccccaaccccttgttttatttttaaagcaaacgagatacataatttcaaccatgataatttaagattatcttgaactcttaaggaaatgtatatactaagctattatagtttttattttccctaattcagtggcataataccttaccttgagtcgtttactactttctttggtttctaaaaactctactgctaaattacaatgtaaaaacatagggctcgtatatactgtagagtgctgtagatgtcctcgtcatcaactatgcaataacagtctgatcgacacatttcaggagcgatcactctttggtgtgcttctttaaatactttcagaagcttaggatgtgcaaagcaggaagaccgtgggtgtaaatgtttacttatttctttgagagtgttagtaagtcttttctaaattgcttttctcttcaaaattatcgttaacttaaatgataattatctttgaggttaaacagaagctcattgacaaactaaagtgaccttttagggcattctttgagatcatagtcttatatctggggactaaaatgtcattagaccctaatagactaacttgtatgtttgtgtggggaaacgttttcctctctcattcaaggtaactgtttgctgcctgttgttacttgtgtagcattctagaaaatggctaggtttttttataagatttaagacaatagaagtagttttatattattatagttctgttggaatgtgatcctgaaattattactgaaaattagaatttttatttcgctaatgacaaccttgactctcagagatgcagtgtaaattgatacctcatctttccgagagttcagagcacagggcggcagtatgtgaagctgcttttgcactgacgcattttgataagtttggctactgtaatggtaaaaggctcctcaggcactgactgcatttgggttcttccgatgggggatgatccgttctcgtggtgctgctggacttatgcattttggaggtactgcatgtatcttccacactgcttgacattttctctgatctgtgtgtttgcaccaactcattaaaagaaatatgcagaaatatcttctaattcgttgatcttcgctgtatgacagttataatattaaacacttgggttgatccac

SEQ ID NO: 46 - DNA, 2295 bp, Rattus norvegicus
aatccgggctgagagaaggacgcgcacggagtggtgcggccgccacccggggcagacagcagcgggtgctctactgtgcccggaatcccggtcagaacatccagtctaccactgttctctgatgccatgccagcaccaactcaactgttttttcctctcgtccgtaactgtgaactgagcagaatctatggcactgcatgttactgccaccacaaacatctctgctgttcaccaccgtacattcctcagaatcgtctgagatacacaccccatccagcatatgctaccttttgtaggccaagggagaactggtggcagtatactcaaggaaggagatatgcttctacgcctcagaaattttacctcacacctccacaagtcaacagcatccttaaagctaatgaatacagcttcaaagtaccagaatttgatggcaaaaatgtcagttccattcttggatttgacagcaatcgtctgcctgcaaatgcacccatagaagaccggagaagtgcaacaacctgcttgcagaccagagggatgcttttgggcgtttttgatggtcatgcaggctgtgcttgctcccaggcagtcagtgaaagactcttctattatattgctgtttccttgttaccccatgagactttgctagagattgaaaatgcagtggagagtggtcgggcactgctacctatccttcagtggcacaagcaccccaatgattacttcagtaaggaggcatccaaattgtatttcaacggcttgaggacttactggcaagagcttatagacctcaatactggagaatcagctgatattgatgttaaggaggctttaattaatgctttcaagagacttgataatgacatttcattggaggctcaagtcggtgatcctaattcttttctcaattacctggtgcttcgggtagcattttctggggctactgcttgtgtggcccatgtagatggtgttgacctccatgtggctaacactggcgatagtagagccatgctaggtgtgcaagaagaagatggctcctggtcagcagtcacgctctctaatgaccacaatgctcagaatgaaagagaactacaacgtctaaaactggaacacccaaaaaatgaggccaagagcgtggtaaagcaggatcggctgcttggcttgctgatgccctttagggcttttggggatgtaaagttcaaatggagcattgaccttcaaaagagagtgatagagtctggcccagaccagttgaatgacaatgaatacaccaagtttatccctcctaactatcatacacctccttatctcactgctgagccagaggtaacttatcaccgattaaggccacaggataaattcctagtgttagcaactgatggattgtgggagactatgcatagacaggatgtggttaggattgtgggtgagtacttaactggtatgcatcaccaacagccaatagcggttggggggtacaaggtgactctgggacagatgcatggccttttaacagaaaggagagcaaagatgtcatcagtctttgaggatcagaatgcagcaacccatctcattcgccatgctgtaggcaataatgaatttggagctgttgatcatgaacgactctctaaaatgcttagccttcctgaagagcttgctcggatgtatagagatgacattacaatcattgtagttcagttcaattctcatgttgtaggagcatatcaaaaccaggaacagtaagtgatactatcctggcagttcttcattttgaagggcagatcaatgttcaaaagaaactaatacagtaaatatcccagtgggtcattctaaacacatcatgtttagtactctagctagcccagtcttcatatctactgcatcagatgatagcatcatgagtgtctgttctgtcctgttggacctcagggtacctgcacttgaggcagcttgtttccttacccaggtgtcttttaacaatgactcaccaactaagaatatggataggtagatacgatcttgaataggtcaaaagcaaggaacttctgggagtattagtaaaggtagaaaacatcaccatgcccacctgcagactccttccatcataagactcctaatgtacatgagaatagttatttactgcatttttcagatgaacagttcaggtattcacatacatttatgttagcctaaagtgcaggtccagtattttcagccatttatgaccatgaacaaagaaatctggatttgtaagtttttgtgattgtgtgctaaagtatgcttcctgaacggaaa

SEQ ID NO: 47 - DNA, 386 bp, Rattus sp.
gaggtataaaaatctgtatttatattacaatgacataatgacacagcacagccccatctgttagacaggtgggatggggtcccattccagtctgcagctcaggtctcataactcacccactgcactggcaccaggccccttgtgtagccccagactctggcacggaacctgccctagaacagcaagctcaaccacctgcaggttagaaatacagaggcagacctagggcccatatggagacaaaacctccgtgtcctctcctgctagacaagctgggtctgcagcaggatggtgggtctggtacatctggaccacctcctacgaaagaggcccaagcctgagcctggagttgcagactctgttatggagcacacgtggctcacatg

SEQ ID NO: 48 - DNA, 387 bp, Rattus sp.
catatatagctgtgttcagacacatcagaagagagcatcagatctcattacatatggttgtgagccaccatgtggttgctgggatttgaaccaggacctctgtaagagaagtcagtgctcttaacactgagccatctctccagcatttgttttgttttaaaacaatgaccataagaggaatggtggacttctagcttgttctgcctacagtaacaaaagcacatcacacccccagctcacaaacctgtgtgcaatgtcactaccaccacacaacggtactgcgttacactgttggaaaccgcatttccactcagtgcagataaaagcaataggaaacacccagcccagagtcagattattttctgttgtgaagtcaggcctcgtgcc

SEQ ID NO: 49 - DNA, 425 bp, Rattus norvegicus
tttttttttttttttttacacatgcgcgggctttattataaagcacgtttcttgacctggggcctccagctaggatgataggacagcacccagtctcggtttaccaggactcgcaacttatcacaggcgcctgagggctgtccagtaacggagagggaacatagccacttccggaggcgtatggggtaaggtgtgtggaaaactttgaaaactccaaagcaagggccacccctccatggctattgcacgcaccggaaggaactcaacgctgtacgtcagctcttctccctgttaaagggacacagcgatccttttttcctaaggcctcgaaagacttccgcccacacccaatatggcgggcgatgggagcgggtagaattccgggaatgtggagcagatcactgggacggagtggagcctcgtgc

SEQ ID NO: 50 - DNA, 647 bp, Rattus sp.
ggattcttttcttttattattggcatcagctggactatatgtggccttaaacatcatgcactggacaggaaagaaagaacgagaaaaaaggacacaggaagggaacacgagtagcggcgagattccgtaataaaaactgtaattcattcaataggttaagttttggcattatgaaatcaaacaccccttcccacccccaaaagttgaaacttaagtgagagggtcgcccgtgggacccagccaggtgttctgcgttccctcacaggctgtgacaggcagcgtgtgggtgtaattcttgtgaacggtctcaacttttcactttctttaaataatctcttttgcttcttagacgttccggttcactggggtgacataattgcggggaaacatgccggtctgcccatggcaagcccctttccaccaattgggatctgagttatccatgacatgaatgaagtctcctcggcgaaagcccagctcaccatcctcctgggggtcaaagtcaaagagtgcctggacgtacgttggctgtggcacctgttctatgtcccggaggaatatctgctggttcctggacacggatgttgatctgtggtaatctaccagctcattcaaagaattaaacttcaccacccacaggaagtact

SEQ ID NO: 51 - DNA, 613 bp, Rattus sp.
aagaagtcattggagtttattcacagttaatcactacctaccaaattgctattcgcagaagttagaggcgtaagtacataggtttgtttttttatttaaacactgatctttaaatatatacacacaaaacttagttcagcaaggcttcatgatatacaccaattccaaaataaaacaatcaaatggcccaggtgtaactccagagataaatttttatcatcagcagggaaagaggcagcagaaccaggaggggtgggaacaggctccgcccaggacgcctccggggcctcaggggtgctccgagctgagtccatctcacaaaacagagtccaggggggaccccaccctcaagagtccagcagcccacggacgccgccaactccaagggagagcctgggagtagccatgactctgctgtggggaggaggctgcgtggagagaaacggtggacaggcacatgactacggaggactgagagctcacaggagacaagatttaaaaagctttgcgttgctcttggtcttttctacctcaaaaggcttcatgggatggggcagcagagcagctgcaaacaccactgttgtgcacagagatgcaaccatgcaaccagccc

SEQ ID NO: 52 - DNA, 458 bp, Rattus norvegicus
ttgaatgcattttttaaattttattgtttttcagtatgacagtgaaaattttacatagcccatgttgaacttagatattcaaataagactttaccaataataaaggagattaatacattgctagagttctacatttgactttatgtcaaaagagtcaatttagctctatgaattacagaagactttgtattctgttccttttagctttattaaatgttgacatctatgattacatcagacttcgatacctcagttcacatctacagaaaattttaaaagtgattctgaatactacagataaaatatttttattgtaggtaattattcaatctgtatatttggcttgatgcatgataagtcatacgggaaaggcatccgcgctatacttgaatttccagaatctagcattctttctttttcaaaatatttttttctttttagcaataatattgacagat

SEQ ID NO: 53 - DNA, 6820 bp, Rattus norvegicus
ccgagaacggctgcagtcctctgacctgagaccaatagctgtgtctacccggactcagcgtccagctcaccgccactaacgcgccgcgcattggacacctgatccacacaccttcgggcaccagtgaaaaaccgcgacttgattttctggaagaacgcccccagggtgtgggagcggtcgtggaggaccagcaggaggaagcggaggggagaggggcagtagtggaggcagagaaagcgttgaaccagctgtgttggccgaaggcacgaaacggcaaaaggcagcggtgagcatctgtgtggttcccgctgggaacctgcaggcaggaccggcgtgggaacgtggctggcccgcggtggaccgcgtcttcgccacaatggtccggctcctcttgattttcttcccaatgatctttttggagatgtccattttgcccaggatgcctgacagaaaagtattgctggcaggtgcctcgtcccagcgctccgtggcgagaatggacggagatgtcatcatcggagccctcttctcagtccatcaccagcctccagccgagaaggtacccgaaaggaagtgtggggagatcagggaacagtatggtatccagagggtggaggccatgttccacacgttggataagattaacgcggacccggtgctcctgcccaacatcactctgggcagtgagatccgggactcctgctggcactcttcagtggctctcgaacagagcatcgaattcatcagagactccctgatttccatccgagatgagaaggatgggctgaaccgatgcctgcctgatggccagaccctgccccctggcaggactaagaagcctattgctggagtgatcggccctggctccagctctgtggccattcaagtccagaatcttctccagctgttcgacatcccacagatcgcctattctgccacaagcatagacctgagtgacaaaactttgtacaaatacttcctgagggtggtcccttctgacactttgcaggcaagggcgatgctcgacatagtcaagcgttacaactggacctatgtctcagcagtccacacagaagggaattacggcgagagtggaatggatgctttcaaagaactggctgcccaggaaggcctctgcatcgcacactcggacaaaatctacagcaatgctggcgagaagagctttgaccggctcctgcgtaaactccgggagcggcttcccaaggccagggttgtggtctgcttctgcgagggcatgacagtgcggggcttactgagtgccatgcgccgcctgggcgtcgtgggcgagttctcactcattggaagtgatggatgggcagacagagatgaagtcatcgaaggctatgaggtggaagccaacggagggatcacaataaagcttcagtctccagaggtcaggtcatttgatgactacttcctgaagctgaggctggacaccaacacaaggaatccttggttccctgagttctggcaacatcgcttccagtgtcgcctacctggacacctcttggaaaaccccaactttaagaaagtgtgcacaggaaatgaaagcttggaagaaaactatgtccaggacagcaaaatgggatttgtcatcaatgccatctatgccatggcacatgggctgcagaacatgcaccatgctctgtgtcccggccatgtgggcctgtgtgatgctatgaaacccattgatggcaggaagctcctggatttcctcatcaaatcctcttttgtcggagtgtctggagaggaggtgtggttcgatgagaagggggatgctcccggaaggtatgacattatgaatctgcagtacacagaagctaatcgctatgactatgtccacgtggggacctggcatgaaggagtgctgaatattgatgattacaaaatccagatgaacaaaagcggaatggtacgatctgtgtgcagtgagccttgcttaaagggtcagattaaggtcatacggaaaggagaagtgagctgctgctggatctgcacggcctgcaaagagaatgagtttgtgcaggacgagttcacctgcagagcctgtgacctggggtggtggcccaacgcagagctcacaggctgtgagcccattcctgtccgttatcttgagtggagtgacatagaatctatcatagccatcgccttttcttgcctgggcatcctcgtgacgctgtttgtcaccctcatcttcgttctgtaccgggacacacccgtggtcaaatcctccagtagggagctctgctatatcattctggctggtattttcctcggctatgtgtgccctttcaccctcatcgccaaacctactaccacatcctgctacctccagcgcctcctagttggcctctcttctgccatgtgctactctgctttagtgaccaaaaccaatcgtattgcacgcatcctggctggcagcaagaagaagatctgcacccggaagcccagattcatgagcgcttgggcccaagtgatcatagcctccattctgattagtgtacagctaacactagtggtgaccttgatcatcatggagcctcccatgcccattttgtcctacccgagtatcaaggaagtctaccttatctgcaataccagcaacctgggtgtagtggcccctgtgggttacaatggactcctcatcatgagctgtacctactatgccttcaagacccgcaacgtgccggccaacttcaatgaggctaaatacatcgccttcaccatgtacactacctgcatcatctggctggctttcgttcccatttactttgggagcaactacaagatcatcactacctgcttcgcggtgagcctcagtgtgacggtggccctggggtgcatgtttactccgaagatgtacatcatcattgccaaacctgagaggaacgtccgcagtgccttcacgacctctgatgttgtccgcatgcacgtcggtgatggcaaactgccgtgccgctccaacaccttcctcaacattttccggagaaagaagcccggggcagggaatgccaattctaacggcaagtctgtgtcatggtctgaaccaggtggaagacaggcgcccaagggacagcacgtgtggcagcgcctctctgtgcacgtgaagaccaacgagacggcctgtaaccaaacagccgtaatcaaacccctcactaaaagttaccaaggctctggcaagagcctgaccttttcagatgccagcaccaagaccctttacaatgtggaagaagaggacaataccccttctgctcacttcagccctcccagcagcccttctatggtggtgcaccgacgcgggccacccgtggccaccacaccacctctgccaccccatctgaccgcagaagagacccccctgttcctggctgattccgtcatccccaagggcttgcctcctcctctcccgcagcagcagccacagcagccgccccctcagcagcccccgcagcagcccaagtccctgatggaccagctgcaaggcgtagtcaccaacttcggttcggggattccagatttccatgcggtgctggcaggcccggggacaccaggaaacagcctgcgctctctgtacccgcccccgcctccgccgcaacacctgcagatgctgcccctgcacctgagcaccttccaggaggagtccatctcccctcctggggaggacatcgatgatgacagtgagagattcaagctcctgcaggagttcgtgtacgagcgcgaagggaacaccgaagaagatgaattggaagaggaggaggacctgcccacagccagcaagctgacccctgaggattctcctgccctgacgcctccttctcctttccgagattccgtggcctctggcagctcagtgcccagttcccccgtatctgagtcggtcctctgcacccctccaaatgtaacctacgcctctgtcattctgagggactacaagcaaagctcttccaccctgtagtgtgtgtgtgtgtgtgggggcggggggagtgcgcatggagaagccagagatgccaaggagtgtcaacccttccagaaatgtgtagaaagcagggtgagggatggggatggaggaccacggtctgcagggaagaaaaaaaaaatgctgcggctgccttaaagaaggagagggacgatgccaactgaacagtggtcctggccaggattgtgactcttgaattattcaaaaaccttctctagaaagaaagggaattatgacaaagcacaattccatatggtatgtaacttttatcgaaaaaaataataaaacgtaaaaataaaatcaacaaaaataatctcttcttttgctcaatcgtgcatacatatatctgcccacactcccgtggtaaaactagaagcgaagcaggccctgcgatggtgccaactgaatcctaagttcatcatcctagtgagcagatggagagagggcaggaggcgggggtaggttcggacaacagctcccatctcagaccttgactgtgctgagtcttcagactcctggactaaggaagacccggggactgaccttatgagggtccctttccactgctgtgatccattgccagcctgtagtcacccgggataaaggcacagtaaccttttgcattcctgtgattccctgtgtttaaggaaaaggaaagtatgagcaaagctatcaccaaaaagagcgccattagaagttacgggggagaaaaaaagagaagcaagatgatatataagcacagggccttgaacaaggtgagcgtgcttcacagattccgtattaatgtacagatacttttggagaggagaaagataacaaggagtgtcaggccgtttgtgaactcacttgcactgtgccaaccaggttctccgctgcccttcagcaaaagaggacaagccgcgttgccaggttttaccttccatttactgtagcaaatactatcaaccagtcggacttctaagattcagtttcagtttcagtacaatgcggtgccactgtttctcccatgtgctatggaaacgaatctatctttgaacttaatgatgtattcatagcaactattactggtttagattttttccttttgtcacaggagtccctggaactagtaactgaaagtgttttcctgcgtttcttgtatacatgtgattatgaaattcgtgccatttaatgtcaatttagctgtcactagaagactgtcttttggatatagtataaatatttttatgtaccagtgatgttctccataccacggttaccatgtttctctggaggttgggtctgtggtctgatgtttctcatgtgcagcttcgatgggaattcttctaagtgggatttatttttcagatattttatgatatgagaatgttattaatgaagtaatttgaaagtgcattgtataaaaatggtcaccaagcaatgcgtgacagtaaaaggtccgtttttataaacctgcgcacattgttattaaaatgtaaggttgaaaaggcaatatttagaatatttcagatatatttttaaaaagtttttccacagctacttgagtttcatggtcttctagtatataacaacactcaagtctacccagagtgtctcaactatctgcttgtcaattctgcttaattttattttcatgcatttaaacttttatatctttgttagcatctcttccttatgatcctcatgtgtactattatgtaataaccacatacatgtaatatccacatacatgtaatatccacacatgtaacattcacatacatgtagtccagttattccatcttgaccctaccttttcgaacccaaaagaaaattgttcttgttatttttatttcttctgttatttgtgagatgaacccgttccctttaaataatctttgtttgtgccttatgttcagtcattttaatttgctgtcttcatgtcgaagctgctggtttctcagccaaaaagcatcatcttagactctctaaatagccaaagcatcatgagtttggaatttaacatcagctcccatgtcagagttgtgctcctcatgtgatcccacattctactgcccagtgtagtgaattcctttccaagaactcttgcctttgctttccaagttatttttgagcatcttggttgcagagatctcaagaatttacgtcttggattccacgttttcactacgaagaaacagaatgagaagaagaagaaaaattaggcagtgtagagctgggcgtagtggtccaggtctttaagcccaggctagcctgatttagccaataaattctaggcctaaaaagagagacctgtctcaaaactcaaagcacacaacagatgctaagtagatgggtctccataattgggaagccaatgagagaatgcatatttcttcctatgttctttaaaacttgaagcagttacatccgtctttcatcattacgggactcgtgcattcagagccttttgttgttcttttgccagaatagatgaggcaacatttgcctattcgaatgctgtaacaggcaagttgactctagggttttggtctgagacatttggtgaacaccttcaacactgattaaaatattactgaatgcctactcttatcctgattatgaatcttccagaataaatagaatattagctcatataattgttcagaattggagatgtatgcctactaccctgtacctaaagggcaaaaatatcttcactgtaatgtgtgtgcttcttcaaggtgttttgcttcttgtaaaagtgttttcctttggcttgttactgccttttgtcagataatcttgatgacgctgtatcataataaatattttctatttatt

SEQ ID NO: 54 - DNA, 759 bp, Rattus norvegicus
cctcaggctcagacacctgctctactccaagcaaatggctgctcttccaatgctgtggaccgggctggtcctcttgggtctcttgggatttccacagaccccagcccagggccatgacacagtgcagcccaactttcaacaagacaagttcctggggcgctggtacagcgcgggcctcgcctccaattcaagctggttccgggagaagaaagagctactgtttatgtgccagacagtggtagctccctccacagaaggcggcctcaacctcacctctaccttcctaaggaaaaaccagtgtgagaccaaggtgatggtactgcagccggcaggggttcccggacagtacacctacaacagcccccactggggcagcttccactccctctcagtggtagaaaccgactacgatgagtacgcgttcctgttcagcaagggcaccaagggcccaggccaggacttccgcatggccaccctctacagcagagcccagcttctgaaggaggaactgaaggagaaattcatcacctttagcaaggaccagggcctcacagaggaggacattgttttcctgccccaaccggataagtgcattcaagagtaaacacaggtgagagaagtcagtcacaggtaacacatggtgatgtggcctcaggactcccgtgctctgtcactcttgagacccaagccctggctccccaaagaccttctccgccctccagctttgccttggtggagaaataaaatccaaagcaagtc

SEQ ID NO: 55 - DNA, 2591 bp, Rattus norvegicus
gaggaggaaggcgctgctggcgagcctcagccggccgctcagggggctaaggaggtctggagggctcggcgcgactgagggccggaccactagaggtatcgggtggcgctgcggcagcagcaggtgcgggtggcggaggctgcggctccagtgccatcccttattccacctgcgctgtcagcatgcaccttgcagaaccagaagcagcttgctggctttgaacgtgtggcaaatatttcagaaagcttcaagatcaagttggaggaaaggacggtttttcttctaaattcatctgcttcaactattattcttactgggaatggacaatggaatgctctctagatttatcatgaccaaaacgctccttgtcttctgcatttccatgaccttatccagtcactttggcttttcacaaatgccaactagttctgtacaagatgagaccaatgacaacatcacaatattcaccaggatcttggacgggctcttggatggctatgacaacagactgcggcctgggctgggagagcgaatcacgcaggtgcgaacagacatctatgttaccagctttggcccagtgtccgacacggaaatggaatataccatagatgtatttttccgtcaaagctggaaagatgaaaggctgcggtttaaggggcctatgcaacgtctccctctcaacaaccttcttgccaggaaaatctggaccccagacacattcttccacaatgggaagaagtccattgcgcacaacatgacgacacccaacaagctgctgaggctggaggatgatggcacacttctctacaccatgcgcctgacgatctctgctgagtgtccaatgcagcttgaggactttccgatggatgcccatgcttgtcccctgaaatttggcagttatgcttaccctaattcggaagttgtctatgtttggaccaatggttccaccaagtctgtggtggtggcagaagatggctccagactcaaccagtaccacctcatggggcagacagtaggcactgagaacatcagcaccagcacaggtgaatatacaatcatgactgctcattttcacctgaagaggaagatcgggtactttgtcatccagacgtaccttccctgcatcatgacagtcatcttatcccaggtgtctttttggcttaatcgagaatctgtcccagctaggacagtttttggagtgaccacagtgctgaccatgacaaccctcagcatcagtgcccggaattcgctgcccaaagtggcctatgccacagccatggactggttcattgctgtctgctatgcatttgtcttctctgccctgattgaatttgccacagtcaactactttacaaagagaggatgggcctgggatggcaagaaggccttggaagcagctaaaatcaagaaaaaagaacgtgaactcatactaaataagtcaacaaatgcttttacaactgggaagttgacccatcctccaaacatcccaaaggagcagcttccaggcgggactgggaatgctgtgggtacagcctcaatcagagcatctgaggagaagacttctgagagtaaaaagacctacaacagcatcagcaagatcgacaaaatgtcccggattgtgttccccattttgtttggcactttcaatctagtttactgggcaacatatttgaatagggagcccgtgataaaaggggctacctctccaaagtaagacaggaaaccatacttgcacagaaatgaacctgaggagaggtcaagctcacagagactatttgggcgcctgtctttcaggaaattttgcatgtttaataatatgtacaaataatattgccttgatgtttctatgtgtaacttcaatgtttcaaggatgtcccttaataaaccaagcaaatggccttctacaacaacgggaggcaatgactgactctcagatgctcagcgtcctaacatcaatagtttacaaacaagataagtatatttttaactgttctggtatatgacgtttttatacttcgaatgccatttcgtaccatttttcccagccaacagaacattttagggaatccctgtgatgaccacttgacaggtgaaaaagcaaagatcctcgggtacacaaagtccatgaagagcaaactgtggacatttaagtccagtacgaattgcctttaacaattcttcttgttctgaaattagaaaaatactgcatgaactgacattaagaagtagataagcaaacatttatgcagacaaatttaatgacaagcccatagtgtcttagattagtagatcaaataattccccaaggaaaagaaatcaactgattcaaaattaattttgttgttttttgtgaaaaatgaatttatttctcaccccgccccaacacctttacaaccttaataatgactaagaaagcaaaatcttaaaaccttaaagcaacaaggccttggtctttggtggtggtattctgtggccattgtttctgaccctgggtcctcttgctgctgcttcagcgctgagaaattgtaattgagttattttctgttttatttccctgtacatatttcatggttggattatcgctctgttag

SEQ ID NO: 56 - DNA, 2977 bp, Rattus norvegicus
gaattcggcggatggaagccagctgtcccgagaagcagtgaactgtggcgtcatcccgagcagtgccttaccggtattgtgctgcttcacctgcctcgctcggcgttctcctcaggccccgccatggagcgacaggtccaacgacttcgccagacgttccggtccggccgatcgcggccgctgcgtttccgactgcagcagctcgaggccctccggaggatggtgcaagagcgagagaaggacatcttggcagccatcgcagcagacctgagcaaaagtgaactcaatgcatacagtcatgaagtcattaccatccttggggagattgacttcatgctggggaatcttcctgaattggcctctgctcggccagcgaagaagaacctgcttaccatgatggacgaggcctatgttcagccagagcctctgggagtcgtgctgattattggagcttggaactatccttttgttctgaccctgcagccactggtgggagccattgctgcaggaaatgctgccattgttaagccctcggaactcagtgaaaacacggctaagatcttggctgaactcctccctcagtatttagaccaggacctgtacatgattgttaatggcggcgttgaagaaaccacagagcttctgaggcagcggtttgatcacattctctacacaggaaacaccgcagttggaaaaattgtcatggaggctgctgccaagcacctgacccctgtgaccctggagctcgggggcaaaagcccatgctacattgacagagactgtgacctggacgttgcttgcagacggataacctggggaaagtacatgaattgtggtcagacctgtattgctcctgactatatcctgtgtgaagcctcctcccaggatcaaatcgtacagaagattaaggatacggtgaaggacttttatggggaaaatgtaaaagcttctcctgattatgaaaggatcatcaaccttcgtcactttaagaggataaaaagtttgcttgaaggacagaaaatagcttttggtggggagactgatgaagctacacgctacatagccccaaccatactcactgatgttgaccctaactccaaggtgatgcaagaagaaatttttggaccaattctcccaatagtgtctgtgaaaaatgtggaggaagccataaatttcataaatgatcgcgaaaagcccctggcactctacatattttctcacaacaataagctcatcaaacgggtgattgatgagacatccagtggtggagtcacaggcaatgatgtcatcatgcacttcactgttaattctttgccctttggaggtgtgggtgccagtggaatgggggcttatcatggcaaatacagtttcgataccttttctcatcagcgcccctgcttgttaaaagggttaaagggagagagtgttaacaaactcaggtaccctcccaacagcgagtccaaggtcagctggtcgaaattcttcctgctgaaacagttcaacaaaggaaggctgcagctgctgcttctcgtgtgcttggttgcggttgcagctgtgatcgtcaaggatcagctgtgatgacttccttgtagcctctactgaagtacccctcggccaaatggttaacacaccaatgcttttaaaattgtacccaaaccaggaaatgaaattcacaggtgaactgcagtcaaacctaagttgttgccacaaaccactgatgaaactcagtgcttcagccaaatcccagcatttgtcagccgtgcaggtgctgagagggtggagactgggaggggcgacacctagtccatggcagcgggatgtcagggagactcgacaactgctcccgcactctttgctccaggacatagctctcccacccggtgtcaacaccctccaggctttccagctgtcctctgattgctgaggttcctgttagggacccaggtactaaacctgggcgggtggatttgtcggcctcatccattgtggctcgagaccggccttcgggagtcggctctcagtctaaacatcctttctcattcatagtgtgtcacccgaagatgcttgtttgtgacattgtgacagtctgtcatgactgtcccggtgcctttgtgatgacttaaactacactgaggagcttgccaacttgtgaatgcccttcagagggtctggcagtcacagctgttccagagcccgagggacgaagattccggagcccggagtttgaggccaacctaggcaacataatgggaccctctcattattattcctccataacaatcccctcgagaccctcgatttgaatgttatataggtcttcaggataaatctgcttattttcacagcacaacacaaaaaaaatttacttttgaaatcttagagagattcctacagatcttagcatggagctgttcctgtagtgaaaggggggttattagacatgaggcttcagaactcatggggcagggttgttggagactaccgtgagctgagggggcacactgaagcgatgggatggccagaagcgcacctgagcaagcggggcagcattctctgtcagaccctaacatggctacacggggatgtggcagagagatctgtgccgttggctgccagcgctggttaggcctgaagctccaagctgcagaggtctcattgccttcccaggatccaaattaagactgcccactcaatgagaatgtcacttgcgtatgtacaaccatgtttgctgagtaacctgttccaccgttgaggctgtctgaagtgtattgtatgaggtatcaagaacgagtcattggcccatttggcaaatagttgcttatgtagcaattgtcatggactaatcataaaatattttgcacaaaatttcaatgttgaacttgcactcactgttgttaaattataaatcacagcttctagttaggccaaaatatttacatactctactaatcttcaaaataaatgtatcccggaattc

SEQ ID NO: 57 - DNA, 584 bp, Rattus norvegicus
ttgactcagaagcagacgtttatttacttaaaacatttcacaattatattgacaattatttatttaaaataatattctaaatacttacaaaaataaatcaagtattgcttctagttttcaagtgtgacagcatttgccatagaaaatagaagggaccttttgaataaactaaacgctacactgaaacttcctcaagtttaataactacacactatttcctctgatgacttgtgctttataaatgtccaaactatttctgtgcaacaaacaggatcacagtaagactgaatgtataacaagccatatacattcatttttacacaaaatgtcagtactctggcccaaaccaccatcatccccaaaatgccagatacaccattatacagaagtacatgtggttgtgaagtttttaacatccagtcatcaaagaccagatatttattattaaagtatactcagctaaaggttgctaggcaagacattttttacttacattaaatcattagtcaaatcattaaaaaaatagaataaacttcaaatatgtctcaatacattaagttaatcccaagataaacatcagtgtc

SEQ ID NO: 58 - DNA, 1780 bp, Rattus norvegicus
tgtgctgggtgtgccccagagctggctttgactgtacgctgtcaggctctccctggacctcacggaacagcattgccagccacacggcttccaacaaatcacctcttttcatgctgtttggcacagatcgaatctacaggttatacaatggtcgatggagcaatgatcctttctgtgctaatgatgatggctctcccttccccgagtatggaagatgaggagcccaaggtcaacccgaagctttacatgtgtgtgtgtgagggcctctcctgcgggaacgaggaccactgtgagggccagcagtgtttttcctccctgagcgtcaatgatggcttccgcgtctaccagaagggctgctttcaggtctatgagcaggggaagatgacgtgtaagaccccgccgtcgcctggccaggctgtggagtgctgccaaggggactggtgcaacaggaacgtcacggcccggctgcccactaaagggaaatccttccctggatcgcagaacttccacctggaagttggccttatcatcctctccgtggtgtttgcggtatgccttttcgcttgcatccttggcgttgctctcaggaagtttaaaaggcgcaatcaagagcgcctgaaccccagagacgtggagtacggtactatcgaagggctcatcaccaccaacgtcggagatagcactctagcggaattactagatcactcatgtacatcaggaagtggctccggtcttccttttctggtacagagaactgtggctcgacagataaccctgttggagtgtgtcgggaagggccggtatggagaagtgtggaggggcagctggcaaggcgaaaatgttgctgtgaagatcttctcctcccgtgatgagaagtcgtggttcagggagacagaattgtacaacacggttatgctgaggcatgagaatatcttaggtttcattgcttcagacatgacctctagacactccagtacccagctgtggctcattacacattaccacgaaatgggatcgttgtatgactaccttcagctcaccactctggacacggttagctgccttcggatcgtgttgtccatagccagcggccttgcacacttgcacatagagatatttgggacccaggggaagtctgccatcgcccaccgagatctaaagagcaaaaacatcctcgtgaagaagaacggacagtgctgcatagcagatttgggcctggcagtcatgcattcccagagcacgaatcagcttgatgtgggaaacaacccccgtgtggggaccaagcgctacatggcccctgaagtgcttgatgaaaccatccaagtggattgctttgattcttataagagggtcgatatttgggcctttggcctcgttctgtgggaagtggccaggaggatggtgagcaatggtatagtggaagattacaagccaccattctatgatgttgttcccaatgacccaagttttgaagatatgaggaaagttgtctgtgtggatcaacagaggccaaacatacctaacagatggttctcagacccgacattaacttctctggcgaagctgatgaaagaatgctggtaccagaacccatccgccagactcacagctctacgtatcaaaaagactttgaccaaaattgataactccctagacaaattaaaaactgactgttgacattgtcaccggtgtcaagaaggagagtcaatgctgtcattgtccagctgggacctaatgctggcctgactggttgtcagaacagaatccatctgtccccctc

SEQ ID NO: 59 - DNA, 3032 bp, Rattus norvegicus
agtgctctggcgagtagtcctccctcagccgcagtctctgggcctcttcagcttgagcggcggcgagcctgccacactcgctaagctcctccggcaccgcgcacttgccactgccactgccgcttcgcgcccgctgcagccgccggctctgaatccttctggcttccgcctcagaggagttcttagcctgtcccgaaccgtaaccccggcgagcagatggagctggaccatatgacgaccggcggcctccacgcctaccctgccccgcggggtgggccggccgccaaacccaatgtgatcctgcagattggtaagtgccgagctgagatgctggagcacgtacggaggacccaccggcatctgttgaccgaagtgtccaagcaggtggagcgagagctgaaagggttgcacaggtcggtgggcaagctggagaacaacttggacggctatgtgcccacgggcgactcacagcgctggaagaagtccatcaaggcctgtctctgccgctgccaggagaccatcgccaacctggagcgctgggtcaagcgtgagatgcacgtgtggagggaggtcttctaccgtctggagaggtgggccgaccgcctggagtccatgggcggcaagtacccagtgggcagcgagccggcccgccacactgtctctgtaggtgtggggggtccagagccctactgccaggaagctgatggctacgactacactgttagcccctatgccatcaccccgccacctgccgcaggagagctgcctgagcaggagtcagttggggctcagcaataccagtcttgggtgccaggtgaggatgggcaaccaagcccaggtctggatacccagatctttgaggacccacgggagttcctgagccacctggaagagtacctgcggcaggtgggtggctctgaagaatattggctgtcccagatccagaaccacatgaatgggccagccaagaagtggtgggagttcaaacagggctcggtgaagaactgggtggagttcaagaaggagtttctgcagtacagtgagggtacgctctcccgcgaagccattcagcgggagctggacctgccacagaagcagggtgagccacttgaccagttcctctggcgtaagcgggacctgtaccagacactgtatgtggacgctgaggaggaggagatcattcagtatgtggtgggcaccctgcagcccaagttcaagcgctttctgcgccacccacttcccaagaccctggagcagctcatccagaggggcatggaagttcaggacggcctggagcaggcagctgagccttctgtcacccctctgcccacagaggatgagactgaggcactcacgcctgctcttaccagcgagtcagtagccagtgacaggacccagcctgaatagaggggccagcccagggtccccagcctgcctgccacacccagtctgtggcttttgtcaactaggacttgattgagctggggctgacacccaaggggatgccctgtccagccagacaccttctcacccactggcctgactcacaactgccacacaaccatgattcatggacatcaagaagcccctctcccatagggctcccacctgccacctacccctcacctgtctgccctagtcctggccctgtctccagtggcctcaccctctacactctcagaccatcacagaacacctttggcttcctcattctgcatcagtgtccagggccctttgggtagtcaagaaatcaagtgtctgaaaggcaatgaaaagtaggcaccaaacccaaggggcatcccagggcagatgctaaagcagaatcagagatggccgaaggaacctctacttccggggatgcagcccgctcctacagacacagcagatccagctggtgccctacctgcctcccagagcaactggccagtcttgggcagcatagctcccctctcagggtgagctgaagcagcagacctgacgcgctggcgcctcctggcccccagcagtgattcataccagtgaagaaaagcagacttcggctccatgactcagccatgccaggcggagggtcccagaggggctgagtcctcagccccagctgaggcagcagctggagtcttcagagccaggtgaatgacaccaggtctcaagctgctgagaagtctttccggccatgtctggaaggggtaccaccccagcaccagcaccgtcccctcctctcttgaagctgcctgcacagaggttccaagacactttcaaggcagagaaaataggattacaaagaggaggtgccttggcagagggcagcacccagctcagcctcagagctgaaggtgaagacaagccagcgtgaaaccccgggtctgccacgaatgcccgctccgctggccactcaccagctgcctgccacaagccactgcagcttgagcagggtctgtgccctctcagcacagagcccagttcgctgcgtggcctttggcccccgccagaaccttgcaggagccttaaggttcgggccctagcccagcctgaccttacctgctgtgccctgcctgctggtcaagtccagtcccaggagaccccatgccttggctcctaggctgttccaggcacttccctgacctgccgggtgattgcccagctggaacctcatccacaccccagcaccaaccacctcgtgttggtaactgctcgtgtctgtagtctgagtaggccatgttgaggttcctccatctgcctggtccattggtgttctgagaccagttccactgctgttctgacagatcccccaccctgtgcccctgccagcccccacaggtttatttttgcacataaaccatgacccatactaatttggctagctctggggactagggagaccctggagatctcaagagtgtggctatcccctattttcaccaagccttcaatatccagccaggccatctggcccacaccatcttacctcaaagacagacatatatatatatatacatatatatgattttgttaataaaactatgaaatttaaa

SEQ ID NO: 60 - DNA, 474 bp, Rattus sp.
atgaccaagcatcacaaacacttttatttgtggttatcagttatttttacagaataaagaactcattaatttgtaacactgtacattaaattaaaatataacccatccctacatcaaaaattatctaagttgaccaagataaaaaaagtctctaaaagcttatatacattagaagtagcaaaaataataataaaggaagaaattagaaaacagacatcaaagtcagacatctagaagaattctccaacatctgctctcttatctcggcatttgcttcgggcttttgttcgagctttgaaagctgcagagttatataaatgcctttcaaaacgaggaatcttcatggttttaagtgttgcagcatcgagcatcacagggggtcccagctcaaatacattgcgaaggaattcatttgtctgcaagtggtactgcatccctgatccaagagcctctttaaacgtgtcataagtgtgc

SEQ ID NO: 61 - DNA, 1614 bp, Rattus norvegicus
tactctctcgctctctttctgtctcttcctcgctccctctctttctctcctccctctgccttcccagtgcataaagtctctgtcgctcccggaacttgttggcaatgcctatttttcagctttcccccgcgttctctaaactaactatttaaaggtctgcggtcgcaaatggtttgactaaacgtaggatgggacttaagttgaacggcagatatatttcactgatcctcgcggtgcaaatagcttacctggtgcaggccgtgagagcagcaggcaagtgcgatgcagtctttaagggcttttcagactgtttgctcaagctgggtgacagcatggccaactacccgcagggcctggacgacaagacgaacatcaagaccgtgtgcacatactgggaggatttccacagctgcacggtcacagctcttacggattgccaggaaggggcgaaagatatgtgggataaactgagaaaagaatcgaaaaacctcaatatccaaggcagcttattcgaactctgcggcagcggcaacggggcggcggggtccctgctcccggcgctttccgtgctcctggtgtctctctcggcagctttagcgacctggctttccttctgagcacggggccgggtcccccctccgctcacccacccacactcactccatgctcccggaaatcgagaggaagagccattcgttctctaaggacgttgtgattctctgtgatattgaaaacactcatatgggattgtgggaaatcctgtttctctctttttttttttttaatttttttttattttggttgagtccttgtgttttagttgccaaatgttaccgatcagtgagcaaagcaagcacagccaaaatcggacctcaccttaagtccgtcttcacacaaaaataagaaaacggcaaactcacccccatttttaattttgtttttaattttacttacttatttatttatttattttttggcaaaagaatctcaggaatggccctgggccacctactatattaatcatgttgataacatgaaaaatgatgggctcctcctaatgagaaagcgaggagaggagaaggccaggggaatgagctcaagagtgatgcccacgtggggagcatctggtgaataatcgctcacgtctttcttccacagtaccttgttttgatcatttccacagcacatttctcctccagaaacgcgaaaaacacaagcgtgtgggttctgcatttttaaggataagagagagaaagaggttgggtatagtaggacaggttgtcagaagagatgctgctatggtcacgaggggccggtttcacctgctattgtcgtcgcctccttcagttccactgcctttatgtcccctcctctctcttgttttagctgttacacatacagtaatacctgaatatccaacggtatagttcacaagggggtaatcaatgttaaatctaaaatagaatttaaaaaaaaaagattttgacataaaagagccttgattttaaaaaaaaagagagagatgtaatttaaaaagtttattataaattaaattcagcaaaaatttgctacaaagtatagagaagtataaaataaaagttattgtttgaaaaa

SEQ ID NO: 62 - DNA, 6075 bp, Rattus norvegicus
  misc_feature (1)..(6075): where n may be a or g or c or t/u, unknown, or other
agcgggtcggtccacccgagaaaggggngcggaggaactgggaccgacccgggaagactgcgccagaggtgcacagagatcgcgcggggagcgaggagccgggcctcggcgctcagcccggatccacgtcccgcgaagcaccggctccggccggccgcggggtcatgacgtacagcgagctctacagccggtacacgagggtctggatccccgacccagatgaggtgtggcgctcggctgaactaaccaaggactacaaagatggggatgagagcctacaactcagactggaagatgacacaattctggattacccaatcgatgtccaaaataaccaggtgccattcttgcggaatccagatatcttagtaggagaaaacgacctcactgcactcagccatctccacgagcccgcagttctgcataacttaaaggtccgtttcctggagtctaaccacatctacacttactgtggaattgtgcttgttgccattaatccctatgagcagctgccaatctacgggcaggatgtcatctatgcctacagtggccaaaatatgggcgacatggatccccacatctttgctgtagcagaagaagcctacaagcagatggccagagatgaaaagaaccaatccatcatagtcagcggagagtctggagcagggaagaccgtgtcagccaagtatgccatgcgctattttgccacagttgggggctcagccagtgataccaacatcgaagagaaggtcctggcttccagtcccatcatggaggccatcgggaatgccaagacaactcgcaacgacaatagcagccgatttgggaagtacattgagatcggctttgataaaaagtaccacatcatcggggccaacatgaggacctatttgctggagaagtccagggtggtctttcaggcggatgatgagaggaactaccacatcttttaccagctctgtgctgcggccagccttcctgagtttaaggagctcgcactaacatgtgcagaggactttttctacaccgcccatggaggaaacacgaccatcgagggtgtagatgatgcagaggactttgagaagacaagacaagccctcacactccttggagttcgggagtcccatcaaatcagcatctttaagataattgcttctatcttgcaccttggaagtgtggagatccaggccgagcgggatggggactcctgcagtatatcgccccaggacgaacacctgagcaacttctgccgcctgctaggaatagagcacagtcagatggagcactggctgtgtcatcggaagctggtcaccacctccgagacctacgtcaagaccatgtccctgcagcaagtggtcaacgcacgcaatgccttggccaagcacatctatgcccagctgttctcctggattgtggagcacatcaacaaggccctgcaaacctcccttaaacaacactccttcatcggggtcctggatatttatgggtttgagaccttcgagattaatagcttcgagcagttttgtatcaactatgccaacgaaaagctccagcaacagttcaactcgcatgtgttcaagctggagcaagaagagtatatgaaggagcagatcccgtggaccttgattgacttctatgataaccaaccgtgcatagacctcatagaagccaagctgggtatcctggacctgttggatgaggagtgtaaggtccccaaaggaactgatcagaactgggctcagaaactctatgaacggcactccaacagtcaacacttccagaaaccacgcatgtccaacacggccttcattgtcatccactttgcagacaaggtggaatacctttcagatggttttctggagaaaaacagggacacggtatatgaagaacagatcaacatcctgaaagcgagcaagtttccgctagtggctgatttgttccgtgatgacgaggactctgttcctgccaccaacacagctaagagtcggtcatcttcaaagatcaatgttcgttcttccagacccctcatgaaggcccccaacaaggagcacaagaaatccgtgggctaccagttccgcacttccctaaacctgcttatggagactctgaatgccacaacgccccactacgttcgatgcatcaagcccaacgacgaaaagctccccttccacttcgacccaaagagagctgtgcagcagctcagagcctgcggcgtgttggagaccattcggatcagcgcggcaggctacccgtccaggtggacctaccatgacttcttcaaccggtatcgggtgttgatgaagaagagagagcttgccaacaccaccgacaagaagaatatctgcaagtctgtcctggagagtctcatcaaggatccagacaagttccagtttggccgcaccaagatcttcttccgggcaggtcaggtggcctacctggagaagcttcgggcagacaagttccgggaggccaccatcatgatccaaaagacggtcaggggctggctgcagagagtgaagtaccggaggctgagagcagctacgctaaccctgcagagattctgccgaggatacttagcccgcagactgactgagcacttgaggagaacccgggcggccatagtgttccagaagcagtaccgcatgctgaaggcccgccgagcctactgcagggtccgcagggctgcggtcatcatccagtcctacacgaggggccatgtgtgtacgcagaagctaccgccagtcctcacggagcacaaagccaccatcatccagaagtacgcccggggctggatggcacggagacattttcagcggcagcgggatgcagccattgtcatccagtgtgccttccggaggctcaaggccaggcaggcactgaaggccctcaagatcgaggcccgttctgcagagcatctgaaacgcctcaacgtgggcatggagaacaaagttgtccagctgcagcggaagattgatgaccagaacaaagagttcaagactctgtcagagcagttgtctgcagttacctccacccatgccatggaggtggagaagctgaagaaggagctggcgcgttaccagcagaaccaggaggctgaccccagccttcagctgcaggaggaggtacagagcctgcgcaccgaactacagaaggctcattcagagcgcagggtcctagaggatgctcacaacagggagaatggtgaactgagaaagcgagtcgcagacctggaacatgaaaatgcactcttgaaggatgagaaagaacaccttaaccaccaaatcctgcgccagtcaaaagctgaatcttcacagagctctgtggaggaaaacctgctgattaagaaggaactggaggaggaacggtcccggtaccagaacctcgtgaaggagtactcccagctggagcagagatatgagaaccttcgggacgagcagcaaactccaggccacaggaagaacccatcaaatcaaagtagcttagaatctgactccaattacccctccatttccacctcagaaatcggagacaccgaggatgccctacagcaggtggaggagattggtatagagaaggcagccatggacatgactgtcttcttgaagctgcagaagagagtgcgggaacttgagcaggagaggaagaagctgcaggtgcagctagaaaaggaacaacaggacagcaagaaagtgcaggtagaacaacaaaacaatggcctagatgtggaccaggatgcagatatagcctacaatagtctgaagagacaagagctcgagtcagagaacaagaagctgaagaatgatctgaatgagcgctggaaggctgtagccgaccaagccatgcaggataactccactcatagctcccccgacagctacagcctcctactgaaccagctcaagctggccaacgaggagctggaggtccgcaaagaggaggtgctgatcctcaggacccagatcatgaatgctgaccagcgcagactgtccggcaagaacatggagccgaacatcaatgccagaacaagttggcccaacagcgagaagcacgtggaccaggaagatgccattgaggcctatcacggggtctgccagacaaacagccagactgaggattggggatatttgaatgaagatggagaactcggcttggcttaccaaggcctaaagcaagtcgccaggttgctggaggcccagctgcaggcccagaacctgaagcatgaggaggaggtggagcatctcaaggcccaggtggaggccatgaaggaggagatggacaaacagcagcagaccttctgccagactctgctgctctccccagaggcccaggtagaatttggtgtccagcaggagatatcccggctgaccaatgagaacctggactttaaggagttggtggaaaagctggagaagaatgagaagaagctgaaaaagcagctgaagatttacatgaagaaggtccaggacttagaagctgcccaggcattggcacagagtgacaggaggcaccatgaactcacaagacaggtcacggtccaacgaaaagagaaggacttccaaggcatgctggagtaccacaaagaggacgaggcactccttatccggaacctggtgacagacctgaagccccagatgctgtcgggcaccgtgccctgtctgcctgcatacatactctacatgtgcatcaggcacgcggactacaccaacgatgacctcaaggtgcactcgttgctgagctccaccatcaatggcattaagaaagtcctcaagaagcacaatgaggactttgagatgacgtcattctggttatccaacacctgccgcctccttcactgtttgaagcagtacagtggggatgagggtttcatgacacagaacacggcgaagcagaatgagcactgtctcaagaactttgacctcactgaataccgtcaggtgctaagtgacctttccattcagatctatcagcaactcattaaaattgctgagggcctgctacagcctatgatagtttctgccatgctggaaaatgagagtatccaggggctgtcaggggtgaggccaactggctaccggaagcgctcgtccagcatggtggacggagaaaattcgtattgcctggaggccatcatccgccagatgaatttctttcatacagtcctgtgtgaccagggcctggaccccgagattatcctgcaggtgttcaaacagcttttctacatgatcaatgcggtgactcttaacaacctactcctgcggaaagatgcctgctcctggagcaccggcatgcaactcaggtacaacataagtcaactcgaagagtggcttcggggaaaaaaccttcagcagagtggagcggttcagaccatggagcccttgatccaggcagcccaactccttcagctgaagaagaagacccaggaggatgctgaggccatctgctctctgtgtacctccctcagcacccagcagattgtcaaaattttaaacctctacactcccttgaatggatttgaagaacgggtgacagtgtcctttatacgaacaatccaggctcagctacaagagaggagtgacccccagcagctactgctggactccaaacacatgtttcctgttctgtttccatttaatccatctgctctgaccatggactcaatccacatcccggcctgtctcaatctggagttcctcaatgaagtctgaggatgcgttgtttccaaggcaagcgagaaggaagtgcgtgctgtcggctgaaggagtgctaggtctgttaaatatgcccagcgtagatcaaaccatgttagagacctgtggggagcactgaactaaacagcggggtgcactctggtcgttagcttttgtacagactgctcagaaaacacctgaagtgaggacgattgttgcagtgggactttcaggttaaaccaagacacgtcagaacggacggccgctgtgtagctccagtcaccatacaaagatgccagttctacagagtggaagtgcctagctttgagctgtgtatataacttaagaatgttcaaactaagattatattaaaaacacatgacataaa

SEQ ID NO: 63 - DNA, 474 bp, Rattus norvegicus
attatgtgccgaaaggggaaggttggaccgcctggcctggacatcggaaggaaggaagatcagctgattgtccacatatttcaccctaaggtcaatgtgagtcaggaaaccatgtttggtgacggaaatacctgttacacattcgactacactgtgtttgtgaaacattacaggagtggggagatcctacatacagaacatagcgtcctaaaagaagattgtagcgaaactctgtgtgagttaaacatctcagtgtccacgctgaattccaattactgtgtttcagtagttggaaagtcgtctttctggcaagttaatacagaaacatcaaaagacgtctgtatcccctttctccatgatgacagagaagaatcaatttggatgctgctcgttgctcctcttctcttccttacaatagttgtcccggcacttgtgtgttgttacattaagaagaatccatttaagagaaaaagc

SEQ ID NO: 64 - DNA, 5028 bp, Rattus norvegicus
gcctggcaggcgggagaacgctccggagttgtggccgtgggcaccgggctcgcggcaagaggagcggagagcgggcatctcctgagcgccgtcatggctgcttaggctgcgcctgccagcggaccgacggtgtcgcccgaatccggctcggataggtctggttggagtctgtgcctgcttgcttggcgtgtggttgttcctgcttgattggcacggtgccattggcttcgtatttgggaatcggaggagttaatcttgtctcttctcacaggttcgagtcctcagaccttctgcaggactccatccatatctgcctcgcagctgactctcctgctcacacagaagacggccatcctagatccccagctattgtgctgaccatccccttcctgctccggatctcgcctggctgctaggctgtggtgctgccttttcagagtcaggctgtagcgactccccgccttcgtcccggctgggcttaggtggaacagtggttcatctcatctcatcagcacttctgaagaagaaagtgtgagaagcagaggccatggctccttttcgctgtcaaaaatgcggcaagtccttcctcaccctggagaagttcaccatccacaattattcccacaccagggagcgcccattcaagtgctccaagactgagtgtggcaaagccttcgtctccaagtataagctgatgagacacatggctacgcactctccccagaagacgcaccagtgcactcattgtgaaaagactttcaaccggaaggatcatctgaagaatcacctccagacccacgatcccaacaagatgatctacgcctgcgaagattgtggcaagaaataccacaccatgctgggctacaagaggcacatggccctgcattcggccagcagcggcgatctcacctgcggcgtctgcaccctggagctggggagcaccgaggtcctgctggaccacctcaagtctcacgcggaagaaaaggcccaccacgcgcccagggagaagaaacaccagtgcgaccactgcgagagatgcttctacacccggaaggatgtgcgtcgccacctggtggtccacacaggatgcaaggacttcctgtgtcagttctgcgcccagagatttgggcgcaaagaccacctcactcgtcacaccaagaagacccactcccaggagctgatgcaagagagcctgcaagcaggagaataccagggcggttaccaacccattgcgcctccgttccagatcaaggctgatcccatgcctcctttccagttagaaatgccccccgagagcgggcttgatgggggcttgcctcctgagattcatggtctagtgcttgcttccccagaggaggttccccagcctatgctgtctatgccgccaatgcagccaatgccagagcagcctttcactctgcaccctggggtagttccctcctctcctcccccgatcattcttcaggagcataagtacagcccagttcctacctcttttgccccgttcgtaagcatgccgatgaaagcagatctcaagggcttttgcaacatgggtctctttgaggaatttcctctgcaagagtgtcagtcgcctgtcaagttcagtcagtgctttgagatggctaaggaagggtttgggaaagtcaccctgcccaaagagctgctggtagatgctgtaaatatagccattcctggctctctggagatttcctctctcttggggttctggcagctgccccctcctcctccccagaatggcttcatgaatggcaccatccctgtgggggccggggagccgctgccccataggataacttgtctggcacagcagcagccaccacctctgctacctccgccgccgccgctgccgctgccagagccgctgccacagccacagctgccgccacagtttcagttgcagctccagccccagccccagatgcagccccagatgcagctgcagcctctacagctgcagctgccccagctgctgccccagctgcagcccgagcctgagccagagccagagccagaggaagaagaggaagaagaagaagagatagaagaagaagaagagatcgaagaagaagaagaagccgaaccagaagcagaagaagaagaggaggcagaagacgaagaggaggcagaggaagaggaagaagagccacagccagaagaagcccaaatagcaatgagcgctgtgaatatgggccagcccccgctacccccgacccctcatgttttcacagctggcaccaacactgctatcctgccccatttccaccacgcgttcagataaattggttttttaagagggtgcttctcttctggaagatgtttcaaacaccagttccagttccagacatcagttacagtttgaagagaagcgttggaaaaacaggaatggggtttctagcttattgccatgagtagattgagaaaaagaactctcttaactgcatgcactgtgccaatacatatatatatatatatatatatatatatatgtatatatatatatatatatatcatccttagtattcatgctttgtaccaaacttagtgagtgcgggcgttctccgtaatcgaactgcaagtagtatcatattattaccctgatattgttagtctcatattattagccttgtattattctcatataatcaaaaccaagatccaaaacatgagctgctaatttgtaaatatcgtgttgagtgttagccgtcgtagtgatgttagctgcgtagttgcgtgttagcactgcctaggaagggcacgagggccaagttgggcttctcccacttggaagatgttttgaagagaagggggtgatctccgtagggcgtccgtaactaggccgtgtgttcttttcagggacccgtctaccttcaggattggatgtagtttagtcgctcttcttcttagctcgctttgtagtttgtccttctggtagcctactgtgtgtgtctgtgtgtagctttataggaaagttccgtgtgaagctgtcggtgtcttcgttttcaaaagtgaattttaaatgtatttttcaatatttttcatgtgatgttgtaccaatgtgaattatgacttcgtttatcttaaagacaaaactggttgtcagtcatatctgacaggaagaaagaaatccctgtgggtaggcaagtcaagtggccaactaatgagaagaagcatcaatcgaaagtgttggctgactgggacactcatgattctcacaggactttgagaaacgtactggaattaaaaaaaaaaaagcttaagtacattagataagaattttctttgcctagcttaacctactacttaagcctcttaagttctgaagtattgtgatcaaccaataggaaaatgtatctgtagttgatgaatttcagtccttgttactttgtatcccaagaggtttgtgttttgggaatgtaaccgtacttgtaatctcagttggtatcttgctaatcgatttgaaagtgtaaaacctaacccttgaagactctgtatttccttttttgagactgtatttcccagcatgtataccctaacctttggagactctgtattctgtttttgagactttccccccgccccccagcatatgtaccccgacccttgaagactgtatttcgtttttgagagcgtatttcccagcatatatacactaacccttgaagactctgtatttccttttttgagactgtatttcccagcatatatacactaacccttgaagactctgtatttccttttttgagactgtatttcccagcatatatacactaacctttgaagactctgtattctgtttttgagacccccccccagcatatgtaccctaacccttgaagactgtatttcgtttttgagaacgtatttcccagcatatatacactaacctttggaagactctgtatttcatttttgagactgtgtttcttagtatacataccctaacctttgaaagactccatttttgagacttccccccccccagcatttgtgccctaacccttggaggctttgtattttttttttgagacttttccgccagcatatatacactaacccttgaagactctgtatttcatttttgagacttttttccccagcatatataccgtaacccttgaagactctgtattccgtttttgagatttttttccctcagcatatataccccaacctttgaagactctgtatttcatttttgagactttttcccagcatatataccctaacctttgaagactctgtattccatttttgagattttttccctcagcatatataccctaacctttgaagactctgtatttcgtttttgagattttttcccccagcatataaacactaacctttgaagactctgtatttcatttttgagacttttttcccagcatatataccctaacccttgaagactctgtaatctgtttttttttttttttgagactttttcccccagcatatatacactaacctttgaagactctgtattccattttttgagacttttttccccagcatatataccctaacctttgaagactctgtatttcatttttgagactttttccccagcatatataccctaacctttgaagactctgtattccgtttttgagaccccccccccggcatgaataccctaatctttgaagactctggtatttcatttttgagatttttttcccctcagcatatatacactaacctttgtagactctgtattccgtttttgagactttccccccccagcatgtataccctaacctttgaagactctgtatttccagcatttgtaccctacccttgaagactctgtatttcccagcatttgtaccctaacccttgaagaccctgtatttcgtttgtaagacttttccccagcatatatatcctacatataataaacgctaagcatctagcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 65 - DNA, 2648 bp, Rattus norvegicus
gctggggagacccagagcgccgcgctgtctgctgggctcacggagacgcggaccacctttggtcgccccgcgccgtcctctccgcgccgcctggcccgccgcctcgagcgccctcagtccctcaagccagatgatgaacttcctgaggcgccggctgtcggacagcagcttcattgccaacctgcccaatggctacatgactgaccttcagcgcccggagccgcagcagcccccacccgcccctggccccggtaccgctaccgcgtcggcggccacctcagctgcctcgcctggccccgagcgcaggccgccacccgcccaggcacccgcgccgcagccggcgccgcagcccgcgccgacgccgtcggtgggcagcagcttcttcagctcgctgtcgcaagccgtgaagcagacggccgcctccgccggcctggtggacgcgcccgcgccttcggccgcttccaggaaggccaaggtgctgctggtggtcgacgagccgcacaccgactgggccaagtgctttcggggcaaaaaaatccttggagattatgacatcaaagtagaacaggcagaattttcagagcttaacctggtggcccatgcggatggaacctacgccgtggacatgcaggtactccggaatggcacaaaggttgtcaggtccttcaggccagacttcgtgctcatccgacagcatgcatttggcatggcggagaatgaagacttccgccacctggtcattggcatgcagtatgcaggcctccccagcatcaactcactggagtccatttacaacttctgtgacaagccatgggtgtttgctcagatggtggccatcttcaagacactgggaggggagaaatttccactcattgagcagacctactaccccaatcaccgagagatgcttacacttcctacgtttcctgtcgtggtgaagattggccatgctcactcgggcatgggcaaggtcaaagtggaaaatcactacgacttccaggacattgccagtgtggtggccctcacccaaacctatgccacagcagaacccttcattgacgccaagtatgacatccgggttcagaagattggcaacaactacaaggcttacatgaggacatccatctcagggaactggaagacaaacactggctccgcgatgctggagcagatcgccatgtcagaccggtacaagctctgggtcgacgcctgctctgagatgtttgggggcttggacatctgtgcagtcaaagctgtgcatggcaaagatggcaaagactacatttttgaggtcatggactgtagcatgccactgattggggaacaccaagtggaggacagacaactcatcactgatctagtcatcagcaagatgaaccagctattgtccaggactcctgccctgtctccacagagacctttaaccacccaacagccacagagtggaacacttaaggaaccggactccagcaagacccctcctcagcggccagccccccaagggggccctgggcaaccccaaggaatgcagcccccaggcaaggtgctgcctccacgtcggctcccctcgggaccatcactgccaccttcttcctcttcctcctcctcttcctcctcctcttcctcggctcctcagaggccgggcggccctacctccacccaggtgaatgcatcctccagcagcaactccctggcagagccccaggcacctcaggctgctccaccacagaagccccagcctcacccacagctcaacaagtcgcagtccctgacaaatgcgttcagcttctctgagtcctccttcttccggtcttcagccaatgaagatgaagccaaagcggagaccatccggagcttgaggaagtcctttgccagcctcttttcagattagctcttcagatacacgagggcatccggcccaaccaggaaaggcatctaagacattcatcaacagcagtccgccatgtttggtggcaatgtcccatgacttggacatgtgtggccccttcttctgttcctttgtgctcagtaatttgtgcagcccagaaatgaccatgtgatggtctcagggcaggtggctaccctgagaaagacctgacagtagaggaaagggctgtttccctgtagtcttaagagctttattctgaagtcatcgttgctgtgaattcagtggccttattgtgacagagccatcgtgtcttactcttccctgtgaagccaggatctctgtaacgtaggactcctggcagcttacctatctcagcatatcctagtagtcagtgctacctgtgggaaatatattcttaccttagaagtattgttggctctaagttttaggagcttctccccaaagctgggtctcctgtggtccctgttgaaagcacattcacctgaccctgcactccctagactgctgtggaggtcaagttccttgtgactgtttgaaacaagagaacaaggatccacagtgtctcctttctcccctctggagagcagttctgttgtgtgagcatcagtaaccttgacctttcgaagctgtactgtagaagagaatgcatgccgctataacagcagtccaccaacctttgtgttcctcaccttatgaatgtcaagaccttggtgtaataaaagcagcagctttaaccagc

SEQ ID NO: 66 - DNA, 598 bp, Rattus sp.
cataatactgtcattacaaaaaaatacaaaaaactactataaaaacattcaggggcttgtcaaagtgagaaaacctaaagaccccaccccaggaccaactgaagcagttttctcccagctccttcactattcgacttttatacaactgtgggagtggggtggggtgagggtcacacaggcagagaggctggaaatttccgacacagcctccaagaaaagtaagaaataagtagcttcacatatcgcaaaagtggggcttggaagtttggggtggctaggccctgagttcagagatatgggaaggaacctgtgatcctgaatctcttggtggggaacagctgccacctgatcccaaagcccttttccttcctggtgatggctgggagatgggtcctgccccacctcttagctgtggtggatggttcaagccctcctttcttctctccacccctgaacactccccagagcagctgagggggacagatcctccaactctgtggggtgtaggcaaggcagctgttgggaaggctcctaagggttggctcaactccctgccccaaatgaaaagtgtctcgtgctcaaatgacagccttgttca

SEQ ID NO: 67 - DNA, 3105 bp, Rattus sp.
aaattaaatctaaggactttcagatttatggctttgatcacactgtttctagagaaatctaaacctggaaggctgagttaagccagacattccagatggctctctcctcatagtccttggaatcacgaaggaagcagggcagagagctaccagaagtagtaaacattgatcacaggctcctagttcatcgtgaccaaatcaaaaggaatgtttctccatggcccattaactgtctgttagtttgaacgtaacatggtgatagccagactggagctacctgagtcctgttccagggaatcttagggcaattacctacataacccttctggacctcaactgcctgatcttagggattaataacatctatttaccagagcgactgcattgtgaagggttccaaacactcctggcacagagtaagcactgtctgggctttggatagaaatctcttctgcaccatgagctcatttataagactttccaggtctggaattgtacaacccaaacagctcatatcaatgtcacaagctcttcggtttggcaaaatgtctgggagtcaccaaatgcagagaatgccatattcaacaaagcctgataaccaaggactcagtggactaattggcagtcctatcccagatccaaggttccttgagccaggggcaagctaggatatgctcccaggtatcttctcccttaggactttaggtttcttggccacttcctcttatttcagtgaaagcagatccactccattgacacttgtggtcacagtctagcacgactgctcccttccttcttttctccctccctgcgcagcttcatttgctcccagtagtggctggaaaaacaccaaattccaatccgcggtttctcccttctacttcctggaaacatccaagggctcggcacttactcagcagattcaaaccttccactttccatcactcatcgaggatgatgctgctccttggcaccaaccaccctgcctgactccaccctctggcttacaataaaaggctgaggcagagccgctagaaatgcagagacacagacagaggccagcccagaaaccagccaactctcactgaagccagatctctcttcctccaccactatgcaggtctctgtcacgcttctgggcctgttgttcacagttgctgcctgtagcatccacgtgctgtctcagccaggtgagaccccagtttccttctccttctagcatttcaccccattttttaattgttgtgggccatcatagtgggccttacctagtaaaatacttttttttttttaccaaggtaaggagcatagagccaacccaattacaggggttgcttctggaaagcaactaggattttaatcgttagatcaaagtttagaatcgcaccttcatacagttcctgctcccctatttcctgagtatttgagaacctggttgatcaaagaagggcttgggttggttcatttttccagatagaggagaatcaggaagagacccaggatcttgatctatgtttcaccagcttccagagatagcagctcagcagaggtagttggtatcagagatactcatgattcgatatagggtttttttttgtaacctatagtaatgtactcggtaatcttctcagaccctagtaatttgacttctaactaccctcaaatgacagtccctagctttaatggcatccctctgtccaagattgtgaacttactttaagtgtgtcagagatcaccttccagctctgatgtattggcatttacatcccaatctgctgaaactgccttctcctcatggtccttttcttctctaaggtcagaagcacctttccagttctaatgtgctccctgcttctcttttattctccagatgcagttaatgccccactcacctgctgctactcattcactggcaagatgatcccaatgagtcggctggagaactacaagagaatcaccagcagcaggtgtcccaaagaagctgtagtgtgagttatacaccccagccctccctggtccaatatttttcctcgagaacaagggatggtcttcatagacttagaatcagttacatgctcagctccaatatcaagtggttcccaatggggaaactgaggccaagaagggaaagttaattctcagcagcactgtctctatggctgctgttcggggccttccatttgcatgagcttattgtagtaaacttgcagaagaggaaggtcactttgagtccccctttctacctgccctcccacctcgagccctacacagtccctccatgtatagcaggttaaacttcatctaaccgtgtcttctctctttccacagatttgtcaccaagctcaagagagagatctgtgctgaccccaataaggaatgggtccagaagtacattagaaaactggaccagaaccaagtgagatcagaaactacagtcttctataaaattgcatcaaccctaaggacttcagcacctttgaatgtgaacttgacccataaatctgaagctaatgcatccactctcttttccacaaccacctcaagcacttctgtagaagtgaccagtatgacagagaactagtgtgatttggaatgtgatgccttaagtaatgttaaacttatttaacttattgatattacactattcccttccatgaatactagaaatccttaaatgcaagatgtagatccatttttttatttctctgtgaatcctggttcaacactttcaatgtatgagagatgaatgggtaaactttgtgtttgagagtccaaggtattgtttaaaatattattatggatattcctaattattaaaagaaatatattatttttgtacacaagtctgactttcggtgttttctgagggaaatggcaaagctaagagtacataagaacacacaggaggacatcacaagatgggacacatattgagggggggatgggggaatgaatgctgcactcttttgtattgagtggtctcatgtgagtgtcataaactctttgagacagggtccagtcagggatgctagtaccatagttccaatccccaggactgcttctcagacacatgctcgataaaagccccagtccttcccagtcatg

SEQ ID NO: 68 - DNA, 2619 bp, Rattus norvegicus
  misc_feature (1)..(2619): where n may be a or g or c or t/u, unknown, or other
ggcaccaggctcgcctcacgccggcggcgcggactgggaagcggcgccctcggctgcgctcggttcggaacccgctctgtggagtcagaggcagcgacgggcgccaggccgcccgggccggcggaggcggccgctgaggggagtgtgaggaggcagcggcggccctgggaatgtgaggcggcgcggagcgggagagaagagccggggtgggggcacgagccgcggctgccgtgcctcagacgagccgcgagcctctcgcttcagcgcgcgaccgccgggagcttgggggctgggtgcgggggaccgcgcggggctgggttctctgattcattcattctccgcggcccggagcccgaggccgcgggtgctgcgaggagaggcgggaagagggggcggccgcgagcggggctcctttgtgctcggcgggggccgggaagccgtgggaggtgggctcgggccccctgcgctgctccccggcggccgctgcgcccccagctagccgccagcctggaaatggctccgctgatgctcctcgtgagaacgaatcgatccttcccagccttctctgcctgctctccacctcctctctgctccgagtcttaggagaacgaacattcaaaggacagattccaatgtggtgtgctgtgcacatcgcgagcggctggggtttgcacttcgagatttcttctttataattttttttttttaatgtaagggagacagtggaattgctacccgtagaatttttattcaagtgcacgtcgcgttgggttgcacgctccacccccagggacctggtgtggtgaaatttgaacccaccgccttagcccaaaggccgagtaacctggctgcttgagtgtcgtggaagacgtgagcgaaatgatcagcgaactcattttttatcagactcactgaagctggcttttgcgtttttctacacgtacactaattttatggaatagttaaagtgctatattctccgcgcaaccttttcaaattccaaatgtttgaacgttttggtgtcagcgcgagtgaaatcattttaccgacaagaactaactgaattgtctgcctcgttgagttgcctccggaaaagatctcgggggtggaaaagcaactgcaaaataacagacggagaaaattccttggaagttatttctgtagcataagagcagaaacttcagagcaagttttcattgggcaaaatgggggaacaacctatcttcagcactcgagctcatgtcttccagatcgacccaaacacaaagaagaactgggtacccaccagcaagcatgcagttactgtgtcttatttctatgacagcacaaggaatgtgtataggataatcagtctanacggctcaaaggcaataataaatagcaccatcactccaaacatgacatttactaaaacatctcaaaagtttggccaatgggctgatagccgggcaaacactgtttatggactgggattctcctctgagcatcatctctcaaaatttgcagaaaagtttcaggaatttaaagaagctgctcggctggcaaaggagaagtcgcaggagaagatggaactgaccagtaccccttcacaggaatcagcaggaggagatcttcagtctcctttaacaccagaaagtatcaatgggacagatgatgagagaacacccgatgtgacacagaactcagagccaagggctgagccagctcagaatgcattgccattttcacataggcagccaattttctttgctggagaacttctctggccttggttatttttgaaattagtgaagtcaccatttcccttggaggaagaggatctgcagtttattctgattgaaaagatacaaatgacccctgactgagagaaacccctggaagaaaggagacagcctaaaaagacaactaatgtcgtgcacttttaacattgaagaatgaaaagaagggagttctttttaaagaagacacctgcttgaattctgggtgaggaggacggctaaactagagccatctagagaaagaaaagatggatttgggaagttttcatgctttagaacttaggagagtatcatggtccttgcttcactgatatccagaagactgagaagcccttgatgaagggaggaaaatctgctggaaatcaaagtaactttgcgatgaagaaaagtaaaaaatccaagtggctggctttcttggacttctaactagactggttcatggattttatttcttttgaaacttaacaaactaggatctcttggctatgtaatcttggcacatgtaactgcccaagttcatgtattgtctgtcatgaatcctccgccctacctcacagggatgttgtgaggcccatgttaatgtctgtaagtagtaattcatgcttagctctttgttgatagtctctgtgtctttttaaaattataagtgctttattacaccttcaaacaaacattaggtaatattttttaaatggaagataatggcattgtatgtggttcatcactgagcatccttgtatgaagaataatttggctgcatgagatgttaagttgattcttttacgattgggcctttatatgatagtaataataataaaagctatgctaagaaaaaaaaaaa

SEQ ID NO: 69 - DNA, 1057 bp, Rattus norvegicus
cggcaacagacgattgttctcagagccactttgatggcagctatgaggctcaccctgttccgcattgtgtgtctgctgccaggctgcctggccctgccactgtcccaggaagccggagaagtgaccgcacttcagtgggaacaggcgcagaattatcttaggaaattttaccttcacgactctaaaacaaagaaggccaccagtgcagtggacaaactgagggaaatgcagaagttcttcggtttgccggagactggaaagctgtccccccgtgtcatggagataatgcagaagcccaggtgtggagtgccagatgttgcagaattctcactaatgccaaacagtcctaagtggcattccagaactgtcacctacagaatcgtgtcctatactacagacttgcctcggttcttagtagatcaaatcgtgaaaagagctctcagaatgtggagtatgcaaatcccactgaacttcaagagggttagttgggggactgcagacatcataattggcttcgcaaggggagatcacggagacaacttcccatttgatgggccaggaaacactctaggccatgcctttgcaccggggccaggcctcggcggagatgctcactttgacaaggatgagtactggacggatggtgaggactcaggagtgaacttcctgtttgttgccactcatgaacttggccactctctgggtctgggtcactcttctgttcccagttctgtgatgtaccctacctatcaaggagatcattcagaagacttcagtcttacaaaggacgacattgcaggcatccagaagttatatggaaagaggaacaagctgtgatagatgcagacagtttctggaatgagcaaacgcccttcctgagccacacttactcctttcttccttgtactgtggatgggttttgcacatccctctgagggtcattttgatggaatgagtctgacaaatctcaggtaacacgacagacaccagcaataaatgtcatgtgacatcagcaataaatgtcatgtgtgcaaataaaaaaaaaaaaaaaaaaccg

SEQ ID NO: 70 - DNA, 1912 bp, Rattus sp.
cagattaggatcagcgagcacttgaggacttagggccacaaaaaaccgcacaagatcgacagactatttctggagagctgcagaacgggcacgctggggtcgctggtgctggccatggtgatggaggtgggcatcctggacgccggggggctgcgcgcgctgctgcgagagcgcgccgctcagtgcctgcttctggattgtcgctccttcttcgccttcaacgccggccacatcgtgggctcagtgaacgtgcgcttcagcaccatcgtgcggcgccgcgccaagggcgccatgggcctggagcatatcgtgccgaacaccgaactgcgcggccgcctgctggccggagcctatcacgccgtagtgctgttggacgaacgcagcgccgccctggacggcgccaagcgcgacggaaccctggccctggccgcgggcgcgctctgccgagaacgccgctccactcaagtcttctccctccaaggaggatatgaagcgttttcggcttcctgccctgagctgtgcagcaaacagtccacccccatggggctcagcctcccgctgagtactagtgtgcctgacagtgcagaatccggatgcagctcctgtagtacccctctctacgaccaggggggcccagtggagatcctgtccttcctgtacctgggcagtgcttaccatgcttcccggaaagatatgctcgacgccttgggtatcactgctttgatcaacgtctcggccaattgtcctaaccactttgagggtcactaccagtacaagagcatccctgtggaggacaaccacaaggcagacattagctcctggttcaacgaggcgattgactttatagactccatcaaggatgctggaggaagggtgtttgtgcactgccaggccggcatctccaggtcagccaccatctgccttgcttacctcatgaggactaaccgagtgaagctggacgaggcctttgagttcgtgaagcagaggcggagtattatctcccccaacttcagcttcatgggccagctgctgcaatttgagtcccaagtactggcccctcactgttctgcagaagctgggagcccggccatggctgtccttgaccggggcacctctactacaacggtcttcaacttccctatctccatccctgttcaccccacgaacagtgccctgaactaccttcaaagccccatcacaacctctccgagctgctgaagggccaggggaggtgtggagtttcacgtgccaccgggacgacactcctcccatgggaggagcaatgcaataactctgggagaggctcatgtgagctggtccttatttatttaacaccccccccccaaacacctcccgagttccactgagttcccaagcagtcataacaatgacttgaccgcaagacatttgctgaactcagcccgttcgggaccaatatattgtgggtacatcgagcccctctgacaaaacagggcagaagggaaaggactctgtttgagccagtttcttcccttgcctgttttttctagaaacttcgtgcttgacatacctaccagtattaaccattcccgatgacatacacgtttgagagttttaccttatttatttgtgtgggtgggtggtctgccctcacaaatgtgattgtctactcatagaacaacgaaatacctcactttgtgtgtttgcgtactgtactatcttgtaaatagacccagagcaggctttcagcactgatggacgaagccagtgttggtttgtttgtagcttttagctatcaacagttgtatgtttgtttatttatgatctgaagtaatatatttcttcttctgagaagacattttgttactaggatgactttttttttatacagcagaataaattatgacatttctattgaaaaaaaaaaaaaaaa

SEQ ID NO: 71 - DNA, 4665 bp, Rattus norvegicus
attgcttgcttcctaggggccagggctgttctgagcacgtcacaccaatccaagcttcacaaacacctatgggcagttgaagagggggaggcctcggacttctcgctggcctgggattcctctgtggcagcagcgggaggcctagaaggagagtcagagtgtgatcggaaatccagccgtgcgctggaagacaggaacagcgtgacaagtcaagaggagagaaacgaggacgatgaagatgtggaagatgagtcaatttacacctgcgatcactgtcagcaggacttcgagtctctggcagacctgacggaccaccgggcccaccgctgtcctggagatggtgatgacgacccacagctctcctgggtggcttcatctccctccagcaaggatgttgcgtcacccacgcagatgatcggcgatggttgtgaccttggcctcggcgaggaggaaggcggcaccggcctgccgtacccttgccagttctgcgacaagtccttcatccgcctgagctacttgaagaggcatgaacagatccacagcgacaagctgccgttcaagtgcaccttctgcagccgcctcttcaaacacaagaggagccgggaccggcacatcaagctgcacacaggcgacaagaagtaccactgccacgagtgcgaggcggctttctcccgcagggaccacctcaagatccacctgaagacccacagctccagcaagccgttcaagtgcagcgtgtgcaaacgcgggttctcctccaccagctccctgcagagccacatgcaggcccacaagaagaataaggaacacctggctaagtcagagaaggaagccaagaaggacgacttcatgtgtgactactgcgaggacacctttagccagacagaagagctggagaagcatgtgcttaccctccacccgcagctctcagagaaggcggacctccagtgtatccactgccccgaggtctttgtcgacgagagcacgctgctggcccacatccaccaagctcacgccaaccagaaacacaagtgccccatgtgccctgagcagttctcctccgtggagggtgtgtactgccacctggacagccaccggcagcctgattccagcaatcacagtgtcagccccgaccccgtgctgggcagtgtggcttccatgagcagtgctacacctgactcgacgcccgaccccgtgctgggcagtgtggcttccatgagcagtgctacacctgactcgagcgcctctgtggagcgcgggtccacgccagactccaccttgaagccgctgagggggcagaagaagatgcgggatgacgggcagagctggtccaaggttgtctacagctgcccctactgttctaagcgggactttaccagcctggctgtgctagagattcatctgaagaccattcacgcggacaaacctcagcagagtcacacgtgtcagatttgcctggactccatgcccacgctctacaacctcaacgagcatgtgcgcaagctgcacaagagccacgcttaccccgtcatgcaattcggcaacatctccgccttccactgcaactactgccccgagatgttcgcggacatcaacagcctgcaggagcacatccgagtctcgcactgtggccccaatgccaacccccccgacgggaacaatgctttcttctgtaaccagtgttctatgggctttctcactgaatcctccctcacagagcacatccaacaggcacactgcagtgtggggagcaccaagctggagtctcccgttatccagcccacacagtccttcatggaggtctactcctgcccttactgtaccaactcccctatctttggctccatcctgaagctcactaagcacatcaaagagaaccacaagaacatcccgttggcacacagcaagaagtccaaggcggagcagagtccggtctcctctgacgtcgaggtgtcttccccgaaacgacagcgcctctcggggagtgccaactccatctctaacggcgagtacccctgcaatcagtgcgacctcaagttctccaacttcgagagcttccagacccacttgaagctgcacctggagctgctgctccggaagcaggcctgcccccagtgcaaagaggacttcgactctcaggagtccctcctgcagcatctgaccgtgcactacatgaccacgtccacccactacgtctgcgagagctgtgacaagcagttctcctcagtggacgacctgcagaagcacctgctggacatgcacacctttgtgctataccactgcaccctgtgtcaggaggtcttcgactctaaggtgtccatccaggtgcacctggccgtgaagcacagcaacgagaagaagatgtaccgttgcaccgcctgcaactgggacttccgcaaggaggctgacctgcaggtgcacgtcaagcacagtcacctcggcaacccggccaaggcccacaagtgcatcttctgtggtgagaccttcagcaccgaggtggagctccagtgccacatcaccacgcacagcaagaagtacaattgcaggttctgcagcaaagccttccacgccgtcctcctgctggagaagcaccttcgggagaagcattgtgtgtttgacccagctgcagagaatggcacggccaacggggtgccccccacctccaccaagaaggcagagcccgccgacctgcagggcatgctgctcaagaaccctgaggcaccgaacagccacgaggccagtgaggacgatgtggatgcatcagagcccatgtacggctgtgacatctgtggtgcagcctacaccatggaggtgctgctgcagaaccaccgactccgggatcataacatccggcccggagaggacgatggctcacgcaagaaggcagagttcataaagggcagccacaagtgtaacgtgtgctctcggactttcttctcggagaacgggctccgggaacacctgcagacgcaccggggccctgccaagcactacatgtgtcccatctgtggcgagcgcttcccctcgctgctgacgctcactgagcacaaggtgacccacagcaagagtctggacacaggcacctgtcgcatctgcaaaatgcccctgcagagtgaggaggagtttatcgagcactgccagatgcaccccgacttgcggaactccctcactggtttccgctgtgtggtctgtatgcagactgtcacctcaaccctggagctcaagatccatggcacctttcacatgcagaagctggctggcagctcagctgcttcctcccccaatggccaggggctgcagaagctctacaagtgcgccctgtgcctcaaagagttccgtagcaagcaggacctggtcaggcttgacgtcaatgggctgccctatggcctatgtgccggctgcatggcccgtagtgccaatggacaggtgggtggcctggccccacccgaacctgccgaccggccctgcgctggcctccgctgccctgaatgtaacgtgaagtttgagagtgctgaggacctggagagccacatgcaggtggaccaccgtgatcttaccccagagaccagtgggccccggaaaggtgcccagacgtcaccagtgccccggaagaagacgtaccagtgcatcaagtgccagatgaccttcgagaacgagagagagatccagatccacgtcgccaaccacatgatcgaggaaggcatcaaccatgagtgtaagctgtgcaaccagatgttcgactccccagccaagctcctttgtcacctcatcgaacacagcttcgagggcatgggtggtactttcaagtgccccgtgtgcttcacagtcttcgtccaggccaacaagctgcaacagcacatcttcgccgtgcacgggcaggaggacaaaatctacgactgctcgcagtgcccacagaagtttttcttccagacagagttgcagaaccacacgatgagccagcacgcacagtgagggacctcgcgacaggacacctctccgcagaaggcttgccggagacgccgtggggagggccatttgaacattacatccaatcaaagtgtcatttgcaacccagatgtaaaactctaatgatttggccatgaggcgctgctattataagcagctggaaatgaatattaatggcagagattaaaagtattccatgctcagtgttttttattgtcctgctacagctagtgtgcttttagagttttcgccgcagactacatttctagtgttagagaaacctgcttttttgaggctattgtcctttgttccttcatgtattatattgatagttttttttaaaggattagtgtgattttttttgcttcttttctatttctttctttcttgtttttctttctcccccttcagttaactactttttaattgaaattctaggtaattgtgcatcgtgatgtgattgcttggctattgtctgaatatttccttttaattttttaattaaagactaatgctttgattggatttgccagttcaccggacagtgattaaaactctgtaatgaaaataatcggtttccgtgcaactggatggtctgcttttaaatgtgacttgatctgactgcagtaactagttcagctcaataaagggaatccatgcgttcacccccaaaaaaaaaaaaaaaaaaaaaaaaaaaaa

SEQ ID NO: 72 - DNA, 508 bp, Rattus sp.
ctcttgtttcaaagtcttgtcgttcaggtaatctggataatcacctctcctctgtcccaaacactcccaagagagtgattgatgaactcttctttcctagcggtgtggtgtctcttggcggtgcacaccgacaggcacctgtggccacgggcctagatcccaagatgcaggaatcggagcccaaccttgaagggatgtcctctcggagcatgaacttgtcccaggcctacttttgttgctactacgagtctgtgcttttaaggcccgaggggagctcactggactccttctctcaactctgcgtctcttgctgtttgttgttctatgaggaaggggcacgttccggtctcgatccggactttttagactttctgtcaactagcctaacttcccccacattctttaaatccaggtaacttcctaaaaccgcatagatcacatcacgaatggcctcagatcatgaattcatctcaacagcatcgtctcaagaacggtcctcttcctgttg

SEQ ID NO: 73 - DNA, 543 bp, Rattus sp.
  misc_feature (1)..(543): where n may be a or g or c or t/u, unknown, or other
acaaaacaaccatgttaggaaagacacaaaagtagaagtaaatgtctaagtaaaaactaaataaacatgccaagagctatgacttaaaaatcctaatgatgcactcggctacatccagacccaagacacaaatttactttgccacttaacaaaaaagaagcagaatgcccttcatcttcaatgtatttttaaatgtttgcacattgtgtgtctgcaggcaatggcagtatcatcaagtatttaaacaaagtcacctttcaggttgaatgaacaagtcacatcgaaatgtgcaatatattccaggacagccagggctgttacacagacatgctgtcaaaacaatacaaactccccaaacacctcaatataaatgtttaagaaaacaccagtattatataggcaatataattcattacaaaatacttttcttcataaaactatctgctttagacacaaaaggggtatgtggcccttctgtggtgctaacattaccagcttacacagattcgtctaacactgctctangaacagtgctgagaatgt

SEQ ID NO: 74 - DNA, 4562 bp, Rattus norvegicus
ggctccttgctgagttctgctccctctgcttccgaacccgctcgggcgccggggcccgcatccaaggtgctgggccccggacgtagtgccgctgaggtccggagccccgggaaccgggcggggacgttgtcaggctggagctgctcgggccttctgactcagccgctcatccagatttcaggatttctcttctgggcggggagcgcgtaacttcctcattcccgccgggaccctcgcttccagtcagtctggcctgactttgagccctgctgtcaccagggctcctcccttggacttaacctcgccccagttcgaggccatcccatcttggctaggatgactgtaacccagacggacctctttccctatggggactacctgaactccagccagttgcacatggagccagatgaggttgacactctgaaggaaggagaggatccagctgatcgaatgcatccctttctggccatctatgaccttcagcctctgagagcacaccccttggtgtttgctcctggggtccctgttatagcccaggtggtaggcaccgaaagatacaccagcggatccaaggtgggaacctgtactctttattctgttcgattgacccatggcgactttacctggacaaccaagaagaaattccgacactttcaggagctgcatcgggacctccagagacacaaagtcttgatgagtctgctccctctggctcgctttgctgcggcccattctccagcccgagaggcagccaatgagaatattccctccctaccccgaggaggttccgagggctctgccagacacacagccagcaagcagaagtacctggaaaattacctcaaccgcctcctgaccatgtctttctatcgaaattaccatgccatgacagaatttctggaagtcagtcaactttcctttatcccagaccttggctccaaaggactggaaggggtgatccggaagcgctcaggtgggcatcgagttcctggcttcacctgctgtggtcgagaccaagtttgttatcgatggtccaagaggtggctggtggtgaaggactccttcctgctgtacatgcgtccagagactggcgccatctcatttgttcaactttttgaccctggctttgaggtccaggtggggaaaaggagcacagaggcacggtatggggtgaggatcgacacctcccacaggtccctgatcctcaaatgcagcagctaccggcaggcacggtggtggggccaggagatcacggagctggcacagggtccgggcagagattttctacagctacaccagcatgacagctatgccccaccccggcctggcaccctggcccggtggtttgtgaatggggcaggttactttgctgctgtggcagatgccatcctccgagctcgagaggagattttcatcacagactggtggctgagtcctgagatttacctgaagcgtccagcccactcagatgactggagactggacattatgctcaagaggaaggcggaagaaggtgtccgtgtttccatactgctgtttaaggaagtggaactggccttgggcatcaacagtggctatagcaagaggacactgatgctactgcaccccaacataaaggtgatgcgacacccagaccttgtaacattgtgggctcatcatgagaagctcctggtggtagaccaagcggtggcattcttgggtgggctggaccttgcctatggccgctgggatgatgtgcagtaccgactgactgacctgggggacccctccgaatctgcagattcacagactcccacgccaggttcagatcctgcagccactccagacctctcgcataatcacttcttctggctgggaaaagactacagcaacctcattaccaaggactgggtacagctggaccggccttttgaagatttcatcgacagggagaccacacccagaatgccatggagggatgttggagtggttgtacatggagtagctgcccgggaccttgcccggcacttcatccagcgctggaacttcaccaagaccatcaaggccagatacaagatacctcagtacccctacctgctgcctaagtccgccagcactgcaaaccatcttcccttcataatcccaggcgcgcagtgcgccactgtgcaggtcttgcggtctgtggatcgatggtcagcagggactttggagagctccatcctcaatgcctacctacataccatccgagagagccagcactttctctacattgagaatcagttcttcatcagctgctcagatgggcgtacagttctgaacaaggtgggcgatgagattgtggacaggatcctaaaggctcatgaacaggggcagtgtttccgagtctacgtgcttctgcctttgctccctggctttgagggggacatctccacagggggtggcaactccatccaggccattctgcacttcacctacaggaccctgtgtcgtggggaatattcaatcttacatcgtctcaaagcagccatggggacagcatggcgggattacatgtccatctgtgggcttcgcacacatggagagctgggcgggcacccgatctccgagctcatctatatccacagcaagttgctcattgcagatgacagaacagtcatcatcggctctgcaaacatcaacgacaggagcttgctggggaagcgcgacagtgagctagccatactgatcgaggacacagaaatggagccatccctcatggatggggtggagtaccaggcaggcagatttgccttgagtctgcggaagcactgtttcagtgtcattcttggggcaaatacctggccagacctggatctccgagaccctgtctgtgatgacttcttccagctgtggcaggaaacagcggagaacaatgccaccatctatgagcagatcttccgctgcctgccgtccaatgctactcgttccctgcgggctctccgggagtacgtggctgtggagtccttggctacagtcagcccttctttggctcagtctgagcttgcccacatccggggccacttagttcacttccccctcaagtttctggaggatgagtccttgttgcctccactggggagcaaagaggggatgatacctttagaagtgtggacatagctggggcttccgctcagcggcagttgctagccgttgggccctatcgtgcctggatccttgccccacaccctgagttctgagggcagtgccctttgatccttggggaggacatctctgaggactcctagagaatcacagaggacctttacttgagaagtagccaaagggagcactcccaagcctggcctgggaaagcaggagagagttctagagaggtttgccttcttgtcaccatgttcagaccactatgccacagaaccctagtcctacgaggaggcctgaggtcaagccttttattccaggaaaagggactcctgccctgggtcgtcttcatctcactcttcctcctgcccttgaacccctacatgccctagggcctctcccagcccgttgctgcaaagatgggggggggggagtatagagccactttgactgcagtccccaccagcggtggtgaggacaccttaactgcctccaccagcctgctgacagacactaactctgtaccggttcaccaagcatttcataaataaatgtgtagaaaaggccatgcttcttcttggagatggatgttgtcttaggtgctccctgcctcagttctaacttctttcttctgcttgcatctctgtgcttgcttgccccccctttctccctccctccctttccttcttccttttccttccttcctccctccctttccttcttccccttccttccttccttccttccttccttccttccttccttccttccttccagggggtggggtggggttgatttccaaatagagtttctctgtgtagccctagctgtcctggaactctgttaacaaggctgccctcgaactcggagagatccacctgcctctgcctcctaagtgcttggacataggcttgggccacagctccatctctgtgcttctcagcagcttctgagttcattcacctaactcctgaagatctgggctaattctgtgtgtgtgtgtgtgtgtgtgtgtgtgtgtgtgtgtgtgtgtgtgtgcgtgatacatatcttcataacttttcatatgtctgaagtattttgcaattagaaaactgctgaacacagatagccacagaagcaggaagaaaataaggagaatggagaattaggaaggcatcatagaaggcagtgggggtgggtgagaatgtgaccagaaggtattaggttgctctcccatccctctagttctcttgagaagtgaattctagggttagtgagagatgcttcagcagaggaagatgcttcctgttaggcttaacaacttgagttcgatccccaggacccacagtctaggaggagaaaacagtctcctgaaagttgtcctctgacctccacacacagaccatggcattcctgtgtgtgctcacaataaataaattcattttcaaaatcaaaaaaaa

SEQ ID NO: 75 - DNA, 727 bp, Rattus norvegicus
agctgggactggttcctgccacctccccgagcccctgcactcttgtctctctctgtctccacagcagcgtcaccggctcccggatcctgccagccgtccagctctacaggcaccatgaggagcgccatgctgttcgcggctgtcctcgccctcagcttggcatggaccttcggggctgcgtgtgaggaacctcaggagcagggtgggaggctcagtaaggactctgatctctctctgctacctccacccctgcttcggagactctacgacagccgctccatctctctggaaggattgctgaaagtgctgagcaaggctagcgtgggaccgaaggagacatcacttccacagaaacgtgacatgcacgacttctttgtgggacttatgggcaagaggaacagccaaccagacactcccgctgatgtggttgaagagaacacccccagctttggcgtcctcaaataggccagcagtgcagaaaagcactccactctcagaccctggactgcatcataaagacagggttcctgtggcggtcccagtgcctgcgctcctgcttccctgcctgcaaggtcctcctgttggctcccttccctactctgcacagatgctgcatatgaacagcctctacccccatatcaattatggtttctgtagtgtcctgcattaaaaataccatgtctcctcctcaacaataaagggtttttacaatggagtgactgaaaag

SEQ ID NO: 76 - DNA, 458 bp, Rattus norvegicus
ttggattcccaatactatttttattgcaactgcatgagagttttttaaattgggcattacaccatattacacaggattacgaacaagattacagtacatgtccattccagtagtaccagcatcacatcgaaaacagcacttattctggactgcattttacatgcaatagctattgttctaattatggatcaaaaggtttgaattttatttaaactactgtactaattgactacacaggtttaaaccccaacatcaacaacctgaataaatttaggctcaaatttattacatgaatagatgacaagccaccatttgtttgactatgtataaaatcatgcatttatatttttttgaaacatcaataacgtcagacactccataaaaatcattgacttcaaagaagtgaaagactaaaaagaaagagtgcaaattgggggtgatagccgagcaccttttga

SEQ ID NO: 77 - DNA, 452 bp, Rattus sp.
aaaacatgattacaagaaaagaatacttcatttacgtgtaacactgtcttcatggacgtaccgtggtcacggagtagaaatacaacatgagtatacaggaaaggagaggagagaccggtgtttggccctggtggccccctcacagagacacagaaagcagcagacacctctgtgcattcaatgggacagtggacacttgtgtttggagcagccagagagtcagggaggatgggcaaagccacctctgggaccctaatttcgtgtggtaaccccaaagagatactggtctgcactggtggaggctggctcctgtcaggttcttcgcagccttcagggacaagctgaggctcctgagtgcccagtggtttgtcgcatagcttgctgggcccatgggctccatggtgtgaagagaaaaaaggccaaagtgggcgactggctgatctctaggtgac

SEQ ID NO: 78 - DNA, 2280 bp, Rattus norvegicus
cagcaagccaccttttcactctgtccctactggaaagcactgggaagagctgcaccggatagacatgaatgaggacaaagacaagagagactctatccagatgagtatgaagggatgccggacgaataacgggtttgtccaaaatgaagacatccaggagcaggacccagactccagggacactccacagtccaacgctgttagtatccctgctccagaggagcctcaactaaaggtggtgcggccctatgcagggatgcccaaggaggtgctgttccagttctctggccaggctcgctaccgggtgcctcgggagatcctcttctggctcaccgtggtctccgtgttcctgctcatcggagccaccatagccatcatcatcatctctccaaaatgccttgactggtggcaggcaggtcccatgtaccagatctacccgaggtcttttaaggacagtgacaaggatgggaatggagacctgaaaggtatccaagagaagctggactacatcactgctttaaatataaagaccatttggatcacttccttttataaatcacctttgaaagattttagatatgctgttgaagatttcaaagaaattgaccctattttcggaacaatgaaagattttgagaatttggttgctgcggtccatgacaaaggtttaaaattaataattgacttcataccaaaccacaccagtgacaaacatccttggttccaatcgagtaggacacggagcgggaaatacactgattattacatctggcacaactgtacccacgccaacggtgtaaccacccctcccaacaactggctgagcgtgtatggaaactccagctggcagtttgatgaagaacgaaagcaatgttattttcaccagtttttgaaagagcagccggatcttaatttccgaaatcctgctgttcaagaagaaataaaggaaataataaagttctggctctcaaagggtgttgatgggtttagtttcgatgcagttaaatttcttctggaagcaaaggacctgagaaatgaaatccaagtaaatacatcccaaattccggacacagtgacccgctactcagaactgtaccacgacttcaccactacccaggtgggaatgcatgaccttgtccgggacttccggcagaccatgaaccagttcagccgggagcctggcagataccggttcatggggacggaggtgtcagctgagagcaccgagaggaccatggtgtactatggcctgtcatttatccaggaagctgacttccctttcaacaagtacttagccacactagacactctttccgggcatactgtgtacgaagctatcacatcctggatggaaaacatgccggaaggaaaatggcccaactggatgattggcggaccagagacttctcggctgacttctcgagtagggagcgagtatgtcaatgccatgaacatgcttctgttcacactcccaggaacccccataacttactatggggaagaaatagggatgggagatatttccattacaaacctcaacgagcgctatgacactaacgcccttctctccaagtcaccgatgcagtgggacaatagttcaaatgcggggtttactgaggccaaccacacctggctccccacaaactctgactaccacacagtgaatgtggatgtccaaaaaacccagccgagctcagcactgaggctatatcaggatctgagtctactccatgccagagagctgcttctcagcagaggctggttttgccttttgagggacgacaatcactctgttgtgtacaccagggagctggatggcatagataaagtcttccttgtggttctgaattttggagaatcatcaactgtgctaaatctacaggaaactatttcagatgttcctacaaaactgagaataagattaagtaccaatccagcctccaaaggcagtgatgttgatacccatgccgtttctctggagaagggagaggggctcatcttggaacacagcatgaagactctcctccatcaccagaaagctttcagagacaaatgttttatttccaaccgtgcatgctactccagcgtgctggaccttctgtatagctcgtgctaggcagctctgtaagaggtggccaccctgcgtctggtatgcttgccatcacacatgcaagggcctcaggaatggcatcagttcttagatatttctgtagcacgaatgcattgttttaggtaagattctcaaatgtttggaaggacaataaaatgtttaaaagatta

SEQ ID NO: 79 - DNA, 1673 bp, Rattus norvegicus
ggcttaaggaaacttgctgcggaggaaagacattggctggacacctctagatctaccttaagacttgagagctaggttgggaccatggggacagcgaaggtgaccccatctctggtgttcgctgttactgttgccacaatcggttctttccagtttggctacaacaccggagtcatcaatgcgcctgagacaatcattaaggactttcttaactacacgttggaagagcggttggaagacctaccaagggaggggctgctgaccacgctctggtcgttatgtgtggccatcttctccgttgggggcatgattggctctttttctgtcggactctttgtcaaccgctttggcagacgcaactccatgcttctagtcaacctgattgccatccttgggggttgccttatgggcttcgccaagatagcggagtcggttgaaatgctgatcctgggccgcctgattattggcatcttctgtggtctgtgcacgggctttgtgccgatgtacattggagaggtgtctcccactgccctacggggtgcatttggcacgctaaaccagctgggcatcgttgttgggatccttgtggctcaggtctttggtttggactttattctgggctctgaggagctgtggcctggactactgggcttaaccatcattccagctatcctacagagcgcagcccttccgttttgccccgagagtccaaggttcttgctcattaacagaaaggaggaagaccaagcaacggagatcctccagcggttgtggggcaccccggatgtgatccaggagatccaggagatgaaagacgagagtatcaggatgtcacaggagaagcaggtcactgtgctggagctcttcaagtctcctagctacttccagccccttctcatctccgttgtcctccagttgtctcagcagttctctgggatcaatgctgtgttctattactcgacgggaatcttccaggatgcgggtgtccaggagcccatctatgccacgattggagcgggtgtggtcaacactatcttcactgtagtctctctgttcctggtggagcgggcaggaaggagaaccctgcacatgataggcctgggaggcatggctgtttgctccgttttcatgacgatttctctgttactgaaggatgaatatgaagccatgagctttgtctgtattgtggctatcttggtctacgtggccttctttgagattggccctggccccattccctggtttattgtggctgaactcttcagccagggcccccgaccagctgccatggctgtggctggctgttctaactggacctccaactttttggtgggaatgttcttcccctcggctgcggcctacttgggagcctacgtttttatcatcttcgctgccttcctcgttttcttcctaatcttcacctccttcaaagtcccagagaccaaaggcaggactttcgaggatattacccgggccttcgaggggcaggcgcactctgggaaaggctctgctggtgtggagttgaacagcatgcagcctgtcaaggagacccctggcaatgcctgagccggacctcctccctcacctccctccactgtggaaagccaacctcccctgaagtggcgagacctcatcaggatgaaccaggactgcttctgagcgctcgtatcaca

SEQ ID NO: 80 - DNA, 2042 bp, Rattus norvegicus
gaacataaagtcagattgctaaacttctgtgtcgactgaaaaacatggtgaagcgagttgcaattgtgggagctggggtcagtggcctggcctccatcaagtgctgcctggaagaaggactagaacccacctgcttcgagagaagctgtgacttgggaggactttggagattcacggaacatgttgaagaaggaagagccagcctttacaactcagtggtttctaacagcagcaaggagatgtcttgttactccgatttcccttttccagaagactacccaaactttgtgccaaattctctgttcctggaatatctccagctgtatgcaacccagttcaaccttctgagatgcatctatttcaacaccaaagtgtgcagtataacaaaacgcccagatttcgctgtctctggacaatgggaagtggtcactgtctgtcaagggaagcaaagctcagacacctttgctgctgtcatggtctgcactgggtttctaactaacccacatctgcccctggattcctttccaggcatacaaacttttaaggggcagtacttccacagccggcagtataaacatccagacgtatttaaggacaagcgagtccttgtggttggaatgggaaattctggtacagacattgccgtggaggccagtcacttagcgaaaaaggtgtttctcagcaccaccggaggggcatgggtgatcagccgagtctttgattcagggtacccctgggacatgatattcatgacgcgatttcagaacatgctcagaaatcttctcccaactccagttgtgagttggttgatatcaaagaagatgaacagctggttcaaccacgtgaattacggtgtggctccagaagacaggactcagctgagagagcctgtgctgaatgatgagctcccaggccgcatcatcactgggaaagtgttgatcaagcccagcatcaaggaggtgaaagaaaactctgtcgtctttaacaatacaccgaaggaggagcctattgacgtcatcgtctttgccactggatactcctttgcgttccccttcctcgatgaatcaatagtgaaagttgaggatggccaggcatcactgtacaagtacatcttcccggcacatctgccaaaaccaactctggccgtgattggcctcatcaaacccctgggttccatgatacccacaggagagacacaagctcgatgggttgttcaggtcctgaaaggtgcgactacattaccacccccgagtgtcatgatgaaagaagtcaatgaacggaagaagaacaagcatagcggatttggcttgtgctactgcaaggctttgcaatccgattacataacgtacatagatgacctcctgacctcgatcaacgcaaaaccggacctgcgggccatgctcctgactgacccacgcctggctctgagcatcttcttcggcccatgcacaccttaccatttccgcctgactggtccaggaaagtgggaaggagccagaaaggccatcttgacccagtgggaccgaacagtgaacgtcaccaaaactcgaaccgtacaagaaaccccatctacctttgaaactttgcttaaactctttagttttctggctttgcttgtggctgttttctttattttcctgtaagtgaaagatctaactggctttccaaatgtgtggagtataaccttccaacttctctaatgtaacaatttcaccttcgtaattgtaaaccacgtccagagacacccaacccctacctctccccaactcacctcattggcaccttcattgctgggtctcttgctagtccatcaggtttagtgcaagaaaataatgtccagcaattctgttcacttaaaatgttggaaggatccaggccccctttcaggaagaatctgcccccagagaggactctgagcattctttcaatctaaaaaactgctttccctagatcttaatgaaaagcccaacttcgcggaatattggtctgcactaaaatagttctctgtgtattagttgactacaaataaaatggaagaaact

SEQ ID NO: 81 - DNA, 650 bp, Rattus sp.
gaatagcctgaggttctctctctgtgagatgggtccatcgatcacatccaaggagaaaccagaacccttgcccccaccagtgatgaggaagtcactgagtttggttggagttggacctcggtcagctctgtcgtctttatagccctttaacaaaccgaagaagctttctccggatgtcccctgcttgtcgattgaagatgtgctaccatattccctgtgcaatgatggccccgacttgtaggttggggagatacattcatcaaaactatccacatcaagctcacttatggtgatatcactgttgctacgctgacggattcggcgaagggctttcctgggggagctggggtaggcttcaggcatgaggaatctcgagtccatgctctccgctggccccgtcttgttcttcagggtgttctggatgctcttcagcatggctgagtcattggaattgaggctaacagaactcccctgactcacaggactgcctttggaggacaggctctcaaggcaacttgaggtttctatttcctggcttgaacggctagattcttttacattttcctttcttggaggccaatctgcaattcttgccctaacccccatcttggggactccgggggttgaggttatatggtgaggaccttcac

SEQ ID NO: 82 - DNA, 3300 bp, Rattus sp.
cggtcgcacaccccccggtgtcccctcgcctccctcgccgccgcccccttcccccgctcgcgataagaagagccggcggcaggagaggggatgaagatggcggacgcgaagcagaagcggaacgagcagctgaagcgctggatcggctccgagacggacctcgagcctcccgtggtgaagcgccagaagaccaaggtgaagttcgacgatggcgccgtcttcctcgccgcctgctccagcggcgacacggacgaggtcctcaagctgctgcaccgcggcgccgacatcaattacgccaatgtggacggactcaccgccctgcaccaggcttgcattgatgacaatgttgatatggtgaagtttctggtagaaaatggagcaaatatcaatcaacctgacaatgaaggctggattccactccatgcagccgcttcctgtggatatctggatattgcagaatttttgattggtcaaggagcacatgtaggagctgtcaacagtgaaggtgacacacctttagatattgcagaggaggaagcaatggaagagctacttcaaaatgaggttaatcggcaaggtgttgatatagaagcagctcgaaaagaagaggaacgcataatgcttagagacgcgaggcagtggttgaacagtggtcacatcagtgacgtccggcatgcaaagtccggaggcacagcactccacgtggcagcggccaaagggtatacagaagttttaaaacttttaatacaggcaggctatgatgttaatattaaagattatgatggctggacacctcttcatgctgcagctcactggggtaaagaagaagcatgtcggattttagtggacaatctgtgtgatatggagacggtcaacaaagtgggccaaacagcctttgatgtagcagatgaagacattttgggatatctagaggagttgcaaaaaaaacaaaatctgctccatagtgaaaagcgggataagaaatctccactgattgaatcaacagcaaatatggaaaataatcaaccacagaagacttttaaaaacaaggaaacgttgattattgagccagagaaaaatgcatctcgaatcgagtctctggagcaagaaaaggctgatgaggaggaggaaggcaagaaggatgagtccagctgctccagtgaggaggatgaggaggatgactccgagtccgaagcggagacagataagacaaaacccatggcttctgtaactaatgctcacactgccagcactcaggcagctcctgccgctgtgacaacacctactctgtcttccaaccaggggacccctacatcacctgttaaaaagtttcctacatcaactacaaaaatttctcccaaagaagaagaaagaaaagatgaatctcctgcatcctggaggttaggacttagaaagactggcagttatggtgccctggctgagatcactgcatctaaagaagctcagaaggagaaagacactgcaggcgtgatacgttcagcttcgagtcccagactctcgtcctctttggataataaagaaaaggagaaagacaataaaggaacaagacttgcatatgtcgcccctacaatcccaaggcgactaggcagtacgtctgacattgaagagaaggaaaacagagagtcttcaaatttgcgaacaagtagttcttacacaagaagaaaatgggaagatgatcttaaaaaaaatagttcaatcaatgaaggatctacttaccatagaagtacctcaaatcgtttgtgggctgaggatagtactgagaaagagaaggacagtgctcctaccgcagcgaccattcttgttgctccaactgttgtaagtgctgcagcttcttctaccacagccctgaccacaactactgctggcactctttcctccacatcagaggtcagggagagacgcaggtcatacctcactcctgttagggatgaagagtctgaatcccaaaggaaagcaagatctagacaagcaagacagtctagacggtcaacacagggggtgacactgactgacctccaggaagccgaaaagacaataggaagaagccgttctacgagaaccagagaacaagaaaacgaagaaaaagacaaagaagaaaaggaaaagcaggataaagagaaacaagaagaaaagaaggagtcagaagtatctagagaagatgaatataagcaaaagtattccagaacatacgatgagacttatgcacgttacagaccagtgtcaacttcaagttcaagcactccgtcgtcctcctcactttctactctaggcagttcactctatgcctcaagtcagctcaacaggccaaacagccttgtaggtataacctctgcctactcccggggattaaccaaagacaatgaaagagagggagagaaaaaagaagaggaaaaagaaggggaagataagtcacaacctaaatcaatcagagaacgacggcgaccaagagaaaaacggaggtctactggagtctccttctggacacaagatagtgatgaaaatgagcaagagcggcagtcagacaccgaggatggctccagcaagagggacactcagacggattctgtttccaggtatgacagcagttccacgtcatcaagcgatcggtatgactccttgctgggtcgttctgcctcatacagttacttagaagaaaggaaaccatatggtagccgactagaaaaggatgactcaactgacttcaaaaagctttatgaacaaatcttagctgaaaatgaaaaactaaaggcacagctacatgacacaaatatggaactaacggatctaaagttgcagttggaaaaagctacccagagacaagaacgatttgctgacaggtcactattggagatggaaaaaagggaacgaagagctctagaaagaagaatatctgagatggaagaggagctcaaaatgttaccagacttaaaagcagacaaccagaggctaaaggatgaaaatggggccttgatcagagttataagcaaactttccaagtaggacagaaaacacacaagcgaagcagcgggacttgcacacactccccagtggaccacattggcagtcactggacgccagaaagaacccctggagactgtcattttccgatatcctgccaaacgccctcttatctaggagttttgtttcgtttaatcttctgccccacccccttggttatcaagaccattgtttcatgttaaagccgctgctgagaagattttttttcaatgactgagaaaacttgtttacagctccagcaaataaagaaagtgttcaagg

SEQ ID NO: 83 - DNA, 1952 bp, Rattus sp.
gcggcacgagcgcccacgcagaaggcaaggtgtcccgaggctccagggttatgagatcgtcactattcagaaccttttaacaacaggaagtggaaacatgaccaaatcatacagcgagagcgggctgatgggcgagcctcagccccaaggtcccccaagctggacagacgagtgcctcagttctcaggacgaggaacacgaggcagacaagaaggaggatgagcttgaagccatgaatgcagaggaggactctctgagaaacgggggagaggaggaggatgaagatgaggatctggaagaggaggaagaggaggaggaagaggaggatgatcaaaagcccaagagacggggccccaaaaagaaaaagatgaccaaggcgcgcctagagcgttttaaattaagacgcatgaaggccaatgcccgcgagcggaaccgcatgcacgggctgaatgcggcgctggacaacctgagaaaggtggtaccctgctactctaagacacagaagctgtctaagatagagacactgcgcttggccaagaactatatctgggctctgtcagagatcctgcgctcaggcaaaagcccagacctcgtctcctttgtacagacactctgcaaaggtttgtcccagcccactaccaatttggtggctggctgcttgcagctcaacccccggactttcttgcctgagcagaatccggacatgcccccacacctgccaaccgccagcgcttccttcccggtgcatccctactcctaccagtcccctggactgcccagcccgccctacggcaccatggacagctcccatgtcttccacgtcaagccgccgccacacgcctacagcgcagccctggagcccttctttgaaagccccctaactgattgcaccagcccttcctttgacggacccctcagcccgccgctcagcatcaatggcaacttctctttcaaacacgaaccatccaccgagtttgaaaaaaattatgcctttaccatgcactaccctgcagcgaccctggcagggccccaaagccacggatcaatcttctcctcgggtgccgctgctcctcgctgtgagatccccatagacaatattatgtctttcgatagccattcgcatcatgagcgagtcatgagtgcccagcttaatgccatctttcacgattagaggcacgtcagtttcaccattcccgggaaacgaatccactgtgcttacagtgactgtcctgtttacagaaggcagcccttttgataacattgctgcaaagtgcaaatactcgaagcttcaagggatatatgtatttattgtcgttactgcctttggaagaaacaggggatcaaagttcctgttcaccttatctattgttttctatagctcttctattttaaaaaataatagtacagtaaagtaaaaaaggaaaatgtgtaccacgaatttcgtgtagctgtattcagatcgtattaattatctgatcgggataaaaaaaatcaagcaataattaggatctatgcaatttttaaactagtaatgggccaattaaagtatatataaatatatatttttcaaccagcattttactacttgttacctttcccatgccgaattattttgttgtgattttgtacagaatttttaatgactttttataacgtggatttcctattttaaaaccatgcagcttcatcaatttttatacatatcagaaaagtagaattatatctaatttatacaaaaataatttaactaatttaaaccagcagaaaagtgcttagaaagttattgcgttgccttagcacttctttcttctctaattgtaaaaaaaaaaaaatagaaaagaaaagagaaaaacaacaaattgcacaatttgagcaattcatctcactttaaagtctttccctctccctaaagtagaaaccagacccataacactcaagaggaaaaaaaaaaa

SEQ ID NO: 84 - DNA, 1017 bp, Rattus norvegicus
ttcaaagtcctaaaacgcgcggccgtgggttcggggtttattgattgaattccgctggcgcaggatcctctgcagaaagagagagcgcgagagatggagatggacaaacggatttatttagagctgcggaacaggacgccctctgatgtgaaagagctggtcctggataactgtcggtcaattgaaggcaaaatcgaaggcctcacggatgagtttgaagaactggaattcctaagtacaatcaacgtaggcctcacctccatttccaacttaccaaagttaaacaaactcaagaagcttgaattaagcgaaaacagaatctcaggggacctggaagtattggcagagaaatgtccgaaccttaagcatctaaatttaagtggcaacaaaataaaagatctcagcacaatagagccgctgaagaagttagagaatctcaagagcctagacctgtttaactgtgaggtgaccaacctgaatgcctaccgagaaaacgtgttcaagctcctgccccaggtcatgtacctcgatggctatgacagagacaacaaggaggcccctgactctgatgttgagggctacgtggaggatgacgacgaggaagatgaggatgaggaggagtatgatgaatacgcccagctagtggaagatgaagaggaagaggatgaggaggaagaaggggaggaagaggacgtgagtggagaggaagaggaggatgaagaaggttacaacgacggggaagtggatgatgaggaagatgaagaagatgctgctgaagaagaagggagtcagaagcgaaaacgagagcccgatgacgagggccaagaggatgactaaggggaattaacctgtttggggaaattcctattgtgatttgactgtttttacccatatcccctccccctcctattcctgccccccgaaacttatttttttctgattgtagcgttgctgtgggaatgagaggggcaaagtgtactggggattgccgggggtggggtgggggtgggaggggag

SEQ ID NO: 85 - DNA, 614 bp, Rattus sp.
gtttgattgtataatttaatgacaatataaacagtatagtttgttttttttctttttctttttttaaaaaaaaagactaaagcaaaaatgattaaaagctaacagaagctaccgtgacattggtttgacattcaaagtttgagtcttagcaaaacggccaaaggtatcttgacttgatacagagtatataatataaagacttttagacctaaaaatcttcaaacgttatttgaatttagcaaaagcaaaaatttcatatcaaagtgctaaacagtgctgccttaaaggtcactgcaaacaaaggataaaataactgcgtgggaagtgacatttattgtacaaatggttaataaaaagacacattataaatatatatgtaacctgctatgtttttatatatatatatatgcttatttaatttctaaccggtgtatccaagtcaccacgaacaccccatttgattattctgtaactcagcctccaggagctggtggtcttgcaataaatacaggcaaagcgattacaatagaacgtgcatacaaatgttcatacaaataaggacactatgcaacgaatcatttcataataatatgccatcaacagtagaaacatag

SEQ ID NO: 86 - DNA, 481 bp, Rattus norvegicus
ttccaaggcaagtaaagttgattttaatattgttctcttacaggcagattaaacaaacaggcaaagtacatacatagtacatggcagtcttaagtgacctcaaggtgtattattaactcagactgtgctagcaaaaattccagtctcttaggatgtaagatatttttatcacaatgcattgccacagttcctcctttttgttttattaaaaaaaccatgtgggtggagggggtcactggtgaagaactaagaactgatcaagcatggtactgtagggatagcctgaatgcaccacttcattctaggtgatccttctgaaaagttggcaaaggcataacaggacttgagaaggaaaacagaatgcattacctgtagaagatcacacttaagatttattccctgtactaacttaaagaatgaattcatcttctccaaccttcccctcccaagaagattcactggaatgacttacaggctgcag

SEQ ID NO: 87 - DNA, 458 bp, Rattus sp.
ccggagctggggaccgaattttaggctttcttgattgactggctggcacggctggagaatccgacttcacgaagagcagagggctggcaagaggagggcagttgagcagcagagcttccttagccaagtcagagggctgccacgtctctgcttccaaaggtctcctgctgctgggagcagggtgatggccccaggttagcggaagtctgcgttgaaagctctgccaatgactagccgcctcacctcactggtcccacctacgatctcgtacagtttggcatctcgaagaaagcggcccatggggaagtcattgatgtagccatttccacctaaacattgaatgccatccagggctacttgtgtggcacactcggctgtatacagaatcacaccagcacagtccttggcagtgatgtggccctcgtcacaggccctggcgacattgtagacatactgtc

SEQ ID NO: 88 - DNA, 30000 bp, Rattus norvegicus
ggatcctcaagggccccagagaacttttataaggaagaagaccaaaatatcccaaccacagctttcacctagccctcctgagttctttttccagagctattttagaggagtatctgactgggaagaaattgggcttggtaccttgagctggaaggccatggagtcattcttaaagagcttatcccagctctgggaggacagagaacaccccaactgctctcagactatccaagtgacctttagtgctctgaacaggacaggcaccccacacaggtggaataacatactgaaggactgggcaggagccagaactccacttctcaggaatggccagttgcaagttctaagcaaggtagccatgagagaggtccagggctggggtctcctattacttctcagtccctcctcccagacaggtagggcctctcatcagatttcttgaacttaactgaaaagccacataacatctagatctttggggaatctcaacatagagtcccactctttagcactgagcccagactgggtgtcacctgtccggcaaatgagagattagagggctagggtgatcctggtcaccccaagggctggctgacttgcctgccactcaagccaaatccatctgtgtcttcctgaaagctccacccagagccagggtgacagacctctgaactagacataggtcttcttgctagtacttggtggttgggaagcctcctccaacagtgttaagaaccccttcctagtcaccccctctccacaggcccacctaaagaaatgtcacaggtccctagtgagtcctgtccctgacaaaggaagactagaagtgagcatgaggttggatggatagacattagacccaaaagaagaaagaaggaggggcctgggacctcctacaggaacaccccagaggtaagttagcacagcctgaggtgtggtgccatgccgggatggagctacgtgagctctgcgagcacaagcagaagcaggggctggaaattgggatggttgaagtccgttgagcctccagagaaagccactggctgtcgctgcctaggtcttgggtgggtgagcagatagggcacaccactgctcaagtgccacaaactctctggagggcatatgagtcatgggtaaagggaacaagaatctttgggtccttgggccttgtggggcaagctgaacttttcaggaaattcaatggactcatgctatggagttcttggggcacagaagtatggtccagacgtccagatctctatctatagcctcactcctgcagtctttcaggcctcaagggagaggaaggttgctcccaagactcttaatctgcccaagctgaagcacatcccccataccttcctgagacctcctgcaactctagtttagggttctccttcctactggtcttgaggagaggaagcaaaatactagagataggcttgttctgactcccaactcctccaggagtggacaaaaagcagtcactcaaatggaatcagtgcccagggagcagaggtcgaatcagactcagtctttgatcagcatcaccctgagtgctaaccccagacatcccagccagatgttcctttaaggcccaggttactccatggaggtcagttctagacctggctcagatactcctgctggcctcgctgaggtccttcagcacagccctgcagcccaggagctccttcctgccttggaaccaggggtcctcttgtctatgaggcctgcagtgataatcgtgtttacccagctagctctgttgttgctttggtatttgagctgggactgggtcgtttcaggacctgctccctaacaactcccagccttgctggggcccctcatcgcctctgcctaccatctggagggccactgtattactaaggatagccagtatacctgtgctgtactcttgcctggattgatgaggcaattcagatccacatatgtgggagagactcatactaataccacacatggggagactgaggcctggagccagggcagccactcttctgttctgctcagtactattctccctcacagtataagttccccagacacctaatatggtcttccaatgtagctgcctccagtttggcttccacagattgtattgtctctgggtacaagtgagctggcacctcaggctagccccagggactgtatttgctttctcgcattatcaataaatgttcactgtacttgtgttctcccaggctgaacaggccatgagtgatatgagaggtggggtcaaggatgctgaggtccatttttaccctttgagatccttatacccagcaaggttctgaaccaaggagcagaaccaggtagctctcaagtcaacctgcccagaaaagttggcactaaagagtatcaagcactatgtggctctctagcctaatgtaaatgagggagacccctgaggagcctttccacaaactcagatagggtgcccctcatgctggtaactactgtttgggatttgtgaaatctggacaacgtagccagaaatggctagtcgtgaaagtgtccagcagagggcaccaaaaccttgcttttgcagtactccaagtccgaaccgcctaccataagtccttctaggggctgcatagtgtctccctttgctcccctgaagtcacaggaacaaagaggcttcccaagaagctggctgccctgaattctggagagcatgaggtttgggtaagaaagactggatgtgcacctttggggtttagtttcttagatacccatggcggaggatgaacaaatgtccgggtgtctgcattattcctagaatgtcccgggttagagtcatctcctacctgtctagaattacagccgcccagaggagagtgtaaggggcaatccatgcagcacctcacactacggctaagcaaacaggtcaggggagccgactgtctgtttccagctgggacctttgtggttcccagagccctagtggacacagagaccgggagccagaggagccagtaatccctctctttgcttggagttttcttaggcacataggctggaagacaaaactctagttttcacatggaataggggctacagggaccccagggtgcaggatgcttcatgcagagtccatctgggacggggtcagccctttcccatgtctaccttccgctcagtgctgagaagggaaggtcagttttgcaaagaatgctggggggtggtgtggccaggggtaggagagctgaagaacagcctggataccgtgtctcttttctcttcccccatccaactattgtttctggctgggccacagaacagcaagcgattccttcgaaagtgaagctgtggggtggccctgtgtggtccagtcatgctgtgctggcaccaagttacccagtggctttgagccagcccccttggaatcactggctcactgcctggctcccaggtagcttgttccaccaccaccaccaccaccttggtacaaacacctcacctataacaccttctgctgcaggtagtcagaattctgtgtctgatctgcagacccctgaggcagacccttgaatctgggcctaggctgagctataggtttaccagcatgctgctctctccaggggcggggggctggggagtaagccagggctcctcaaactaaaagttgtcgtggtgatgcatcacccactaggtgctccgtccatatactcaaactgactagattcagttatctgacctctgggctaaggccactgactcttggtgtgccagccaagtcccaaaggctatgtgtggctcccacctgtagactaatgatagattctttaccattttgccaacaacagaagtatgtcttaaagctgcctggaggtccacagaccctgatgtttcctagaggcctcctcccctgcagtcctgcgaagcctttagaatcctgctgctaacattccaccaggacctccctctggctctcccagcccatctgtgtttgtggacaacagagtggagtccctcagccctgccagcttgtggctgttcatgctgaggccctggcactcccccctctgctcttcaaaggctaccctggcttagaattgagtcctgctccagctgggtctccagtcttctgtccaatccctaagacccactgtgacctggactgctgaggacccggaagactcctcctagcttttatgggccaggtcttgagcatgggtcagcaacacctggtgacccagaagcattcagaatgggcctgaggaccagaggtaaactgaggcacccaaatctttgccctggcaaatgcttcctgactttcccccagcctgacatctggggatgaagaacagcttcagttggctagggaacctgaaaaagaggacgttcccaacctggacccaaatattgaaaaggagatgggaggggctggttgggtgaggaaaaaagctgacagagatctagcaaggaaggctccctatatcccagctctagctgcacaggtcagatttaaatatgagtgggagcttcctgaagcaatgttcatattctatagttgcctcctcatacggccagacactgctgtgattggactcatcctgagttcttctctaagacctggcatatgggctggccatggcacaagcaagaggtggctatagctacaaattatacaaaagatgtgtattgacctcagccttcttgggttgtgggtctaaagaatgaaagctagtcagtgagtgtgaggctaatgtcttggatatgagagtctctggctttcatcttttggagatagccctggctctcatgtgggtattgtagagaagagaaaacgggtacacttcctgtatatgctcccaacatagccatgagtatacaagaggtgtcattttctaagggacaggatcccccagagatggaggagtgaggcatccactcatcagtggagaaaagttgaatcagggagaaagggtttagtcaagagtaaactcttgggagagaccagctgcaccatgctgcagatgaggattcctctggtcctgaggttcattgtgtatcttgggacaagaaggtctttgccattctttgaagtcctagctcaatactcaaggcatccctagccagagttccaccccaaatcctagagttccctctagaatgaagttttgtttgaacaggtagaagagcctttatggacaggtgccccacaactataggttctggactgttccaaatccatttccacatggttccagaaataatctgtgtgatcagaaggaaaaatggaggttcaaggtaaactgattgcccagtgtaatgagcagaataggtatttgaacctaggcagtcatcagccccctgtacaaactctattcccatgtgatgcccagtgggtagaagtttatagaatagaccccttatctccaagcatgagttttccttggttctcagatgtggagttgtagcttttaagaaggttgactcagccttgccaatcagccaaggggaggaaatcaatgtgcaaaggttaggaacaagttatatgaggtaggatgagaaggatttttgttttttcctgtgttatgggtgaatcagaacctcatattggcaagattttctaccactgaactacctcctcagccctaacaccttccccttctctccattcccaaggcttgataaagcaccttgtagttatgattgtggggaaagaggcacgttctgaagagtcaatgcaatttattaaatgaccaccagatgtcaagctcgagccgggtttgcaaattcctttcgggtgcaggatggtggggaagccaatgggtattaagaacagtgtttaatccactcttgttcatcccagggacccccacctcttatctagaatccaagaattatacctagaaagagtccataataaatttattttaatataaatatgacaaagtttgcttagaactagagttagaacttagttcaaggtgtttggggtacaggggtcttaggacaggaagatatgcaaagggaagactgcctgataagataggggaggggaagcctcagtctcaagtacaagactacagaactcacttataccatgatgctatagaaggtctcctctctgtaggagaaagaattacagcatcgcctcttaccactttccttccctgagtaatcccaaatccctgcagtaagaactttaaattgtgtattgtaattaaagatatacctaccagatgtcagcatgaggccactttagcagtccaggaaggaaaactggattctgggaatttctagaggtacttcagctttgtttttcataaatgagagtccaggcagattcccctgaattagatgactctcctctctccaagtctcaagtgagtagatttttttgtagtgtctttatagttacatcttttttttaaagttacatttttttaaagatttattcatttattatatatatgtccactgtaactgtcttcagatacacacgaggtacagacgaggacatcagatctctttacagatggttgtgagccaccatgtggttgctgggaattgaactcaggacctctggaagagcagacggtgctcttaaccactgagccatctctccagccctatagttacatcttaatatttgttcatttgttttttgtttgtttgtttaatggtcccttcctccttcctcctgtgtcccctagactgtgtccatagatcatccatgaaatataattttgaagggtcatgccctttaaaatttaattcactgataccttgtgccccattcttgcctcagtggcaccatggtttagttgctcctccatgggcactcattgcctgcattcttccttccttaaacattgaatattctaaataaaataaaataaaaatagctctggacctattccaaggggagttcaaggggacagagttttgattcatttaggaagagtagagttcttcttctctggctgtgacctctgggatgtagccagtttttccttatggccccttatccttctatctgctcagagggacctccttttgtggagcttccatggaatttcttcatgggtttagagaaagccttaacttccctcatgccaacctctagctttattaaatcccagccaaatgttctcagaaggccttagtgatagcagcttgcaacatccagtgatgttgcaatggacaatagagggcagcacttgtcacatacttctagtcacagggcccaaagtagatctagaatcttcacaagaaaaagactgcctcactctgataagcgtgaaatgccttttgctaagtatcccatatatgatttggagtcatccacaagcatagtgaaaaatcttacttgcccttctcccatggaataggatatctgtatcatgctgaactacctccatcagatatgaattgccataaagtttatcttcccctacagaaagtctcattctccctaaacattcctatctctaccactaagtaagtgacttcaggtggaacttggacaacaaatggagagggaatgattaagtcttagcttggactgataagctgaataagataaaagattagctggaatgcaaaagtgcaaaaggtagagacttgtgggtttttgtggggtgatttatttatttatttacttatttatttatttatttatttattgagcctttgctgcaaaagtgcaaaaggtagagactcgtgggtttctgtggagtgatttatttatttatttatttatttatttatttatttattgagccttgctgcaaaagtgcaaaaggtagagactcgtgggtttctgtggagtgatttatttatttatttatttatttatttatttatttatttattgagcctttgctgcaaaagtgcaaaaggtagagactcatgggtttctgtggagtgatttatttatttatttatttatttatttatttatttattgagcctttgctgcaaaagtgcaaaaggtagagacttgtgggtttctgtggatttatttatttatttatttatttatttatttattgagcctttgctgcaaaagtgcaaaaggtagagactcgtgggtttctgtggagtgattaattaattaattaattaatttatttatttattgagcctttgctgcaaaagtgcaaaaggtagagactcgtgggtttctgtggagtgatttattaattaattaattaattaattaattgagccttggctgcaaaagtgcaaaaggtagagactcgtgggtttctgtggagtgattaattaattaattaatttatttatttatttattgagcctttgctgcaaaagtgcaaaaggtagagactcgtgggtttctgtggagtgatttattaattaattaattaattaattgagcctttgctgcaaaagtgcaaaaggtagagactcgtgggtttctgtggagtgatttatttatttatttatttatttatttatttatttattgagcctttgctgcaaaagtgcaaaaggtagagactcgtgggtttctgtggagtgatttatttatttatttatttatttatttattgagcctttgctgcaaaagtacaaaaggtagagactcgtgggtttctgtggagtgattgattgattgaacctttgctctactctctgttctccctcaaagccaggaacaaaggtttcagagtttaaatgagacttcaggcaatacttgtatctgagccccatcccagctgctgtctgtgccttaaagggcgtttgagtctgtgccttaaatggcgtttgagtcccgtgccttaaagggcgtttgagtccgtgccttaaacggcactgcagctaggctaaaacggggtgagaacccacccaatcaacatccccaaaagggtgaacaagtgtcactatacatgccatctgcaccagtatgcttagtgcttggcatcactcataaatggtaggggggcaggtcagatgactgatgacagaggattctaaatcgagtatccatgaaacatgagacaacaagatggcgctgtgccacaaggatacaccacacccagaaggccttagaacgaaattttaatatttattttgaatcccgatcctaatttgcataagccacgcccctttttacctgtgccacgcccacagacattccagggtgtcaagtgactgtcaggtgtcaatctagtgaggccccaccccctccccacccctgcacatagtccctaccccctagctaacaggaagtgcttctagcttaattcaaagccacatagacgccttcctgtctatcaggcaccaaagccccaccctctaatgcccccataccctagtgtgggaaagcgccatagtcagctgccccagaagtctttgcagtactttgctggcatatcatttcccaaatttggagggggctggaaatgggcgtggaggggacgaggtaagggataaaacctcgtagtgtcatttgagcaggtgccttgcttggtggtagagagcagaagccacttctaggggctctgttatcatgcaagctctaaacatccctctcaccgtctcttcagactcagcctcgtcccctcccagccccgccgcaacttcgtcgccccggctggagggtctgggctccacaaccagagcaccccctgctttggaggaggctgctaatattggcccagccagcggatcatcgtccaggcaatttcggaagagaatcttgggcaccagtgattccccggtcctctttatccaccgtccgggaacttcgggaactacgcaacgactagagtacaggtaactaactgatcttcctttgttcatgccttttgtgtttgaatgaaagtgcagtttaaatattctggtatgggtaggtgggctggggacttgggagagttaggatccttccattagtctctgaaaaggggaaggctgccgctaaactgcatggtcccgctaaactgcacggtctaagagtgacttaaacttctgaggggacagaaaggtcaactgtgacttacaaggcttcacaaagctacaaggaaagtacgttttccttcactaatctaccatgcaaaggggatgggcagctctctttctcttcccagcaggagtacacagctgttttgcaaatgtgaaaagttttttctgtgtgaaatttttcccttggtgcatgcctcacctcccaacccccaccgtcccgctcccggctacactgtgcggaggggacagagattctggagcagtgtgtggtgtgatatatatataatatatatatatatataatcttaaaatgatatatatatatataatcttaaaagcaaagtttcctactgttttatatcctgtacctcctaactaccaccaggatactggagggtgggtaggaccctgaaaggatgatgtgccttgctattccctagcagggtttaaagtacaaaagctgcttttctcctggctgtgcctatccttaagcaccctccctccaggttccctgtccataggcagggatttggagcagcagcagtagcactctcaggtgaacattcatttgtgcacaaagcttttcttccctgcatctggcattccagactccttaatccacccagagtagtgtttatggggagaagccctgccaccccttccagaggttgccttccaaaagcagcagagccatgtgtcctttggcttttgctcccatccaaggtgcaggtgtgaggaaaggaatgggagacattcacagttttagctgtgacccttcaaaaaaaaaaaaaaaagccttttgtgtgcagcagcagccttactcaagcctcccccccgcccccaacagggcaggagccagtttgtccttcccaaaggagggtctcctgcctctccctgaacagttgtcaaacttggcaaagtgacttttgaccaactgcttcctcccttctattgagaagggggtggggagagtaagcagctgaagagaccgggcaggggggagtgctccaaccatgacaaggtgacctttgggcttccccatccagcagcaaggggaggggagggggccagactcagctggagacttgtttttgagttatctcaaggatttttgcttctcctcctcaggggccgaatagttaccactgagctcactgtgcacagggaggaggaggaggaggaagaggaagaggaggacgactacgacgactgcagacccttaacccagggtcctgcatctcctcgggctaaacttgcccagccagacccgcagaagcctaagtcaaggcctgtaccaaggcctgagccaaggcctctgccaccgaggcctgagccgaggcctgtgccacaggagcctgagccacaggagcctgagccacaggagcctgagccaccaaagcctgagccgaggcctgtgccacaggagcctgagccaccaaagcctgagccgaggcctgtgccacaggagcctgagccaccgaagcctgagccaaggcctgtgccacaggagcctgagccaccaaagcctgagccgaggcctgtaccaccaaagcctgagccaaggcctgtgccaccgaagcctgagccaaggcctgtgccaccgaagcttgagccgaggcctgtgccaccgaggcctgagcggaggcctgtgccaccgaggcctgagcggaggcctctgccactaaggcctgagctaaggcctctgcagccaaggcctctcccaccgaggcctgagctgaggcctctgcagccaaggcctctgcagctaaggcctctgccaccgaggcctgaggcaaggcctctgccaccaacgcctgaggcaaggcctctgccaccaacacctgagcctgagcctgagcctgagctgaagcctgaggcaaaaccagagaagaaacaggctagagcatcccgagaatccagccccgtgcccaagtgctgtgcctgtggcccaggagatccccacgctcccagaactgaatgaaacctccgaaacctcccaccagccccagtccttcctcagaggtgagtgcccttcaggcatacaacagagcagaggtaactctagcacagacacatcagggctcctgcaaatacaggatctagccaagtgccctgtagccaaggaaccagccaaggctgggaagctcagtcagtcacagtatctagaagtcaggtgggctccaggtgcctctccttgggaacaaggggcgtaggcaaacagacacaggcgggcagcaccagtctctggaaaatgtaggtccttgctatgctcccaggagtctaggggcaaaggtagcaaatgctgtggggaggtcagacagcttccagagggaaaggcaaggttccagatgaagaaagaatctgaggaaagagaaaatgaaaacaaactgttagctaaaaagaatcgggaggccgagaaagctgggtttgaggggccctgttcccccttcccctggctatgcagtaacagctgtggcgcccctgcaccagtggcttgacctctctgtgtctatccccatttgtcatagtgtgtgacagtctcaagcttcctggctgaactgtgaacagcaaatgacataatgaccaaagagacattctgagacccgtgaagggccttaggagagagcttgtgtcctaattctattgtgttctgaaggccatgggtttcctgtatgtgaatggggaagggaagcagctagaaagggcgtgggaccagaagcactgtggactcgtccgaggaagctctgctgtttggtggccctgcagttgactggtcaggtactgggtggggatctgggaggcagaggctgcgtctggtggaatacctggggatttggggggcagcatgtaaggtctgtgtcctgtgcctaagcctcaaggggcccaagcctcccagcacgtgaactcccaccccgcttccagactattatatgcccaaaagaagccaagaatctccttttaaacattggttcagacagtaaatcaataacatagtggtatggcctcagtctatccctacctaggacaccggccttccctcctgccccctctccagtgcccccacagcaactcttacttacttttggtgtgggtgtgttgatcggtttgatggtgtgatcttcatgttgctggttcagttagtgacttcccaggcttgcagactgagacagagcattgcttgggtcttcccggtgtggagtccctatgtggtgtccaagtccgcagcccagttgagtgtgtcctctgcagcaggcatccagcacactagggacccttggagagaagttgcgcgtgtaaaatgcttgccttgttccccgaggagagggagagcagttagctctgtggctacaggaaaagtcacaggggggacaaggaagacccactgcctggagggagagaggggcgagaggcagagaccctggctaagggtgccagccaggcttagtgggtaagtctgaatcttaaagggtatttagtttcttcagaatttgagcttttaagcccagaaaggagacctaggctgccaggatggtgggcagggcagggcaggggagggcagggaccggggatcagcatggggctaaagtctagtgggcccagccagggcaggggagggcagggaccggggatcagcatggggctaaagtctagtgggcccagccctgcctagaagaaacaatagacctcatgacatgcaaggtaggtccctttctaccttgggggggggcaaaaataagggaaaggagagggccttagataaagaatcttgtgtccgctgaacctggctggggattaggctgtgtgcttttgagccctaggttgacccggaactgtaccagcctctttccctgccccttgagccacactttgactaaataaggtcaggtgaaggctctgtgggcagccacacagaggaagaagaacacatgcataccctgtcccccaccctaccccgcctgcatgccctggtggctcttcagtgggcacctcaaggtgacctcagcattttcctacctggcaaagtccaggagtgcatctccggggtgtggttctgcagcttctccaaaatacctctgcagggagtccaggagtgcatctccggggtgtggttctgcagttctccaaaaatacctctgcagggcagaaagcagagattaggctctgaaacaggccaatagtgtgattgctgccctctcgtatgcagaaagcagagattaggcttgaaaacaggccaatagtgtgattgctgccctctcgtatgccccttaggaaactgctctggcctcacaaggactcgggggtgttcacagacaccttttcaatgtcccttactcccaacaaattctcaagtctccccggaaagaggaacatcatcgtaccccagtcttcctagctggaagcttcctcaaatccataggcacacactcagagaggccagaaaagccagggatagaggctggtggatgggggaaggggggcagcatgggtgtgtgtggggggagacaggccagatgttcttggaatggggacacgggggtgattgatgcggacctgaatttgaaaggggaacattccccacgtgcttcatgctccgggtggaaaatgggtggggtgggggctctctcagtcctgccaagataatatggagatgcctcgccctgtctaggtccccacacctgtccactgactttaacctgccttcccagagagtcagcctttgagagtcctccctccctgcatacatcctctacgggtattatagcgacaagccctcttacagccatggccccagggttttcctcattctcctgctctccagctgctcccctatctaacaccccaccccaacccccaggtcctgctagaatcatggcccttcacctccagcattgcccaactttgagggggtggccttgttcccagcaacagtacggtgagcctgtgtgtgatgtgtgggtttaatatttgcctttaaggagctggactttccccagccaggattgggaggctagtggcaaaaatttttctgagaaaacgaaatgagggtcctagcattcggccacggccaccactgtgccgttttgagacgctccttccccgactttctcctctactccccaattttctaccactttccacactgctcaaacaaaactgtttcctgacgttgttagtcgttagggagcgtacaaggccggataggcgtgtgccgagcgcaggggcggtggcaggggtaaccactggggtgccccgggcccccaaactgagccagggatgagtgctgcccccggcccggccccgactgtcgcctcgcggcgcctgaggctggggcgaggggcagtggggaacgagcggggcgcggaggcccgatgagcctggggagcagcgcgctgctccccgacgttccccctccctctcagaagccggtcccgctcatcctctgccacccaaacccctggttgtccagagaaggggaaccccccccccaaaaaaaaaaagccaaaaccggaaaatacagctggctcaggcgcgtgctccgtggagtcggtgctcccagtctgcgtcgcctgtgcccctttccccgcccgcgttcccccgtgagggcgcccctgctccgtgagagttccttggcaccgcggatccctggcgccccgtccttcccagcccggagctcagtttgtcaaattcagctctacttctctgtggttcccctaaagccggagaacttgctggcttctctgcacctcaggcattagtttcactggaatccttgaaatttaagcattgctttccgggggggcctccaaaatcattcagcattgttatgttcacagggaaaggttttgttattttgtttggttggctgttttgttttgatacttgatttccaaatatttttaaaggtgggaattgcgcctctgctgactcctcaaaattaggttatttggcttgggccctaagtttgcttctggctcctatccgttttatatgcaagctcctatagagataaaaacaccaccctcagttccctaaaatttaaagtaagccggatccttagtcctttttgacagaatttccttacttggtgatggacattttgacagcatttagacactgtccccaggggatgaggtagtggggtgttttccccgtatccctatcattttatatctgcaataatgtttctgctgactctttccccaccaccggccctgtagtttttattaacgtggaacataatacactagattctgtggtctattgccccaaccccgaattttacaagttgaatcccacccagacctctttaaacaagtttccaggaatccacaaagtcccaaccttttttctatgtcctcccctaaacgtaggcacctcagtagcctacactctcagttctcggattccagaaaaatcaagggagtgcattcaccagtcacacctaaaattcactcaacaagaccccccctaccccccacaccccccccaaaaaaatccaggcatacacactggtcactgggctttggggtttcctagagctcatgcaagctttctggccaccgttaggttctttaaaatataaatgttctcactaaagcccccaacacagactgagcaatgatctaagcatatgtgatcctcatatgtggtggagggttttccatgctctcccaagtgatatatgactgtaccatgtctctctaggtcacagaaggcacaaaattaactcttggccatgctttttctttctactagaatgctgaatgtccccaatccttgaacactgagctcttccccacccccggcaccaaatattaagagtccctgggtccctttaatttaacaagttttttaaaaagtttttaactctaacaccaattgcaaaattcagtaccatcctgtatccttctcatccccctgttggatcatacttggtgtcctcaaacattaggcagtatttttccaggcccccaaaacacacttagtgttagccttgggtcacagagaggttctggtttctcccactgtaataccagctatacacccactcagccccctacatttcaatccccccaacccctgtcattccacacaatctctgcagattctcaggccctacactttaggaactcataatttctattccttgtaagggagggattggctctgaagcgctccaaggagtgagaactttagattcagaaattatttttctggggcccccaaattcaagtactccaaaacaaattgagattttttttcttccaaagagttgggtttttattaaactttaatgaactttattgaatttatgacttctcccggaatctcccaatatttgggtaatgttttatggtcccaaagcacactggaaaaaactccagatccccaatctaggtttaatatctgtgtctcacaattctagctactatctgtcctgtaattagattcacatccattgggtaatttttattgatccccaaaacagggagtatgtttctctaagaacaaacattgtccaagaaaacataccccccttgtttaacaaaaagatcaataggaatctgtttaagaaattaaaatattaaaaggcattgatttttctgtccttctcaaatggactgattatactcctaattccccacatttagacagcatttcccactctctctctagaggacttctctgaagccacagaaattagaggtgacttttcccgggtgctgtagggcccactggcttcagggtgaaacttagcctccactcccacccccaaaagaattgacccttaattccctaaaacccatctgttgtttttttcactctcccatttttaggcaattaaaattagctccccccccccaaatttcatgttgggaattttcttaaactcaacagcaacagcaaaccacctaacttctataaaatagcctaagattttgtgggttccccaaacttgctgactaagaccttaggttccccacgttaggcttggatcaagatgttccccttttttgcatagaataattttcctcatttcccaacttggattctaatttttctggaatctcaaatttagtcgtttgccacagttttcccatttatggagtctcattcaagtcctcctactttgggccactcattcccagggccgcaaatcagacaagggtctgatttgggatccccgaaatttgatcattgtttccgctggccactttcggcagcttcccgtcctgcgcataacatcgatatcggcctcttcacttctcctacggtgtcccagcggcagctcagattttggaaaagtgtgtgtgccccaaacacgcaccggacactgcgcggcggccaggacgtgggcagtgctgctcccgtgtccaggaaaaccactgggcattgcccccagtttcccccaaatttgggcattgtccccgggtcttccaacggactgggcgttgccccgggacactggggactgcctttggggtctcgctcaccttcagcaacgtccacttcagcagctcccacttctgcagctctcccgcccttctccgatcctcctgcgccacggacccgaccttcggccttgcggtgagctcccggtggcgcgctcccttgtgctccagctcttccgttgcggggcaccggctacctcgcagtttgtcttcagggctgctgcggggtagcgcgggagcgggctgctggcttctcccttggcgaccgcgctcctttgggccgggcgctccgaaacacgcgccactttgtatgcactctgcaaacaacgcggtggtagggaaagcgcctgagcggccaggaagcgggaaaggcagagcgctccggcggcttggatcctccagccctacaggtcgagggcttggggaagcaagcgtagagggcgcgcggtgattgatggtcgggccgagaggttagccccgggtgcaccggtttccgccagtagtagccctttagaggcctagagatgcggaggtcgggagagggaaacagggtgcacgggtttaagagtgattttgacttggtaaaactctcaatggcttcttatatctgaaaccacccgagatagcttcctaacaaccccaaattatcctgttgcccctggggccgaacctgcaactaagcaaattcaaagtgctaaggacagggaactgttatctggaccttctgggtgggtcccaaaggcgcgccgctgagaagcaatttagtgagctctggcccccaggtctggtttgagctacgattcccgcccctgcttctttttctgcggaagggcgcagacattgcccggggctctacgcagggcccagatctgtcatgccaagccagccttcccttcatggtacacctggtggccccccaaaggctgctaggagatcccgcgcaaagcacgaggctaggggtgtttaggatctggctgctattcgaggtgccttgcagcccgtcctgcttgctcagacaggtggaggctgtgcgctctgcaggagccgatccaggggactgtcccctggttggcaggtaggtccttgtaggacctcgatccagctacctgcccgcgccttaactctgcctgctgggatgcgcctaccttgtaatcctctaactgggcacaggtcccctcactaccactgaggcctggggtgccacgcagttggttcgcaccacgcgcccatgttgcacaagcacagggcaccccggaaacatgacatgtggcctcgattttttttgggatggacacccaggatttagtgggcctgctggggcgcacagctccccatctttgctaccaggcacccattcccccctcccccaacggcaacccccgtgcccagggccccggacccgcattccccccgtggctcgagttgcgggggcggtcccggggcggggcaagggccctgcggacgcccattggcgcgggcgtaaggccagcggggcccgagcgggcgccgagccgcggggtggcgcggctataagaaccgggcgttggcgcccggagttcgcctgctctccggcggagctgcgtgaggccaggccggcccccggcccccccttccggccgcccccgcctcctggcccacgcccgcccgcgctcggcccgccagcgcctccatccgggctggcggccccgcgtcgacgccgtccgccacctcgctgctaactcccgtgcagggcgccgtcggcggggcctcgctccgtcgggcctgcggatctccccaccgcctcctcctctatctacctcaacaccccattcctgcttcgccagaggaggcggtccccaccgcaggcagtccggcttgcaggtcgccggcgttgtcatcccccgcgctccccctcccagccctccccggcgcgcagcccggcagctcctctcttttcgctgcagtcccgagcagccgcggcgccgccacgcctgacccccccacaagaagccggggcttacgacggctgagggctccgtcggccctaaccgagctgggtgcccgtggccggggtgacgcctccattcctcccccctcaacaccgtcctcgatccttcgaagttgcatcctttcctctgcttagagtgcgccccccctcgcgcactcgcttacccgccacctttcctaggctcccctcctgccccctccccgttcctcctcgcctcagactccctccccctcacgtccgccctctgccttcgcctacccaagtggattaattatacgctttctgtttctctccgtgctgtcctctcccgctgtgagcctacccgcctctcgctgtcctctctccctctctccctctctgtgtcccccccctttcacgttcactctgtctctctcactatctctgccccccaactatccttgatacaacagctgacctcatttcccgatacctttcccccccccgaaaatacagtatctggcccgccccagccctaagataccctaaagaagcagaagagacgcccccctccccatcaaaaaaagccatctccccgttctgtcccgtcgcacattcggcctctgcgacttggacagagcggcgctggcagaggagtgcccggcagaagggccttcgcccgctgttcggtttgcatacccgcagcagggagatgggcggcagcgtcgccggcttccaggtaagggctctcaggggccctggggtagggggtgcgcagggcccggcctgggctccgcgggactggtgtggtgggtggggggctgggggctcgttccctaaccaaactcttgttcccccttgcgccatggaatggggaaggggagggggtatgggtccgttggggaggggtgtgtaagttgaactcacaggagactttctggtgctttgctgtgtattggggaagggggaaatgaactgcttagaatgtatgatttgtattgtgcagaccccgtaccccctccgcagggtgctgggtgatgaggaggggggcagagtctctgaagcccaccctggtatgttgactttgtgccaataggacaggaaagaaaactgggtgggcggggccagaagcaccccaaaattggcagaagtcaggcgagaccccacagttaactgtttctccccacccacacacgccccaatctgtccccaacattcccagccaggaatgttgggggggcggtttcgggctcaaaagggcagaaatgttacagttttgagagtaactgcccctgccttttactgggtggaactttccataggatgatgtgggaaggacccccctcccgcccccattggtctgtgcagaaagggctgggggtgcacgatgaggccccctcccactggtggtgctttgctaaggaatggtccaaggctagctcttgggggtgcaggagaaaagggactggctggaaggagggagggggcgggtgcaaagggggcgaggggagtggtcagcaaggagggggggtgggggtagggtggagccgggactgggaggagccgactcagacataaaaagcggaggcactgaccagttcgcaaactggacatttgcttctcctgtgagaaccttccagccttttcctgtcttcatcctcttccagccccagcggcctccttatccaacttcaggtaaccagggccatggagccaggaccctgctgccatcccccctccggcctgccatggggtgtcagggcaccgggtagcctggggcctctgccattgcagccgggcctgctacctcttccaagccttcgccctccctcggcctctgtcctgtgttcccactagccccaggcttcctcacctgtcccgccttctacttttccttcccctttgcctccccactccccacaaattcagcccttccctgggccttcacctatcccctacctcctggtcagtcccctgcttgcttctcctggacacctggctggaccagccagtgtgatctggctttgggaagggacttcgaggtcacgtccgcctgccctgctccacggcaccccctcacccctaaggattaactgctgtagctatatagcctgctgatcacaagtggttggtggcccagagtgcattgggtgggccgttctgggtcttgttgaggaggtgggcaaagataactttagttatccgagggagcgcgagcaaatttgtttcttgattattccgcactggtctctcacttgtttctttgcagcaagaaatcccctgggcctgtggagcagaggctggggctgggcctgggggctctacttactgcagggtcaagagtggcttactgtggggcatcgtgacctcccaccactaaaaaaaagctggctaaccattgttctgtagagccacacagcctgtggagggggtgtgtccctccaaaatggggtgtctagcctcccaggaactcaaactgcttcctctagtaccctgggtaaatttagctcttatcagaaagacggtcctatgggccatagaggtactctctagggtccctgaccatggatggtaagagttgaggtctggataggactttaggttccttttgaagctctgaggtgcccaggaaaatctatacttggaggactgggtgaccctggagagcaaggctcacaggagcgctggcaggcccattgcgctaaccattggattcttgacctgggccctgctcatcacgacctgggaggcggggtggacgggtggggttggctctgttgcttagggaagggtggccagctgggggcgggaggttggcgactggtccctttggatgcacatgctccacggtgggtggggtggagctgtctgccatcttggccagtacttgggaggccagagtaggagtggcagtggtgatgtgcccttggactggccccacccccatgatgtcatagcacggaaaccaggtccagtgtccagcactggcccctgctgctgtgggtgtggttaaactgcaattgcgcccatggatgggtggtcctcaagggtttcatacagttcaaagccaccacagggtgcctttgtgggctccattgagtttctgcacgcccagagccaggttttttttttttttttacctcagcaattcaaaccctcctgtttttgcaggttaaataaataatgagacggacgccgtctttcaaattccaattacatttttaattaagagatgataggtgtctttggtggcggggagaagccagactcttggggataggggtgtggcttggggcctgtagctcaaaacctgcgtttctttcaccaggtaccaatggggatcccagtggggaagtcgatgttggtgcttctcatctctttggccttcgccttgtgctgcatcgctgcttaccgccccagcgagactctgtgcggaggggagcttgttgacacgcttcagtttgtctgttcggaccgcggcttctacttcagtaagtaactccagagagacgggggaggcgggagcaagcggggtgggggggaggtggtaacagcacagtggtctggctaagctaccaccccacccccccttgggcaaggcggtaatctcacactcacaagtctgatagctttaaaagtttttttcaaagttaataaaagcaaaacctaatgtggttcccaggtcctagccaggttgagagtgtacacatagtctggggctccaaggagggagagctggaccttggcctacaccatggggtgcttacctgcttttcaatgttcatgcttctcttgatttcccagttgggggaggggctggacctgggctcactgctcacagaaggcagtgatgggggtggggtgggggacacacgggtggggcatgcctgggaggggcaggggcaccagaatggatgactgtccttgctggcctgagccactctatcttcctcacctggtcctggacatgcagcctcctcctcttcacttctgcctacctgcctgtgaactgctctgagtgctcaaacctctggaaactacttctgctcctgggtactgcaggaccagtccttgttcagggagccaatcctgcacggaggggcttcacagataggagggccccaagcccagcctcggaccgtgggggagagggggaagacgggagaagagaagggagtggtttttgggtgcctcactcctcccctcccgtcttgttctctcctgccctatcttcccttcctgtcacagttcagcgatgggggttgagggtgggcccctcaggctcaaggtgacaccaggctggggggccccaagtccagggaccacacctgtgtccaccatgtcccctcgaggggctcaccccgctccctgtttttcaaaccacttcagtgtggtctctggtcttcttggaatctgggacagagagactgtgctgtgttagctgccaggcaggaggcaccagatgccaggggctggtcctgtagcacgcaccttcgtttttcctttctgggcatcttggcctgtctggctcccactgccaccacatccttgcagggtaacctagggtccagccagggcctagtagaagttcaggggaagttcctttcttcttcagccttcccaagggggagggtttggggaggccaccccagtgggtgctgaccccagtgaacactaactgaagctgtctgtcctgtggaactttcaggcaggccttcaagccgtgccaaccgtcgcagccgtggcatcgtggaagagtgctgcttccgcagctgcgacttggccctcctggagacatactgtgccacccccgccaagtccgagagggacgtgtctacctctcaggccgtacttccggtaggtaacagggtggggtcgaaggaattgtggggtgagacaaagaaatcacttgctccttgatttggggtgccatgaagtccacccacgttttactgttggcctctgcccattaacatacatgtttctatagactctagtggggtgggtatattgacacctggatgggagctcaggctaactcgataccctgaaacctgctgactagcacctcctctccaggacgacttccccagataccccgtgggcaagttcttcaaattcgacacctggagacagtccgcgggacgcctgcgcagaggcctgcctgccctcctgcgtgcccgccggggtcgcatgcttgccaaagagctcgaagcgttcagagaggccaagcgccaccgtcccctgatcgtgttaccacccaaagaccccgcccacgggggagcctcttcggagatgtccagcaaccatcagtgaaccaaattatgtggtaattctgcaatgtagtaccatcagtctgtgacctcctcttgagcagggacagctccatcatgtcccacactaaggtctctctgctccacttcccttcccaggtttctccccacccacccccatgccccgcctccccacatcaggctgctccccttgccccacaccatcgggcaaggggatcccagcaactcttcaaaaccaaatttgattggctctaaacaacccaattggcaccctccaaattatatatgaacattaaaaaaaaactttaaagcatatagtccctttacaacaaattggcttaagaaactccataactgataatctaaaaattaaataaccaaagaaattaattggctaaaaacatactaaaaattaattggcttaaaaacaattggcaaaaatcaaataatttggcccgcccccccccccttcatcttctttccatttagatctttagtcaaattggctcagacttggatctcagaacccaagaagaaaggaaggggacccaaaattttgcaggtagcatgtcattgcttcagtgctctctccttgtcactagtcacttttagcataatctggctgtgaacaacaatagccgcccaaactctttcttcactggtcattccatcacaaatgtcacccatgtcaccaaggggctgggtgaaggaacccaaggagaggaacagaacatgaaaactgaaaatagaacctaattggcacaagcccccagtcccaaaaatctcacttttcatacctactctaaaaagcacatgattatacccacacgtacatgcacacacacatgcacacaggcatgcatacacacacacacacacacacacacactattagatgagaacattgaaatggctgagcaacttcgattggaaccacattgcccaatccaaggcccatcttaaattccctgagcagtttgcatggtttgagctcctctctgaatccatctagtttctgctgccagtgtagagtcagtttggccagataaggagatggcactgccaagtgatacatgctacccgagtagcctgacccctaggtgtgctcctgggaggaaagatctgggggacaacccctaccccaagcacacctatgggccatctctgtcaatctcctggggagcccccactttttaggggctccccaggagactcacactgatgtggggagtgtgggaagtctggcggttggaggggtgggtggggggcagtgggggctgggtggggggaaactatgggtaggaagtggtcccagagaggtcttaggtggaacagtcaggaggaggcacaggtcaacttgcagaattactgaagaatcaggaccccaaattttatgtcaattgatctattcccctctttttatgtctggggcaggttttttccttttttttttttaatccctccttagcttttaatgcgctcataatcccattccctatgtaacgggggcagcgatcaagtaatgaatgcatcaagccatcaataccagcgagagccagtaacaccggctagagccatcaacaccggcttccaccatgtcctgctcccaaccatttatcaaccttttttttttttttatctgtctctatcgcttggcctgagttgggagtggagtctctgtggggtgctggccacgcacccacagagaaataaaaggaattgagaaggccgctacctggcctgacttctggggacagtggctggtccccagaagttctgaggagtggagggggcgtggggcagtgtcccctcaggtgttaggaaggtgctcggaggccacaaagatggggccccagctggccctgccagttgggggggaaggggatgtagatgtaagactagagaggttccatcaggcgggagcaagtggctgccttctgagcacttgggggaggtcctccccgtgcccctcagtgtcatcttgcccactcctcagcaccccatcttaccctcaggaggtctggagctctacagacctcctgggggcaaggtggggtgaggcctggagctggggaagcgaggaggctttaaagccttcagagccaggagaactgtgtacatggggttgtctgggccctggggcccgagggtctggtgagccgtagcagccactccacggtgcctaggactgcggcggggaacagggcggctggaggtttacctcacccccacttctgcttccagtgcagtccccctgcccaacagtcctactagtaatctagaggcctgaggcttctgggcccaggtgacaggactggcaccaccctgggggcggtgtgtgtcagccagccatggcacagagggttctcagcaagtgcctaaagaatgggccatttggaacattggacagaaactcaaagagtaaattgttataattggagaatatgaattggcctggtacccaaaatatctcgaggcaccctaaattacctgcccatttgactggacatccacccagtgttaatatgcctcgtgggatgggtgttttcaggggcatttgctgaccatcctctgtgtccccagatttgcagttctccccatcataggtcaccctgatgcaggcacctccctggcctcccatgcctagtgtggccctccatcttgttttgtctcttccctactgtcttcggtgggatcccctcttgggtcccccaatttgtcatcctgtgaagacttcccacgcgtcgaatgccatatgtcacctgtgccactgcccatgtcatccagcagtggccccgggtatttgccccaactcagtccttttaacatgcattttctggcaaaatccaaagcttgggttttgtttttaacctgttaacgcttgcaaacctaataaagcattcaaaatactatttattgagttctttgctctttcacttgggggagggggcaaaggaaaggaagggtggcagggagggaaggagggagggagggaaagagtaaagggtgcctgatgcctgggggaacatggggagcagcagatgaaaagcctttgaagcatgagaagaagtggggttcaataaggcactctgaggaactcgagagcagaggaccacatttgccctcaaagatggggctaaagggatcccatgatgttttcgaatgagctgtgtaccatgaatacctaccccagggaacattgcccttagccttgggggttggggagcaggataggtgaagagtcctggttggtgatggacatggggactcagggtgaaaaacacctaggaatcccctgcgcacacacagtcgcccccattgttccctctatcaaagctatgctggagcctgtccccagtagttactagatagacctgtccagtggtaatggtctttgggattcctgaccctgggagtaaagtgaagccaacccctagatgcttgaaacctggcctggatatcttccttctgcacctttatagtccttagtacccccactttcctagtgcagatgggcatgtacccttcccccatgtgggaggagcccaggctggggcagctgctgaggccctgtgcctgggaccctgctccttttccccagttaatgctacacactagcatttctttttattgtgtttagctactttttaaaataaagagtctcacactctaccccaggcaagcttgaactgcaaatggatgtatgtccatgctggcctggaactcaacagcagtctcagtcctgcctcagccacctgggtgttgaaattacaggtgcaagccaacacaggggttagcattagctttcagttttgtttgcactccagggtctctgctgagtgtgttaaagtgggtcttcagggcagccttcaaatccaagggctgcttttaaactcagggtacacagtggtcatgggggactgcttagaagacgaagttcatccttgaagaggaagactggatagcagtaaggtgaaaatgaagggcactggccagcctctggcttgacagagttgaggttaagacaaggcctggatctgggcagaccaccttcccacccagtgagtctttcttatcagatgcttcagtgtcttctatgtgggtggaagaaggcccagagctgcccactcctttgaggacaattcagtgtgttctcatctgtcccatcatctcctgagagcccactataggtctgtcccacccccctgtcagctcctcagttgactaggaccctaaggagcaggccttgggatatccaggttccagactcttaccccttttcccaagctggttccttcctgacttgctaaccaacatcacctgaacatggagtccttggcacctc

SEQ ID NO: 89 - DNA, 806 bp, Rattus norvegicus
gcttgaatctaatacgtcgatcataccatgttgaagatgagcgggtggcagcgacagagccaaaataacagccggaacctgaggagagagaaaccggtccaattacagtcatggcagctgagtctctgcctttcgcctttgagacagtgtccagctgggagctggaagcctggtatgaggatctgcaggaggtcctgtcctcagatgaaattgggggcacctatatctcatccccaggaaacgaagaggaagaatcaaaaaccttcactactcttgaccctgcatccctagcttggctgactgaggagccagggccagcagaggtcacaagcacctcccaaagccctcgctctccagattccagtcagagttctatggctcaggaagaagaagaggaagatcaaggaagaactaggaaacggaaacagagtggtcagtgcgcagcccgggctgggaaacagcgactgaaggagaaggagcaggagaatgagaggaaagtggcacagcttgctgaagagaacgagcggctcaacgaggaaatcgagcgcctgaccagggaggtagagaccacacggcgggctctgatcgaccgcatggtcagtctgcaccaagcatgaactgttggcatcacctcctgtctgtctctcccggagtgtacccagcaccatcacgccagtgccaagcatgtaatctccagtgcacatgctgaggaggggactgagggtagaccaaaggagaggggcttgtacactgtacattctttattcattccatacccagtaaagtgactttgtgtgaaaaaaaaa

SEQ ID NO: 90 - DNA, 437 bp, Rattus norvegicus
tttttttttttttctttggtttttaattctttttttttttattattattattggtttgtgtgagagaggttgagaaggtttggtttacactgagtatatgttgtcaagtggccaaagtccacatagctctcctgttttctgtatacgttcacagcctcaaaaaaaaaaaaaaaaataattgaaatggctttaaaaaccgaacagaacacctccatcctgcgataagtacctcgaatggattcagctttacccctctgtgactcatcctcacattctcagcatatttaacagaccaacaaaaccaagtactagtagtgaggggctgacccacgtggctttgcagtgctcttcgtccagaagcatggcacacgatgcttgtgcatgtggaaacttagcgactgtcaacatacattctcagggatttatccctcgtgccg

SEQ ID NO: 91 - DNA, 2340 bp, Rattus norvegicus
ggatccagatgagagattctggtacggaggtgtgtgtgtgtgtgtgtgtgtgtgtgtgtgtgtgtgtgtgtgtgtgtgtattgcacaagaatgaaaactgaaaaacaagcagtatataaatggctcccggagattctgagatgctgaggcttgcttgtatgttgctatagtgtatgttggtgcttgggagccactgtcatggataggtatgttgctgggtcatccaagccagtgtgtggacactcaggtacaggaagcaaagtgaaggcatcagcaggcatttttgttttacgatgtttaaattacacttattttatttgtgtgtacgagtgtatgggtggggatggggcaaatgccaaggggcacttcttgtgagagtcaatctgttccttctagcatgtgggctctggagatcaaactcaggtcattgagcttggtggcaagcacctctacctactgagccacctgttcaacacccacctgtaggcatttgtgttcatagtagttcatagccctatgaacatatagcacctaggccaagagagcctggcttccccaccccctccccttgtaccccaacctctgccacttcatctcactcctactaggcagctgggttttttccctcactgtaggcccctgggcaggcagccagcagccgcgcccaacgctgggagggagaagaatgggtcagaggctggagcttgtggttgagttggggagtgagtaagctgagtgagggatggaaaactgctgttgttgaggccaggcctgggggggaggcacagaaggctgctggcatgaatttctagagtttgagtggtaagttttgcaagtttcagagcttgaagcacatatgagcttcttgccatcagtgggtaccactcctctgatctccctgggagtgaggtcggtctctggaagtgctcttagagagtaggttggagtagagcactaaaaacggggacagactgagtgtgacttgagtgatgcctagcaacatatatccagctctcaacacactgttggtgtgggttggagaaggctacttttgtgtctcctgcccctaggtctcaacggtcaccatggcgagacccttggaggaggccctggatgtaatagtgtccaccttccacaaatactcaggcaacgagggtgacaagttcaagctgaacaagacagagctcaaggagctactgaccagggagctgcctagcttcctgggggtgagtggatcctgtctgtgtattgcatatgtgatgcatccccaggaggaggctggggctggagatatctatctatctatctatctatctatctatctatctatctatctatctatctatctatctatctatatctatatcatctatctatatctatctatctatctacatatatatatatatatatatatatatatatatatatatatatatatatatctctccctactcctggcgcttggtatggaaccacaatgaaccatctacttcacaccagccccccgttgagacaaggcttagaatgaagttaactgaagtggcacaggaaaaccacattaggtagtcagtgtctgaaagcacagcctagatcaggacagtctttccggtgatgtgcaacagaaatcgagtttctgcttgtgaagacatgattgtggaggcacacaaatgcctgcagatcttcccctcaatgacaccttatcttagttaacacctccttgtcatgacagttacctatagacatagttaaaacaagcgtggggaagatgtggtcacatcctttcccagctagcccatgtgctcatctcacagttgagccctgaggctagcacggtgtctgcaagccttcctgagctcctggctggaggtggcgtctaactgtacctcttctacctccagagaaggacagacgaagctgcattccagaagctgatgaacaacttggacagcaacagggacaatgaagttgacttccaggagtactgtgtcttcctgtcctgcattgccatgatgtgcaatgaattctttgagggctgcccagataaggagccccggaagaagtgaagactcctcagatgaagtgttgggccagtgggggaatcttccatgttggctgtgagcatagtgccttactctggcttcttcatacatgtgcacagtgctgagcaagtttaataaagagttttgaaactatgtctgagagactggagattgtgggtgggtgggtgggctgagggagggtggtggcagagatggctggaagttgacctggagctttgtggggccaactagaaaaggttggggaggggtgagtgactatctgagtcatacagtgtat

SEQ ID NO: 92 - DNA, 423 bp, Rattus norvegicus
tttttttttttttttttgacatagtaattattttttaattcaaaagctgcaggtgggagttttacaaaatcaagataagcttagtaaaaaaagttttctaaaattaaaagaaacaaacaaaagaaggtgatttttctcagtaccttagtataacttaactgttgctaaaactttgatatgtctgtttccagtctcctttttccgcatattattttgattttctgttatttcatgtaattttatcatatatgacatacatgtatacacatacatacttaatatctgggcctatgtaagtggtaactagtttgcatgtggtgaggaaaatctggtttaaggactgagtagacaaggggttaaaatttaagctttaacgtctgttagcgcttatcacctaaaaccactttgtacacatgaatacac

SEQ ID NO: 93 - DNA, 1030 bp, Rattus sp.
gggcggaaggccgcactgttgccacaatgatttcctgttcttcctttttcaggttgtcgaccatgtgtcaagctgtgtagggaaactgcctggagatggacattctgactgctaatggggacatgggacggcagttacctctgatcacttgcgtggattctcctggagcagaaagacagaagccgccagtcactgctccggatctagcactgggattctgtgctgaagggtctaaaaactcgttttgatttggatctgggagaatgtctgagcaaggagaactaaccccgaccatactggaagaaggccggacagagccagaatctgccccagaaaatggcatcctcaagtcagaaagtctggatgaggaggagaagctggaactgcagcggcgactggcagctcagaaccaagagagaagaaaatccaagtcaggagcaggtaaagggaagctgaccagaagtcttgctgtctgtggaagagtcttcagccagacctggaggggaaagtcaccaggatcagactctctgagaactgcaaacggaaaggaactcaaaagaacttagattaaaacttaacttacaaatgataaaataaaataaaataataaaatccagtaagcacggaagtgctcgatgtccactgaggctctctcttgataaggctgaaccaaatacaccccaagcatcctgtctctatggagacagcttacctctcagctccccaaatgaacctgcctataaataacacaccactgttggctcctaggagatgagaaatagtgtctaagcaccctgaactgtggagaaatgctgctatggccttcgggaggttgttggtttttggtttggtttgtttttttttttttttttttttttaactactgcaaaaccggcccagaagagcgtctgaactggtagcgaatttgatattaaccagctgctgcacctccactctgtgatcccccaaagcttaaagttccagggcccttcgcatcctcttcgttacccttttttttttccttttttaa

SEQ ID NO: 94 - DNA, 1616 bp, Rattus norvegicus
atggcgctgagtgacctcctggaactcactctgctgctgctgctgcccctgctggagcggctctcggccgaggactgcccgtgctccgaggcttctctctgccgaccgatccgccaccaccgcgacttcgaggtctttgtgtttgatgttggacagaaaacctggaaatcttatgactggtcgcagattacgactgtggcagtttttggaaaatatgactcagaacttatgtgctatgctcattcgaaaggagccagggtagtgctaaaaggtgatgtggccctgaaggacatcattaatcctaccttcagagcgtcatggatagcgcaaaaagttgccttagccaaagcacaacacatggatggaattaatatagacatagaacaggaagttgattgctcgtcacctgaatatgaagcattgacagctttggtcagagaaaccacagagggtttccatcgtgagattgagggatcacaggtaacttttgatgtagcttggtcaccaaagggcatagacaaaaggtgctataattatactgggattgcggatgcctgcgactttctttttgtgatgtcttacgatgagcaaagtcagatctggtcagaatgtattgcagccgccaatgctccctacaatcagacattaactggatatggtgactacctcaggatgggcatcagccccaggaaacttgtaatgggtattccctggtatggctatgattacatttgcctaaacctctcaaaggatgatgtctgcgccattgcaaaggtccctttcagaggggctccttgcagtgatgctgcagggcatcaggtgccctacagggtcatcatgaagcaagtaaacagttctgtttctggaagccagtggaaccaagaccagcaagctccgtactataattacaaggatcccactggccgtttgcatcaggtgtggtacgataacccccggagcatctcactaaaggcagcattcgtcaaacactacggcttgcggggcattggcatgtggaacgcgaactgtctggactactcagatgacgctctagccagagagcaaacggaggaaatgtggggagccctgagaccacggctgtgatacatacagaggaatgtcttttgtcagacggttagatttgcaggactggcctgtgtaagcagatctacctcttcaagagataaaaatatatattttgtcatgttaccctaacttacttattggtctactcaatacacaggaaagaaataaagaaactattcatttgtacttggaaaatacatacatatcttaaatatccaggaatgtaaaagcaaaaacaagtcaacttactctattaaatattcctctgtttcagtattataattatattatattataattagaaaagttgcttttagaattccatgctgaatttttcctcctgttattaaaatatactgttaaaaaccaatagggatttttaaaaatgtataactagtagacaagtgaagatactggagaagggagtgcacatgaccatagtgtgttacacacatgtatagactatcatagtaaagcccattatttttataattaatatatgctaataaaagatgtgtaacttgaaatgg

SEQ ID NO: 95 - DNA, 574 bp, Rattus norvegicus
gacagttcgtgagagagaagctttattaaaactagtggcagatctggactgctactgttgcgcctttggtgcatgttgagcctgggcaaggatgggtacatagggagatacaggtgggatgaggagcaaagagggagggcgctgcctactgagactgtgctcaactgttctcccagaagaagttgttacaagccactgtgagagcagccaccagcacaacaaactcctggaagtccacttccccatctccattctcatccagttccttcatgatcttgtccacagcatctgcatccttctggacatccaggaagctggagagttcagtttgtagcaggtctttcagctccttcttgctcagcttatatttgtccccttccttgcccgagtgggcatggaacacattgatgagggtctccatggcggtctccagctcagagcccatttcagcagcacacggttggcctcggcctgacgtcggagcaggttcttgacagaagggcgcgctgcaaatgttgccccggagcagtgtggggagacctgtctttactgccgaccctcctccccttgtgcc

SEQ ID NO: 96 - DNA, 1312 bp, Rattus norvegicus
gagtgccagtcatgtcgtccatcctgcccttcacccccccgatcgtgaagcgcctgctgggctggaagaagggcgagcagaacgggcaggaggagaagtggtgcgagaaggcggtcaagagcttggtgaagaagctcaagaagacggggcagttggacgagctggagaaggccatcaccacgcagaacgtgaacaccaagtgcattaccatccccaggtcactggatggccggctgcaggtatcccatcggaaggggctcccccatgtcatctactgccgcttgtggcggtggcccgacctgcacagccaccatgagttacgggccatggagctctgtgagttcgccttcaacatgaagaaggatgaagtgtgtgtaaatccttaccactatcagagagtagagacaccagtgctacctccagtgttggtgccacgccacaccgagatcccggccgagttcccccctctggatgactacagccattccatccccgagaacactaacttccccgctggcattgaaccccagagcaatattccagaaaccccacctcctggctacctgagtgaagatggagaaactagcgaccaccagatgaaccacagcatggacgcaggctctccaaacctctccccgaatccgatgtccccagcacacaataacttggacctacagccagtcacctactgtgagccggccttttggtgctccatctcctactacgagctgaaccagcgagttggggagacattccacgcttcacagccgtccatgacagtagatggcttcactgatccctccaattcagagcgcttctgtctgggcctgctgtccaatgttaaccggaatgcagccgtggagcttacaaggcggcacattgggaggggtgtgcggctctactacattggaggggaggtctttgcagagtgcctcagtgacagtgctattttcgtccagtctcccaactgcaaccagcgctatggctggcaccctgccactgtctgcaagatacccccaggctgcaacctgaagatcttcaacaaccaggaatttgctgccctcctagctcagtctgtcaaccagggctttgaggctgtctaccagttgactcgcatgtgcaccatccgcatgagcttcgtcaaaggctggggagccgagtacaggagacagacagtgaccagcaccccctgctggatcgagctacacctgaatgggcccttgcagtggcttgacaaggtcctcacccagatgggttcccccagcatccgctgttccagcgtgtcttagagacactgggagtaaagggatcg

SEQ ID NO: 97 - DNA, 1200 bp, Rattus norvegicus
catgagcgccgctcttttcagcctagacagcccagcacgcggcgcaccctggcccacagagcccgcggccttctacgagccaggcagggtgggcaagccaggacgagggccggagcctggggatctgggggagccgggctccacgacccctgccatgtatgacgacgagagcgccatcgacttcagcgcctacattgattccatggctgccgtgcccaccctagagttgtgccacgacgagatcttcgccgacctcttcaacagcaatcacaaagcggccggcgcgggcagcctggagctgctgcagggcggccctacgcgacccccgggtgtggggtcaatcgccaggggcccgctgaagcgcgaacccgactggggcgacggcgacgcgccgggctccctgctgccggcgcaagtggcagtgtgcgcgcagacagtggtgagcttggcggccgcggcacagcccacaccacccacttcgcccgagcctcctcgaggcagccctggaccgagccttgcgcctggccccgtccgagagaagggcgccggcaagaggggtccggaccggggcagccctgagtaccggcagcgacgcgagcgcaacaacatcgctgtgcgcaagagccgggacaaggccaagcgccgcaaccaggagatgcagcagaagctggtggagctgtcggccgagaacgagaagctgcatcagcgtgtggagcagctcacccgggacctggccagcctccggcagttcttcaaagagctgcccagcccgcctttcctgccgcccaccggcaccgactgccggtaacgcgcggtgtgggccttagagactccgaacgaccgatacctcagaccccgacggcggggagcagacgccgcccgaattgctacagtttcttgggcactggactgcgagagaagctatatgaatcccccttaaattatttttttataatggtagcgttttctacgtcttattaccattgcagctaaggtacattgtagaaaagacttttccgacagacttttgtagataagaggaagagactgcgcatgctttttatattcatttttacagtatttgtaagaataaagaagcatttaaattgcaaaaaaaaagaggcaccagctctgactggcctctttctaggctacggtgatcctgagcatcttttgttacctgctggtagaaatgatcct

SEQ ID NO: 98 - DNA, 593 bp, Rattus sp.
ccgggctcattaggaaacctttattgtcaccattagagataaaaacatcggagtcccacacgccacaccacacaaggaggctaactgaagggttactgattgtcacagcagcccagacaaagggctgaacctcggtccttagccaccccctcccctgcacgatcagtggcagagggcaatggtctccatgttgaggaagcggatgtgcatcttggtctcgatgtcgatgccctgccagatcttcaggaagtcctcaaaggtgatcccctcgtacacctggtccggttccatctgtcccacgcacacgctggcagcctccatcatggctccgtcggcgatggaccgagctgactccttctcgatgtgaggatttccagagagcagttcctccaccacatttcgatactcttccagggtgatgcggccgtcactgtccgagtcatacatatggaacagaaatttcagcttctccttccgagacagctccacttgttcctcacccagggtagtgtcaatgggccggaagtaggacatgatagtcaggaagtcctcgaagttgatctcatcggccaggccgctggaccccttacgca

SEQ ID NO: 99 - DNA, 281 bp, Rattus sp.
acgccggacgccgcccacgtctgaacttcgggagacggagggccccggggagcggggccctgcgagcagactcccagccgcgcgacgcgatcccccccgcggcggcgaaaccgcaagggggggcggcgattgatcgtcaagcgacgctcagacaggcgtagccccgggaggaacccggggccgcaagtgcgttcgaagtgtcgatgatcaatgtgtcctgcaattcacattaattctcgcagctagctgcgttcttcatcgacgcacgagcccctcgtgcc

SEQ ID NO: 100 - DNA, 573 bp, Rattus sp.
ttcatcaccaatgctaagtaaggttgaatggagtggtggagtggggaatttactgataccctaacatggtttgtacaccgtgagtcattgcatcagagagctcttggtctccagaagaacgtggtatcctggttgttgccttactagctgatgaatcagatttactaagttgatcgaagttttgccgctgtgcttccccgtgaatcccctttagcagatatggctgtgagctgttttcttgggttggtatctttgagcataggtgagttgggttccttatctcttgtcaagactaaaatgggggaatgaaaatgcagacagtttaatgattatttttaggagctggcatgatatgggctttggagatgaaatagaaacaacacaagattcaggtctctgaagtcaataaaaatggaattttggaggtgcaggttttaaaatatatttcttttcatgagaagcacagtatgctgcgttggtgcactgttcaagtgacatggagatccttggttgaagcagccattattctaactctctaacgacttgttctgaaataaaaccgtggtttgtatg

SEQ ID NO: 101 - DNA, 528 bp, Rattus norvegicus
tttttttttttttttttactggtactaaaggttttactgtaataaaccaaatatatttacaatttatacaaatgttgaaacaacaaaaatacaaaaagaaaaaaaaatcacaagatgcaaaaccaagaaacaagttcactggagacaccactgctctacaaagactcaaggtgaattgaactgcgagaaacagaacggtaagctcgcagtcagcaaaggcaggacggcagacagcagcttgctcctctattccacacctgtggcctcggtgtgattgtggtgtccaaagttagacaggtgagtgtctactgatagacttggtgccttctttttgtagacgcaaagcagttctgtgtcaagatttggaaacaggtttcttcagtttttttgatgttgtttttgttttttttaaattataagttctatttctacaaattccaaacatttggcccaattcaactgtaaatatgaagccatggtattctcagtagatgacctcataaacggtagcctttttgtcccctgagc

SEQ ID NO: 102 - DNA, 3509 bp, Rattus norvegicus
cgcccgcctgagtggccggacctcgcgcctcgcgcctcgcgtcccgcgctgcagccgcagtcggcttttgttgtctccgcctcctcgtcggtccccgactctggaccacgagcggcgcgcgctgggaccttggctctgcccttcgcgcagactgggctgagcgggccgggcggccatccagggaggcgcgcacgggcggtcggggagccgcgggccccgccatggagctccgggcccgaggctggtggctgctgtgcgcggccgccgcgctagtcgcctgcacccgcggggaccccgccagcaagagccggagctgcagcgaagtccgccagatctacggggctaagggctttagcctgagcgacgtgccccaggcagagatctcgggagagcacctgcggatctgcccccagggctacacctgctgcaccagtgagatggaggagaacctggccaaccacagccggatggagctggagaccgcactccacgacagcagccgtgccctgcaggctacactggccacccagctgcatggtatcgatgaccacttccagcgcctgctgaatgactcggagcgtacactgcaggatgcttttcccggggcctttggggacctgtacacgcagaacactcgggccttccgggacctgtatgctgagctgcgtctctactaccgaggggccaacctacaccttgaggagacactggccgagttctgggcacggctgctggagcgtctcttcaagcagctgcacccccagctgctgctgcccgatgactatctggactgcctgggcaagcaggcggaggcactgcggccgtttggggatgcccctcgagaactgcgcctgagggccacccgtgcttttgtggcggcacgatcctttgtgcagggcctgggtgtggccagtgacgtagtccgaaaggtggcccaggttcctctggccccagaatgttctcgggctgtcatgaagttggtctactgtgcccattgccggggagtccctggtgcccggccctgtcccgactattgccgaaatgtgctcaaaggctgccttgccaaccaggccgacctggatgccgagtggaggaacctcctggactccatggtgctcatcactgacaagttctggggcccgtcgggtgcggagtatgtcattggcagtgtgcatatgtggctggcggaggccatcaacgccctccaggacaacaaggacacactcacagctaaggtcatccagggctgcggaaaccccaaggtcaatccccatggctctgggcctgaggagaagcgtcgccgtgccaaactggcactgcaggagaagtcctccacaggtactctggaaaagctggtctctgaggccaaggcccagctccgagacattcaggactactggatcagcctcccagggacactgtgtagtgagaagatggccatgagtcctgccagcgatgaccgctgctggaatgggatttccaagggccggtacctacctgaggtgatgggtgatgggctggccaaccagatcaacaaccctgaagtggaggtggacatcaccaagccggatatgaccatccggcagcagatcatgcagctcaagatcatgaccaaccgtttacgtggcgcctacggtggaaatgatgtggacttccaggatgccagtgatgacggtagtggttccggcagcggtggcggatgcccagatgacgcctgtggtcggagggtcagcaagaagagctccagctcccggacccccttgatccatgccctccccggcttgtcagaacaggagggacagaagacctcggccgccactcgcccagagcctcactacttctttctgctcttcctgttcaccttggtccttgctgcagccaggcccaggtggcggtaactgccccctagcccaaaggactgtcttggccaaaacatgcaacagaccatatttacttcccttggccttcgaggcccagggcaggataaggagacagtagctctgagtgctggggcagggcgcatggggtcctggccttcctgggtctggccacgcctgtcaccctagcttctagttgttgtatcaggtcagctgcgagccagtgtccccaaaagccatgtttttcagggacctcaggggcacctctggctgcacactcctcccctaccctcctgtaccaccccagaagctcacgaggtcacccagaagggcggttattagctacaacccatcggagacctcaagtgagtctgtgtcttcctctcctgcctcttcccggtggggactccccaccagaccccatgggacacagatgtcagaaattgaggcccatcccgcagctccccaggaagcctggaagggatgccagtatgtcgctgaccaggctctggcagggcctacaagtttatgcatgataccttctcctcagaagaagctctgcaggcaggatcccaacacacaccagaccaggaccctgtgccactgtggagctcagtgaatctggttctcaaagaagacccactgtggggttcctctagtgtgacataggtgaggtggcagctggcaaggccgcactaggctgccacactgtttgccccctcagatgggctcataaagactgggcttgagggtccacatgaagagcctcacttcaggggaatagccggccacgtctagcccctatcccacttagaaaggtcacctggccacggtgatgctgggtggctgatgaaacttaagctcagggtcagtgggacctggcactgcaggtcaagaggacgcctgggccccttcctgacccaggcagctctgcaggagctgggggagcattgacagtcaagggcttttatagacatacacattcagaccctcggtgtccttgtccactgagtgctgtatctcatcgtatctcaatcttcattgacagcactggagaggctcggggcaccacttggagccttgtatcccgcaggcttgagacctgggggcacctggcttaccaaggttggcaaggctccatcttcccttccaggggcttggggatgctaagttgctgtatccaggaagacagtagtcctcacatgcagaaggcctgggaaaaggctgcttgtcgctttttttttttcctttctttttgtcactgggttagagaggctcccgccagcacagcaccaaggcctgcccagtagtcaggtctcctggtccaggatgggtgtgctctgtagttggtctgtgggtttcttaggcccatgccctgagcacattcacccagccacatccctgctagtgacactcaggcaggggcactgggaaccagacctgggctctggcatcaacgagtgcctaggtgtcaggacagcggcacccatcctgtacagggcagccaggatagtggccaagctactgtgtcctttctccatgaggctccctgtcactcggtgccagtgggtaatgtgtgttctttgagtccttatatgaataaaaggctggagacctaccaaaaaaaaaaaa

SEQ ID NO: 103 - DNA, 3044 bp, Rattus norvegicus
tgcactccagccatccagccatggctccctcacagctcgcgtggctgctgcgcctggccgcgttctttcatctgtgtactctgctggcgggtcagcacctcggcatgacgaaatgcaacatcacgtgccacaagatgacctcgccaatcccagtgaccttgctcatccactatcaactgaaccaggagtcctgcggcaagcgcgccatcatcctggagacgagacagcacagacacttctgtgctgacccaaaggagaaatgggtccaagacgccatgaagcacctggaccaccagaccgctgccctgactagaaatggtggcaagtttgagaagcgtgtggacaatgtgacacctaggatcacctcggccaccaggggactgtccccaactgccctggcaaagcctgagtcagccacagtggaagaccttactttggaaccgactgctatttcccaggaggcccgaaggcccatggggacttcccaagagccaccagcagcagtgactggatcgtctccctcaacttccaaagctcaggatgcagggcttgccgccaagcctcagagcactggaatttctgaggtggctgctgtctctaccaccatttggccgagttctgctgtctaccaatctggatctagcctctgggccgaggaaaaagctactgaatccccccccactatagccctatctactcaagcgtccaccacttcaagccccaagcagaatgttgggtctgaaggccagcccccatgggtccaggagcaggactccactccagagaaatctccagggcctgaggagacaaacccagttcatactgatattttccaagacagaggacctggcagcacagtccatccctcagtggctcccacctcctctgaaaagacccccagcccggagctggtggcctcgggcagccaggctcctaaggtagaggaacccattcatgccactgcagatccccagaaactgagtgtgtttatcactcctgtccctgactcccaggcagccacccggaggcaggcagtggggttactggcctttcttggtctactcttctgtctgggggtggccatgtttgcctaccagagcctccagggctgtccccgcaaaatggcaggggagatggtggaaggcctccgctacgtcccccgtagctgtggcagtaactcgtacgtcctggtgccggtgtgagctgcctgcttgcctgcctgtgtccagagtgtgattcagacagctgtctggggacccccatcctcatacccaccttcatccacgctgggagaatgggaatgcagaagctagatcctccaggggccatgtgctccaacccccgaggagtggccctggaggccaccctagaccattattcacttatcagagacagacagagcaggtggccttccagctcccccatatttgaaagaatcctctgctgctggctggttagaggggcccttgacatcccaaactctatgagcaattatttattggattcccagccccctgagacacccgtttccccgtgcgcaccgtggtccacccatcttacaagcagcagccaggcctctctgcctgtcccctgacctcctcgtgtctcctggctttgctgcagtcgccagccctgtctcctccccggccagctgcggtgctatctatcccggtctctctctctcgtgtacagagccatcaccaccatcaccaccaacaactcgttctgtctttgcttgcatgaggttaatgctgtgttttctggagctctctgggacgggagatgagcttctgtgagggtttaaagtgttcctccccagactttgatgtgccgtgaagcatgctgcctctgaaggaaggctctggtccccactccgctgccagcacaacaaagtgccccacctgtaaaggaaaagagactcggcccagagctggcaaaaacccatggccctgacatcatcactttctctgagatctttgtccccatccctggatgccaccccccccccccagcccttatcaacatgaatagtcactgccattccactggactgacacttttgtatgctgtgattctgagggctggcaagggatgacttgagagtgcagatcctacccatgggcccccaaatggaggctgagctggggacctgcaggaagagaggccaactcagaaggctccgctgtgttctcactggcacccctcccctgtgcaccaaggtgacagtcacaggtctgccctgcctgaagaacaagccacagaaggaagattatgacaagtccctgggaggccaaggatccagggcaaaccctggagtggccacacacccaatttcagcttagggacttgtgcatgtgtgtacttgcacagcccagatcattcagaagctgccaggatcctttcctacatctgagagcgcagttcctgccaaggtctcaccttcgcctcacttcaggcagggcagaactcccataacattctccaagagccctgtgacgtgttctggaagggactctgccctgggcacaaagtgtctactgaagcagagagcagcgccccagccccagccccagcgccccagcgggagctgtaccggcagaccacgccctgggggggagggccctccgtggcagctttccgctctgaatagctccaaccgtcacctttggagcctcccaggggcgggcttcacccagccagtgactcactccttgataggtggaagctcagaacaggtggtctcgtcccagagtgaggaagccagccccttggcgaccctcctcttgggaagcttgtgggaggctctggtctggctccagagtactagttgtaggcctgaggagcagcagcccgagtgcactatatcctggttcttcggtggggagccttcaagggttgggacacccatggttggactttgttggttcccgcgcctcgtgggccaaaacaatctgaggaggactttggaaggagttggtttttcaagcatcattaccaatgtctgtgccattttgtattttactaataaaattttaaagtcttgt

SEQ ID NO: 104 - DNA, 684 bp, Rattus sp.
cacagttcagcttttattaaaacgccgtgtacagtgtggggagctggaatgggctcctgtgaaaatgatacgtttgaggggattttcccgctcttaccgtactgatcacgtggcaagtgttcttttacaaggtatatggtccctaacatccatgcagaaaatatcaggtcaagtgctttactgtcccagtgtccagtctggtgacccatatcagaggcagggcattcgcagagatattctcattccgtgtcttgagttttctttcgaagaaagtattttacgagtcatatacaaattaaacaagaagtccaacgagggccctttattaccaccaagggggggaaggggtaacttacaatcttataaccaaaggcaaaaacagcacaagaacagtttattcctgcagatgccgcagccctgtgcaatcccgcctcggctgctgctacagccagaagtcagagccccgcagtgaaggctgcacgggaactctaggctggattacccacacagaacaagtcgctcctgcccccgtacgttggaagtgctgctggattttctgcttgctcagtggataacagtgcagctgaacatgaggggcttaaatagcacgctcgcgcgcacacacgcatgcatacacacacgtgcacacacacacacacacacacacacacacacacacacaca

SEQ ID NO: 105 - DNA, 3471 bp, Rattus norvegicus
ggcacgaggaccggctgaggattttatggttcttaagcggacttaagagcgttgtttcggattgttaagattcccgtttgctgggttttcctccctcaatcgtgctctcccgcggctgcctggggactggctcggcgaaggaggatggagagggggctgccgttgctgtgcgccacgctcgcccttgccctcgccctggcgggcgctttccgcagcgataaatgtggcgggactataaaaattgaaaacccggggtaccttacatctcccggctaccctcattcttaccatccaagtgagaaatgtgaatggctaatccaagctccggagccctaccagagaatcatgatcaacttcaacccacatttcgatttggaggacagagactgcaagtatgactatgtggaagtgatcgatggagagaatgaaggtggccgcctgtgggggaagttctgtgggaagatcgcaccttcacctgtggtgtcttcagggccatttctcttcatcaaatttgtctctgactatgagacccacggggcaggattttccatccgctatgaaatcttcaagagagggcccgaatgttctcagaactatacagcacctactggagtgataaagtcccctgggttccctgaaaaataccccaacagcttggagtgcacctacatcatctttgcaccaaagatgtctgagataatcctagagtttgaaagttttgacctggagcaagactcaaatcctcccggaggagtgttctgtcgctatgaccggctggagatctgggatggattccctgaagttggccctcacattgggcgttactgtgggcagaaaactcctggccggatccgctcctcttcaggcattctatccatggtcttctacactgacagcgcaatagcaaaggaaggtttctcagccaactacagcgtgctgcagagcagcatctctgaagatttcaagtgtatggaggctctgggcatggaatctggagagatccattctgaccagatcactgcatcttcccagtatggtaccaactggtctgttgagcgctcccgcctgaactaccctgaaaacgggtggacaccaggagaggactcctacagggagtggatccaggtggacttgggcctcctgcgattcgttactgctgtggggacacagggtgccatttccaaggaaaccaagaagaaatattatgtcaagacttacagagtagacatcagctccaacggagaggactggatcaccctgaaggagggaaataaagccattatctttcagggaaacaccaatcccacggatgttgtctttggagttttccccaaaccactgataactcgatttgtccgaatcaaacctgcatcctgggaaactggaatatctatgagatttgaagtttatggctgcaagataacagattacccttgctctggaatgttgggcatggtgtctggacttatttcagactcccagattacagcatccaaccaaggagacaggaactggatgccagaaaacatccgcctggtgaccagtcgaaccggctgggccctgccaccctcaccccacccatacatcaatgaatggctccaagtggacctgggagatgagaagatagtaagaggtgtcatcattcaaggtgggaagcaccgagaaaacaaagtgttcatgaggaagttcaagatcgcctacagtaacaatggttctgactggaaaatgatcatggatgacagcaagcgcaaggctaagtcttttgaaggcaacaacaactatgacacacctgagctccgggcctttacacctctctccacaagattcatcaggatctaccccgagagagccacacatagtgggctcggactgaggatggagctactgggctgtgaagtagaagtgcctacagctggacccacgacacccaatgggaaccccgtggacgagtgtgacgatgaccaggccaactgccacagtggcacaggtgatgacttccagctcacaggaggcaccactgtcctggccacagagaagccaaccattatagacagcaccatccaatcagagttcccgacatacggttttaactgcgagtttggctggggctctcacaagacattctgccactgggaacatgacagccacgcgcagctcaggtggagggtgctgaccagcaagacggggcccattcaggaccacacaggagatggcaacttcatctattcccaagctgatgaaaatcagaaaggcaaagtagcccgcctggtgagccctgtggtctattcccagagttctgcccactgcatgaccttctggtatcacatgtccggctctcatgtgggtacactgagggtcaaactgcactaccagaagccagaggaatatgatcaactggtctggatggtggtcgggcaccaaggagaccactggaaggaagggcgtgtcttgctgcacaaatctctgaaactgtatcaggttatttttgaaggtgaaatcggaaaaggaaacctcggtgggattgctgtggatgatatcagtattaacaaccacattcctcaggaggactgtgcaaaaccaacagacctagataaaaagaacacagaaattaaaatagatgaaacagggagcaccccaggatatgaagaagggaaaggcgacaagaacatctccaggaagccaggcaatgtgcttaagaccctggaccccatcctgatcaccatcatagccatgagtgccctgggggtgctcctgggtgcagtctgtggagttgtgctgtactgtgcctgttggcacaatgggatgtcggaaaggaacctatctgccctggagaactataactttgaacttgtggatggtgtaaagttgaaaaaagataaactgaacccacagagtaattactcagaggcgtgaaggcacggagctggagggaacaagggaggagcgcggcaggagaacagtggaggcgcagggactctgttactctgctttcactgtaagctgggaagggcggggactctgttactccgctttcactgtaagctcggaagggcatccgcgatgccatgccaggcttttctcaggagcttcaatgagcatcacctacagacacaagcaggtgactgcggtaacaacaggaatcatgtacagcctgctttcttctcttggtttcgtttgggtaatcagaagccagttgagaccaagtgtgactgacttcatggttcatcctacttggccccctttttcctctctttctccttaccctgtggtggattcttctcggaaactgcaaaatccaagatgctggcactaggcgttgttcagtgggctctttcgatggacatgtgacctatagcccagtgcctagagcatattagcataaccacatttcaggggacaccaatgtccgcttttgcatcgctacgtgcagcgagcacaggaaaaagaaaaaaaaaaaaaaaaaaactcgag

SEQ ID NO: 106 - DNA, 528 bp, Rattus sp.
  misc_feature (1)..(528): where n may be a or g or c or t/u, unknown, or other
gggaaggagagggtttatttcagcttacagttcaacatcacagttcatcactgagagaaaacagggcagaagctaatgctggggtcacagagagtgcatctgtgtaggggatttggcaggtagcctgcagtctggtcactcttgaggacagcattttgctagctcagccacccagccacaactcagccgggcccaggaagcgacccagcgtcattgctatgggtcgtttacacaaaccacaccagcatcaattaacacttttttctgtacctgatgccacagtttaaagaaaaaacaaaacaaaacaaaacaccaacacacgactatacatcatcacagttgggcagcccatgggtgaggtttccacagctactccacaaaggtacccatcctgcgtcaggagatccagcaccttgccacagactgcagctcacaggtgcctgtacactgaatattttggtctaaaatttgacctttatgggatggggagagcnctctttgttcttaaacctggatatccttcaagtc

SEQ ID NO: 107 - DNA, 437 bp, Rattus sp.
gaagtaaactaaatttattatcttcttacaagaatgctttagttgtttggttttgtttcaagtcctgtgccttttaactttcaccataaagtattattttttacatgtccttgacaaaggcttgataagcagttcaactctttgggcaaagtcactagctcttatccgaattagaatacaggacacagaataagtcaagacaatccatgacatttttattgagcctttagtttatgaggtaaaggaaaagcagctctcttgaatactttacatagctgtaggaaaatatttgagacttaaagggagttacaggggattatgtttctcaaaagttgagatcaagtaaacaagctttaaattgtttaaattttccagttgatctttccttacaatagtaacaagctcgaattagccacattaggtttttatgtttgcag

SEQ ID NO: 108 - DNA, 385 bp, Rattus sp.
tgtgtttggtaaagggccctgggtgaggatatagctcctctgggcttcctccatttttatcaagctggcattgatatagtcattatcttcctgatgcaatttaatccgactgtggtcaaaagggctgacatctcggtacctgttccggtttttgttcttaggaagtttcgctattctgcatgggaagtcactggcttcatgtcgaatatcctggtaaatagccgcccagttcccagccttatcgatctgctcgaattccttctccatttccatggcgggacgggacgactgcccgagttccctcggccgtccgccgcgctaggccgcgtcgctccgcgtctcgaggcccgtcgcccgccacctcgtgccgcctcgtcctcgtgcc

SEQ ID NO: 109 - DNA, 646 bp, Rattus sp.
attttttttctattttttttttttattatggtttacagctactttatttacaactatacatttaacacaatgagataaacactgatagactgaaggactaaggtttgcagctactttatttacaagtatacatttaacacaaggagataaacactgatagactgaagcctaactaatagtactgtaacgtgtaccattttgatgactacattattttaaacaacaaactacactgaaaaattaatgccgataaaattcttggtcataatattaagaaatacaatatataaattgaaaatatgattgtttaaaatttgaaaatggaagtgaactcatttggacagagtcagacgttaacataatctgagaggggaggacctctgacccaaatgacatcgttcaggttaacagaacaaaacagaagcctagttttatcttcaaggatgacaggcagcttgcttcctcaggtggaatacactcaggctctgcatcacgcgcgtcacatcaccgagcacgaagacttcccttgtgaagccgctccgtcaatcttttctgcttccaaaattatccttcgaaaaacatccacagcagtctgattttcttttgcagaagattccaaaaaagctgcattccaagattctgccaa

SEQ ID NO: 110 - DNA, 484 bp, Rattus sp.
atagaaagagatatttaatacttttaaaaaaaaaaaaaattaggagtaaagtccacttcacaggctggaggacagtgttgcccatctagggggcatgggtgtggccccacgtaccagggacaagccacgggttgtggggcagctgtagagaagagtgtaactgagtccagccaaagcctccctcatgatgcagagccgcggctgtggccagcctcgcatgctggttgaaaagggacctgccaactgcctgtggccagctgtagagtcacatgagcaggtagcagtggagctggctcaggggaaggtgccagggacctgggaatgtccactcttcagggcctttgtgcttggtcttcaacagggctgagctgggtctgtggtcccatgggaagtgtacacgttcatgccttgtgcacttctgcatgagcagcaggtgccaggtgtctccatatgctgagctggctgtgcttgcagcctcgtgccg

SEQ ID NO: 111 - DNA, 711 bp, Rattus norvegicus
gggactcgcacttgcaatatgactttggaggaattctcggccgcagagcagaagatcgaaaggatggacacggtgggcgatgccctggaggaagtgctcagcaaggctcggagtcagcgcaccataactgtcggcgtgtacgaggcagccaagctgctcaacgtagacccggacaacgtggtcctgtgcctgctggctgcggatgaagatgacgaccgggacgtggctctgcagatccatttcaccctcattcgtgctttctgttgcgagaacgacatcaacatcctgcgggtcagcaacccgggtcggctggcagagctgttgctactggagaacgacaagagccccgctgagagcgggggcctggcgcagaccccggacttacactgtgtgctggtgacgaacccacattcatcacaatggaaggatcctgccttaagtcaacttatttgtttttgccgggaaagtcgctacatggatcagtgggtgccagtgattaatctccccgaacggtgattccccgaacggtgatggcatctgaatggaaataactgaaccaaattgcactgaagttttgaaatacctttgtagttactcaagcagtcactccccacgctgatgcaaggattacagaaactgatgtcaaggggctgagttcaactacaggagggctaggagatgactttgcagatggacagagaggtgaaaata

SEQ ID NO: 112 - DNA, 608 bp, Rattus sp.
  misc_feature (1)..(608): where n may be a or g or c or t/u, unknown, or other
cggccaacttgatgttctagtgctgaagggagcaagggccaggcatgtggtggagatgatgctgaaatggtttatccaataccatgcaaatcaagtcctttggatagaggtgaagaacttggacatggctgtttcaggcagctgaagtcaaaaggaccgggactggggagggcagggacgctaagtgaaagggctggtggccaacgggccttctactgactagaaggacttggctctgagatcttccatctgacctaagaggcagcccctcttgcgggccatctcttccctcctgtcctttattgtctctcgcactcccctccctttgctctctttcattccctttagcaaatttcaattgtcctaggagaaaaggttgctgtcatgtctgaaagacccgttgaggtgctgaaggagttgctgacccctgcagactggaaggtgctgacctgggcagggaaagcaggtgggacggaggcataggtggtggccaccgagggcgatgggaagccactgtgcgcaggagacgggtaggtagaggagcccggagaggagtaagaggtgggcactggggatggaaatgaggtggtggcggnggatgggttaga

SEQ ID NO: 113 - DNA, 539 bp, Rattus norvegicus
cctcggcctccgggctgcgggaacgcccggactgggaacgccacgtcgagaggcgttcgcggaaggcgcgggatccaggacgtgctggtcacccccaaaccccaggccacccattatcgccttggttcgcccatcagagttgtaagaaaatggcagacaagccggacatgggggaaatcgccagcttcgataaggccaagctgaagaaaaccgagacgcaggagaagaacaccctgccgaccaaagagaccattgaacaggaaaagaggagtgaaatctcctaaaagcctaggaagatttccccaccccaccccttcatctccaagaaccccctcgtgatgtggaggaagagccacctgcaagatggacgcgagccacaagctgcactgtgaacccggcactccgcgccgatgccaccggcccgtgggtctctgaaggggacccccccactaatcggactgccaaatttcaccggtttgcccagggatattatagaaaattatttgtatgattgatgaaaataaaacacacctcgtggc

SEQ ID NO: 114 - DNA, 292 bp, Rattus sp.
gcccgttgaccagatccactagaactgtctgcattatctatgcagcatggggtttttattatttttacctaaagatgtctctttttggtaatgacaaacgtgttttttaagaaaaaaaaaaaaaggcctggtttttctcaatacacctttaacggtttttaaattgtttcatatctggtcaagttgagatttttaagaacttcatttttaatttgtaataaagtttacaacttgattttttcaaaaaagtcaacaaactgcaagcacctgttaataaaggtcttaaataata

SEQ ID NO: 115 - DNA, 2299 bp, Rattus norvegicus
ccactgcagcttttctctgttgcttaaggctttcggttacttctctttctctaagcgagcgttctttgttggtgacccgagttgctagtccagaaggaacagactgagtgagcgaggcgccatgacaaccctggatgacaagttgctgggggagaaattgcagtactactacagcaccagtgaggatgaggacagtgaccatgaagacaaagacagaggcaggggagccccagccagtagttccacgcctgcggaggctgagctggcaggcgaaggcatttcagtcaatacaggtccaaaaggggtgatcaatgactggcgccgcttcaagcagttggagacagaacagagggaggagcagtgccgggagatggagcggctgatcaaaaagctgtctatgagctgcaggtcccatctggatgaagaggaggagcagcagaaacagaaggacctccaggagaaaatcagtgggaagatgactctgaaggagtgtggtatgatggacaagaatttggatgatgaagagtttctgcagcagtatcggaagcagaggatggacgagatgcggcagcagcttcataaagggccccaattcaagcaagtgcttgagatccccagtggagaaggatttttagatatgattgataaagaacagaaaagcacccttatcatggttcatatttatgaagatggtgtcccagggactgaagccatgaatggctgcatgatctgccttgccgcagagtaccccactgtcaaattctgccgagtgaggagctcggttattggggccagcagtcgttttacccggaatgcccttcctgctctgctcatctacaaggcgggtgaattgattggcaattttgttcgtgtcactgaccagctgggcgaagatttctttgctgtagaccttgaagctttcctgcaggaatttggattgctcccagaaaaggaagtcttggtgctgacatctgtgcgaaactctgccacctgtcacagtgaagacagcgatctagaaatagattgaactgataatccagttctgtagctgtctcattgtttgggctagaggacacatgtctgtatttatttctgtccttcctgtcttctggctttacagctgctctttgtagtctggtttagtatgtggaaagtcaagaaactcagattaaatcagaatcctgactcactttgtggctagcagtaaagcgatttctaattatatagacaggaagctgggttcttgagctgtttacatctctagcgtgacatctctgaaattgtttccagtcaatattgacatggcacccttgaaggcaatgtcttgaaaattgtcttctgatgacctcagaattccaccaggtctgagagtagaattccctagtgagtgtgtttctgtgcagtgtaaacagtgcatttccataatcacttgattgcaaatcatgtttacttgcaatcagactgtacttattttctccagatccttttctaccagggtccatacaaggttgggaatatagctcagtggtgaactcttaacctagcatacacgtagctccaaggtatctgattcccacccagcactgtcaaaactaaaggttccagatggtgcctgatgctgtagttagcagtggtctctgctgagggagtcactggtccagtccagcataatgaagtatgaacaggaagggatgttttcttgctgactgctgtcatgatgttgggaagcaggcatcctattgactgacactggtgtgtatagagcttgaaaacctcagtaggaggacctttctgtatcctgctcatcagcatcttttcctctgttatttagcaaggtaatgctttgtttaacctttttaactttttaaaaagctttttttgcctattagaaaaaaattcatatttactagaggaaaattggccaaaatacagatgcaaaaatggtttaaaaatataattctgccacctgaatactgtatgttttggtaccattcatatatagggttttttttttaatgtattctagggttttttttttatggattcataggctttggtttgtctttaaatgacattgggatcattctgagcatactgtcagatagtgtgatcagttactaacagaagtttttcatgctattaaatactcttctgtaatattttaataactgttgatattccattgatttgctgaaatctggtgtttgggttttagaaagatagcaaacttttttattataaactttctattatgaaacatgtttatcatagaacatacaataaaaattaagttaaaaag

SEQ ID NO: 116 - DNA, 1739 bp, Rattus norvegicus
ggaggacggcacgtcgcgggtggcattgtgtgtcccagtgtgcaagaatagccccagaagaggaaaggctgagcccagagcgcttcagcagggaagattcccttccccccgcttcaggctgctgagcactgagcggcgctcagaatggaagccatcgccaaatatgacttcaaagctactgctgacgatgagctgagcttcaagaggggggacatccttaaggttttgaatgaagagtgtgatcagaactggtataaggcagaactcaatgggaaagatggcttcatccccaagaattacatagaaatgaaaccacatccgtaagtcagactctatggttcccctaagcaaggggtttgcctgcttgtctccagcactgttttgatttggttttggggggatgatgtagaaggctcttcactatataggccttgttcccctaagctgtagccagcctgtagaccaggctggcctcacagagacccacctgcctctgcctccctactgttccagcatgcccagattggtttggttttgtgagataggcttaaaagtcttaataaagacatcaaaatcaaaataataatattgaagaaatcaaaatcaatttaaatggcaaatatatgttagattactggacctctgtccttggttcagaatgccccttgcctgcgatctctgcagcctaagattgggagccagcgtacagccagtccaggttactcagaggcagactcgaatggggataagtccgaagaagctttgagactccaagcatgaagtcctcacagcttcctgtggtgggataagcccacttggcactcctacagccatttagccttgggttttttgtcttttaggtattttttgctttagggagttttgtgggcttttcacagacaaaacagtaatacgctggtggagaagcacgaagcagtgaggatgacccctagatcagacctagactcagcctgtgcatctcaccaagagtccctacgcgtctgtctccttcatccatgctcaaaaagcttctgtgccgcatggtatcacagaccgtgtgggtcccagctcctaggaggcagaggcaggggatccctgggagtcgggaccagcctggtctgtagtattgagattagccaggactgcatggtgagaccctgtcttaaagaaaagaaagaaaggatggagggaagcaaggagggagaatataaaagtgtacgtgggaaaacacaggctggagaggtggcttagtggttaggagcattgtctgctcttccatagaatttgggtttgagtcctagcaccaacatcatggctcacagccatctgtaatttaagttctagagacccaagtcctcttctggccaccaaagtcacagacatacatgcaggcataacactcatgtatatactttttattttttaaataagaaagaaaagcagaaggtcaggagacagtttgtaaaggaggaagttttctttctttctctccctttgtttatttcttacttggtcaaggccttactatatggctctgactagaacttttgttgaccactctggccttaaactcaaagatctgcctgcctctgcctctgcctctgccttccaagtgtgtgtattattaaaggtccatgccaacacacatgtcctgccttcttttaaaaagtaaattgtgggggttggggatttagctcagtggtagagcgcttgggccctgggttcagtccccagctccc

SEQ ID NO: 117 - DNA, 2376 bp, Rattus rattus
cgcggccgtccgctctcccaactcgcagccagtcggcgcgtcccgcctactgagcgcagcctccaccaggatccgcggggaccagctcgggatcagccggcgacccacttctgaccaacccaggagcggcccgatacccactcccgaccaacccgcgaccgacccagggacccactccggacctgctccttacaggggacagcgcctcgccgcttcccgccgcccagcgcccgcacgctcctcgggacacagtgccaaccatccagaggacaagatggattggggcacactacagagcatcctcgggggtgtcaacaagcactccaccagcattgggaaaatctggctcactgtcctcttcatcttccgcatcatgatcctcgtggtggccgcgaaggaggtgtggggagatgagcaagccgattttgtttgcaacactctccagcctggctgtaagaatgtgtgctacgaccactacttccccatctctcacatccggctctgggctctgcagctgatcatggtgtccacgccggccctcctggtagctatgcacgtggcctaccggagacacgaaaagaaacggaagttcatgaagggagagataaagaacgagtttaaggacatcgaagagatcaaaacccagaaggtccgtatcgaagggtccctgtggtggacctacaccaccagcatcttcttccgggtcatcttcgaagctgtcttcatgtatgtcttttacatcatgtacaatggcttcttcatgcagcgtctggtgaagtgtaacgcctggccttgtcccaatacagtggactgcttcatttccaggcccacagaaaagactgtcttcacggtgttcatgatctctgtgtctggaatttgcatcctgctaaacatcacagagctgtgctatctgttcattaggtattgctcagggaagtccaaaagaccagtctaatgcattgcctggctgttaagcaaagatgagggagaggatgaggcaacctgtgcttagttatcagagttcagctaccagcatctcccgggcaaacattcccaccttaaatgccgccatttgaagtcccccgcaggcctcccatgaaactccagaagcctccatgggcctcccttcccccaaagctcccaaacaaaggcccaattctatgcctgtattaatgggttctaaagttagttagaccccgtgctggtgtgactatgctttaggatacattcacagtttaaacaaagggatctcacattgtttctcttcctctgaggacaggagacatgagcccagtcctgaggaaggtacagagaaagttccttcttccgggtccccttccccaagttgcccccagttaagggtaaagaatcttcgttctgttattttctttcatagtttaagtttgcaacaatggacaaaagctatttaatgttcaagctagctgtgtccttttttttttttttaaatgaaaaccttaaaatgataggttcttttgttcttaaaatgatctggaaagcattatacattcctcctatttcagaggttcggtttgtgatgtgagcatggtgtataaccagatctcacaaggtctttaaaacgttggccttttggttatgggaaacctgggctgtggctgagagcccacctactgtattcatccttaggtgtgctgagtacagcccgcaacaacgttacagcctgtctcaaatgagacaaactggaagcttctcgtgttagcttctgacaagaagaggccttgattaaaattttcaaccgtaattttgtgtaagaggcagataggttatgcctacaactgccccctgccatgagcctaactcagcccccctccacccccagctcgtctactctgtagctgtgggatgtggcagtcagtatcaaaagacttcatgagtttgcttgggaatttcactgccatggtacaatttaatggtgcagaaacaagatggggtggttttcaaagaaccgatgaaacttctagactctaaatcctgttgattaaaactgagtttttctactttgaatgtctgtttgcctcccttttcagcattgccttctaaactggaaacagaaatgttgatatttggaaaaaatagaagaaactagtttaggtcaatgtgtaacttttctaggacaagttgaaccttagcattgtcattctgcctgatgtgttgtccacaagatgacagtcaacaaatccaacaggggacacttcttcctgccaagaatgtcgttgggaagccattctgtaacaataaataagagttgtggtttaaagtctacactattttacctaatgaagaacttattgctgatgttcagaaattcgacattgaaaggtgttttgccaatacggg

SEQ ID NO: 118 - DNA, 623 bp, Rattus sp.
aacggtgtcataaataagtaatataactttattaaaatgaaaagacaatattcaaaataatgcaacaaaatgaacaaatcctttgtccaatactgtacacacagtgcggagatcagtgcattttctaaagcatgttttaaccttcatttagttcatactaaaaataagctttaaatagctcaaataatgtcattcagcagtttaaactgaacagcttgttgggacatggcagcggtgtccctctgttagcaagcaccttctctttgtgcttatctatacaagataaacaatcagaggatgtaaaaattgaacacaagctacacgtctcactgactctcagggcagtgaggcagccagctgtgagttttctaagcaggaagatgctgaagtgacctctggcattaagacgttctgtgctattggtcagaagtgtttcacttaaaaagcaaacaatccccaggaaatactgaataggaaccaacaacacaagaccagcttgtgttgtaatttaaagtctcaaaaacaaaacaacatcccatacaccccacgataactctcaatcatggctaatcactgggctggtctataaaacttatacccaacactgacggccagcaccactc

SEQ ID NO: 119 - DNA, 365 bp, Rattus norvegicus
ttgccttaaatgttttattacaattaaaatttcacaaacacagatcaattaatctcaatcaaaataactcatgtttacatcatatttatagacaagctgtacaataaatacataaatgcattcacagtctgtgcttcagtcatcctcctcctcctcttcatcgctgatcacatacttcttatgctttttattggccttgacatcgtcctctgctttcctcttcccagaagactcaccttcctcgtcactgttgagcttctttgctttgagcagcctctgagctttgtcttcttccgagccctcgtcgctgtctgaggagtagattctggcccgttcctcgcggatgcctcctttgtagcggtttt

SEQ ID NO: 120 - DNA, 1095 bp, Rattus norvegicus
aaattgcaagcgtattcttttaatgactccagtaaaattaagcatcaagtaaacaaaagtggaaaagaacctacacttttaacttgtctcactagtgcctaaatgtagtttaaaggctgcttaaattttgtgtgtagttggattttttggaagctgaaggtatccatctgcagacattgaggcccaagttgaatttggattcgagtggattcttaacacttctgcctgtgctgaagagaagcttcataaggaacaagcaagttgaatagagaagatagtgatcaataagaggcatttagtggtctttttaatgttttctgctgcgaaacatttcaagatttattgatttttttttttcattttccccaccacactcacacacgcacgctcacactttttatttgccataatgaaccgtccagcccctgtggagatctcgtacgagaacatgcgttttctgataactcacaacccagccaatgcgactctcaacaagttcaccgaggaactcaagaagtacggagtgacaactttggtccgagtttgtgatgctacctatgacaaagctccagttgaaaaagaagggatccacgttctagactggccatttgatgacggagctccgccccctaatcagatagtagatgattggctaaacctgttaaagaccaaattccgggaagaaccaggctgctgtgtcgcagtgcattgtgttgcaggattgggaagggctcctgtgctagttgcacttgcactgattgaatgcggaatgaagtatgaagatgctgttcagtttataagacaaaaaagaagaggagcattcaattccaaacagctgctttacttggagaaataccgacctaagatgcgattacgctccagagataccaacgggcactgctgtgttcagtagaagcagaggaaggccggctggatcgtggcattagagggaactctgggtacctggaaatgtgaatctggattcttacctgtgtcatcaaagtagtgatggattccgtactcctcgactcctcatgattgagaagaaggcaaacgataaagaaatccctctataacacgaataaaatgtttaag

SEQ ID NO: 121 - DNA, 516 bp, Rattus sp.
aatacaagtaaaagggggcagggcaactccttccccctccaggtcaggaccaggagaatctgctgggctgtccccgggaccaaagaggaaaagagtgacatagaaactgaagcaaaggaagcttagtcacactcaggtgagggtgacagctcctcctggattttgtttccatttattaaaaaaaaaaagaaaagaaagaaagaaaaagccaccccctcactcccagcccattcctcacagccagggtcagaaagcagcatcagtgaggcgggttcctcacctctggttatctctggcccaggtcagcttgagccacctgccctcaccaggagagggtttcagttggcagttaggcttggggaagtctctacctggaccccccagaggcctgggagcacccccctcctcccaggaaagggaatgcagtgtctactgggctcagaggggtggcctcacccacctgacatgagtcctgattctcccatctcgaggacggcaggaagtttattgcaccag

SEQ ID NO: 122 - DNA, 484 bp, Rattus sp.
aaaataactgcatcctttaatggcagtaatacaattactggattaagagaccacatgagaaggcaggtgaggtttctggaagacagatactgagtacaaaaggggcgggagagccatgcgacgatcattgtaaaaatacagtacgttatatacatatttgcaccatcgactttcaactcagaaatactatttacacgtttgttacaatcctggttagagaacaatctttctttacgagtgtggcctgacggcaagtggggattcaagcggatgtccttgttccatgagggccttcagttaactgttccggaggaaggctccagactccacctcacccacagatttcagccttaaaaattcatttaattttttctttagcaaaaggacagcttttttctaatatagaccaggctggctttgaacctgtgatcctcctgcccctcctcctgagtgccgggatgacaggcgcactaccaagcctggc

SEQ ID NO: 123 - DNA, 278 bp, Rattus norvegicus
ttgcaagctttctgagcttctgcattcagaccacacagaacatgtaaatatttatacacagaccggagaactagcacacgccttacagcctgtgtgtcccctgccttcctctctcttctctcttaagaagtgcggagtgtttttttttcttttttgcggatttgcctagaggtgccccaggcccccgctcctagagagggtgacctttctgtttctccttctccttgttctccagaggggcaatgtgagggtgatggagccgggtcccttctggtgcc

SEQ ID NO: 124 - DNA, 569 bp, Rattus norvegicus
tttttttttttttttttccaaaggcgcataaaaatatatttcaactttaaaaataacttagttacagtaataccttgcttgtttttaaccaacatgtagctgacagtcaaacttttgcaacacggaaatagtatacagagatatataagaaccacaaggaaaaaaaaatcccaagtcccgtaaggttgttcaaatatgaaaagagtaaaatcttaacactggagaacttgctatggtgaacccaaaagatacaatatacaaaagagtcaaggaaaaaaaaaaggcgtaggtgcgaagtaagttttgatttccttcttccaacatgctgtaaatcgccttggagagaccacggacccagccctactagacagcctggatgtgtgcacatctacaaacaaacaaaaccaagtgtgtttgagcaccgacaatgcctgcttaaatgagctctgactcacttcctcatttccttttttctgccagaacaggttttttttttttcttcaatttttaaaaattgttgttttcaaaatttttgcaaacagggcatgatggttaaaaaaatattttgt

SEQ ID NO: 125 - DNA, 486 bp, Rattus sp.
  misc_feature (1)..(486): where n may be a or g or c or t/u, unknown, or other
aaagccatatgctacacatataacaccttctctctctgtaaggttgacattattcagagagacagtcagaccacaaaccacaaaacaacagtcagacaacaagcactttacagcgcccaccacagtctgtacactaagaacagtcctaagacggcatccgccttctccttcttcaatcaggatgaagctgtcactgacattccagtaacatgctgacgtggttgtcattgtttcaagtcacaccaaactgacatcagagagtacaaggttctttaatcaaacaaaaaattagtcttatttaaattaagagaaattcacttaaactaaaaataatttatttgagctgagagttttaaatacctaacacattttttcagggaaatgttggtaccatctctgagccagctttccttcctccagactgtatctgtataagaatatataatttacttatctctcagtcattttcagannacacgtatctct

SEQ ID NO: 126 - DNA, 2452 bp, Rattus norvegicus
cggaggcgcatcggcactgcctggttctgggggctggggtagggaagagtggggctcactcctacacccaccgatgaagtctgtgagcctcctgatccggtcttgctgagagtgtgggcctatgcccctcccatgtgggaggtgggatagaaagtccctttcctcagcctctctgagtgggtttggtatctcttcgaaagggtgaggtggctttgaccccgggttgcccgccagcgcgaccgaggaggtggctggacagctggagaatgaacggagaagccgactgtcccacagacctggaaatggccgcccccaaaggccaagaccgctggtcccaggaagatatgctaactttgctggaatgcatgaagaacaaccttccatccaatgacagctccaagttcaagaccacagagtcgcatatggactgggaaaaagtcgcatttaaggatttttcaggagatatgtgcaagctcaaatgggtggagatttctaacgaggtaaggaagttccgtactttgacagaattgatccttgatgctcaggaacatgttaaaaacccttacaaaggcaaaaaactcaagaaacacccggattttccaaagaaacctctcaccccctacttccgcttcttcatggagaagcgggccaagtacgcgaaactccaccctgagatgagcaacctggacctgactaagatcctgtctaagaaatacaaggagcttccagagaagaagaagatgaaatatattcaggacttccagagggagaaacaggagttcgagcgaaacctggcccgattcagggaggatcaccctgaccttatccagaatgccaagaagtcggacatccccgagaagcccaaaactccccagcaactgtggtacacccatgagaagaaggtgtatctcaaagtgcggccggatgagattatgcgtgattatatccagaagcaccctgagctaaacatcagcgaggaaggtatcaccaagtctaccctcactaaggccgaacgccagctcaaggacaaatttgatggacgacccaccaagccacctccgaacagctactccctgtactgtgcagagctcatggcaaacatgaaggatgtgcccagcacagagcgcatggtgctatgcagccagcagtggaagctgctctcccagaaggaaaaggatgcttatcacaagaagtgtgaccagaaaaagaaagattatgaagtggagctgctgcgtttccttgagagcttgccagaggaagagcagcagcgggtcctgggagaggagaagatgttgaacatcaataagaagcagaccaccagtccagcctccaagaagccttcacaggaaggtggcaaaggtggttcggagaagcccaagcggcctgtgtctgccatgttcatcttctcagaggagaagagaaggcagcttcaggaggaacgacctgagctctcagaaagtgagctgacccgcctgctggcccgcatgtggaacgacttgtccgagaagaagaaggctaaatataaggcccgggaggccgcgctgaaggcacagtccgagaggaagcctggcggagagcgtgaagataggggcaagttgccagagtcgcccaagagagctgaggagatctggcagcagagtgtcatcggagactatctggcccgctttaagaatgaccgggtgaaagccttgaaagccatggagatgacgtggaacaacatggagaagaaggagaagctcatgtggattaagaaggctgcagaagaccaaaaacgatatgagagagagttaagtgagatgcgggcccctccagctgctacgaactcttccaagaagatgaagttccagggagagcccaagaaaccgcctatgaacggttaccagaagttctcccaggagctgctgtccaatggggaactaaatcacctgccactcaaggagcgcatggtggagatcggcagccgttggcagcgcatctcccagagccagaaggagcactataagaagctggctgaggaacagcagaggcagtacaaggtgcacttggacctctgggtcaagagcctatctccccaggaccgcgcagcatacaaagaatacatctccaataaacgtaagaacatgactaagcttcgaggcccaaaccccaagtctagccggaccaccctgcagtccaagtcggagtcagaggaagacgacgatgaggaagatgatgatgatgatgacgaggaggaagaagaagatgatgagaacggcgactcgtccgaggacggtggggattcttctgagtcgagcagtgaagatgaaagcgaggacggggatgagaatgaggatgatgacgacgacgaggatgacgacgaggatgacgatgaggatgaagacaacgagtctgagggcagtagctccagctcctcatcttcaggggactcctcagactctgactccaactgagg

SEQ ID NO: 127 - DNA, 320 bp, Rattus sp.
cagctttgaaccctgaaactgcttgtggcaggttcagttactgacagcacaaattccattgagtctattcaaggattgaatgacttcacaagccctggtctcaggagattaactttcatattggggtagtggttcacttgagaatacaaaatcttcagattaactgatgtccaaaatactgtcttaaatcatgagatccatcaatttttagtattgtctagacctgcaaatagaattacattgtaaaatctttttcagcatgtgttaagatgtatgtgaaatttttttgtttaagtgtaaacttcataccatactgtcag

SEQ ID NO: 128 - DNA, 2190 bp, Rattus sp.
ctgttagccagaactaagtcactgcccacacagcaattacaccatgaatctctaacatcacaaccttctttcaaatactgtaaaaaaaaaaatactggaaaacctttttagatttgtttcaatctaccacgcttctgaaatgttcagctattaaggggaatgtcaatatatatatacagcccaaattagcgaagcccaaattagggaagcacatagcatggcatacttgagaggttggcaaacaagagggaagcaggtactttctgcaaaaacagggactgtgcgccacaatgagaccgctgtgaccttacttcaaactgtgaaactgtgtgcagcagtaggatttggagtcttaaattcagaaggagcagaaagaaagagagttagacgtttgaattcctggtgcttatgctgacgttttctctcatttgccagcattaaaatctcaagtaaggtgtctcctgtgccaggacgtgtcaggtagacctttctcagtgagctggaatcctgccttctgtgtcatagagttgtaagagcagcagtttctacccatcatgctgtgtactcactgctgcagagcaagtgggaggggccagagatgggccgtccccaggggcggatagcgcggtacacttaattctcttctgcctgaaagcacacttccccgtaagcgaatacagacagaactttgattgagctggtatctgaccgataggattgcttctttgatgtacctctttgacctagctagcatttcagctttgatactgcagaacgatcttttcaaattataatcattctgatagagatatcttaatagacgtgcttttaaaaacaaaacggaaactactgtcagtatgaatactgagccagactggcattcatagatttaacaacttgtatttctaagattcttaactctataaaagtaatatggcttttagatatataggataataatttcagtgagaccgttatctctttactcaacattatgttagggacagtataagccaagcacttacctgttacacattggagactaaaacgactgcccccaaccttagtaagtatgaaaactagactcacattatttcatttttaactgctaaaagtatgtctatagaatttaaaatttaagcactactatttgtcctggccacatttttcaaaaattaagttaaaagttattaattatatacaggtgtgtatttctaataattaaaatacctttcaaatccatggaatgtctgccttttaaatgtaatttgggcttttttgtttgattctttttcacttaactagctgtttatttgtaaacatttctttccacgatttaaaaacacttccaatcggctttacttcctgatttagcacttcctattttgacccttgggcatctgttcttcgtttaattggtgtagaataatgataaaaatcccaagctagtctaaccaaactgcattcttaagagtactaagtgggaatgatgcaaagttcatcaagaattgaaaagaagccggtctttgcactaaggaagacagcataattaagtcttctcgttgccatctgagtaatatcatgatatggggaccgcagaatccatctccggtgggatgatggtatagctttcctagttccagaaaaaatctccttcacattttatataataatttaatcacaatgtccatcgaattcttccttaggcattctttgtaacagtagtgtgctgctctttaaaatattaacctctgacacatgtgtgaacctcattccctgtggtcactaaaatttcttccccactgagcatcaatctttagcagattttaggaaaatactgaattcttagtcaggaaatattttaggaataatcttgctaacagtatattaagtaataaaattacccactctatgtgtgtttcattttttttaatttacatagacaatggtttttataaagcaatgatttcaatttttcccagctacccaaagtcctggttaatttgtgaacacaaattttgttagccttcaagtttaaacggtgctgaattgtcttaacttgaatgtaaaaatggattaaggcttatctcatgggacacaaacatgtcccatatgactgcttgctgcgtactaggggacatgccatttggtgttaaattgtctataataaagttcggtttctcccaaaaa

SEQ ID NO: 129 - DNA, 1592 bp, Rattus norvegicus
ccaacaccatgcgcgagatcgtgcacatccaggcgggccaatgcggcaaccagatcggccctaagttttgggaggtgataagcgatgagcatggcatcgacccgacgggcagctaccatggcgacagtgacttgcagctggagagaatcaatgtgtactacaatgaagctgctggcaacaaatatgtacctcgggccatcctagtggacctggagccaggcaccatggactcagtgaggtcgggaccattcggccagatcttcaggccagacaactttgtgttcggtcagagtggtgcaggaaataactgggcaaagggccactacacagagggtgccgagctggtggactctgtcctggatgtggtcaggaaggagtcagaaagctgtgactgtctccagggctttcagctgacccactcattggggggaggcactggctcaggcatggggaccctgctcatcagcaagatcagagaagagtacccagaccgcatcatgaacaccttcagcgtcatgccctcacccaaggtgtcggacactgtggtggagccctataatgccaccctttccgtgcaccagctggtagagaacacagacgaaacctactgcatcgacaacgaggctctgtatgacatctgcttccgcaccctgaagctgaccacacccacctatggcgatctcaaccacctggtgtcagccaccatgagtggagtgaccacctgcctgcgcttccctggccagctgaacgcagacctgcgcaagctggctgtgaacatggtgcctttcccacgcctgcacttcttcatgccaggcttcgcacctctgaccagcaggggcagccagcagtaccgagccctgacagtgcccgagctcacccagcagatgttcgactccaagaacatgatggctgcctgcgacccacgccatggccgctacctgaccgtagccgccattttccggggccgcatgtccatgaaggaggtggatgagcagatgctcaacgtgcagaacaagaacagcagctacttcgtggaatggatccccaacaatgtgaagacggccgtgtgtgacatccctcctcgtggcctcaagatgtccgccaccttcattggcaacagcaccgccatccaagagctgttcaagcgcatctcggagcagttcactgccatgttccggcgcaaggccttcctgcactggtacacgggcgagggcatggacgagatggagttcaccgaggcggagagcaacatgaatgagctggtgtctgagtaccagcagtaccaggatgccacggctgatgagcagggcgagttcgaggaggaggagggtgaggatgaggcttgagttcccaggccaagcaggttagggaaagctgaggcgaaaggagggggtgggggtcttaatctgtgaaaataccttggcagttggaagaaggagaatggtcttaggtttgtgctgggtctctggtgctcttactgttgcctctcacttttttctctttttgtaatatcgatgacgtgatgtgatgcttgagatctttctgaactcctgttgtgatggctgaaatcgcctgaacctttgtgtcctaaa

SEQ ID NO: 130 - DNA, 3043 bp, Rattus sp.
ggaagacagaagcgtgggagtggagaaagtggcacaaaccagagccctggagatactctggaaacgcggagcagattgtgttggaaagctggcaacagcggtggtctcgggcgcggcgggagggcaggctgggtcgggggcgggcagtgtgtcgtcaggagctgggcggccccgcggcggccgcgtcttcctagcccatcccctcaagcccagctcgggctccggcgccccagctccgaccgtgcgctctcctgggtcaagttagagcccaccgagagccccgcgccactcgcgcactctcgcacccgggcagaaggactgcggaaggggccgccccacgggctgggtgagggctgcagcgtccagagaaggaagcgttgacagctggagccggctgctggaagggtttgcgccgggacgcgggggttgccgtagcgctacgcaagacggggcttaccctctgaaaagcaaacataagaggctgtacaaccaggttatctctgcgatcagcctcaagcagagaagaaagatggccaaagacaagggcctcatcagcccagaagactttgcccagctgcaaaagtacatagactactccaccaaaagtgtcagcgatgtgctgaaggtctttgagatgaacaaatattgccaaggagatgagattgggtacctgggatttgaacagttcctgaaaatgtatctggaagtggaggaggttccccatcacctatgctggactctgttttggtccttccatagcagtcaagacttggatgaggagactgagtcaaaagccaatgtgatctgtctcagtgacgtctactgctacttcaccctcctggaaggcggcagtccggaagacaagctagagttcaccttcaagctgtacgacatggacagaaatgggatcctagacagcacagaagtagaaaaaatcatcctgcaaatgatgagagtggctgaatatctggactgggatgtgtctgagctgagaccgatccttcaggagatgatgaaagagatggaccgggatggcagtggctgtgtctccctagctgagtgggtccgggctggggctaccactgtgcccctgcttgtccttctggggatagacatgactatgaaagatgatggacaccatatatggagacccaagagattctccagaccggtctactgcaacctgtgcgagttgagcattggcctcggcaaacaaggcctgagctgtaacctctgtaagtacattgttcatgaccactgtgccatgaaggcccagccttgtgaagtcagcacctatgccaagtctcggaaagacattggtgtccagccacacgtatgggttcgaggaggctgtcattctggacgctgcgaccgctgtcagaaaaagatccggacctaccacagcctaacgggactgcactgcgtgtggtgccacctggagatccacgatgactgtctgcaggctgttggtcccgagtgtgactgtggactgctccgtgatcatatcctgcctccgtgttctatctaccccagggtcctggtatctggacaggagtgcaaacagaagaccacagatgttacgagcctgtgcacccctgaggcttttcggattgaacctgtttctaacacccacccccttctggtcttcatcaatcctaagagcggaggcaagcaggggcagagcgtgctttggaagttccagtacattctgaaccctcggcaggtgtttaacctgaaggatggtccggagccagggctcaggtttttcaaagacgttcctcagttccgggtgttggtgtgtggtggagacggcaccgtaggctggattctagagaccattgacaaagccaactttcccattgtgcctccagtcgctgtgttgcccctgggcactggaaatgacctggctcggtgcctaagatggggaagaggttatgaaggtgagaacttgagaaagattctcaaggatatagagataagtaaggtggtatatctcgatcgatggctcctggaagtgataccccaacaaaacggagaaaagagtgatccagttccctctcaaatcatcaataactacttctccattggtgtggatgcttccattgctcaccggttccatctcatgagagagaaataccctgagaagttcaatagcagaatgaagaacaagctttggtacttggagtttgccacatcggagtccatcttctcaacgtgcaaaaagctggaagagtcagtgaccgtcgagatatgtgggaagctgctggatctgagtgacctgtccctcgaaggcattgcggtattgaatatcccgagcatgcatggtggctccaatctctggggtgacaccaagagacctcacggggatacgtgtgggatcaaccaggcactgggcagtgtggccaaaataatcacagaccccgatattctcaaaacctgtgtgccagacatgagtgacaagcggctggaagtcgtaggaatagagggtgtgattgagatgggtcagatctataccaggctcaagagtgctggacaccggctggccaagtgctccgagatcacgttccagaccacaaaaaccctccctatgcaagttgatggagaaccctggatgcaagcaccctgtacaatcaagatcacccataagaaccagatgcctatgctaatgggtccggctcccagttcctccaatttctttggcttttggagctgaggatggatgccatctgccttgagcccacctccctgttcctggagatttcccactatctagatgctgccacaccttcctgccagcccagaaggatgttccatcaccttcacagtatttattatcctccgccacctcactgctcccacacagacatccttacacaaccagcgatgcgtaaccttgaaaatgcctcatctaataaagtgactttttccatcactgggatatctgttaaaatgagcgacacatctctttttacaccttcacccctctacagacagatttaaaagcagacagaacaaataaatgaacaaagaaagcctaaaaaaa

SEQ ID NO: 131 - DNA, 383 bp, Rattus sp.
ggagttaaaaggtgtttaatgaggggaagaatattgaacatatacttgtcttattccccacccccattcagatgaatcctgagccatggaaagattagccattcaatgagactttgggtaatgttgagtggctcgagaactcttgaggatgaggatgagagacaactttttaggtggtgcaaggatggaggattaagactggaggtcacagcatgtgtagcacaaggcaatgggactcataagcaggatggtaagcagcctcagcatctggtgatcagatcttcaggcttggctatgggcaatattacttctgctttgtcttctgctggtatggtgatggagtggcagttggggggcagggctcatgaacaacaggctggcat

SEQ ID NO: 132 - DNA, 672 bp, Rattus sp.
  misc_feature (1)..(672): where n may be a or g or c or t/u, unknown, or other
caactaccaggattgcatttattataacagactgaaagtgcaagccgagagagaggtctgagaagcagcgtaaacacagggagcactcctgaggaagctcagatgcactccttcccagggactgcccctgaagaacacccagccctcggaattaaggcagttaggatgtgggcactgttgtgccaatattggtggtgttttcacccagcccctaccagctgtccctttctatccctcctaactggacattattgagctttctccacgagtaatgctttcccacacaaggccacccagctcaagaactttccttccagggacggttccagccaagtacctcaatgttaactattaactacagcaacaaccacccagagactgtggtttgggggtactgcccacccccaggagctgccaaatgtccaggctactgtgttctaaccaaatagaaacagagctctacacttcagttccacaaccacttctggccctcactgagccctgccaagtccttactctgccctacatgtattcccttttcacacgaggcctccaccctgcagacttacagaaggccgggatatggtttgtgctccttcnctgcgggccttacataaagtgctcagaatcagagattcttgcactgaattgcagactccctcatgccgaatt

SEQ ID NO: 133 - DNA, 367 bp, Rattus sp.
  misc_feature (1)..(367): where n may be a or g or c or t/u, unknown, or other
tttgaactctgaattagacagtttgtattctaaaacacacacaaatctgtaccaattaaatacaaagtcgaaagggaatataaattacagttttngttttcaacggatgatagtaaaacactaccgtcagctcaccacacacacaaacagcagcttttcaatagagtctcccaggcctccctcaccaatgctaaggacttaaaaaataaaaagtacagcaggctgcacatgctctctcacttcctcacaatacacggatagctgtacactgaggtccattgtacaggcatctacagagtaagtacataaaatatatttgtaaaattcttccacgcacgatcctacaatgtctctcctcgcaggatac

SEQ ID NO: 134 - DNA, 2225 bp, Rattus norvegicus
tttcagggatttttgcgattcctctctgtagacttctacttgttctctaagggagttcttcatgtctttcttgaagtcatccagcatcatgatcaaatatgattttgaaactagatcttgcttttctggtgtgtttggatattccatgtttgttttggtgggagaattgggctccgatgatggcatgtagtcttggtttctgttgcttggtttcctgcgcttgcctctcgccatcagattatctctagtgttactttgttctgctatttctgacagtggctagactgtcctataagcctgtgtgtcaggagtgctgtagaccttttttcctctctttcagtcagttatgggacagagtgttctgcttttgggcgtgtagtttttcctctctacaggtcttcagctgttcctgtgggcctgtgtcttgagttcaccaggcagctttcttgcagcagaaaatttggtcatacctgtgatcctgaggctcaagttcgctcgtggggtgctgtccaggggctctctgcagcgggcacaaccaggaagacctgtgcggccccttccggagcttcagtgcaccagggttccagatggcctttggcgttttcctctggcgtccgagatgtatgtacagagagcagtctcttctggtttcccaggcttgtctgcctctctgaaggttcagctctccctcccacgggatttgggtgcagagaactgtttatccggtctgtttctttcaggttccggtggtgtctcaggcaggtgtcgttcctgcgccctcccccatgggaccagaggccttatacagtttcctcttgggccagggatgtgggcaggggtgagcagtgttggtggtctcttccgtctgcagcctcaggagtgccacctgaccaggcggttgggtctctctctgagaatttcatttttaaatcattcattaaaatgtcatgacttgatgtcctgctgtccgtctcacgccctcagctgtaacagtgccgagggagtcactgaagaagagactgaatgaccagagtatgggcagcacagacaactcaacaaaaatgtcttcagaggtggagactgcggaggccgtagatgagtcagagaagaactctatggcatcagagaaggaaaaccattccaaaatagcagacttttctgatcttctgaaggaagggacaaaggaagcagatgaccgggcagaaaatacccagtttgtcaaagacttcttgaaaggaaacattaagaaggagctatttaagctggccaccactgcactttcatactcagcccctgaggaggaaatggattcactgaccaaggacatggagtacttctttggtgaaaactgggaggaaaaagtgaagtgctctgaagctgcccagacgtatgtggatcagattcactatgtagggcaaaatgagccagagcatctggtggcccatacttactctacttacatggggggaaacctttcaggggaccaggtactgaagaaggagacccagccggtccccttcactagggaagggactcagttctacctgtttgagcatgtagacaatgctaagcaattcaagctattctactgcgctagattgaatgccttggacctgaatttgaagaccaaagagaggattgtggaggaagccaccaaagcctttgaatataatatgcagatattcagtgaactggaccaggcaggctccataccagtaagagaaaccctaaagaatgggctctcaatacttgatgggaagggaggtgtatgcaaatgtccctttaatgctgctcagccagacaaaggtaccctgggaggcagcaactgccctttccagatgtccatggccttgctgaggaagcctaacttgcagctcattctagttgccagtatggccttggtagctggacttttagcctggtactacatgtgaagggcctgtcaagttgtttgcatcctatctcaacatcctaccacttgttccttccccacctccacctctgcctagaactaccacctcaggtgacatttttaatgttgggtttgagaaaatgagcaaccaataaaagacagaccctagaaaaaagtcatgacttaagtggcacggggacacctaaagtcacactttgtgcttcagacatactttctttctctatttcaacactgaattcgggaagtaacctactactattaataataaatgctacacaatgcataataaaaa

SEQ ID NO: 135 - DNA, 467 bp, Rattus sp.
caaggtcacacgtgatttaatgtaggtggcaatgaaactgggtttggtgagctacatcgttaaaacggaaggcagccttcctagaatctcaatgcattggcttaggagtaagcaatactgaaaaaagttaaagcatctgttggcctctttcccatcacagggtacaacaacctcttgtagtcagcttcctctacagtaggctctaaagagttcagcatgaaccaagatgggaataatttggtgaccaaactgcattttccaaagtccttttgtggcagaatctagtaacttattgagcttcaggatggatctagtttccttttcttcaggtcctggctcttcctatgagattcagagttttctctgctacctaggacatgctgattagtgctgcaggtctgcaggtgcccaggtccaggtttttcttgtactttctggtggcaatggatatgtgggacactgtcctg

SEQ ID NO: 136 - DNA, 822 bp, Rattus norvegicus
ggctgtggaggcaatggcctggtgctgtcggctacggacagcagggcttgccggaaggtagccgtgaagaagattgtgctgagtgacgcacgaagcatgaagcacgcgctccgagagatcaaaatcatccggcgcctggaccacgacaacatcgtcaaagtgtacgaggtactgggacccaagggtagcgatctgcagggcgagctctttaagttcagcgtggcttatattgtccaagagtacatggagactgacctggcatgcctgctagagcagggcacgctgaccgaggagcacgctaagctattcatgtaccagctgctgcgtgggctcaagtacatccactctgccaacgtgttgcacagggacctgaagcccgccaacattttcatcagcacggaggacctcgtgctgaagatcggggatttcgggctggccagaatcgcggaccagcattactcccacaagggttatctgtcggaagggttggtgacaaagtggtaccgctctccacgactgctcctgtccccgaacaactacacgaaagccatcgatatgtgggcagctggctgcatcctagcggagatgctcacggggaaaatgctctttgctggggctcacgagcttgagcagatgcagctcatcctagacaccatccctgtagtgcgggaggaagacaaggaggagctgctcagggtgatgccgtcctttgtcagcagcacttgggaggtgaagaggccactgcgtaagctactcccggatgtcaaccgtgaagccattgactttctggagaagatcctgacgttcagccccatggaccgg

SEQ ID NO: 137 - DNA, 512 bp, Rattus norvegicus
ttgtcttaacacttttttattgattcatttgtgtttacccatgtttcccctcatttaaaaaaaaaaatcagtgtttctaatttcttggattttcaactttaagttccacgttgacaaatctatttttatgtacacctttgattcttacattacaacagaagtcacatgtttaaaaatacccactccttaaaacatagtcattgtcaatattttgtttacagtacagatcctgaagacaaacacttgactatattaagatttattagtctgaaaacagaatgtaggatattttgtatataccgtcggtatatactgaattctggcagcaaagtgtttatgagaaaatttcaattttccctggtagtataatataaaggccaatctacttctcacttgttacaactatatttaactgaattatggtttaaaatcacaaagtgataaaatataaatacaaaatactaagaaaagtttaacatataaagagaaagtgtcttatgtattgtaatttt

SEQ ID NO: 138 - DNA, 451 bp, Rattus norvegicus
  misc_feature (1)..(451): where n may be a or g or c or t/u, unknown, or other
tttttttttttttttttatattaaatgactattttatttacacaccctatcatatccataaataattgcgccctctttgtctgcacaatggttccttgacatctaacgattacagagtgaacatctaaaagtagatgggggcttactccggcttaggacatggtctcttgtgtacactcaatatgcacacccagtaacacagacatagaccagagagggtggctcccagggcctcctgggctcccagcaccaatgggaaaaccttgcccatacacaggtgagttcctgacaactggagaacagagagatgcaggccagggctccgccccacccaccgcagggccccgcccacctctggagcagctccacctttgttcaccctggaaggcatctctttggattgcaaatactttaattcacagaggggatggggtangggctggggtggtgt

SEQ ID NO: 139 - DNA, 3208 bp, Rattus sp.
  misc_feature (1)..(3208): where n may be a or g or c or t/u, unknown, or other
gaattccgcctaagaatagcaaagtagtaaagagagatttggatgatgatgtcattgaatctgtcaaagacctcctgtccaatgaagactcagtggaagatgtttctaagaagagcgaactaattgttgatgttcaagaagagaaagatacagatgctgaagatggatctgaagttgatgatgaaaggccagcttggaacagtaagctgcaatacatcctggcgcaagttggattttctgtaggtctggggaatgtgtggcggttcccatacctctgccaaaagaatggcggtggtgcatatcttttgccatatttaatactacttctggtgataggaattccactcttcttcttggaactttctgtgggtcaaagaattcggagaggcagcatcggggtttggaattacataagccctaaactgggtgggattggattcgcaagctgtgtagtgtgctattttgtggctctctactacaatgtcatcattggctggacattgttttacttttctcagtcttttcaacaacctctcccttgggatcaatgccccctggtgaaaaacgcatctcatacttatattgagccagaatgtgaaaaaagttctgccaccacttattactggtaccgggaagcactggctatcagcagctccatctctgaaagtgggggcttaaactggaagatgactggctgcctgctggctgcctgggtcatggtttgtctggccatgatcaaaggcattcagtcctctggaaaaatcatgtattttagttctctgttcccctacgtggtacttatatgcttcctaataagatctctccttttaaatggttcaattgatggcatccgacacatgttcacccctaagcttgaaatgatgctggagcccaaggtttggagagaggcagcgactcaagtcttctttgccttgggtttgggatttggtggagtcatcgcgttttccagttacaacaagagagacaacaactgccacttcgacgctgtcctcgtgtcttttatcaactttttcacttcagtcctggcaacactggtggtgttcgcagttctggggttcaaagccaatatcgtaaatgaaaagtgcatttcacaaaattctgagatgatcttgaaacttttgaaaacgggaaacgttagttgggatgtcattccccgtcacatcaacctttcagctgtcactgcggaagattatcatgtagtttacgacatcattcaaaaagtgaaggaggaggagtttgctgttctccatctcaaagcctgtcaaattgaggatgagctaaataaagctgtgcagggcactggcttggctttcattgcctttacagaggccatgacacattttcctgcatctcccttctggtcagtgatgtttttcctcatgctgataaatctcgggctcggcagcatgtttggaaccattgaaggaatcatcactcctgttgtggacacattcaaagtgaggaaggaaatactcaccgttatctgttgcctcctagcattttgtatcggcttgatgtttgtgcagcgctctggaaattacttcgttacaatgtttgatgattattctgccacattgcctctgttaatcgtggtcatcttggagaatattgctgtaagcttcgtttacggcatagataagtttctagaagacctaacagacatgttaggatttgctccaagcaaatattactattacatgtggaaatacatttctcctctaatgctagtaacattgttaatagctagcattgtgaatatgggattaagtcctccgggatataatgcatggatcaaggagaaggcatccgaagaatttctgagctacccgatgtgggggatggtggtctgtttctctctgatggtgctggctatacttcctgtcccagtcgttttcgtcattcgtcgctgcaacctcatagatgatagttctggtaacttggcctctgtgacctataagagaggaagagtcctgaaagaacccgtgaacttagacggagatgatgcaagcctcattcacggaaagataccaagtgaaatgtcgtctccaaattttggtaaaaatatctatcgaaaacagagtggttctccgaccctggacactgctcccaatggacgctatgggattgggtatttgatggcagatatgccagatatgcccgagtctgacttgtagctgagtgaaagcaaagggttgagcttggttcatttttatcaatgagcattggttctactatgagaagcagtgagcttcacttgtcacaaggtgatctcaggtgtccatagccgccatctttaatctcaacagtttaagacagttccagaagagcaatcctcagtttacaattacaaagtaacaattgcagacaaagcttacattgactggggtcctttgccaggatttttttaaaaagcactttgacatactttcaagtatttctatctcttaaaaaaaaggtgttacctcagtttctaatagtttccggatttaatattattggcgatttgaaaaaaaatccctgttatatcttacaattcataattttgccttcggagtaagttccagtattaccatgaacagttgtgtgtgagcgggtgcttctcagcacattgccatgagtacgttctgtagatagcctgtacttatctttggtagcattgaaaccttaggcacttagttggagaaaacttcaaagtattttcttatatgatagccgtctagagcaatagtattaaagagtaaaaaggcactgatggtggatgagaggttaagtccagatgtgatcgagggtttcctggagtggccatatattttgtgtaaaataggtgtgtgcaaatgattgaagggactctcgagaattatgcagactgcatttttcttatgccgtgtgcctaataaacctacttaatatttattgtggttttgagatcacttatagtatatttatataatatacttgcaatgtatagagatgcgcatcaggactcttaagtgctggtttgaaaacttgaagcaagatagcatctgatttcatatgtttctgttttgcttcattttatgcaaatacgaattcttttttttaagtgattgttaaaactgtatggcattacattttaacctacaaataaacgaagtttancaaaaaaaaaaaaaaaaaaaaaaaaaaaaaaaaaaaaaaaaaggaattc

SEQ ID NO: 140 - DNA, 436 bp, Rattus norvegicus
tttttttttttttttttacaaccattcatagaaattttattggacaaagcaaaatgctcttcaataagcaaacaaccataaaaacatggtgcgtccgcaccactcagcagggaagtcatgcaatttctggaacagagcacaaggtattacactgagtgagagcatctccaatgcttctgtcaacacgcttccatctcagcgctgcagagctggaggagagttggtatggtagcgaggtatgaagagttaggtggtagtgaggcagctgggtgactataagacagaatcaacgggccctctggagtactgagctcgttctatacattggtcatacataattgcatgaatcggcacacatgataaaatagcacgaagccacatacatactgttccaattaacaagtccttgcttttgatatgttacaccatatgttct

SEQ ID NO: 141 - DNA, 636 bp, Rattus sp.
  misc_feature (1)..(636): where n may be a or g or c or t/u, unknown, or other
agcattttcaaactttatttacaactgtcacagtgacaaaaagtagtttggaaaaaaaaaatgctagtttctccctgagcctcgatacagaacagacagaagtcacaggaggttcatctcacaacaggcatgtcactgaaatactaggattttttttttcaatacgatcagttagaaatacacacaaattacttaaagaaagaagaggaggaagaggggaagagaaagagaaagagaaagagaaagagaagaagaggccagacaggagctcagccacttgtccaagagcagctgggtccccccaacaggctcaaccgctgaggggcctgacgttagctatcagcccctgacctgctcagacaacacacggttgtacaacatggtctagtgaccggcaaaaggaagaagccaccccacagacacacatatacacaaagctgattgatactggatttacaagcacatcccgtcacacggcaagaccaagacagatcggggtaggggtgagaagacaccaanacacaggaatttcaaaggccaaacacctgtccataaagggaggcgagggacgagaaatgccgtgggagaaggggagaaggatgaacagatctctgtagccaaaacagaacagagtgg

SEQ ID NO: 142 - DNA, 437 bp, Rattus sp.
acaaaaataaatgtagtctttattaccaagtaataaaatagaagccataattaactatagcgtagggcagcaggatgaggtgattaaatgaataatgttacattgtcttggggggaaactaggttttcagaattacagtgtctggaattttagtgcttaaaaaaaaaaaacatattttaggaggaataaggaactggtagaacaaggaaatggcttaactaatctgagttaagagcacctctgagggccacatggtctgtaatcacagctccagaggctgaaaaacaggagatctacagtttaaggtcagccatggctaggctgaatctgttgtctgtttaaagagcaccaagaaattccctggtcaaacgagagcttctggtgaaagactaaaggaatggcagtaaggacatagaagtggctccttgcctcgtgcc

SEQ ID NO: 143 - DNA, 592 bp, Rattus sp.
cccgaagacaaccaaagcttccagtacgatcatgaggccttcctaggcaaggaggattccaagaccttcgatcagctaagcccggacgagagcaaggagaggctggggaaaattgttgatcgaatcgacagtgatggagacggccttgttactactgaggagctgaaagtttggatcaaacgggtacagaaaagatacatctacgataatgtggctaaagtctggaaggattatgatagggacaaagacgaaaagatctcctgggaagaatacaagcaggccacctatggctactacctgggaaaccctgctgaattccaagatagctctgatcatcacacctttaaaaagatgctgccacgggatgagaggaggtttaaggcttcagacctcgatggcgacctgacagctactcgggaggagttcactgcctttctgcacccagaggagtttgaacatatgaaggagattgtagttctggaaaccctggaggatatcgacaagaacggggatggttttgtggaccaggatgagtacattgcggacatgttttctcacgaggacaatggccctgagccagactgggttttgt

SEQ ID NO: 144 - DNA, 3027 bp, Rattus norvegicus
ggcagtagctggatgagggcgttgcttgcctcctcccttttttttctccgattggttctgaggggtatattcgagttgcaaaatggcggccccgagcgctctcttcagcgttcagtagcagcttcaggctgagcggatgtctcttctcctcagtttcggactcagagacacgcggctccctactcctgctgatcacgaagtccttgaaggcgctcaacgcaccggaatctcccagcggccgcgaccgccgcctcggccctgctctccgcggcgccggaactcagcgtgatctgcggcggccgtctaggaggttcacaaaaatggcgaagagagttgcggagaaggaattaacagataggaattgggatgaagaagatgaagttgaagagatgggaacattctcagtggccagtgaggaagtcatgaagaacagagccgtgaagaaggcaaagcgtaggaatattggctttgaatctgatagcggaggagcctttaaaggtttcaaagggttggttgtgccttctggaggaggagggttttctggatttggtggtggctctggagggaagcctctggaaggactgacaaatggaaacagcacagacagtgccacgcccttctccagtgcaaagacagcagcggagcctaaggcagcctttggttcttttgctgtgaatggccctactactttggtggataaaaagatctccagtcctaaatgcaatagcagcaatcagccgccctcctccggcccagcctccagtacctcctgcactgggaatacctatcataagcagttggctggcttgaactgctctgtccgggattggatagtgaagcatgttaacacaaacccactttgtgacctgacgcccatttttaaagactacgagagatacttagcgacgatcgagaagcagctggagaatgggggtagcagcagctcagagagacagacagacagggcgacggctgcaatggagcctccttccctttttggttcaacaaaactacagcaagattcaccattttcatttcatggcaacaaagcggaggacacatctgaaaagttggagtttacagcagaaaagaaatcggacgcagcacaaggagcaacaagtgcctcgtttaattttggcaagaaaattgagagctcagttttgggctctttaagctctggctccctaactgggttttcattctctcctggaaactctagtttatttggtaaagatgctgcccagagtaaagcagcctcttcaccgttttctgctaaagcatccgagagtcaagcaggaggcagcagcagtgagtgcagagatggtgaagaagaggagagtgatgagccacccaaggtggtggtgactgaagtgaaggaagaggatgctttctactccaaaaaatgtaaactattttacaagaaagacaatgaatttaaagagaagggtgtggggaccctgcatttaaaacccacagcaactcagaagacccagctcttggtgcgggcagacaccaacctaggcaacattctgctgaatgttctgatcccacccaatatgccgtgcacccggacagggaagaacaatgtccttatcgtctgtgtccccaaccccccacttgatgagaagcagcccactctcccggtcaccatgctgattcgggtgaagacgagcgaggatgctgatgagttgcacaagattttactgcagaaaaaggacgtctgagcactgaggctgaccagggcacgtcaccatgttgctgcttccctttgcccctaaacttagtcacattctttcctctttgtactgtgacattctgagaacttctaggtaacttgaaacttttgtgaggaagattaaggccaataaatcctttcagtgtgtcgaagctgttctcccttcctaagaactaagcaaaatacattggagtgaaaagtttgggaagattttttaatgtcgattcattgagtaaactaacctaagtgattcttacggactgtaatcagggtaccagttagctctccaaaggctccctcaggcagccacgggtgccactctcttcctgccctgggagactcaatggcagtgtccacagagttccagaagacgcctgctcccctcctgtgggcttgtttggtccgtgactagcactcctgccaaataccacaccggcacactgtaaccgcgcttgttctgtttccttctgagcggaactgtgggtcctggggatctctgtccttagcctgtttttgacaggtgctggcctttgacctggaactgcttgactgaaccaggcactgcctttccatgggaagagagggcaggtagtggcttgtcggggagctggcgaggtatagactgggttttgtcgtttatccatgaggtgctcttacttgcttacctccctagttaacatggatgggggctgtcaggaataatggattttattaaaagcacaaatttggtagcattttttaaatatcatttctataccacaaacaagcctttattttaaaagaaaaaagtgaaattgtgctgaaaagggttgtatgctcgtggccgtttgtgccccggggacctggtggtcacagtctgaatggagccctgtcagagggtgctcgtgagaaggaagagtgtgggagtggagacggctcattcggcctgtgacttcagacagcagcatgttattcagcgtctgaaaagvccaatttcatttttacctttttacggatgaggtattcttgagtctgcccacttggaagagagccatggttctacacgccattcatgccacttgagagcggtgaggttaccgatactagcattctctcaaggccagacatcagttaaatgcaaggtttgttgacgagctggtcacggctccctcctaaccccagtgccggaaagctgaggcaggaagaccgtgtgagttcacaacagcctgagctacgcagtaggaacccctccccctaaaataaaataaaagtttggtgtctaaaaaaaaaaaaaaaaaaaaaaaaaaaaaaaa

SEQ ID NO: 145 - DNA, 535 bp, Rattus norvegicus
cggccgccttcctggcccagcaggagagcgagattgctggcatcgagaatgactcgggtttcggggcacctgccgccagccaggtggcctctgcgcagcccggactcgcgagcgggggtggttcggaggacatggggactacagtcaatggagatgtgtttcaggaggctaacgggcctgccgatggctacgctgcgattgcccaggcggacaggttgactcaggagcctgagagcatccgcaagtggagagaggagcagaagaaaaggctgcaggagttggatgctgcctcgaaggtgaccgaacaggagtggcgggagaaggccaaaaaagacctggaggagtggaaccagcgccaaagtgaacaggttgagaagaacaagatcaacaacagggcatcggaagaggcttttgtgaaagaatccaaggaggagaccccaggcacagagtgggagaaggtggcccagctgtgtgacttcaaccctaagagcagcaagcaatgtaaagacgtgtcccgcctgcgctcggtgctcat

SEQ ID NO: 146 - DNA, 2046 bp, Rattus norvegicus
cgccgcggcggccgcagaggcggaggccgaggccgaggcgcaggggggcgcgccccgggcccaggcccggccccagctgccgctgcggagcccgccgggaggccccggagcgcggccacagcgcagctgctgccatggcgcagaccctgcagatggagattccaaactttggcaacagcatcctcgagtgcctcaatgagcagcggctacagggactgtattgtgacgtgtcagtggtggtaaagggccatgccttcaaagcccaccgtgctgtgttggccgccagcagctcctacttccgggacctattcaacagcagccgcagtgctgtggtagaactgccagccgctgtgcagccacagtcattccagcagatcctcacgttttgttatacaggccggctgagcatgaacatgggggaccagttcctgctcatctacacagccggcttcctgcagatccaggagatcatggagaaaggcactgagttcttcctcaaagttagctctccaagttgcgactcccagggcctgcacccggaggaggccccatcctcagagcctcagagtcctgtagcgcagatattgggctggccagcctgtagcacgccactgccccttgtgtcacgggtcaagacagaacaggagttggactcggtgcaatgcacacccatggccaagaggctatgggatagcagccagaaggaagctggaggcagtggtggcaacaatggcagccgcaagatggccaagttctccacgccagacctggcccctaaccggatgccccagccagtctctgtggccacagctacagcagcagtggctgtggttgcagtggggggatgtgtgagtgggcccagcatgtcagagcggaccagcccaggtacctccagtgcttacactagtgacagccccagctcctaccacaacgaagaagacgaagaggaagatgcaggtgaggagggcacagatgagcagtaccgtcagatctgcaatatgtataccatgtacagtatgttgaacgttggccagacagttgagaaggtggaggctcttcctgagcaggtcgtccttgagtcccacagtcgcattcgagtgcggcaagacctggcatctctcccagctgagctcatcaaccagatcggcaatcgctgccacccaaagctctacgatgaaggcgacccctcagagaagctggagcttgtgacaggcaccaatgtatacatcacaagggcacaactcatgaactgccacgtcagtgcaggcacgcggcacaaggtcttgctgcggcggctcctggcttccttctttgaccggaacacactggccaatagctgtggcaccggcatccgttcttccaccaatgaccctagacgcaagccactggacagtcgtgtcctccatgctgtcaagtactactgccagaacttcgcccccaacttcaaggagagcgagatgaatgccattgcagccgacatgtgcaccaatgcccgccgagtggtccgtaaaagctggctgcccaagaccaagccgctacacctggtggagggcgataactacagcagcttcatcagcgacactggcaagatagaaccggacatgatgagcatggaacacagcttcgagacagccagccacgatggcgaggctggcccttcagctgaggttctccagtaacatacatgtgacacccccttaccggatgtcacattccccctcctatcacacccccacctaccacctacctggtcacgatctactgtctgtccctccccagaacctgtgagggggtgttcagtgccctctatcggatgcaagagctggctgaccaaggccaaagcactgtacctagcagagggcagtgccgacagcagctttctcaatgaccatgaggaagagctgaacttggtaggcatagaatacagcttccacacagacagccccctcagcagaggtcctccagtacctgcccaggaccctcccatggatgtcacactcccctcctgtcacacacatacccccaccttagtcacgagctactgtg

SEQ ID NO: 147 - DNA, 312 bp, Rattus norvegicus
gtctcacgtcctctctgcactctggaccctgacttcaccgacatgaaaactcattactttctcctggtgatgttattttttctcttctcccagatggagctgggtgctggcattctcacaagtcttggacgcagaacagatcaataccgatgcctccaaaatggaggattctgtctccgctccagctgcccatctcataccaaactacaaggaacatgtaaaccagataagcccaactgttgcaggagttgacagtggtttgaagaatggacataaaggacaagcaagggattgtaaaattagtgttttaat

SEQ ID NO: 148 - DNA, 3822 bp, Rattus rattus
cttcactgggggcccttaggcgagaggagtttccaaattgggtaaaaggcagagtggaggaggggaggtgataattagcaaagttgtagacctctgaaccttctgggtctgaagcccctccctgtgagcgtgggggagactcactctccggtgggggggccgtttgggtcccccccacccctactccctcgctcctttacaccccgggctctctcctggcctcctacccctgcaccctgcatccatcatgacggtgatgtcaggggagaatgcagacgaggcttcggccgctccaggtcacccccaggatggcagctacccaaggcaggcggaccacgacgaccacgaatgctgcgagcgcgtggtgatcaacatctccgggctgcgcttcgagacgcagctcaagactctggcccagttccccaacacgctgctgggcaacccgaagaaacgcatgcgctactttgaccctctgaggaatgagtacttctttgaccgcaaccggcccagcttcgatgccatcctttattactaccagtcgggggggcgcctgcgcaggccggtcaacgtgcccctggacatgttctccgaggagattaaattttacgagttgggcgaggaggccatggagaagttccgggaagatgagggcttcatcaaggaagaggagcgccccctacccgagaaggagtaccagcgccaggtgtggctgctctttgagtatccggagagctcaggacctgcacgggttattgccattgtatccgtcatggtcatcctcatctccatagtcatcttttgcctggagactctccctgagctgaaggatgacaaggacttcacgggcaccattcaccgcatcgataacaccacagtcatctacacttctaacatcttcacagaccctttcttcattgtggaaaccttgtgtatcatctggttctcttttgagctggtggtgcgcttcttcgcctgccccagcaagacagacttctttaagaacatcatgaacttcatcgacattgtggccatcatcccttatttcattaccctgggcacagagatagctgagcaggaggggaatcagaagggcgagcaggccacttccctggccatcctcagggtcatccgcttggtaagggtgttcagaatcttcaaactctcccgccactccaagggccttcagatcctgggccagaccctcaaagctagtatgagggagttagggctgctcatctttttcctcttcattggcgtcatactgttttctagtgcagtgtactttgcggaggcggaagaagctgagtcgcacttctccagtatccccgatgctttctggtgggcggtggtgtccatgaccactgtgggatacggtgacatgtaccctgtgacaattggaggcaagatcgtgggctccttgtgtgccatcgctggtgtgctgacaattgccctgcccgtacctgtcattgtgtccaatttcaactatttctaccaccgagaaactgagggggaagagcaggctcagttgctccatgttagttctcctaacttagcctctgacagtgacctcagccgccgcagctcctctactatcagcaagtctgagtacatggagatcgaagaggacatgaacaatagcatagcccactacaggcaggctaatatcagaactggtaactgcaccgcaactgatcaaaactgcgttaataagagcaagctcctgaccgatgtttaaaaaaagcaccaggcaagcaatcaaaagcccccaaacaaaacccttggcgactcctgtcccactctgtagatactttactaaaaccgtagtctttgaatgctttatttaactggcaatgcactgttgcattgtgaatttggggggtgggcaaacctgaagctttcaagatcacatttaaaaaacaaaaccaaccaaacaagcaaaaagaaaaaaaaaacccaacaaaaaatataaaaactcaacccaacccaataaccaactattttcatttttatttaaaaaatgagaaaagaaagaggattttctaaaacgctgcccatgaagtagtctgtgtgaaataagactcatgctttccttgtactgaagtttttccaatcttttggcttaagttgttttgtttgttttttttaaacctaaaaatcagatgaccacttaggaacataaaaattcaaatttgcatggaactccactgtaaaatttttgcaaattgcacagcacatgtcagatagtgtgccccgtggaacaccatgtaacagcctcagtggtcagtggggggaaaaatgcttttattttgatcaactgaattgcatacaaggctaacaaaatccggactcattaagaatggttcagaaagcaccttgcaaatctgttactggtcccaatctgttgggattttccatctgccccgttctcctaaatcccagtctattctctaagaaaagggcaacttgattaaatgagttgtttcatctgtaaggctgctaagttctctcaactgcagatgatccaaatataggtttgttttttttttaaaccaatcctgacccctgaccttcagaagtggatgataaaccttaccctccttattgcaagagcacaagagttcaatggtaagcatgtttgaatccgataacatttattttataatcgcatgctgagaaagttagcccagacaatagtgaataagcttacgttgaaatcgactcttctaaatatagtccgtttcatttgcattcaccaaaagtgcactccttcatttattaactcttttcttagcagctaaagtactgtatttaagtacgtaccttagatggggacagtcccttttccgagctcaaagcatgttctcttagtcagcattatggcctatttgattaagatataccttgaattaattaatgcatggtttcagtaataaaaaaaattagaaaatactaaaaattacaagcctgtgggacgaaaggccaaaggacacgggggtggggggtggggtgggggaactccgtcattttcctgcctttgctcagggaaatgtcaagcttctatgcaggtatagacagagagaggaccaatatgcccatcctttaaggggaaactgtggaaaactaaataaatcattcaaggtatttaatagacctaaaaccaagcattctttctagctgaacataaatacaagcaaaacaaacaaacaaacaaacaaaaaaaaggtgcaatattgcatggtttcttggtgcattcttaggatgtaagtgataacgctgacctcttcatgcatccagagcagagccgatttcttttcgcagtcatgatttgaagtctatagagacttcggccctcccccctgaggctccctgaagaaactcagccaattgatttaatacttgcttagtgcctttatctgtacccacagtgaactgcagaaaagtgcctccataactcagctgggaagttatttaacagaagggaggaagggttggggcacagacctttttgctttttgttttgtttgtttttccatcctcactgtctcacttcaccactgtgagaagacctctccaccctcagagcccccaaagaagagagagagagagagagaaagcaggtgctgtctctcttggctgtctactggacttggtctctttggcagcctgactctggatatgaactgagacccatctttgaagtggacatgaaccataaactggttctattctgttttgttctgttctgttttgtttcttctcgaccagaagccaagagaaatgtttttgggaatgtggaaggccactccggacatacaaagcttc

SEQ ID NO: 149 - DNA, 543 bp, Rattus norvegicus
ttctgtatgaaataatttattgtagcattgtcaagattggcattattttacagtatttttttttctcttcaagaactacgagtctaaagaaataaaggaaaactactttaattagaactatctaaataaaatcttctgttttggtttatatcagatagatttacagacatattgtcactgagaaatagagtgattccattatataaaatatggcaaaaagggtccccccaaatactgttcaacaacactatggtttaatagtttaattatagtttaaattctcatctgagaaacctaaaaatgtactgaatggcttgtgtggggacagtgctgtgttttaatttgtactttgcccaatcatcctcccttgaagaaaactcagggtaagctacttgctaaactctctaagtaactcaatcaagaaaacacaattgctattcaataaaaaaaaaatccaatttaagaaaagaaaaagaaaacaattcctctacaatagtctgtaagaccagaatagatacacaacaaatttaatggttaaattta

SEQ ID NO: 150 - DNA, 410 bp, Rattus sp.
agaacaacaaatcaaaatgtaaacttaaaatataaccaaaagagggacagctctttaggaaaaggaaaaaaccttaaatagtgaataaacaactacaaccacttaaccattgtaggcttaaaagcagccatcaataaagaaagcgttcaagctcaacatacatacttacacacactaattccacaaacctcaataaattcctatattacaaattgggctaatctatagacccatagatgaaatactgttaatatgagtaacaagaaccaattctcctagcacaagtgtatgacaacccggataaccattgtcaattatcgaatcataggtactaacccaacaataaaattacctatccctaactcgttagcccaacacaggcgtgctttaaggaaagattaacaaaaaaa

SEQ ID NO: 151 - DNA, 1214 bp, Rattus norvegicus
gaattccgggccccgcgctgccgctgctcctgccgtcgctgctcttgctgctgctgttgggcgcgggcggttgcggtcctggggtgcgcgccgaggtgctgttccgctgcccaccctgcacgcccgagcgtctggccgcctgcggacccccacccgacgcgccctgcgccgagctggtgcgagagcccggctgcggttgctgctccgtgtgcgcacgacaggagggcgaagcttgcggcgtctacatcccgcgctgcgcccagacgttacgctgttaccccaacccgggctccgagctgcccctgaaggcactggtcaccggcgcgggtacctgtgaaaagagacgcgtgggcgccaccccacagcaggttgcagacagtgaggatgaccactcggagggaggcctggtggagaaccatgtggacggaaccatgaacatgttgggaggcagcagtgctggccggaagccccctaagtcaggcatgaaggaactggctgtgttccgggagaaggtcaacgagcagcaccggcagatgggcaaaggtgccaaacacctcagcctggaggagcccaagaagctgcgcccacctcctgccaggaccccttgccagcaggagctggaccaggtcctggagcgcatctccaccatgcgccttccggatgatcggggtcctctggaacatctctactccctgcatatccccaactgtgacaagcatggcctgtacaacctcaaacagtgcaagatgtctctgaatggacagcgtggggagtgctggtgtgtgaaccccaatactgggaagccaatccagggagctcccaccatccggggagaccccgagtgccatctcttctacaacgagcagcaggagaatgatggggctcacgcccaaagggtgcagtaaaccacagccagtcggtgcctggcttccccaccccaaacaccagcagaaatggagggtgtcagggtgatgggtgtggaggatttcccagttttgacacatgtatttatatttggaaagagaccaacactgagctcagaagcccccctgcgccccccagtggcagttaacctgtacctccgttcctgcttctaatagagagggtggtggcactggggatactgggtacaggcttgggaatggggaaagaaatttttatttttgaacccctgtgtctcttttacttaagattaaaggaaggaaacggaattc

SEQ ID NO: 152 - DNA, 3201 bp, Rattus norvegicus
ccgggtaagaaaataagctgccctatttttctttcttcttctcttacaactggaaccagccatttccccaaactaccaccatggaggtggcaatggtgagtgccgagagctcagggtgcaacagccacatgccttatggttatgctgcccaggccagggctcgagagagggagagacttgctcactccagggcagctgcagctctggctgttgcagctgccacggctgcggtggaaggcactggaggttctggtggaggcccccaccatcatcatcagacacgtggggcctactcctcccatgatcctcaaggaagccgaggtagtcggaggaggaggcgacagcgaactgagaagaagaaactccaccacaggcagagcagttttcctcattgctcagacctgatgcccagtggctctgaagagaagatccttagggagctgagcgaggaggaggaagacgaggaggaggaagaggaggaggaagaggagggaaggttttactatagtgaagaggaccatggggatgggtgttcctacactgacctactgccacaggatgatgggggtggcggcggctacagttcagtccgctacagtgactgttgtgaacgcgtggtaataaatgtgtctggtctacgcttcgaaacccaaatgaaaactttggctcagtttccagaaactctgttgggagaccctgagaagaggactcagtacttcgaccctttgcgcaatgagtatttttttgataggaaccgtcccagctttgatgccattttgtattattaccagtcaggaggccgcctgaagaggccagtcaatgtcccctttgatatcttcactgaggaggtgaagttctatcagttgggagaggaagccctgctcaagttccgtgaggatgagggctttgtgagagaagaggaggacagggctctgccagaaaatgaatttaaaaaacagatttggcttctctttgaatatcccgagagttccagccctgccagggcgatagccatcgtatctgtcctggtcatcttaatctctattgtcatattttgcctggaaaccttgcctgagttcagggatgatagggacctcatcatggccctcagcgcaggtggacacagcagattattgaatgacacctcggcaccccacctggagaactcagggcacacaatattcaatgaccctttcttcattgtggagacagtatgtatcgtgtggttttcctttgagtttgtggttcgatgctttgcttgtcccagtcaagcactcttcttcaaaaacatcatgaacatcattgatatcgtctccattttgccttacttcatcactctgggcaccgatctggcccagcagcaggggggtggcaacggccagcagcagcaggctatgtcctttgccatcctcaggatcatccgtctggtccgagtgttccggatcttcaagctctccagacactccaagggcctgcagatcctgggccacaccctaagagccagcatgcgtgaactgggccttcttatctttttcctcttcatcggggtcatcctcttttccagcgctgtgtattttgcagaggcagatgaacctaccacccatttccaaagcattccagatgcgttttggtgggctgtggtaaccatgacaactgtgggctacggggacatgaagcccatcacagtgggaggaaagattgtggggtccctgtgtgccattgcgggtgtcttaaccattgctttgcccgtgccggtgattgtgtctaactttaactatttctaccacagagagactgaaaacgaagaacagacccagctgacccaaaacgcagtcagttgcccatacctaccttctaatttgctcaagaaatttcggagctctacttcttcttccctgggggacaagtcagagtatctagagatggaagaaggggtcaaggagtctttatgtggaaaggaagagaagtgtcagggaaagggggatgacagcgagacagataaaaacaactgttctaatgcaaaggctgtggagactgatgtgtgaatctctttccccacctgccgtgccgccgcccagctccgaatatattcatacataaagaatgcagttatgaaaatgagatatgcactgcatacagtaatacactgcttaatggcgatacatggcataattgtggcgaaacgtgtattgcatatcaaataagtgatgcatcttggagaagagggaggcattaaaaacagcagatctatctttatattttttaatagaatgcaagaattttgcacataatgggaaaatgttaatagtaaaggtggtcccgaggagagtgagtgtgtgtgagagagtgagagagtgtgtggccatgggagtgtaagtaaattgtcaacattgttgggaattgtgccgtgatgggaaaagttggcattctgaagtatttactatgtaagaactaatgaacttgagcagtcttttaccagtgttttaataacatctcctatgtctttggattctgtagttgttttctagaaattgtaagaattactgtgtagaaaaaagagaaagtaaattatttaatagtatataggtcacaatttaatcttggatttaattaaagtttatttttaactggaaattaacttttgaaaaggctgcagggccttagaaattgattatattttgttattaattttgggagatatactagcaaatgcctaatgttctggaggaaatgtaacaagttttgttcacaggtcttaagactggaatttttttttcttttgcactactttctatgctgaagcccgagagagacttcatactgtgaatgtttactaacgcaccaatcagttcaatgacaatcattggaagaatggtttcttcgtctcatttattgttcttttcattttgtgagactaatgagcacacagataacagcacacgattcctgctttaaaatctgaacaaccgatctacaaagggactacgaagtaacgttcagcagccgaatctttcaaaattggtttgttacaatgatgcttcagaaaccatactattttcaatactcttctgccttttaagtccagaataatttaaccaaagttattgcatgcacagaaagaattccggcattttgttgcatatttaataaaaagatcttaagccataattgctgtagctgctgggcgccttatttctccaa

(19) United States
(12) Patent Application Publication
(10) Pub. No.: US 2004/0152107 A1
(43) Pub. Date: Aug. 5, 2004

(54) Gene signature of electroshock therapy and methods of use

(76) Inventors: C. Anthony Altar, Garrett Park, MD (US); Pascal Laeng, Washington, DC (US); Theresa A. Young, Germantown, MD (US); Vinod D. Charles, Silver Spring, MD (US)

Correspondence Address: DARBY & DARBY P.C., P. O. BOX 5257, NEW YORK, NY, 10150-5257, US

(21) Appl. No.: 10/664,705

(22) Filed: Sep. 18, 2003

Related U.S. Application Data
(60) Provisional application No. 60/411,718, filed on Sep. 18, 2002.
(60) Provisional application No. 60/431,882, filed on Dec. 9, 2002.
(60) Provisional application No. 60/479,970, filed on Jun. 18, 2003.

(51) Int. Cl. C12Q 1/68; G01N 33/53; G01N 33/567

(52) U.S. Cl. 435/6; 435/7.2

(57) ABSTRACT

Gene signatures associated with electroconvulsive therapy (ECT) and/or electroconvulsive seizures (ECS) are disclosed. Such “signatures” are useful, e.g., for diagnosing and treating neuropsychiatric disorders such as major depressive disorder (MDD), bipolar affective disorder (BAD) and psychotic depression. Such methods are therefore also provided here. The invention additionally provides screening methods that use gene signatures of the invention to identify new therapeutic compounds for treating these disorders.

### 1. CROSS-REFERENCE TO RELATED APPLICATIONS

Priority is claimed under 35 U.S.C. 119(e) to U.S. provisional application Serial Nos. 60/411,718; 60/431,882; and 60/479,970 filed on Sep. 18, 2002; Dec. 9, 2002; and Jun. 18, 2003, respectively. The contents of these priority applications are hereby incorporated by reference in their entireties.

### 2. FIELD OF THE INVENTION

The invention relates to methods and compositions for the diagnosis and treatment of neuropsychiatric disorders, including major depressive disorder (MDD), bipolar affective disorder (BAD), schizophrenia and psychotic depression. More specifically, the invention relates to gene signatures, that are associated with electroshock therapy and are useful for diagnosing and treating such neuropsychiatric disorders.

### 3. BACKGROUND OF THE INVENTION

Current treatments for the psychiatric depression, such as that associated with major depressive disorder (MDD), bipolar affective disorder (BAD), and psychotic depression, comprise several classes of antidepressant drugs. Unfortunately, many patients with these forms of depression fail to respond to such drug therapies. In such cases, electroconvulsive therapy (ECT) may be the only avaiable treatment. Indeed, ECT does produce a high level of success in patients where other pharmacological treatment regimens have failed.

Psychiatric depression associated with conditions such as major depressive disorder MDD) and psychotic depression are treated with several class of antidepressant drugs, all of which require several weeks to be effective. Unfortunately, many people do not respond to these drug therapies. In such cases, electroconvulsive therapy (ECT) remains the treatment. Although severe, ECT is recognized as a highly effective and rapid treatment, and it produces a high degree of success in patients where other therapeutic regimens have failed.

Receptor interactions and other pharmacological actions of many chemical antidepressants have been well studied and are characterized to at least some degree. By contrast, the mechanism or mechanisms of ECT remain poorly understood. Molecular effects of electroconvulsive seizure (ECS) in the brains of animals have been studied, as a model for ECT (for a review, see Fochtmann, Psychopharmacology Bulletin 1994, 30:321-444). The effects of ECS are divers, and include increases in levels of neurotransmitters, neuropeptides and synaptic remodeling, including sprouting. Id. ECS also afects several brain regions, including the hippocampus, frontal cortex, neostriatum, entorhinal cortex, temporal-parietal cortex, and monoaminergic nuclei that project to these areas. A summary of the effects reported in these regions is provided in a Table, attached as Appendix to this specification (see, Section 8.1, infra). These changes include, inter alia: increases in tyrosine kydroxylase in the locus coeruleus and diverse monoamine nerve terminal regions (Masserano et al., Science 1981, 214:662-665); decreases in β-adrenergic receptors, typically in the dentate gyrus of the hippocampus and in the frontal cortex (Biegon & Israeli, Eur. J. Pharmacol. 1986, 123:329-334); and increases of neurotrophic factors which can positively modulate monoaminergic neurotransmission (Altar et al., J. Neurochem. 1994, 63:1021-1032; Mamounas et al., J. Neurosci. 1995, 15:7929-7939; Martin-Iverson et al., J. Neurosci. 1994, 14:1262-1270; Siuciak et al., Pharmacol. Biochem. Behav. 1997, 56:131-137). Others have reported that the mRNA for brain-derived neurotrophic factor (BDNF) is upregulated in seizures (Isackson et al., Neuron 1991, 6:937-948; Nibuya et al., J. Neurosci. 1995, 15:7539-7547; Rocamora et al., Brain Res. Mol. Brain Res. 1992, 13:27-33).

Yet, the above-mentioned changes as well as those summarized in the Appendix, infra, were identified by investigations that examined changes of only very limited numbers of mRNA species in any brain region. No large-scale gene analysis following ECS has been reported. Hendriksen et al., (Eur. J. Neurosci 2001, 14:1475-1484) have described measured changes in the expression of multiple genes in the hippocampus, but only eight days after local seizures induced by electrical stimulation had been terminated by pentobarbital. Lukasiuk et al. (Eur. J. Neurosci. 2003, 17:271-279) report experiments where hippocampal and temporal lobe expression profiling was conducted in animals subject to amygdala kindling, but only before they developed spontaneous seizures.

The citation and/or discussion of a reference in this section and throughout the specification is provided merely to clarify the description of the present invention and is not an admission that any such reference is “prior art” to the invention described herein.

4. SUMMARY OF THE INVENTION

The present invention overcomes problems, such as those described supra, by providing ECS signature genes—i.e., nucleic acids that are differentially expressed in individuals undergoing an ECS treatment or in subjects undergoing electroconvulsive therapy (ECT). Preferred ECS signature genes of the invention are set forth in the tables at the appendix, infra. See, in particular, in Sections 8.3-8.5 below.

The invention also provides ECS gene signatures, which represent collections of one or more ECS signature genes and, preferably, expression levels of these genes which are indicative of ECS or ECT. In additiona, the invention also provides methods and algorithms for identifying ECS signature genes and for selecting preferred ECS signature genes, e.g., for use in an ECS gene signature.

ECS genes signatures of the invention, and the ECS signature genes they comprise, are useful, e.g., for diagnostic and prognostic methods of identifying people who have or who are susceptible to neuropsychiatric disorders, including inter alia major depressive disorder (MDD), bipolar affective disorder (BAD) and psychotic depression. Such methods therefore are also provided and considered a part of the present invention. The invention additionally provides therapeutic methods, which use ECS gene signatures and ECS signature genes to treat, ameliorate or prevent such neuropsychiatric disorders. The invention further comprises screening methods, including MPHTS(G methods, that use ECS gene signatures and/or ECS signature genes to identify compounds that are useful in such methods for treating neuropsychiatric disorders.

The methods of the invention include methods for identifying compounds for treating neuropsychiatric disorders, such as schizophrenia, autism, MDD, BAD, schizophrenia and psychotic depression. These methods generally comprise steps of contacting a cell or cells with a test compound, and determining expression of one or more signature genes by the cell or cells. These signature genes typicaly comprise nucleic acids that hybridize to a nucleic acid selected from the group consisting of SEQ ID NOS:1-152 and the complements thereof. The determined expression of these one or more signature genes is compared to expression of those signature genes in a cell or cell that is not contacted with the test compound. Changes in expression of the one or more signature genes (compared to expression in cells not contacted with the test compound) indicate that the compound is useful for treating the neuropsychiatric disorder.

The invention additionally provides methods for identifying and selecting ECS signature genes. In particular, the invention provides methods for selecting one or more nucleic acids that are indicative of an effective therapy for treating a neuropsychiatric disorder. The method comprises identifying nucleic acids that are differentialy expressed in an individual subjected to electroconvulsive seizure (ECS) compared to an individual not subjected to ECS. In various aspects of these methods, the individual may be subjected to either actue ECS or chronic ECS, and the nucleic acids identified by this method are preferably ones expressed in the brain or in a region of the brain (preferably the frontal cortex or the hippocampus). In preferred embodiments, a score value is obtained for each of the identified nucleic acids. The score value is preferably a function of each gene's differential expression in individuals subjected to ECS and can be determined according to an objective method or algorithm, such as the method set forth in Section 8.2 (infra) for selecting ECS signature genes that are most informative and therefore most useful for the assays and other methods of this invention.

The invention further provides kits for detecting an ECS gene signature. Kits of the invention generally comprise a plurality of oligonucleotides, each of which is capable of specifically hybridizing to a different ECS signature gene. For example, in preferred embodiments the oligonucleotides in such kits are capable of specifically hybridizng to ECS signature genes selected from the genes set forth in the appendix, infra (see, in particular, at Sections 8.3-8.5 below), such as SEQ ID NOS:1-152, as well as homologous and complementary sequences thereof. Preferred kits of the invention may comprise, for example, oligonucleotide probes that are immobilized on a solid surface or support, such as in an expression array. In other embodiments, kits of the invention may comprise a plurality of oligonucleotide primers, more preferably a plurality of primer pairs, wherein each pair is capable of amplifying a particular ECS signature gene (for example in a PCR reaction). Kits of the invention may additionally comprise oligonucleotide primers that are capable of priming reverse transcription reactions, for generating cDNA from mRNAs of one or more ECS signature genes. In still other embodiments, kits of the invention may contain other reagents including, for example, nucleotides that are detectably labeled (e.g., for detecting the amplification and or hybridization of ECS signature genes), polymerases, and/or buffers.

Preferred kits of the invention comprise oligonucleotides that are capable of specifically hybridizing to at least five, and more preferably to at least 10, 15, 20, 25, 30, 35, 40, 45, 50, 60, 70, 80, 90, 100, 110, 120, 130, 140, 150 or more signature genes. In other embodiments, a kit of the invention may comprise oligonucleotides that specifically hybridize to a set of ECS signature genes consisting of 150, 140, 130, 120, 110, 100, 90, 80, 70, 60, 50, 45, 40, 35, 30, 25, 20, 15, 10, 5 or fewer ECS signature genes of the invention.

5. BRIEF DESCRIPTION OF THE DRAWINGS

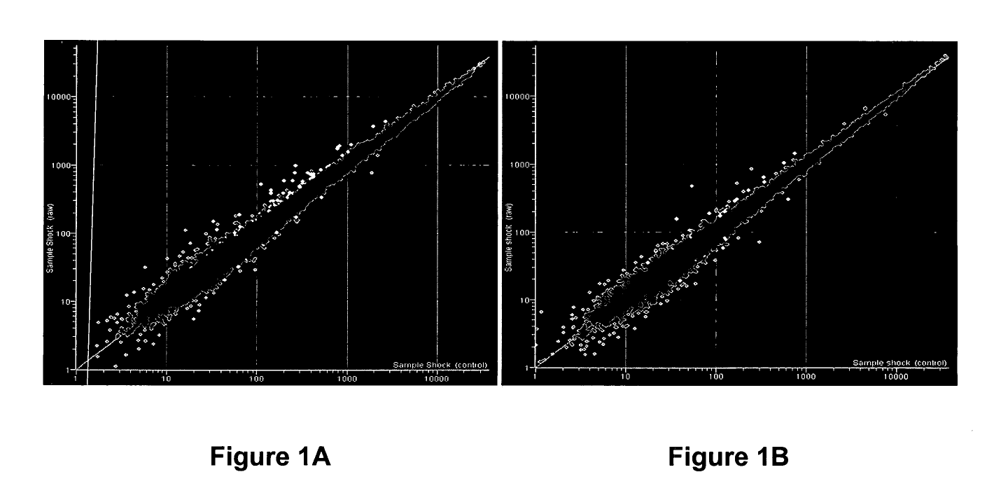

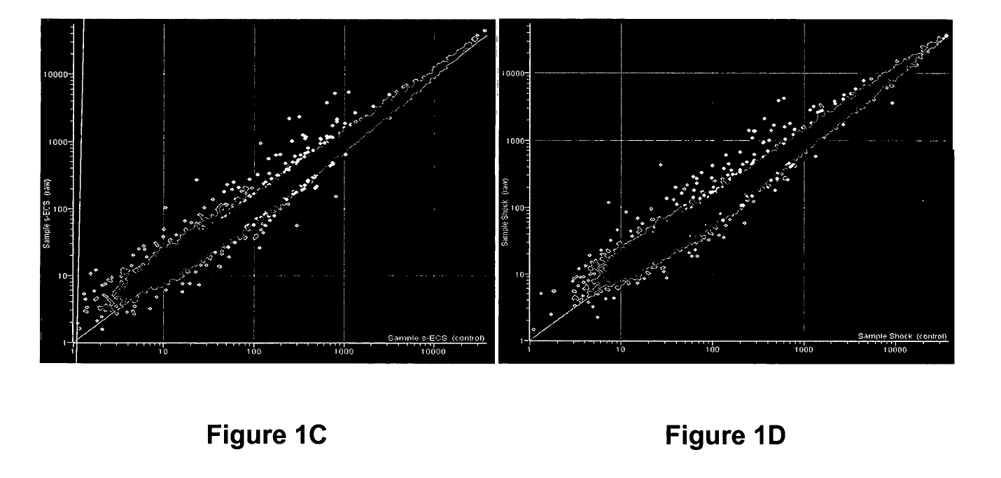

FIGS. 1A-1D plot the mean abundances of each of the 8,799 probe sets measured with the Affymetrix U34A rodent chip in the frontal cortex or hippocampus of rats exposed to either acute or chronic ECS (y-axes) compared to mean abundances of those same probe sets in a sham control group α-axes). Genes denoted by white points on each of the plots are ones passed minimum expression restrictions (described in Section 7.1, infra) whose expression changed (in ECS rats compared to sham controls) by either of 1.5 or more or of 0.67 or less, and having a p values of less than 0.05. FIG. 1A plots gene expression levels measured in the frontal cortex of rats exposed to acute ECS compared to sham controls. FIG. 1B plots gene expression levels measured in the frontal cortex of rats exposed to chronic ECS compared to sham controls. FIG. 1C plots gene expression levels measured in the hippocampus of rats exposed to acute ECS compared to sham controls, and FIG. 1D plots gene expression levels measured in the hippocampus of rats exposed to chronic ECS compared to sham controls.

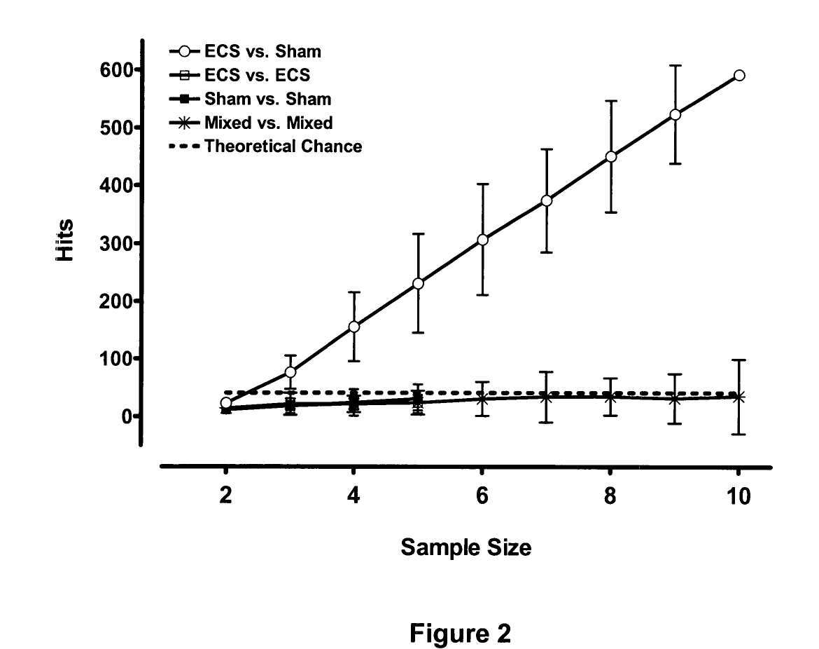

FIG. 2 shows a plot of a statistical evaluation demonstrating the effect of sample size on the number of identified gene changes or “hits”—i.e., measured changes in expression with p-values less than 0.01, regardless of the absolute magnitude of change in expression level. Groups of 2-10 rats were randomly composed from data of gene expression levels in the hippocampus of sham-treated (“sham”) and/or acute ECS-treated (“ECS”) rats.

The number of hits was calculated for each microarray analysis of these samples, and the process was repeated 100 times for each sample size. Values plotted in FIG. 2 are mean values ±1 standard deviation (SD). In the “mixed vs. mixed” condition, samples were randomly selected from all acute hippocampus samples, regardless of the treatment. The “theoretical chance” number of 40 gene changes is based on a p<0.01 for the 4,000 probes detected in the frontal cortex with at least one present call and a mean abundance of >100.

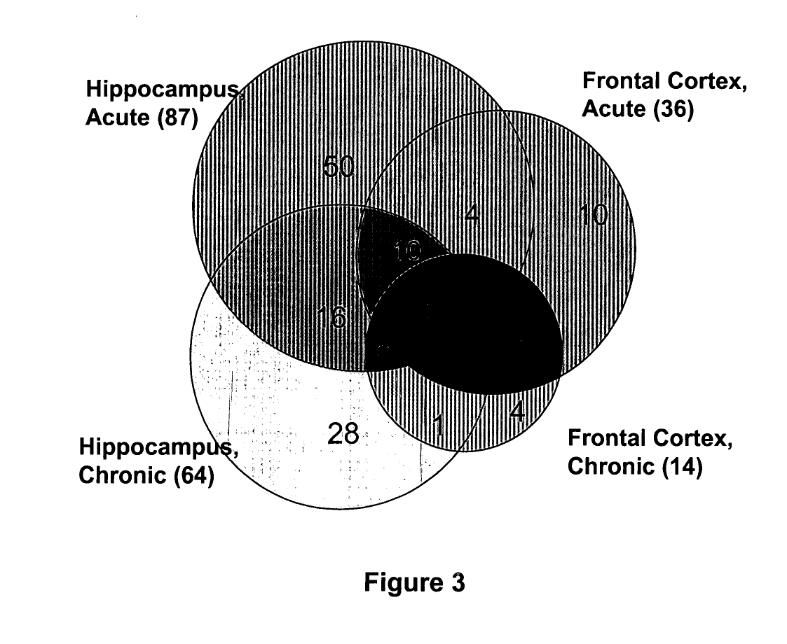

FIG. 3 shows a Venn diagram representation of the distribution of changes in 150 unique probe sets (of which 135 represent unique genes) in the frontal cortex and hippocampus after acute and chronic ECS. The total number of genes whose measured expression levels changed in each tissue are indicated in parentheses.

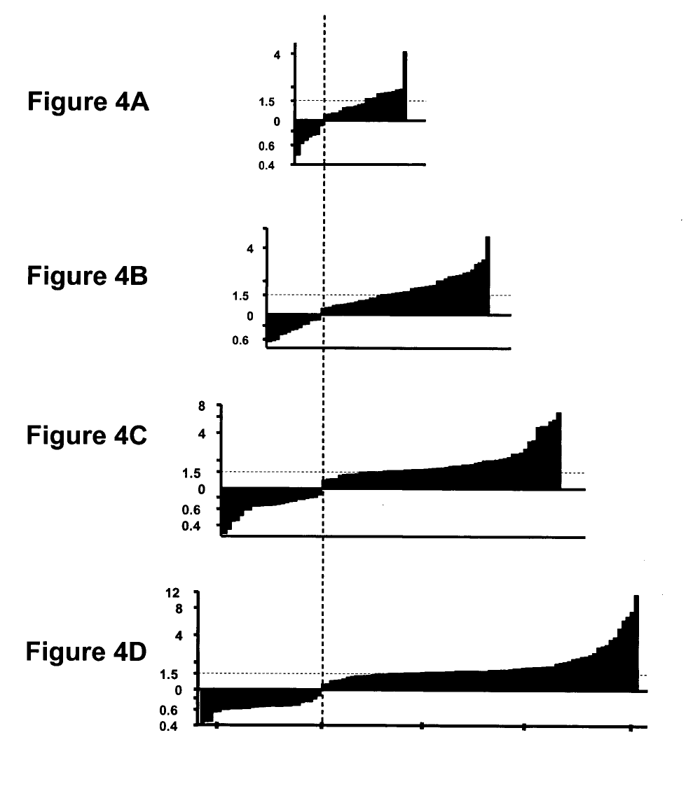

FIGS. 4A-4D show plots of the measured magnitude in expression levels for genes that were differentially expressed in the frontal cortex or hippocampus of rats treated with either acute or chronic ECS. FIGS. 4A and 4B plot changes in expression levels measured in the frontal cortex of rats treated with chronic or acute ECS, respectively, compared to sham-treated controls. FIGS. 4C and 4D plot changes in expression levels measured in the hippocampus of rats treated with chronic and acute ECS, respectively, compared to sham-treated control animals. For each gene whose expression changed (p<0.05) in treated animals compared with sham-treated control animals, the ratio of mean expression in the ECS group over the control group is plotted in order of the magnitude of changed. Decreases in the expression level of genes are plotted below the unity line, whereas increases in expression are plotted above the unity line. X-axes in all four plots are aligned and plotted at the same scale so that the width of the graph represents the number of genes affected in each direction.

6. DETAILED DESCRIPTION

To date, the identification of therapeutic compounds to treat neuropsychiatric disorders has depended almost entirely on serendipity. That is to say, effective drugs and other therapies for such disorders have traditionally been discovered by chance and not as the result of any directed systematic screening method. Indeed, the complex polygenetic nature of neuropsychiatric disorders, the subtle structural and cellular changes that they entail, and the difficulties in diagnosing and monitoring these disorders have made traditional drug screening methods extremely difficult if not impracticable. U.S. patent application Publication No. 2003/0096264 by Altar et al. describes screening methods, referred to in that application as “Multi-Parameter High Throughput Screening” or “MPHTS,” that overcome many of these difficulties and are ideally suited for identifying effective and/or promising therapeutic compounds to treat neuropsychiatric disorders, including schizophrenia, bipolar affective disorder (BAD), autism, major depressive disorder (MDD) and psychotic depression to name a few.

Briefly, the MPHTS approach pertains to the combination of data generated from gene expression profiling coupled with methods for the systematic analysis and/or employment of such data. Using the MPHTS methods described herein, large numbers of candidate compounds may be screened (e.g., in vitro) to identify ones that are particularly promising (and, as such, most likely to be suitable) for treating a neuropsychiatric disorder in vivo (e.g., in an individual). For descriptive purposes, these assays comprise at least two tiers. The first tier involves the determination of genes involved in a particular disorder, which is preferably a neuropsychiatric disorder. However, in another embodiment which is actually preferred here, the genes identified may be ones that are associated with a particular model for a neuropsychiatric or other disorder. For instance, the examples infra describe experiments that use electroconvulsive seizures (ECS) in rats as a model for electroconvulsive therapy.

The second tier of an MPHTS method involves the implementation of systematic methods to screen test compounds. Such screening methods may be either existing assay platforms that are already known in the art, such as those described in U.S. patent aplication Publication No. 2003/0096264, or they may be novel assays that are described here for the first time. Preferably, however, the screening assays will be automated and/or will be high-throughput assays, so that a large number of test compounds can be rapidly screened with a minimal amount of labor and effort.

6.1. Definitions

The terms used in this specification generally have their ordinary meanings in the art, withint the context of this invention and in the specific context where each term is used. Certain terms are discussed below, or else in the specification, to provide additional guidance to the practitioner in describing the compositions and methods of this invention and how they may be made and used. In accordance with the invention, there may be employed conventional techniques in the fields of molecular biology, microbiology, and recombinant DNA technology. These techniques are explained more fully in the literature and are within the skill of the art. See, for example, Sambrook, Fitsch & Maniatis, Molecular Cloning: A Laboratory Manual, Second Edition (1989) Cold Spring Harbor Laboratory Press, Cold Spring Harbor, N.Y. (referred to as “Sambrook et al., 1989”); DNA Cloning: A Practical Approach, Volumes I and II (D.N. Glover ed. 1985); Oligonucleotide Synthesis (M. J. Gait ed. 1984); Nucleic Acid Hybridization (B. D. Hames & S. J. Higgins, eds. 1984); Animal Cell Culture (R. I. Freshney, ed. 1986); Immobilized Cells and Enzymes (IRL Press, 1986); B. E. Perbal, A Practical Guide to Molecular Cloning (1984); F. M. Ausubel et al. (eds.), Current Protocols in Molecular Biology, John Wiley & Sons, Inc. (1994). The term “neuropsychiatric disorder”, which may also be referred to as a “major mental illness disorder” or “major mental illness”, refers to a disorder which may be generally characterized by one or more breakdowns in the adaptation process. Such disorders are therefore expressed primarily in abnormalities of thought, feeling and/or behavior producing either distress or impairment of function (i.e., impairment of mental function such as with dementia or senility). Currently, individuals may be evaluated for various neuropsychiatric disorders using criteria set forth in the most recent version of the American Psychiatric Association's Diagnostic and Statistical Manual of Mental Health (DSM-IV). Exemplary neuropsychiatric disorders include, but are not limited to, schizophrenia, attention deficit disorder (ADD), schizoaffective disorder, bipolar affective disorder, unipolar affective disorder, and adolescent conduct disorder.

As used herein, the term “isolated” means that the referenced material is removed from the environment in which it is normally found. Thus, an isolated biological material can be free of cellular components; i.e., components of the cells in which the material is found or produced. In the case of nucleic acid molecules, an isolated nucleic acid includes a PCR product, an isolated mRNA, a cDNA, or a restriction fragment. In another embodiment, an isolated nucleic acid is preferably excised from the chromosome in which it may be found, and more preferably is no longer joined to non-regulatory, non-coding regions, or to other genes, located upstream or downstream of the gene contained by the isolated nucleic acid molecule when found in the chromosome. In yet another embodiment, the isolated nucleic acid lacks one or more introns. Isolated nucleic acid molecules include sequences inserted into plasmids, cosmids, artificial chromosomes, and the like. Thus, in a specific embodiment, a recombinant nucleic acid is an isolated nucleic acid. An isolated protein may be associated with other proteins or nucleic acids, or both, with which it associates in the cell, or with cellular membranes if it is a membrane-associated protein. An isolated organelle, cell, or tissue is removed from the anatomical site in which it is found in an organism. An isolated material may be, but need not be, purified.

The term “purified” as used herein refers to material that has been isolated under conditions that reduce or eliminate the presence of unrelated materials, i.e., contaminants, including native materials from which the material is obtained. For example, a purified protein is preferably substantially free of other proteins or nucleic acids with which it is associated in a cell; a purified nucleic acid molecule is preferably substantially free of proteins or other unrelated nucleic acid molecules with which it can be found within a cell. As used herein, the term “substantially free” is used operationally, in the context of analytical testing of the material. Preferably, purified material substantially free of contaminants is at least 50% pure; more preferably, at least 90% pure, and more preferably still at least 99% pure. Purity can be evaluated by chromatography, gel electrophoresis, immunoassay, composition analysis, biological assay, and other methods known in the art.

Methods for purification are well-known in the art. For example, nucleic acids can be purified by precipitation, chromatography (including preparative solid phase chromatography, oligonucleotide hybridization, and triple helix chromatography), ultracentrifugation, and other means. Polypeptides and proteins can be purified by various methods including, without limitation, preparative disc-gel electrophoresis, isoelectric focusing, HPLC, reversed-phase HPLC, gel filtration, ion exchange and partition chromatography, precipitation and salting-out chromatography, extraction, and countercurrent distribution. For some purposes, it is preferable to produce the polypeptide in a recombinant system in which the protein contains an additional sequence tag that facilitates purification, such as, but not limited to, a polyhistidine sequence, or a sequence that specifically binds to an antibody, such as FLAG and GST. The polypeptide can then be purified from a crude lysate of the host cell by chromatography on an appropriate solid-phase matrix. Alternatively, antibodies produced against the protein or against peptides derived therefrom can be used as purification reagents. Cells can be purified by various techniques, including centrifugation, matrix separation (e.g., nylon wool separation), panning and other immunoselection techniques, depletion (e.g., complement depletion of contaminating cells), and cell sorting (e.g., fluorescence activated cell sorting or “FACS”). Other purification methods are possible. A purified material may contain less than about 50%, preferably less than about 75%, and most preferably less than about 90%, of the cellular components with which it was originally associated. The “substantially pure” indicates the highest degree of purity which can be achieved using conventional purification techniques known in the art.

A “sample” as used herein refers to a biological material which can be tested, e.g., for the presence of one or more polypeptide or nucleic acids. For example, in one embodiment, a sample is a sample of nucleic acids from a cell (e.g., mRNA, or nucleic acids derived therefrom) and is tested or analyzed for the presence or absence of certain particular nucleic acid sequences, corresponding to certain genes that may be expressed by the cell. Such samples can be obtained from any source, including tissue, blood and blood cells, including circulating hematopoietic stem cells (for possible detection of protein or nucleic acids), plural effusions, cerebrospinal fluid (CSF), ascites fluid, and cell culture.

Non-human animals include, without limitation, laboratory animals such as mice, rats, rabbits, hamsters, guinea pigs, etc.; domestic animals such as dogs and cats; and, farm animals such as sheep, goats, pigs, horses, and cows. A non-human animal of the present invention may be a mammalian or non-mammalian animal; a vertebrate or an invertebrate.

In preferred embodiments, the terms “about” and “approximately” shall generally mean an acceptable degree of error for the quantity measured given the nature or precision of the measurements. Typical, exemplary degrees of error are within 20 percent (%), preferably within 10%, and more preferably within 5% of a given value or range of values. Alternatively, and particularly in biological systems, the terms “about” and “approximately” may mean values that are within an order of magnitude, preferably within 5-fold and more preferably within 2-fold of a given value. Numerical quantities given herein are approximate unless stated otherwise, meaning that the term “about” or “approximately” can be inferred when not expressly stated.

The term “molecule” means any distinct or distinguishable structural unit of matter comprising one or more atoms, and includes, for example, polypeptides and polynucleotides.

The term “aberrant” or “abnormal”, as applied herein refers to an activity or feature which differs from a normal or activity or feature, or to an activity or feature which is within normal variations of a standard value.

For example, an abnormal activity of a gene or protein refers to an activity which differs from the activity of the wild-type or native gene or protein, or which differs from the activity of the gene or protein in a healthy subject. An activity of a gene includes, for instance, the transcriptional activity of the gene which may result from, e.g., an aberrant promoter activity. Such an abnormal transcriptional activity can result, e.g., from one or more mutations in a promoter region, such as in a regulatory element thereof. An abnormal transcriptional activity can also result from a mutation in a transcription factor involved in the control of gene expression.

An activity of a protein can be aberrant because it is stronger than the activity of its native counterpart. Alternatively, an activity can be aberrant because it is weaker or absent related to the activity of its native counterpart. An aberrant activity can also be a change in an activity. For example an aberrant protein can interact with a different protein relative to its native counterpart. A cell can have an aberrant activity due to overexpression or underexpression of a gene or protein. An aberrant activity can result, e.g., from a mutation in the gene, which results, e.g., in lower or higher binding affinity of a ligand or substrate to the protein encoded by the mutated gene.

The term “therapeutically effective dose” refers to that amount of a compound or compositions that is sufficient to result in a desired activity.

The phrase “pharmaceutically acceptable” refers to molecular entities and compositions that are physiologically tolerable and do not typically produce an allergic or similar untoward reaction (for example, gastric upset, dizziness and the like) when administered to an individual. Preferably, and particularly where a pharmaceutical composition is used in humans, the term “pharmaceutically acceptable” may mean approved by a regulatory agency (for example, the U.S. Food and Drug Agency) or listed in a generally recognized pharmacopeia for use in animals (for example, the U.S. Pharmacopeia).

The term “carrier” refers to a diluent, adjuvant, excipient, or vehicle with which a compound is administered. Sterile water or aqueous saline solutions and aqueous dextrose and glycerol solutions are preferably employed as carriers, particularly for injectable solutions. Exemplary suitable pharmaceutical carriers are described in “Reminington's Pharmaceutical Sciences” by E. W. Martin.

The term “polymer” means any substance or compound that is composed of two or more building blocks (‘mers’) that are repetitively linked together. For example, a “dimer” is a compound in which two building blocks have been joined together; a “trimer” is a compound in which three building blocks have been joined together; etc.

The term “polynucleotide” or “nucleic acid molecule” as used herein refers to a polymeric molecule having a backbone that supports bases capable of hydrogen bonding to typical polynucleotides, wherein the polymer backbone presents the bases in a manner to permit such hydrogen bonding in a specific fashion between the polymeric molecule and a typical polynucleotide (e.g., single-stranded DNA). Such bases are typically inosine, adenosine, guanosine, cytosine, uracil and thymidine. Polymeric molecules include “double stranded” and “single stranded” DNA and RNA, as well as backbone modifications thereof (for example, methylphosphonate linkages).

Thus, a “polynucleotide” or “nucleic acid” sequence is a series of nucleotide bases (also called “nucleotides”), generally in DNA and RNA, and means any chain of two or more nucleotides. A nucleotide sequence frequently carries genetic information, including the information used by cellular machinery to make proteins and enzymes. The terms include genomic DNA, cDNA, RNA, any synthetic and genetically manipulated polynucleotide, and both sense and antisense polynucleotides. This includes single- and double-stranded molecules; i.e., DNA-DNA, DNA-RNA, and RNA-RNA hybrids as well as “protein nucleic acids” (PNA) formed by conjugating bases to an amino acid backbone. This also includes nucleic acids containing modified bases, for example, thio-uracil, thio-guanine and fluoro-uracil.

The polynucleotides herein may be flanked by natural regulatory sequences, or may be associated with heterologous sequences, including promoters, enhancers, response elements, signal sequences, polyadenylation sequences, introns, 5′- and 3′-non-coding regions and the like. The nucleic acids may also be modified by many means known in the art. Non-limiting examples of such modifications include methylation, “caps”, substitution of one or more of the naturally occurring nucleotides with an analog, and internucleotide modifications such as, for example, those with uncharged linkages (e.g., methyl phosphonates, phosphotriesters, phosphoroamidates, carbamates, etc.) and with charged linkages (e.g., phosphorothioates, phosphorodithioates, etc.). Polynucleotides may contain one or more additional covalently linked moieties, such as proteins (e.g., nucleases, toxins, antibodies, signal peptides, poly-L-lysine, etc.), intercalators (e.g., acridine, psoralen, etc.), chelators (e.g., metals, radioactive metals, iron, oxidative metals, etc.) and alkylators to name a few. The polynucleotides may be derivatized by formation of a methyl or ethyl phosphotriester or an alkyl phosphoramidite linkage. Furthermore, the polynucleotides herein may also be modified with a label capable of providing a detectable signal, either directly or indirectly. Exemplary labels include radioisotopes, fluorescent molecules, biotin and the like. Other non-limiting examples of modification which may be made are provided, below, in the description of the present invention.

Specific non-limiting examples of synthetic nucleic acids envisioned for this invention include, in addition to the nucleic acid moieties described above, nucleic acids that contain phosphorothioates, phosphotriesters, methyl phosphonates, short chain alkyl, or cycloalkyl intersugar linkages or short chain heteroatomic or heterocyclic intersugar linkages. Most preferred are those with CH₂—NH—O—CH₂, CH₂—N(CH₃)—O—CH₂, CH₂—O—N(CH₃)—CH₂, CH₂—N(CH₃)—N(CH₃)—CH₂ and O—N(CH₃)—CH₂—CH₂ backbones (where phosphodiester is O—PO₂—O—CH₂). U.S. Pat. No. 5,677,437 describes heteroaromatic nucleic acid linkages. Nitrogen linkers or groups containing nitrogen can also be used to prepare nucleic acid mimics (U.S. Pat. Nos. 5,792,844 and 5,783,682). U.S. Pat. No. 5,637,684 describes phosphoramidate and phosphorothioamidate oligomeric compounds. Also envisioned are nucleic acids having morpholino backbone structures (U.S. Pat. No. 5,034,506). In other embodiments, such as the peptide-nucleic acid (PNA) backbone, the phosphodiester backbone of the nucleic acid may be replaced with a polyamide backbone, the bases being bound directly or indirectly to the aza nitrogen atoms of the polyamide backbone (Nielsen et al., Science 254:1497, 1991). Other synthetic nucleic acids may contain substituted sugar moieties comprising one of the following at the 2′ position: OH, SH, SCH₃, F, OCN, O(CH₂)ₙNH₂ or O(CH₂)ₙCH₃ where n is from 1 to about 10; C₁ to C₁₀ lower alkyl, substituted lower alkyl, alkaryl or aralkyl; Cl; Br; CN; CF₃; OCF₃; O-; S-, or N-alkyl; O-, S-, or N-alkenyl; SOCH₃; SO₂CH₃; ONO₂;NO₂; N₃; NH₂; heterocycloalkyl; heterocycloalkaryl; aminoalkylamino; polyalkylamino; substituted silyl; a fluorescein moiety; an RNA cleaving group; a reporter group; an intercalator; a group for improving the pharmacokinetic properties of a nucleic acid; or a group for improving the pharmacodynamic properties of an nucleic acid, and other substituents having similar properties. Nucleic acids may also have sugar mimetics such as cyclobutyls or other carbocyclics in place of the pentofuranosyl group. Nucleotide units having nucleosides other than adenosine, cytidine, guanosine, thymidine and uridine, such as inosine, may be used in an oligonucleotide molecule.

The term “oligonucleotide” refers to a nucleic acid, generally of at least 10, preferably at least 15, and more preferably at least 20 nucleotides, preferably no more than 100 nucleotides, that is hybridizable to a genomic DNA molecule, a cDNA molecule, or an mRNA molecule encoding a gene, mRNA, cDNA, or other nucleic acid of interest. Oligonucleotides can be labeled, e.g., with ³²P-nucleotides or nucleotides to which a label, such as biotin or a fluorescent dye (for example, Cy3 or Cy5) has been covalently conjugated. In one embodiment, a labeled oligonucleotide can be used as a probe to detect the presence of a nucleic acid. In another embodiment, oligonucleotides (one or both of which may be labeled) can be used as PCR primers, either for cloning full length or a fragment of a gene, or to detect the presence of nucleic acids encoding a particular gene product (e.g., to detect the presence of a particular mRNA). In a further embodiment, an oligonucleotide of the invention can form a triple helix. Generally, oligonucleotides are prepared synthetically, preferably on a nucleic acid synthesizer. Accordingly, oligonucleotides can be prepared with non-naturally occurring phosphoester analog bonds, such as thioester bonds, etc.

A “polypeptide” is a chain of chemical building blocks called amino acids that are linked together by chemical bonds called “peptide bonds”. The term “protein” refers to polypeptides that contain the amino acid residues encoded by a gene or by a nucleic acid molecule (e.g., an mRNA or a cDNA) transcribed from that gene either directly or indirectly. Optionally, a protein may lack certain amino acid residues that are encoded by a gene or by an mRNA. For example, a gene or mRNA molecule may encode a sequence of amino acid residues on the N-terminus of a protein (i.e., a signal sequence) that is cleaved from, and therefore may not be part of, the final protein. A protein or polypeptide, including an enzyme, may be a “native” or “wild-type”, meaning that it occurs in nature; or it may be a “mutant”, “variant” or “modified”, meaning that it has been made, altered, derived, or is in some way different or changed from a native protein or from another mutant.

A “ligand” is, broadly speaking, any molecule that binds to another molecule. In preferred embodiments, the ligand is either a soluble molecule or the smaller of the two molecule or both. The other molecule is referred to as a “receptor”. In preferred embodiments, both a ligand and its receptor are molecules (preferably proteins or polypeptides) produced by cells. Preferably, a ligand is a soluble molecule and the receptor is an integral membrane protein (i.e., a protein expressed on the surface of a cell). The binding of a ligand to its receptor is frequently a step of signal transduction within a cell. Exemplary ligand-receptor interactions include, but are not limited to, binding of a hormone to a hormone receptor (for example, the binding of estrogen to the estrogen receptor) and the binding of a neurotransmitter to a receptor on the surface of a neuron.

“Amplification” of a polynucleotide, as used herein, denotes the use of polymerase chain reaction (PCR) to increase the concentration of a particular DNA sequence within a mixture of DNA sequences. For a description of PCR see Saiki et al., Science 1988, 239:487.

“Chemical sequencing” of DNA denotes methods such as that of Maxam and Gilbert (Maxam-Gilbert sequencing; see Maxam & Gilbert, Proc. Natl. Acad. Sci. U.S.A. 1977, 74:560), in which DNA is cleaved using individual base-specific reactions.

“Enzymatic sequencing” of DNA denotes methods such as that of Sanger (Sanger et al., Proc. Natl. Acad. Sci. U.S.A. 1977, 74:5463) and variations thereof well known in the art, in a single-stranded DNA is copied and randomly terminated using DNA polymerase.

A “gene” is a sequence of nucleotides which code for a functional “gene product”. Generally, a gene product is a functional protein. However, a gene product can also be another type of molecule in a cell, such as an RNA (e.g., a tRNA or a rRNA). For the purposes of the present invention, a gene product also refers to an mRNA sequence which may be found in a cell. For example, measuring gene expression levels according to the invention may correspond to measuring mRNA levels. A gene may also comprise regulatory (i.e., non-coding) sequences as well as coding sequences. Exemplary regulatory sequences include promoter sequences, which determine, for example, the conditions under which the gene is expressed. The transcribed region of the gene may also include untranslated regions including introns, a 5′-untranslated region (5′-UTR) and a 3′-untranslated region (3′-UTR).

A “coding sequence” or a sequence “encoding” an expression product, such as a RNA, polypeptide, protein or enzyme, is a nucleotide sequence that, when expressed, results in the production of that RNA, polypeptide, protein or enzyme; i.e., the nucleotide sequence “encodes” that RNA or it encodes the amino acid sequence for that polypeptide, protein or enzyme.

A “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently found, for example, by mapping with nuclease S1), as well as protein binding domains (consensus sequences) responsible for the binding of RNA polymerase.

A coding sequence is “under the control of” or is “operatively associated with” transcriptional and translational control sequences in a cell when RNA polymerase transcribes the coding sequence into RNA, which is then trans-RNA spliced (if it contains introns) and, if the sequence encodes a protein, is translated into that protein.

The term “express” and “expression” means allowing or causing the information in a gene or DNA sequence to become manifest, for example producing RNA (such as rRNA or mRNA) or a protein by activating the cellular functions involved in transcription and translation of a corresponding gene or DNA sequence. A DNA sequence is expressed by a cell to form an “expression product” such as an RNA (e.g., a mRNA or a rRNA) or a protein. The expression product itself, e.g., the resulting RNA or protein, may also said to be “expressed” by the cell.

The term “heterologous” refers to a combination of elements not naturally occurring. For example, the present invention includes chimeric RNA molecules that comprise an rRNA sequence and a heterologous RNA sequence which is not part of the rRNA sequence. In this context, the heterologous RNA sequence refers to an RNA sequence that is not naturally located within the ribosomal RNA sequence. Alternatively, the heterologous RNA sequence may be naturally located within the ribosomal RNA sequence, but is found at a location in the rRNA sequence where it does not naturally occur. As another example, heterologous DNA refers to DNA that is not naturally located in the cell, or in a chromosomal site of the cell. Preferably, heterologous DNA includes a gene foreign to the cell. A heterologous expression regulatory element is a regulatory element operatively associated with a different gene that the one it is operatively associated with in nature.

The terms “mutant” and “mutation” mean any detectable change in genetic material, e.g., DNA, or any process, mechanism or result of such a change. This includes gene mutations, in which the structure (e.g., DNA sequence) of a gene is altered, any gene or DNA arising from any mutation process, and any expression product (e.g., RNA, protein or enzyme) expressed by a modified gene or DNA sequence. The term “variant” may also be used to indicate a modified or altered gene, DNA sequence, RNA, enzyme, cell, etc.; i.e., any kind of mutant. For example, the present invention relates to altered or “chimeric” RNA molecules that comprise an rRNA sequence that is altered by inserting a heterologous RNA sequence that is not naturally part of that sequence or is not naturally located at the position of that rRNA sequence. Such chimeric RNA sequences, as well as DNA and genes that encode them, are also referred to herein as “mutant” sequences.

“Sequence-conservative variants” of a polynucleotide sequence are those in which a change of one or more nucleotides in a given codon position results in no alteration in the amino acid encoded at that position.

“Function-conservative variants” of a polypeptide or polynucleotide are those in which a given amino acid residue in the polypeptide, or the amino acid residue encoded by a codon of the polynucleotide, has been changed or altered without altering the overall conformation and function of the polypeptide. For example, function-conservative variants may include, but are not limited to, replacement of an amino acid with one having similar properties (for example, polarity, hydrogen bonding potential, acidic, basic, hydrophobic, aromatic and the like). Amino acid residues with similar properties are well known in the art. For example, the amino acid residues arginine, histidine and lysine are hydrophilic, basic amino acid residues and may therefore be interchangeable. Similar, the amino acid residue isoleucine, which is a hydrophobic amino acid residue, may be replaced with leucine, methionine or valine. Such changes are expected to have little or no effect on the apparent molecular weight or isoelectric point of the polypeptide. Amino acid residues other than those indicated as conserved may also differ in a protein or enzyme so that the percent protein or amino acid sequence similarity (e.g., percent identity or homology) between any two proteins of similar function may vary and may be, for example, from 70% to 99% as determined according to an alignment scheme such as the Cluster Method, wherein similarity is based on the MEGALIGN algorithm. “Function-conservative variants” of a given polypeptide also include polypeptides that have at least 60% amino acid sequence identity to the given polypeptide as determined, e.g., by the BLAST or FASTA algorithms. Preferably, function-conservative variants of a given polypeptide have at least 75%, more preferably at least 85% and still more preferably at least 90% amino acid sequence identity to the given polypeptide and, preferably, also have the same or substantially similar properties (e.g., of molecular weight and/or isoelectric point) or functions (e.g., biological functions or activities) as the native or parent polypeptide to which it is compared.

The term “homologous”, in all its grammatical forms and spelling variations, refers to the relationship between two proteins that possess a “common evolutionary origin”, including proteins from superfamilies (e.g., the immunoglobulin superfamily) in the same species of organism, as well as homologous proteins from different species of organism for example, myosin light chain polypeptide, etc.; see, Reeck et al., Cell 1987, 50:667). Such proteins (and their encoding nucleic acids) have sequence homology, as reflected by their sequence similarity, whether in terms of percent identity or by the presence of specific residues or motifs and conserved positions.

The term “sequence similarity”, in all its grammatical forms, refers to the degree of identity or correspondence between nucleic acid or amino acid sequences that may or may not share a common evolutionary origin (see, Reeck et al., supra). However, in common usage and in the instant application, the term “homologous”, when modified with an adverb such as “highly”, may refer to sequence similarity and may or may not relate to a common evolutionary origin.

In specific embodiments, two nucleic acid sequences are “substantially homologous” or “substantially similar” when at least about 80%, and more preferably at least about 90% or at least about 95% of the nucleotides match over a defined length of the nucleic acid sequences, as determined by a sequence comparison algorithm known such as BLAST, FASTA, DNA Strider, CLUSTAL, etc. An example of such a sequence is an allelic or species variant of the specific genes of the present invention. Sequences that are substantially homologous may also be identified by hybridization, e.g., in a Southern hybridization experiment under, e.g., stringent conditions as defined for that particular system.

Similarly, in particular embodiments of the invention, two amino acid sequences are “substantially homologous” or “substantially similar” when greater than 80% of the amino acid residues are identical, or when greater than about 90% of the amino acid residues are similar (i.e., are functionally identical). Preferably the similar or homologous polypeptide sequences are identified by alignment using, for example, the GCG (Genetics Computer Group, Program Manual for the GCG Package, Version 7, Madison Wis.) pileup program, or using any of the programs and algorithms described above (e.g., BLAST, FASTA, CLUSTAL, etc.).

The terms “array” and “microarray” are used interchangeably and refer generally to any ordered arrangement (e.g., on a surface or substrate) or different molecules, referred to herein as “probes”. Each different probe of an arrays specifically recognizes and/or binds to a particular molecule, which is referred to herein as its “target”. Microarrays are therefore useful for simultaneously detecting the presence or absence of a plurality of different target molecules, e.g., in a sample. In preferred embodiments, arrays used in the present invention are “addressable arrays” where each different probe is associated with a particular “address”. For example, in preferred embodiments where the probes are immobilized on a surface or a substrate, each different probe of the addressable array may be immobilized at a particular, known location on the surface or substrate. The presence or absence of that probe's target molecule in a sample may therefore be readily determined by simply determining whether a target has bound to that particular location on the surface or substrate.

In various embodiments, an array of the invention may comprise a plurality of different antibodies that each bind to a particular target protein or antigen. More preferably, however, the methods of the invention are practiced using nucleic acid arrays (also referred to herein as “transcript arrays” or “hybridization arrays”) that comprise a plurality of nucleic acid probes immobilized on a surface or substrate. The different nucleic acid probes are complementary to, and therefore hybridize, to different target nucleic acid molecules, e.g., in a sample. Thus such probes may be used to simultaneously detect the presence and/or abundance of a plurality of different nucleic acid molecules in a sample, including the expression of a plurality of different genes; e.g., the presence and/or abundance of different mRNA molecules, or of nucleic acid molecules derived therefrom (for example, cDNA or cRNA).

A nucleic acid molecule is “hybridizable” to another nucleic acid molecule, such as a cDNA, genomic DNA, or RNA, when a single stranded form of the nucleic acid molecule can anneal to the other nucleic acid molecule under the appropriate conditions of temperature and solution ionic strength (see Sambrook et al., supra). The conditions of temperature and ionic strength determine the “stringency” of the hybridization. For preliminary screening for homologous nucleic acids, low stringency hybridization conditions (e.g., 5×SSC, 0.1% SDS, and no formamide; or 30% formamide, 5×SSC, 0.5% SDS) may be used. Alternatively, hybridizations may also be performed under conditions that are relatively more stringent, such as moderately stringent hybridization conditions (e.g., 40% formamide, with 5× or 6×SCC) or high stringency hybridization conditions (e.g., 50% formamide, 5× or 6×SCC). SCC is a buffer solution commonly used for nucleic acid hybridizations and comprises 0.15 M NaCl, 0.015 M Na-citrate.

Hybridization requires that the two nucleic acids contain complementary sequences, although depending on the stringency of the hybridization, mismatches between bases are possible. The appropriate stringency for hybridizing nucleic acids depends on the length of the nucleic acids and the degree of complementation, variables well known in the art. The greater the degree of similarity or homology between two nucleotide sequences, the greater the value of Tₘ for hybrids of nucleic acids having those sequences. The relative stability (corresponding to higher Tₘ) of nucleic acid hybridizations decreases in the following order:

RNA:RNA, DNA:RNA, DNA:DNA. For hybrids of greater than 100 nucleotides in length, equations for calculating Tₘ have been derived (see Sambrook et al., supra, 9.50-9.51). For hybridization with shorter nucleic acids, i.e., oligonucleotides, the position of mismatches becomes more important, and the length of the oligonucleotide determines its specificity (see Sambrook et al., supra, 11.7-11.8). A minimum length for a hybridizable nucleic acid is at least about 10 nucleotides; preferably at least about 15 nucleotides; and more preferably the length is at least about 20 nucleotides.

Suitable hybridization conditions for oligonucleotides (e.g., for oligonucleotide probes or primers) are typically somewhat different than for full-length nucleic acids (e.g., full-length cDNA), because of the oligonucleotides' lower melting temperature. Because the melting temperature of oligonucleotides will depend on the length of the oligonucleotide sequences involved, suitable hybridization temperatures will vary depending upon the oligonucleotide molecules used. Exemplary temperatures may be 37° C. (for 14-base oligonucleotides), 48° C. (for 17-base oligonucleotides), 55° C. (for 20-base oligonucleotides) and 60° C. (for 23-base oligonucleotides). Exemplary suitable hybridization conditions for oligonucleotides include washing in 6×SSC/0.05% sodium pyrophosphate, or other conditions that afford equivalent levels of hybridization.

Preferably, nucleic acid molecules in the present invention are detected by hybridization to probes of a microarray. Hybridization and wash conditions are therefore preferably chosen so that the probe “specifically binds” or “specifically hybridizes” to a specific target nucleic acid. In other words, the nucleic acid probe preferably hybridizes, duplexes or binds to a target nucleic acid molecules having a complementary nucleotide sequence, but does not hybridize to a nucleic acid molecules having a non-complementary sequence. As used herein, one polynucleotide sequence is considered complementary to another when, if the shorter of the polynucleotides is less than or equal to about 25 bases, there are no mismatches using standard base-pairing rules. If the shorter of the two polynucleotides is longer than about 25 bases, there is preferably no more than a 5% mismatch. Preferably, the two polynucleotides are perfectly complementary (i.e., no mismatches). In can be easily demonstrated that particular hybridization conditions are suitable for specific hybridization by carrying out the assay using negative controls. See, for example, Shalon et al., Genome Research 1996, 639-645; and Chee et al., Science 1996, 274:610-614.

Optimal hybridization conditions for use with microarrays will depend on the length (e.g., oligonucleotide versus polynucleotide greater than about 200 bases) and type (e.g., RNA, DNA, PNA, etc.) of probe and target nucleic acid. General parameters for specific (i.e., stringent) hybridization conditions are described above. For cDNA microarrays, such as those described by Schena et al. (Proc. Natl. Acad. Sci. USA 1996, 93:10614), typical hybridization conditions comprise hybridizing in 5×SSC and 0.2% SDS at 65° C. for about four hours, followed by washes at 25° C. in a low stringency wash buffer (for example, 1×SSC and 0.2% SDS), and about 10 minutes washing at 25° C. in a high stringency wash buffer (for example, 0.1×SSC and 0.2% SDS). Useful hybridization conditions are also provided, e.g., in Tijessen, Hybridization with Nucleic Acid Probes, Elsevier Sciences Publishers (1996), and Kricka, Nonisotopic DNA Probe Techniques, Academic Press, San Diego Calif. (1992).

The term “expression profile” or “gene signature” refer, generally, to any description or measurement of the genes and/or nucleic acids that are expressed by a cell or organism under particular conditions. For example, an expression profile may be measured under particular conditions of growth, for example at a particular temperature, in the presence or absence of particular growth media, and/or in the presence or absence of particular nutrients. In preferred embodiments, gene signatures may be obtained, e.g., for cells or tissues that are derived from an individual or individuals having a neuropsychiatric disorder. Gene signatures may also be obtained for a cell or organism exposed to one or more particular drugs or other compounds, such as for a cell or organism exposed to a known therapeutic compound (e.g., with a known use for treating a neuropsychiatric disorder) or for a cell or organism exposed to a “test” or “candidate” compound (e.g., as part of a MPHTS assay). An expression profile or gene signature may comprise a description of particular genes that are expressed by a cell or organism, a description of the level or abundance with which genes are expressed in a cell or organism, or both. Accordingly, the term “signature gene” is used herein to refer to a gene that may be used, either alone or with other genes (e.g., as part of a gene signature) to characterize a particular condition such as the presence or absence of a neuropsychiatric disorder.

Preferably, an expression profile will comprise a list of different mRNA species that are expressed by a cell and their relative abundances. For example, mRNA abundances can be measured using a microarray, as described below. In more preferable embodiments, nucleic acids (e.g., mRNA) expressed by a cell are reversed transcribed into either cDNA or cRNA, and the abundances of the cDNA and/or cRNA molecules are measured.

6.2. Multi-Parameter High Throughput Screening (MPHTS)

MPHTS methods are described in U.S. patent application Publication No. 2003/0096264 by Altar et al. Such methods generally comprise the following five elements. The skilled artisan will appreciate, however, that the invention may be practiced omitting one or more of these elements and without executing the recited elements in any particular order. For example, in certain embodiments, some of the below-described elements may be obtained from another source, such as from an online database. The invention may therefore be practiced without necessarily performing each of these elements, e.g., as a separate step in a screening method.

First, gene-signatures are obtained or provided by measuring expression levels for a plurality of genes in cells or tissues derived from an individual having a neuropsychiatric disorder or from a suitable model. For instance, in the Examples, infra, electroconvulsive seizures (ECS) induced in rats are used as models for electroconvulsive therapy (ECT) in human patients. Gene signatures are therefore obtained by measuring expression levels for a plurality of genes in the cells or tissues (more specifically in frontal cortex and hippocampus tissues) of animals subjected to ECS. In alternative embodiment, the cells and/or tissues are brain cells or tissues can be derived from human psychiatric patients (for example, in post mortem tissue samples). However, brain and other neuronal cells or tissues from other species of organisms may also be used, such as from a mouse, a rat, a primate or another species of mammal. Preferably, the organism from which the brain cells or tissue are derived represents an acceptable animal model for a neuropsychiatric disorder. Preferably, the expression levels measured in the cells or tissues are compared to expression levels from normal cells or tissues (i.e., brain cells or tissues from healthy individuals, not affected by a neuropsychiatric disorder) to identify particular genes that are differentially expressed in cells from an individual having a neuropsychiatric disorder compared to one who does not have a neuropsychiatric disorder.

Second, gene-signatures may also be obtained or provided by measuring expression levels for a plurality of genes in cultured neuronal cells or tissues (e.g., in cultured neurons that are derived from neural stem cells or from other neuronal cell lines). Human neurons and/or neuronal cell lines are particularly preferred. However, the cells may be obtained or derived from any species of organism, particularly a mammalian species such as a mouse, a rat or a primate. Similarly, the cultured neuronal tissues may also be obtained from any species of mammal, such as from a rat, a mouse, a primate or a human.

For example, and not by way of limitation, a mouse neuroblastoma cell line may be used in such methods. Such cells are readily available, e.g., from the American Type Culture Collection (“ATCC”, Manasas Virginia). See, for example, ATCC Accession No. CRL-2263. As another non-limiting example, U.S. provisional patent application serial No. 60/299,066 filed on Jun. 18, 2001 describes the use of rat neuronal cell cultures to evaluate neuropsychiatric drugs. Such cells may also be used in the MPHTS methods of this invention.

Third, drug signatures may also be obtained or provided by measuring expression levels for a plurality of genes in cultured neuronal cells or tissues that are treated with a therapeutic compound. The cultured cells may be any type of neuronal cell or cell lines described supra for obtaining gene-signatures from a cell line. Similarly, any of the types of tissue cultures described, supra, may also be used to obtain drug signatures. Preferably, the drug signatures are signatures for compounds that are known to be effective for treating a neuropsychiatric disorder. Exemplary compounds may include valproate, buspirone, lithium, carbamazepine, clozapine, olanzapine, haloperidol, secretin and vasoactive intestinal polypeptide (VIP), to name a few. Exemplary drug signatures, which were obtained from broth rat and human neuronal cells treated with therapeutic compounds, are provided in the Examples, infra. Other drug signatures may be readily obtained by those skilled in the art.

Fourth, expression levels for the plurality of genes are obtained or provided in neuronal cells that are contacted with a test compound (referred to here as a “drug candidate”), and these expression levels may then be compared to expression levels from gene signatures obtained for the neuropsychiatric disorder (as described in the first element, supra) and/or to drug-signatures obtained the known therapeutic compound (as described in the third element, supra). In preferred embodiments, expression levels or “signatures” obtained from a test compound are also compared to expression levels when the cell or cell line is not contacted with the test compound or any other drug (described in the second element, supra).

Generally speaking, the “signature” or expression levels obtained when the neuronal cells are contacted with a test compound are compared to the gene signatures of the cells when they are not contacted with any test or therapeutic compound (i.e., the gene signature obtained as element two, described supra) to identify changes in the expression level(s) for particular genes. Similarly, the drug-signature (obtained as described, supra, for element three) is also compared to the neuronal cell lines gene signature, to identify particular genes whose expression levels change when the cells are contacted with the therapeutic compound. In instances where changes in expression levels when the cells are contacted with the test compound are identical (or at least similar) to changes in expression levels when the cell are contacted with the known therapeutic compound, then the test compound is identified as a candidate compound for treating the neuropsychiatric disorder. Thus, using these screening methods a skilled artisan is able to rapidly and inexpensively identify compounds that are most promising as novel neuropsychiatric drugs, while eliminating compounds that show little promise and/or are unlikely candidates for treating a neuropsychiatric disorder.

In preferred embodiments of the invention, changes in expression levels when the cells are contacted with the test compound may also be compared to gene signatures obtained for the particular neuropsychiatric disorder of interest (i.e., to the gene signatures obtained as described, supra, for the first element). Preferably, a test compound that is identified as a candidate therapeutic compound will alter the expression of “signature gene” in a way that is opposite or contrary to the expression observed in the disorder's gene signature. For example, where a particular gene is expressed at abnormally high levels in cells or tissues from individuals affected by the particular neuropsychiatric disorder (compared to expression levels in cells or tissues from individuals not affected by the disorder), a candidate compound identified in these screening methods will preferably inhibit that gene's expression (i.e., the gene is preferably expressed at lower levels when the cells are contacted with the test compound, compared to its expression when the cell is not contacted with the test compound).

Fifth, as an optional element of the invention, drug candidate or candidate compounds that are identified as described, supra, may be further optimized, e.g., to account for individual genetic variability.

As indicated above, the MPHTS assays of the invention are useful as an inexpensive and rapid initial screening to quickly identify compounds that are most promising as neuropsychiatric drugs, while quickly eliminating compounds that show little promise and/or are unlikely candidates for treating a neuropsychiatric disorder. In preferred embodiments, the MPHTS assays are used to identify candidate compounds for treating bipolar affective disorder (BAD), depression, schizophrenia and autism. However, the assays are by no means limited to these particular disorders, and may be readily adapted to identify candidate compounds for treating any neuropsychiatric disorder. Other exemplary, preferred neuropsychiatric disorders for which these assays may be used include anxiety disorders, eating disorders, addictive disorders and Attention Deficit Hyperactivity Disorder (ADHD).

Classes of compounds that may be identified by such screening assays include, but are not limited to, small molecules (e.g., organic or inorganic molecules which are less than about 2 kd in molecular weight, are more preferably less than about 1 kd in molecular weight, and/or are able to cross the blood-brain barrier or gain entry into an appropriate cell, as well as macromolecules (e.g., molecules greater than about 2 kd in molecular weight). In preferred embodiments, commercially available compound libraries may be purchased and screened in an MPHTS assay of the invention. Examples of preferred libraries include TOCRIS (Tocris Cookson, Ltd. Avonmouth Bristol, United Kingdom), SIGMA RBI (Sigma Aludrich Inc., St. Louis Mo.), ChemBridge (ChemBridge Corp., San Diego Calif.), Chemdiv (ChemDiv Inc., San Diego Calif.) and Prestwick (Prestwick Chemical, Inc., Washington D.C.), to name a few.

The selection of appropriate small molecule compound concentrations for the treatment of cells in vitro or for dosing of animals in vivo is preferred to discriminate between physiological and toxicological effects of a given compound. As an initial means for determining the deleterious effects of a compound or set of compounds, cells may be seeded (e.g., in multiple-well plates) and treated with a range of compound concentrations.

The compounds' effect (e.g., its cytotoxic or apoptotoic effect) may then be gauged, e.g., using commercially available kits and routine methods well known in the art.

Compounds identified by these screening assays may also include peptides and polypeptides. For example, soluble peptides, fusion peptides members of combinatorial libraries (such as ones described by Lam et al., Nature 1991, 354:82-84; and by Houghten et al., Nature 1991, 354:84-86); members of libraries derived by combinatorial chemistry, such as molecular libraries of D- and/or L-configuration amino acids; phosphopeptides, such as members of random or partially degenerate, directed phosphopeptide libraries (see, e.g., Songyang et al., Cell 1993, 72:767-778); antibodies, including but not limited to polyclonal, monoclonal, humanized, anti-idiotypic, chimeric, or single chain antibodies; antibody fragments, including but not limited to FAb, F(ab′)₂, FAb expression library fragments and epitope-binding fragments thereof.

The compounds used in such screening assays are also preferably essential pure and free of contaminants which may, themselves, alter or influence gene expression. Compound purity may be assessed by any number of means that are routine in the art, such as LC-MS and NMR spectroscopy. Libraries of test compounds are also preferably biased by using computational selection methods which are routine in the art. Tools for such computational selection, such as Pipeline PilotJ (Scitegic Inc., San Diego, Calif.) are commercially available. The compounds may be assessed using rules such as the “Lipinski criteria” (see, Lipinski et al., Adv. Drug Deliv. Rev. 2001, 46:3-26) and/or any other criteria or metrics commonly used in the art.

6.3. Preparation of Neuronal Cell and Tissue Samples

Brain tissue samples. In certain limited embodiments, brain cells and tissues for use in the MPHTS methods of this invention may be obtained from individuals (e.g., from patients) in a biopsy. However, those skilled in the art will recognize that brain surgeries permitting a biopsy are relatively rare and primarily involve surgical excisions (e.g., for the treatment of epilepsy) rather than brain regions relevant to neuropsychiatric disorder such as schizophrenia or bipolar affective disorder. In certain embodiments, however, useful disease profiles may be obtained from cultured peripheral nervous system neurons, such as rhinoneuroepithelial cells. Such cells may be readily obtained from a nasal biopsy, and disease profiles from such cells may be used to identify changes in gene expression that are associated with neuropsychiatric disorders such as schizophrenia.

In preferred embodiments, brain cells or tissues used in the methods of this invention are instead obtained post-mortem, e.g., from cadavers of individuals who had or exhibited symptoms of a neuropsychiatric disorder during their lifetime.

Those skilled in the art will readily appreciate that a large number of carefully collected brain tissue samples should preferably be obtained to assure statistical reliability (see, for example, Torrey et al., SchizophrRes. 2000,44:151; Bahn et al., J. Chem. Neuroanatomy 2001, 22:79-94; and Vawter et al., Brain Res. Bull. 2001, 55:641-650). This is particularly desirable where there is considerable heterogeneity in patient age to permit accounting for age-associated variables (for example, progressive brain degeneration, which may also occur in schizophrenia). However, smaller samples may be used, e.g., for preliminary screening assays where statistical reliability may not be as essential. It is also preferable that the samples be matched, e.g., according to the patients' age, sex, cause of death and post-mortem interval. The brain samples used preferably are not acquired from cadavers under circumstances that might themselves affect the quality of the cells or tissues acquired. For example, samples obtained following a prolonged moribund state, a coma, hpoxia, pyrexia or stroke preferably are not used in MPHTS methods of the invention. A skilled artisan may readily recognize such compromised, ante mortem states, e.g., from the extent of brain acidosis. Generally, measured postmortem tissue pH values that are below about 6.4 indicate that the tissue has been subjected to such a compromised ante mortem state and should not be used. In addition, the postmortem tissue pH value is also critical to the integrity of mRNA obtained from the tissue.

It is understood that a reliable psychiatric diagnosis and cause of death should also be obtained or determined for the individual. It is, moreover, additionally preferably to identify factors such as concomitant medical conditions, medications taken during the patient's lifetime (particularly immediately prior to death), surgical treatments (including cancer treatments) and substance abuse for each patient. The hemisphere and region of the brain from which each sample is taken is also preferably noted and recorded.

Generally, samples that have been subject to such conditions as may affect the reliability of gene expression measurements should not be used. However, in many situations the skilled artisan will recognize that such factors may be sufficiently controlled for and the sample, therefore, acceptable for use in MPHTS. In such cases, however, it is preferable and often essential that the samples be appropriately matched. As an example, and not by way of limitation, it is recognized that smoking alters the expression of many genes in the hippocampus, a region of the brain that is also associated with schizophrenia (Wang et al., Abs. Soc. Neurosci. 2001, 27). However, the overlap between genes whose expression levels have been reported as altered by those two conditions is believed to be minimal (see, Wang et al., supra). Therefore, it may be possible to practice MPHTS methods of the invention using samples from smoking or non-smoking individuals, provided the samples are appropriately matched.

Those skilled in the art will also appreciate that the levels and quality of RNA extracted from post-mortem samples may be influenced by factors such as the post mortem interval (i.e., the time interval between death and RNA extraction), the refrigeration time (i.e., the time interval from death to patient storage in a cold environment), the storage time (i.e., the duration of time during which the cadaver is refrigerated). Accordingly, it is preferably that such factors be appropriately controlled and that the steps of RNA extraction from these tissue samples be as efficient as possible. In particularly preferred embodiments, the brain or tissue samples are unfixed (i.e., are not treated with protein cross-linkers such as formalin) and have not been thawed more than once.

In a preferred embodiment, samples of brain tissue may be obtained, e.g., post-mortem from cadavers of individuals who (during their lifetime) suffered from or exhibited symptoms of a neuropsychiatric disorder. However, single neurons or groups of homogeneous neurons may also be extracted from such cadavers, e.g., by laser capture microdissection (LCM). Using RNA amplification, gene expression profiles may be measured for these single cells as well (see, e.g., Eberwine et al., Proc. Natl. Acad. Sci. 1992, 89:30130-30134; and Luo et al., Nature Med. 1999, 5:117-119). Expression profiles obtained from these cells will therefore be particular for the particular cell types extracted, and may ultimately provide gene expression profiles that are more clearly ascribed to the particular cell population. Such gene profiles will typically be more robust, and therefore preferable, for evaluating a drug response.

Brain cells or tissues obtained from animals may also be used. For example, tissue or samples from animal models for a neuropsychiatric disorder may be used to model disease profiles for that disorder. Alternatively, expression profiles may be obtained from brain cells or tissues obtained from animals treated with a known anti-psychotic drug or with a test compound. In addition, cells from a transgenic animals may be employed, in which one or more genes relevant to a neuropsychiatric disorder have been altered, over-expressed or “knocked-out”. High throughput in vitro screening of candidate compounds may then be carried out using neuronal cells obtained or derived from such a transgenic animal.

Neuronal cells. In preferred embodiments, the MPHTS methods of the invention also used cultured cells or cell lines to screen for candidate therapeutic compounds. Preferably, the cells are ones having an expression profile that is typical of neuronal cells or, alternatively, they may be cells which can be manipulated to produce an expression profile typical of neuronal cells. The cells or cell lines used will also, preferably, give rise to reproducible changes in their gene expression profiles when contacted with known antipsychiatric drugs (for example, valproate). In a particularly preferred embodiment, these changes will be opposite changes that are observed in the disease signature. That is to say, in such embodiments, genes (or their homologs) normally expressed at higher levels in the disease signature are preferably expressed at lower levels in cells or cell lines contacted with the known antipsychiatric drug, and vice-versa.

In a preferred embodiment, pluripotent neuronal stem cell lines are used in these aspects of the invention. Such cell lines are well known in the art, and methods to induce or enhance the differentiation of such stem cell lines have been described. For example, U.S. Provisional Patent Application Publication Nos. 2003/0082802 A1 and 2003/0013192 A1 both describe methods for inducing differentiation in neuronal stem cells by exposure to chemicals (for example, valproate and buspirone). In other embodiments, such cells may be differentiated, e.g., using antisense strategies and/or routine techniques of molecular biology to develop stable, transfected cell lines. Alternatively, however, cells or cell lines may also be obtained from patients having a neuropsychiatric disorder of interest.

A skilled artisan will readily appreciate that cells or cell cultures used in the methods of this invention should be carefully controlled for parameters such as the cell passage number, cell density (e.g., in microplate wells), the method(s) by which cells are dispensed, and growth time after dispensing. It is also preferable to repeat mRNA and/or protein expression levels measured for a cell or cell line under particular conditions, to confirm that the measured levels are reproducible.

6.4. Measuring Gene Expression

MPHTS methods generally, including the particular methods of this invention, may be implemented using any method suitable for measuring changes in the gene expression of a cell or cells. Such methods are well known and routinely used in the art. In preferred embodiments, methods are used that permit the simultaneous measurement of expression for a plurality of genes (e.g., for at least 10, more preferably for at least 100, still more preferably for at least 150). For example, in particularly preferred embodiments expression profiles are measured using “transcript arrays” or “microarrays,” as described below. However, any technique that is capable of measuring gene expression may be used and the methods of this invention are not limited to the use of nucleic acid microarrays. For instance, gene expression can also be measured in alternative embodiments by using a reverse transcription polymerase chain reaction (RT-PCR).

Systems and kits for implementing such assays are commercially available from a number of suppliers, including Affymetrix (Santa Clara, Calif.), Agilent (Palo Alto, Calif.), Promega (Madison, Wis.), Xantnon (Research Triangle Park, North Carolina), Illumina (San Diego, Calif.), Chromagen (San Diego, Calif.), Third Wave Technologies (Madison, Wis.), Aclara (Mountain View, Calif.), Beckton Dickinson & Co. (Franklin Lakes, N.J.) and Luminex (Austin, Tex.) to name a few.

Transcript arrays. In a preferred embodiment the present invention makes use of “transcript arrays” (also called herein “microarrays”). Transcript arrays can be employed for analyzing the steady state level of mRNAs in a cell, and especially for comparing the steady state levels between two cells, such as a first cell that has been exposed to a drug, drug candidate or other compound, and a second cell that has not been treated.

In one embodiment, transcript arrays are produced by hybridizing detectably labeled polynucleotides representing the mRNA transcripts present in a cell (e.g., fluorescently labeled cDNA synthesized from total cell mRNA) to a microarray. As explained in the definitions, supra, microarray is a surface with an ordered array of binding (e.g., hybridization) sites for products of many of the genes in the genome of a cell or organism, preferably most or almost all of the genes. Microarrays can be made in a number of ways, of which several are described below. However produced, microarrays share certain characteristics. The arrays are preferably reproducible, allowing multiple copies of a given array to be produced and easily compared with each other. Preferably the microarrays are small, usually smaller than 5 cm², and they are made from materials that are stable under binding (e.g., nucleic acid hybridization) conditions. A given binding site or unique set of binding sites in the microarray will specifically bind the product of a single gene in the cell. Although there may be more than one physical binding site (hereinafter “site”) per specific mRNA, for the sake of clarity the discussion below will assume that there is a single site. It will be appreciated that when cDNA complementary to the RNA of a cell is made and hybridized to a microarray under suitable hybridization conditions, the level of hybridization to the site in the array corresponding to any particular gene will reflect the prevalence in the cell of mRNA transcribed from that gene. For example, when detectably labeled (e.g., with a fluorophore) cDNA complementary to the total cellular mRNA is hybridized to a microarray, the site on the array corresponding to a gene (i.e., capable of specifically binding a nucleic acid product of the gene) that is not transcribed in the cell will have little or no signal, and a gene for which the encoded mRNA is prevalent will have a relatively strong signal.

In preferred embodiments, cDNAs from two different cells, e.g., a cell exposed to a test compound and a cell of the same type not exposed to the compound, are hybridized to the binding sites of the microarray. The cDNA derived from each of the two cell types are differently labeled so that they can be distinguished. In one embodiment, for example, cDNA from a cell treated with a drug is synthesized using a fluorescein-labeled dNTP, and cDNA from a second cell, not drug-exposed, is synthesized using a rhodamine-labeled dNTP. When the two cDNAs are mixed and hybridized to the microarray, the relative intensity of signal from each cDNA set is determined for each site on the array, and any relative difference in abundance of a particular mRNA detected.

In the example described above, the cDNA from the treated cell will fluoresce green when the fluorophore is stimulated and the cDNA from the untreated cell will fluoresce red. As a result, when the compound has no effect, either directly or indirectly, on the relative abundance of a particular mRNA in a cell, the mRNA will be equally prevalent in both cells and, upon reverse transcription, red-labeled and green-labeled cDNA will be equally prevalent. When hybridized to the microarray, the binding site(s) for that species of RNA will emit wavelengths characteristic of both fluorophores. In contrast, when the cell is exposed to a compound that, directly or indirectly, increases the prevalence of the mRNA in the cell, the ratio of green to red fluorescence will increase. When the drug decreases the mRNA prevalence, the ratio will decrease.

The use of a two-color fluorescence labeling and detection scheme to define alterations in gene expression has been described, e.g., in Shena et al., Science 1995, 270:467-470. An advantage of using cDNA labeled with two different fluorophores is that a direct and internally controlled comparison of the mRNA levels corresponding to each arrayed gene in two cell states can be made, and variations due to minor differences in experimental conditions (e.g., hybridization conditions) will not affect subsequent analyses. However, it will be recognized that it is also possible to use cDNA from a single cell, and compare, for example, the absolute amount of a particular mRNA in, e.g., a treated and untreated cell.

Preparation of microarrays. Nucleic acid microarrays are known in the art and preferably comprise a surface to which probes that correspond in sequence to gene products (e.g., cDNAs, mRNAs, cRNAs, polypeptides, and fragments thereof), can be specifically hybridized or bound at a known position. In one embodiment, the microarray is an array in which each position represents a discrete binding site for a product encoded by a gene (e.g., a protein or RNA), and in which binding sites are present for products of most or almost all of the genes in the organism's genome. In a preferred embodiment, the “binding site” (hereinafter, “site”) is a nucleic acid or nucleic acid analogue to which a particular cognate cDNA or CRNA can specifically hybridize. The nucleic acid or analogue of the binding site can be, e.g., a synthetic oligomer, a full-length cDNA, a less-than full-length cDNA, or a gene fragment.

Although in a preferred embodiment the microarray contains binding sites for products of all or almost all genes in the target organism's genome, such comprehensiveness is not necessarily required. Usually the microarray will have binding sites corresponding to at least about 50% of the genes in the genome, often at least about 75%, more often at least about 85%, even more often more than about 90%, and most often at least about 99%. Preferably, the microarray has binding sites for genes relevant to the action of a drug of interest. A “gene” is identified as a segment of DNA containing an open reading frame (ORF) of preferably at least 50, 75, or 99 amino acids from which a messenger RNA is transcribed in the organism (e.g., if a single cell) or in some cell in a multicellular organism. The number of genes in a genome can be estimated from the number of mRNAs expressed by the organism, or by extrapolation from a well-characterized portion of the genome. When the genome of the organism of interest has been sequenced, the number of ORFs can be determined and mRNA coding regions identified by analysis of the DNA sequence.

Preparing nucleic acids for microarrays. As noted above, the “binding site” to which a particular cognate cDNA specifically hybridizes is usually a nucleic acid or nucleic acid analogue attached at that binding site. In one embodiment, the binding sites of the microarray are DNA polynucleotides corresponding to at least a portion of each gene in an organism's genome. These DNAs can be obtained by, e.g., polymerase chain reaction (PCR) amplification of gene segments from genomic DNA, cDNA (e.g., by RT-PCR), or cloned sequences. PCR primers are chosen, based on the known sequence of the genes or cDNA, that result in amplification of unique fragments (i.e. fragments that do not share more than 10 bases of contiguous identical sequence with any other fragment on the microarray). Computer programs are useful in the design of primers with the required specificity and optimal amplification properties. See, e.g., Oligo version 5.0 (National Biosciences). In the case of binding sites corresponding to very long genes, it will sometimes be desirable to amplify segments near the 3′ end of the gene so that when oligo-dT primed cDNA probes are hybridized to the microarray, less-than-full length probes will bind efficiently. Typically each gene fragment on the microarray will be between about 50 bp and about 2000 bp, more typically between about 100 bp and about 1000 bp, and usually between about 300 bp and about 800 bp in length. PCR methods are well known and are described, for example, in Innis et al., eds., 1990, PCR Protocols: A Guide to Methods and Applications, Academic Press Inc. San Diego, Calif. It will be apparent that computer controlled robotic systems are useful for isolating and amplifying nucleic acids.

An alternative means for generating the nucleic acid for the microarray is by synthesis of synthetic polynucleotides or oligonucleotides, e.g., using N-phosphonate or phosphoramidite chemistries (Froehler et al., Nucleic Acid Res. 1986, 14:5399-5407; McBride et al., Tetrahedron Lett. 1983, 24:245-248). Synthetic sequences are between about 15 and about 500 bases in length, more typically between about 20 and about 50 bases. In some embodiments, synthetic nucleic acids include non-natural bases, e.g., inosine. As noted above, nucleic acid analogues may be used as binding sites for hybridization. An example of a suitable nucleic acid analogue is peptide nucleic acid (see, for example, Egholm et al., Nature 1993, 365:566-568. See, also, U.S. Pat. No. 5,539,083).

In an alternative embodiment, the binding (hybridization) sites are made from plasmid or phage clones of genes, cDNAs (e.g., expressed sequence tags), or inserts therefrom (Nguyen et al., Genomics 1995, 29:207-209). In yet another embodiment, the polynucleotide of the binding sites is RNA.

Attaching nucleic acids to the solid surface. The nucleic acids or analogues are attached to a solid support, which may be made from glass, plastic (e.g., polypropylene, nylon), polyacrylamide, nitrocellulose, or other materials. A preferred method for attaching the nucleic acids to a surface is by printing on glass plates, as is described generally by Schena et al., Science 1995, 270:467-470. This method is especially useful for preparing microarrays of cDNA. See also DeRisi et al., Nature Genetics 1996, 14:457-460; Shalon et al., Genome Res. 1996, 6:639-645; and Schena et al., Proc. Natl. Acad. Sci. USA 1995, 93:10539-11286.

A second preferred method for making microarrays is by making high-density oligonucleotide arrays. Techniques are known for producing arrays containing thousands of oligonucleotides complementary to defined sequences, at defined locations on a surface using photolithographic techniques for synthesis in situ (see, Fodor et al., Science 1991, 251:767-773; Pease et al., Proc. Natl. Acad. Sci. USA 1994, 91:5022-5026; Lockhart et al., Nature Biotech. 1996, 14:1675. See, also, U.S. Pat. Nos. 5,578,832; 5,556,752; and 5,510,270) or other methods for rapid synthesis and deposition of defined oligonucleotides (Blanchard et al., Biosensors&Bioelectronics 1996, 11:687-90). When these methods are used, oligonucleotides (e.g., 20-mers) of known sequence are synthesized directly on a surface such as a derivatized glass slide. Usually, the array produced is redundant, with several oligonucleotide molecules per RNA. Oligonucleotide probes can be chosen to detect alternatively spliced mRNAs.

Other methods for making microarrays, e.g., by masking (Maskos and Southern, Nuc. Acids Res. 1992, 20:1679-1684), may also be used. In principal, any type of array, for example, dot blots on a nylon hybridization membrane (see, Sambrook et al., Molecular Cloning—A Laboratory Manual (2nd Ed.), Vol. 1-3, Cold Spring Harbor Laboratory, Cold Spring Harbor, N.Y., 1989), could be used, although, as will be recognized by those of skill in the art, very small arrays will be preferred because hybridization volumes will be smaller.

Generating labeled probes. Methods for preparing total and poly(A)+ RNA are well known and are described generally in Sambrook et al., supra. In one embodiment, RNA is extracted from cells of the various types of interest in this invention using guanidinium thiocyanate lysis followed by CsCl centrifugation (Chirgwin et al., Biochemistry 1979, 18:5294-5299). Poly(A)⁺ RNA is selected by selection with oligo-dT cellulose (see Sambrook et al., supra). Cells of interest may include, but are not limited to, wild-type cells, drug-exposed wild-type cells, modified cells, and drug-exposed modified cells.

Labeled cDNA is prepared from mRNA by oligo dT-primed or random-primed reverse transcription, both of which are well known in the art (see, for example, Klug & Berger, Methods Enzymol. 1987, 152:316-325). Reverse transcription may be carried out in the presence of a dNTP conjugated to a detectable label, most preferably a fluorescently labeled dNTP. Alternatively, isolated mRNA can be converted to labeled antisense RNA synthesized by in vitro transcription of double-stranded cDNA in the presence of labeled NTPs (Lockhart et al., Nature Biotech. 1996, 14:1675). In alternative embodiments, the cDNA or RNA probe can be synthesized in the absence of detectable label and may be labeled subsequently, e.g., by incorporating biotinylated dNTPs or NTP, or some similar means (e.g., photo-cross-linking a psoralen derivative of biotin to RNAs), followed by addition of labeled streptavidin (e.g., phycoerythrin-conjugated streptavidin) or the equivalent.

When fluorescently-labeled probes are used, many suitable fluorophores are known, including fluorescein, lissamine, phycoerythrin, rhodamine (Perkin Elmer Cetus), Cy2, Cy3, Cy3.5, Cy5, Cy5.5, Cy7, Fluor X (Amersham) and others (see, e.g., Kricka, 1992, Nonisotopic DNA Probe Techniques, Academic Press San Diego, Calif.). It will be appreciated that pairs of fluorophores are chosen that have distinct emission spectra so that they can be easily distinguished.

In another embodiment, a label other than a fluorescent label is used. For example, a radioactive label, or a pair of radioactive labels with distinct emission spectra, can be used (see Zhao et al., Gene 1995, 156:207; Pietu et al., Genome Res. 1996, 6:492). However, because of scattering of radioactive particles, and the consequent requirement for widely spaced binding sites, use of radioisotopes is a less-preferred embodiment.

In one embodiment, labeled cDNA is synthesized by incubating a mixture containing 0.5 mM dGTP, dATP and dCTP plus 0.1 mM dTTP plus fluorescent deoxyribonucleotides (e.g., 0.1 mM Rhodamine 110 UTP (Perken Elmer Cetus) or 0.1 mM Cy3 dUTP (Amersham)) with reverse transcriptase (e.g., SuperScript.TM. II, LTI Inc.) at 42° C. for 60 min.

Hybridization to microarrays. Nucleic acid hybridization and wash conditions are chosen so that the probe “specifically binds” or “specifically hybridizes” to a specific array site, i.e., the probe hybridizes, duplexes or binds to a sequence array site with a complementary nucleic acid sequence but does not hybridize to a site with a non-complementary nucleic acid sequence. As used herein, one polynucleotide sequence is considered complementary to another when, if the shorter of the polynucleotides is less than or equal to 25 bases, there are no mismatches using standard base-pairing rules or, if the shorter of the polynucleotides is longer than 25 bases, there is no more than a 5% mismatch. Preferably, the polynucleotides are perfectly complementary (no mismatches). It can easily be demonstrated that specific hybridization conditions result in specific hybridization by carrying out a hybridization assay including negative controls (see, e.g., Shalon et al., supra; and Chee et al., supra).

Optimal hybridization conditions will depend on the length (e.g., oligomer versus polynucleotide greater than 200 bases) and type (e.g., RNA, DNA, PNA) of labeled probe and immobilized polynucleotide or oligonucleotide. General parameters for specific (i.e., stringent) hybridization conditions for nucleic acids are described in the definitions provided in Section 5.1, supra. When cDNA microarrays, such as those described by Schena et al. are used, typical hybridization conditions are hybridization in 5×SSC plus 0.2% SDS at 65° C. for 4 hours, followed by washes at 25° C. in low stringency wash buffer (e.g., 1×SSC plus 0.2% SDS) followed by 10 minutes at 25° C. in high stringency wash buffer (0.1×SSC plus 0.2% SDS). See, Shena et al., Proc. Natl. Acad. Sci. USA 1996, 93:10614). Useful hybridization conditions are also provided in, e.g., Tijessen, 1993, Hybridization With Nucleic Acid Probes, Elsevier Science Publishers B.V. See, also, Kricka, 1992, Nonisotopic DNA Probe Techniques, Academic Press, San Diego, Calif.

Signal detection and analysis. When fluorescently labeled probes are used, the fluorescence emissions at each site of a transcript array can be preferably detected by scanning confocal laser microscopy. In one embodiment, a separate scan, using the appropriate excitation line, is carried out for each of the two fluorophores used. Alternatively, a laser can be used that allows simultaneous specimen illumination at wavelengths specific to the two fluorophores and emissions from the two fluorophores can be analyzed simultaneously (see, Shalon et al., Genome Research 1996, 6:639-645). In a preferred embodiment, the arrays are scanned with a laser fluorescent scanner with a computer controlled X-Y stage and a microscope objective. Sequential excitation of the two fluorophores is achieved with a multi-line, mixed gas laser and the emitted light is split by wavelength and detected with two photomultiplier tubes. Fluorescence laser scanning devices are described in Schena et al., Genome Res. 1996, 6:639-645 and in other references cited herein. Alternatively, the fiber-optic bundle described by Ferguson et al., Nature Biotech. 1996, 14:1681-1684, may be used to monitor mRNA abundance levels at a large number of sites simultaneously.

Signals are recorded and, in a preferred embodiment, analyzed by computer, e.g., using a 12 bit analog to digital board. In one embodiment the scanned image is despeckled using a graphics program (e.g., Hijaak Graphics Suite) and then analyzed using an image gridding program that creates a spreadsheet of the average hybridization at each wavelength at each site. If necessary, an experimentally determined correction for “cross talk” (or overlap) between the channels for the two fluors may be made. For any particular hybridization site on the transcript array, a ratio of the emission of the two fluorophores can be calculated. The ratio is independent of the absolute expression level of the cognate gene, but is useful for genes whose expression is significantly modulated, e.g., by administering a drug, drug-candidate or other compound, or by any other tested event.

In one preferred embodiment of the invention, the relative abundance of an mRNA in two cells or cell lines tested (e.g., in a treated verses untreated cell) may be scored as perturbed (i.e., where the abundance is different in the two sources of mRNA tested) or as not perturbed (i.e., where the relative abundance in the two sources is the same or is unchanged). Preferably, the difference is scored as perturbed if the difference between the two sources of RNA of at least a factor of about 25% (i.e., RNA from one sources is about 25% more abundant than in the other source), more preferably about 50%. Still more preferably, the RNA may be scored as perturbed when the difference between the two sources of RNA is at least about a factor of two. Indeed, the difference in abundance between the two sources may be by a factor of three, of five, or more.

In other embodiments, it may be advantageous to also determine the magnitude of the perturbation. This may be done, as noted above, by calculating the ratio of the emission of the two fluorophores used for differential labeling, or by analogous methods that will be readily apparent to those of skill in the art.

6.5. Bioinformatics and Statistics

Those skilled in the art will readily appreciate that the MPHTS assays of this invention will, at least in preferred embodiments, track a large amount of data from many sources including, e.g., expression levels for a large number of different genes in a variety of different cell and tissue types and under a variety of different conditions. The invention therefore preferably makes use of methods in bioinformatics and statistical analysis to integrate such data. Such analysis tools include, for example, clustering and class partitioning algorithms that enable a user to summarize and visualize effects of multiple variables on relationships within a data set. In a particularly preferred embodiment, the MPHTS methods of this invention make use of a statistical analysis tool referred to as “Principal Component Analysis” or “PCA”. The technique is well known in the art and may be implemented, e.g., using commercially available software such as the Partek suite of pattern recognition tools (Partek Inc., St. Charles, Minnesota).

By PCA analysis of gene expression data from different brain areas and disease states, a user is able to readily identify whether the major source or sources of variance within the data set are correlated with the particular cells or tissue and/or whether such variance is correlated with a neuropsychiatric disorder of interest. An exemplary figure depicting this analysis is set forth here, in FIG. 2. Those skilled in the art will readily appreciate and/or be able to select appropriate cutoffs (e.g., a maximum significant p-value) for use in such methods.

Statistically significant changes in gene expression may also be identified by coordinately regulated genes in distinct pathways, as well as coordinate changes of multiple genes within a common pathway (e.g., genes involved in a common metabolic pathway or process). These provide an aggregate level of statistical significance that far exceeds the statistical significance obtained for the genes individually.

In preferred embodiments, RNA extraction and/or hybridization experiments are repeated at least once, and more preferably multiple times for each sample to assure statistically robust and reproducible results. Changes in gene expression that appear to be statistically significant may also be confirmed by an independent experimental technique such as real-time polymerase chain reaction (RT-PCR), quantitative in situ hybridization, immunohistochemistry and functional assays of the translated protein(s), all of which are ell known and routinely used in the art.

6.6. Uses of Gene Signatures in MPHTS

Once genes signatures or “efficacy genes” for a particular disorder have been identified and/or selected, they may be readily used, e.g., in screening assays to identify promising therapeutic compounds that may be useful for diagnosing and/or treating such a disorder. In particular, a candidate therapeutic compound may be identified in screening assays of the invention by identifying compounds that produce changes in the expression of signature genes that are similar to the changes observed in the ECS model, demonstrated in the exmples, infra. Such changes may be identified qualitatively, but are more preferably identified quantitatively; for example, by assigning a MPHTS “value” for each compound tested in the screening assay.

As an example, and not by way of limitation, such an MPHTS value may simply be the sum of changes in each signature gene's expression observed for a test compound in the screening assay. Preferably, these changes in the signature genes' expression levels are normalized as a percentage of the “optimal” change in each gene's expression. As used here, the change in expression of an signature gene is said to be “optimal” when it is approximately equal to the change in expression associated with a therapeutic benefit. Optionally, the change in each signature gene's expression can also be weighted, e.g., by the signature gene's score (as determined, e.g., using the algorithm set forth in Section 8.2, infra). The calculation of such a value can be easily represented mathematically by the equation:

$$V = {\sum }_{i} {ω}_{i} {E}_{i} \qquad ( Equation 1 )$$

In Equation 1, V is the MPHTS “score” calculated for a test compound in an MPHTS assay. Eᵢ is the measured change in the expression of gene i in cells contacted with the test compound compared to the expression in cells that are not contacted with the test compound. As noted above, Eᵢ will preferably be normalized to the “optimal” change associated with a desired therapeutic effect. For example, Eᵢ can be expressed as the percentage or fraction of optimal change. ωᵢ indicates the score for the signature gene i. In preferred embodiments, ωᵢ obtained or derived from the score value calculated for gene i (e.g., according to the algorithm set forth in Section 8.2) and is converted to a percentage of the average score value or the signature genes that comprise the entire set used for drug screening.

### 7. EXAMPLES

The present invention is also described and demonstrated by way of the following examples. However, the use of these and other examples anywhere in the specification is illustrative only and in no way limits the scope and meaning of the invention or of any exemplified term. Likewise, the invention is not limited to any particular preferred embodiments described here. Indeed, many modifications and variations of the invention may be apparent to those skilled in the art upon reading this specification, and such variations can be made without departing the invention in spirit or in scope. The invention is therefore to be limited only by the terms of the appended claims along with the full scope of equivalents to which those claims are entitled.

7.1. Gene Signatures of Electroshock Therapy as Modeled by Rodent Electroconvulsive Seizures

7.1.1 Materials and Methods

Subjects. Male, Sprague-Dawley rats (ACE Animals; Boyertwon, PA) were 2-6 months of age and weighted 200-350 g at the start of each experiment. Animals were housed 2 per cage, understandard conditions, including ad libitum access to food and water in a 12:12 hour light/dark vivarium.

ECS Delivery. Animals were handled for 3 days prior to the beginning of the experiment for them to become adjusted to the seizure induction area and to application of the ear-clip electrodes. The seizure induction or sham-seizure induction areas consisted of plastic cages with fresh bedding material. Each cage was placed on a lab bench and a pair of electrodes was hung over the top of each cage. One set of electrodes was connected to the ECT Unit (Ugo Basile # 57800, Comerio, Italy) for use with the ECS animals and the other set (for control animals) was disconnected from the ECT unit such that no current could be delivered. Cage mates were run concurrently: one animal was randomly assigned to the shock group and the other to the control group. Both animals were removed from their home cage and placed in the appropriate testing cage. Each set of ear-clip electrodes were dampened with saline and attached to the deep region of the outer ear. The shock, a square wave pulse (pulse width=0.7 ms, 50 Hz) of 50 mA (Madsen et al., Biol. Psychiatry 2000, 47:1043-1049) was administered for a duration for 1.0 second to provide a tonic phase characterized by extension of all limbs and forward head extension, lasting for 10-15 seconds. Animals, both ECS and control, were returned to their home cage 10 minutes after seizure induction. Acute ECS animals (N=12) were given a single shock. Chronic ECS animals (N=12) were given one ECS per day for 10 consecutive days. Control group rats (N=12 acute and N=12 chronic) followed this protocol.

Visual observation of behaviors, side effects. Visual observations were sufficient to confirm the presence of clonic-tonic seizures immediately following the shock. Side effects during the post-ictal phase, which lasted about 10 minutes, were best characterized by heightened responsivity to auditory and tactile stimulation and decreased motor activity. General behavioral observations made in the home cage for up to 6 hours post-shock administration revealed sedation but no signs of recurrent seizures.

Brain area dissections. Animals were sacrificed by decapitation without anesthesia approximately 3-5 hours after the final ECS treatment. The brain was removed from the calvarium and immersed in crushed ice for 3-5 minutes. Coronal slabs of 2 mm thickness were prepared, from which the frontal cortex and hippocampus were dissected. The brain regions were weighed, frozen on dry ice, and stored at −80° C.

RNA Extraction. Total RNA was extracted from about 70-100 mg of frozen rat tissue with 0.7-1.0 ml of TRIzol reagent (Invitrogen; Carlsbad, Calif.) as per the manufacturer's instructions. The tissue was homogenized with a Polytron (Fisher) for 30 seconds. The homogenate was cleared by a 10 minute centrifugation at 10,000×g, and 0.2 ml of chloroform was added per ml of TRIzol. The samples were vigorously shaken for 20 seconds and incubated on ice for 10 minutes. The aqueous phase was separated by centrifugation at 10,000×g for 10 minutes, allowed to precipitate for 10 minutes, and the precipitate collected by centrifugation at 12,000×g for 10 minutes. The pellet was washed with 70% ethanol collected by a brief centrifugation, air dried, and resuspended in H₂O. The RNA was further purified using an RNeasy column (Qiagen; Valencia, Calif.). The purified RNA was quantified by a UV spectrophotometer, and RNA quality determined by capillary electrophoresis on an Agilent Bioanalyzer.

Gene expression profiling. RNA (10 μg) was converted to double-stranded cDNA following priming with an oligo-dT-T7 primer (Operon). The resultant cDNA was purified by phenol-chloroform extraction, ethanol-precipitated, and resuspended in H₂O. The purified cDNA was subjected to in vitro transcription using T7 polymerase in the presence of biotinylated UTP and CTP (Enzo Life Sciences, Farmingdale, NY). The resultant aRNA was purified with an RNeasy column (Qiagen, Valencia, Calif.), eluted in H₂O, and quantified using a UV spectrophotometer. aRNA (15 μg) was fragmented following the Affymetrix protocol. The quality of RNA fragmentation was checked on the Agilent Bioanalyzer, and 30 μl was added to 270 μl of hybridization buffer and hybridized to the Affymetrix U34A Rat Genome GeneChip®. After 16 hours of hybridization at 45° C. the GeneChip was washed, stained, and scanned according to the standard Affymetrix protocol.

Statistical analysis. Each gene on Affymetrix U34A gene chips is represented by one or more of about 8800 “probe sets”—i.e., sets of oligonucleotides that are derived from the mRNA sequence of that particular gene. Following hybridization and scanning, each probe set was assigned a “present” or “absent” call and an overall fluorescent signal intensity as determined by the Affymetrix Microarray Suite Software 5.0 following global normalization. A filtering of all probe sets was conducted to remove those genes that were clearly not expressed in the tissue, and such probe sets were not analyzed. Specifically, a probe set for a particular gene was retained for all samples only if it contained a “present” call for at least one sample, and only if the mean fluorescence intensity for either the control or treatment group was greater than 100. This procedure filtered out about 50% of all probe sets, consistent with the known expression of approximately 40-50% of rat genes within brain tissue. Absent or present calls are assigned by Affymetrix software on the basis of a comparison between the intensities of perfect oligonucleotide matches and single oligonucleotide mismatches. The finding that mismatch intensities are not significantly lower than those for the perfect match oligonucleotides does not necessarily imply that the gene is not expressed, as it may be a result from cross-hybridization of other mRNA species, other technical artifacts, or measurement noise. Therefore, present calls were counted in a liberal manner to avoid discarding probe sets where there was some indication that the gene may in fact be expressed. The alteration of many genes in more than one tissue or after both durations of ECS, and the confirmation of many gene changes by RT-PCR, confirms that measurements of some genes with relatively few present calls would be detected in independent tissues and by an independent method.

A principal components analysis (PCA) was performed using Partek Pro 2000 software package (Partek, Inc.). The analysis treated arrays as “objects” and probe sets as “variables” to detect outlier arrays. PCA plots mapped the distribution of arrays within a 3-dimensional space defined by the three greatest contributors to the variance in gene expression values. The position of an array on such plot reflects the overall expression profile of the sample as measured by the array. The arrays that appeared as outliers on the plots were removed from further consideration. Such PCA outliers are usually associated with a technical problem, such as marginally acceptable RNA quality, regional blotches in hybridization signals, or weak overall hybridization on the corresponding gene chip. For each of the four experiments, (acute and chronic ECS, frontal cortex and hippocampus), at least seven, but typically nine or all 10, of the rat samples from each control or treatment group were retained for further analysis.

The ability of acute or chronic ECS to alter gene expression versus that of sham-treated controls was determined for each tissue type. The magnitude of expression level change was estimated as the ratio of the means between the treated and control groups. Probe sets were considered as “hits” only when the average normalized signal intensity exceeded 100 in either of the groups, the ratio of means exceeded 1.5 or were less than 0.67 and the p value of the two-tailed Welch t test was less than 0.05.

In a multiple testing setting, the p value of 0.05 does not necessarily mean that the probability of type 1 error is really 0.05, unless the Bonferroni's correction has been applied. Bonferroni's correction was not applied in these experiments, however, because it would result in many type 2 errors and the loss of most valid hits. The uncorrected p value of a statistical test is therefore useful here primarily as a measure of difference between data sets. According to a recent study (Xu & Li, Bioinformatics 2003, 19:1284-1289), a parametric p value may outperform a permutational one as a distance metric. Additionally, the 1.5 ratio of means cut-off used here increased the verification rate of gene expression changes.

GeneSpring® analysis software (Silicon Genetics) was used to evaluate the overlap between the gene changes identified in the four comparisons. Since about 3,500-4,400 genes were present in each sample, a significant number of false positives was expected. To help remove false positives, and also to increase confidence in selecting gene hits for RT-PCR confirmation, each statistically significant gene change was ranked objectively by a biostatistical ECS gene selection algorithm (set forth in the appendix at Section 8.2, infra) according to its statistical and biological significance, and by the robustness of experimental change, including whether it was altered in one or both tissues and after acute, chronic, or both ECS durations.

Real-time polymerase chain reaction assay (RT-PCR). Total RNA (2 μg) were subjected to DNAse treatment in a 10 μl reaction containing 1 μl 10×DNAse I reaction buffer, and 1 μl DNAse I (Invitrogen, Carlsbad, Calif.). The reaction was carried out at room temperature for 10 minutes. One μl of EDTA (25 mM) and 1 μl of oligo (dT)_{12-18 mer} (0.5 μg/ml, Invitrogen, Carlsbad, Calif.) were added to DNAse reaction and heated to 70° C. for 15 minutes in a water bath to simultaneously inactivate the DNAse I activity and eliminate RNA secondary structure to allow oligo dT-poly A annealing. The sample was placed on ice for 2 minutes and collected by brief centrifugation. The RNA in the sample was reverse-transcribed into cDNA by the addition of 8 μl of master mix containing 4 μl of 5×first strand buffer, 2 μl DTT (0.1 M), 1 μl dNTP's (10 mM each), and 1 μl SuperScript II (200U/μl) (Invitrogen, Carlsbad, Calif.), and incubated at 42° C. for 45 minutes. The RT reaction was diluted 10 fold with dH₂O and stored at 4° C.

For each specific gene tested, diluted cDNA (5 μl) was added to 45 μl PCR reaction mixture that contained 25 μl of 2×SYBR® Green PCR Master Mix (Applied Biosystems, Warrington, UK) and 25 picomole of each forward and reverse primer. Each sample was subjected to 40 cycles of Real Time PCR (ABI 7900, Applied Biosystems, Warrington UK) where fluorescence is measured several times during each cycle of 2-step PCR alternating between about 95° C. for 15 seconds and 60° C. for 1 minute. The threshold cycle (Ct), or point at which signal fluorescence exceeds background, for each sample for each gene was compared to a standard curve to determine a relative expression value. The standard curve was generated by real time PCR analysis of five, 10-fold serial dilutions of a cDNA generated from the RNA of rat cortical stem cells. This method allows a relative comparison between samples. The expression value of each gene was normalized to the relative amount of GAPDH expressed in that sample to calculate a relative amount of transcript present for each gene. The normalized expression values for all control and treated samples were averaged and an average fold change determined. A Student's t test was conducted between the normalized relative expression values for each individual control and treated samples to determine statistical relevance.

7.1.2 Results

Four sets of microarray hybridizations to evaluate the effects of acute or chronic ECS treatment on gene expression in the frontal cortex and hippocampus. Plots of the mean abundances of each probe set measured with the Affymetrix U34A rodent chip are illustrated in FIGS. 1A-1D the frontal cortex (FIGS. 1A and 1B) and hippocampus (FIGS. 1C and 1D) of rats that with exposed to either acute (FIGS. 1A and 1C) or chronic (FIGS. 1B and 1D) ECS, compared to sham control groups that received no ECS. At gene abundances above 100, each scatter plot of mean intensities diplayed minimal skewing about the unity line. The distribution of points was generally quite close to the unity line, and statistically significant outliers (illustrated by white dots in FIGS. 1A-1D) were frequently well outside the main clusters of genes. Principal components analysis (PCS) revealeded that the frontal cortex and hippocampus transcriptome segregated into distinct clusters similar to that reported for different human CNS brain regions (Palfreyman et al., Current Drug Targets 2002, 1:205-216). Outlier samples in the PCA analysis could be readily distinguished, and either one sample, two samples or no sample group were removed from the statistical analysis so that the final n was 8-10 per group.

Data quality evaluation. In order to first determine the number of samples per group needed to reliably identify differentially expressed genes, subgroups of 2 to 10 samples were randomly created from the sham- or ECS-treated groups (or from these two groups combined) of gene expression data in the acute hippocampus. Welch t tests were performed for all gene changes comparing subgroups within or between the sham control and ECS groups as well as between “mixed” subgroups, and this was repeated for 100 different comparisons. Each time, the number of “hits” passing p value cut-off of 0.01 was counted, and these data are plotted in FIG. 2. Statistical theory predicted that about 40 genes are expected to appear as hits due to random technical and biological variations from the 3,500 to 4,400 detected genes, independent of any real ECS treatment effect. Comparisons between subgroups of samples drawn from within a group of animals or from the “mixed” animals produced numbers of significant gene changes that were mostly below this theoretical chance level, and gradually approached it with increases in subgroup size, as can be seen from visual inspection of FIG. 2. In contrast, the number of significantly changed genes in the analysis of sham-versus ECS-treated animals increased steeply from chance level at group size of 2, to 15-fold above chance at group sizes of 10. This analysis demonstrates that there is actually a highly significant effect of the ECS shock treatment on gene expression measured by the microarrays. The analysis also demonstrates that, while greater numbers of animal per group increase statistical power, only 8-10 animals per group are necessary to reliably detect large numbers of changes in gene expression levels.

Identification and ranking of differentially expressed genes. A total of 135 unique genes, represented by more than 200 probe sets, were found to be differentially expressed in ECS-treated rats compared to controls in at least one of the four different ECS experiments. These genes are listed in the Appendix, infra (see, in particular, at Section 8.3) along with the Accession number for their nucleotide sequence on the GenBank database. The Table in Section 8.3 also provides the expression ratio and p-value determined for each of these genes in the microarray analysis, as well as the “score” determined using the algorithm set forth at Table 1, supra. Many of these genes, whose names are shaded in the table at Section 8.2, have not been previously associated with either ECS or ECT.

A Venn diagram representing the distribution of changes among the 135 genes in different tissues and ECS treatment groups is shown in FIG. 3. Forty-four genes were identified that had significant differential expression in multiple tissues and/or in multiple ECS treatment groups. The most dramatic response to ECS treatment occurred in the hippocampus, where 87 genes were detected as differentially regulated following a single, acute shock. Chronic ECS treatment resulted in fewer gene changes in the hippocampus (64 compared to 87), about half of which were distinct from those that changes after a single ECS. The frontal cortex was much less responsive than the hippocampus, with 36 genes identified as differentially regulated following acute treatment, and 36 genes identified as differentially regulated by chronic ECS treatment. Twelve genes that were regulated in the same direction in the hippocampus by ECS and following exercise (Molteni et al., Eur. J. of Neurosci. 2002, 16:107-116; Tong et al., Neurobio. of Disease 2001, 8:1046-1056) are indicated in Section 8.3 by an asterix (*) next to the gene name. A supplemental list of genes with p values less than 0.005 and having differential expressing changes less than 1.5 fold was also identified. These genes are listed separately, infra, in Section 8.4 infra. As with the genes listed in Section 8.3, many of the genes listed in Section 8.4 have not been previously associated with either ECS or ECT. These genes' names are shaded in the tables at Sections 8.3-8.4.

Many genes are represented by more than one probe set on the Affymetrix RG-34A. chip. Among genes exhibiting significant change due to ECS treatment, 13 were identified by multiple probe sets. These genes are indicated in Tables of Sections 8.3 and 8.4 by italicized type in those tables. Five of these thirteen genes were selected to determine if all of the probe sets for each gene were in agreement. The numbers of probe sets reporting significant and co-directional change due to ECS treatments were as follows: five out of seven for BDNF; two out of two for Cox-2; two out of four for Jun, two out of three for TIEG, and 1-2 out of four (depending on the treatment and tissue) for catalase. The remaining five probe sets for BDNF, Jun and TIEG, as well as one of the catalase probe sets, produced only absent calls and/or less than 100 abundance units in all rat brain samples. These findings suggests that some of these probe sets simply failed to measure their target mRNA, and that ECS actually did affect at least four out of these five genes.

Twenty-six of the identified genes in Section 8.3 were found to be similarly up- or down-regulated in both the hippocampus and frontal cortex (FIG. 3), indicating that many brain-wide changes following shock therapy may occur independent of cellular heterogeneity of these different regions. In addition, 39 gene were found to change after both acute and chronic ECS treatments. Of the genes that were either up- or down-regulated in more than one tissue type and/or after both acute and chronic ECS therapies, one gene changed in different directions. This gene, solute carrier number 3, member 1, decreased expression in both the frontal cortex and hippocampus following chronic ECS, but increased expression in the hippocampus after acute ECS. The expression of five genes in particular was similarly affected by ECS in every experiment (labeled as “all” in the Table of Section 8.3)—i.e., following both chronic and acute ECS treatment in both the hippocampus and frontal cortex. These five genes are: brain derived neurotrophic factor (BDNF), prostaglandin-endoperoxide synthase 2 (COX-2), neuronal activity-regulated pentraxin (Narp), TGFβ-inducible early growth response, and tissue inhibitor of metalloproteinase 1 (TIMP-1). Each of these genes or their biological pathways have been previously reported to change individually following either ECS treatment or seizure in rats (see, in particular, Zetterstrom et al., Brain Res. Mol. Brain Res. 1998, 57:106-110; Nibuya et al., J. Neurosci. 1995, 15:7539-7547; and Hashimoto, Brain Res. 1998, 804:212-223). However, the combined changes in expression of these genes has not heretofore been demonstrated.

About two-thirds (i.e., 92 out of 135) of genes listed in Section 8.3 are changed their expression in only a single experiment. Hence, acute and chronic ECS can influence gene expression in different ways, and genes in the hippocampus and frontal cortex often respond differently to ECS treatment.

Verification of gene signatures by RT-PCR. The measurement of expression levels for a large number of genes can result in a significant number of false positive results (Zhang, Proc. Nat. Acad. Sci. U.S.A. 2002, 99:12509-12511; Zhou et al., Proc. Nat. Acad. Sci. U.S.A. 2000, 99:12783-12788; and Xu & Li, Bioinformatics 2003, 19:1284-1289). To confirm the validity of gene expression changes in these experiments, the expression levels of sixty genes were retested using quantitative real time polymerase chain reaction (RT-PCR) using an independent sampling of the RNA samples in the microarray analysis. Approximately 1,200 RT-PCR assays were conducted with hippocampal or frontal cortex tissues after actue or chronic ECS, focussing on those genes with the largest fold change and the best statistical signficance in the microarray analysis. Genes were also selected for confirmation by RT-PCR to provide a representation of various protein classes and for their biological relevance to depression or its treatment. The results of this analysis are presented infra, in Section 8.5 of the appendix. Genes were identified with a liberal p<0.05 criteria but required at least a 1.5-fold increase or decrease to be considered significant. The abbreviations “HCA” and “HCC” refer to genes identified in the hippocampus following either acute or chronic ECS treatment, respectively. The abbreviation FCA indicates that a gene was identified in the prefontal cortex following acute ECS treatment.

A “first pass” validation of the microarray results indicated a 50% validation rate. Principal failures in these experiments were apparently mostly caused by low technical performance of individual primer pairs, as evidence by a failure of the amplified signal to reach asymptotic intensity levels characteristic of genes that are efficiently amplified. For those primer pairs that failed, a second and unique primer pair was selected from the same region of the transcript to generate an amplicon the same length as the original primer pair. Non-validated genes were retested with the new primer pairs.80% of these RT-PCR retested genes produced the expected asymptotic signal across their amplification cycles, and validated results obtained in the microarray analysis.

The RT-PCR experiments confirmed statistically significant differential expression for 43 out of the 60 genes tested (i.e., 63%). In many instances, the average magnitude of the change in expression for a given gene was only 30% between the two groups. However, individuals within those different two groups would often differ by a factor of two or more. Hence, the RT-PCR procedure was used to quantify differences between individuals that exceed differences between the different experimental groups.

RT-PCR produces a false negative rate of approximately 30% for any large set of genes that has been found to be significantly changed on a microarray platform. Only a few genes exhibited high, asymptotic intensity levels during RT-PCR while, at the same time, failing to replicate microarray results because of low statistic significance. However, these nevertheless displayed a magnitude and direction of gene abundance change that were similar to those seen with the microarrays. Therefore, it is more likely that greater than 63%, and possible closer to 80% of the genes detected by microarray analysis of ECS tissues (i.e., in Sections 8.3 and 8.4) represent true positives based on RT-PCR validation. Repeated observations of expression changes within different tissues for these genes, confirmation of the magnitude and direction of expression changes for 63 to 80% of these genes by RT-PCR, and the substantial agreement of many observed changes with other literature findings all confirm that the results reported here are valid.

7.2. Selection of Genes for ECS Gene Signatures

To select differentially expressed genes that are most informative and therefore useful in an ECS signature, e.g., for MPHTS type screening assays of the invention, the genes listed in Sections 8.3 and 8.4, infra, were prioritized for overall significance using the algorithm set forth in Section 8.2. This algorithm is based on statistical, biological and experimental considerations that account for factors such as a gene's known relationship (if any) with bipolar affective disorder and/or unipolar major depression—as well as accounting for the magnitude and/or statistical significance (e.g., the p-value) of the gene's differential expression in one or more ECS experiments. The algorithm minimizes biases (for example, favoring genes that change in one particular region or tissue) that are likely to occur when other methods of selection are used. As such, algorithms such as in the one in Section 8.2, infra, provide an objective and preferable way to identify informative genes that can be used in an ECS gene signature.

For each gene listed in Sections 8.3-8.4, a numerical value was assigned for the three aspects listed in Section 8.2—i.e., for statistical considerations (I), for biological considerations (II) and for experimental considerations (III). The numerical value(s) for each consideration were computed as described in Section 8.2. For biological considerations (II) a numerical value was assigned for each gene based on reports from the literature (if available).

The algorithm score thus calculated for each gene is set forth in the far right-hand column of the tables at Sections 8.3-8.4. 18 out of the 20 genes most highly ranked by the algorithm have been identified by prior investigators to change after ECS, whereas none of the 30 most lowly ranked genes have been previously associated with ECS. Yet, prior knowledge about a gene's change in response to ECS accounts for only about 2% of the algorithm's total score, demonstrating that the algorithm is able to independently prioritize genes according to their known response in ECS. In contrast, ranking genes according to only the level of their expression change and/or its p-value produces a more random distribution of the genes that have been previously associated with ECS.

To better identify differentially expressed genes that are particularly preferable, e.g., for use in an ECS gene signature, a biological consideration score (II) was recalculated for each gene—this time using experimental data from homologous genes in human cells and/or tissues. Specifically, expression levels of homologous genes were obtained from “disease signatures” in human tissue samples that had been obtained from individuals diagnosed with a neuropsychiatric disorder. In additiona, expression levels of homologous genes in “drug signatures” from neuronal cells or animals treated with neuropsychiatric drugs (e.g., valproate, clozapine, carbamazepine, and lithium) were also considered. Such “drug signatures” and “disease signatures” have been previously described, as have routine methods and techniques by which they can be obtained. See, in particular, U.S. patent application Publication No. 2003/096264 A1.

Homologos and/or orthologs of the rat genes listed in the appendices, infra, can be readily identified, for example by their level of sequence identity to the recited rat nucleic acid sequences, or by the level of sequence identity and/or homology to amino acid sequences that they encode. Alternatively, homologs and orthologs (including those from other species, such as humans and mice) can be identified by hybridization under conditions of appropriate stringency, such as those described, supra, in Section 6.1. In a preferred embodiment, appropriate homologs and/or orthologs are identified by routine clustering algorithms. For example, the NCBI UniGene database (accessed via the URL <http://www.ncbi.nlm.nih.gov/UniGene>) groups genes into appropriate clusters of homologous sequences from the same and/or different species of organism. See, Schuler, J. Mol. Med. 1997, 75(10):694-698; Schuler et al., Science 1996, 274:540-546; and Boyuski & Schuler, Nature Genetics 1995, 10:369-371.

Section 8.6 below provides a list of genes that are differentially expressed in the ECS experiments (Section 7.1, supra) along with the rat, human and mouse UniGene cluster number for each gene and its homologs. The GenBank Accession number for a representative nucleotide sequence (or fragment thereof) for each gene is also provided. Overlapping expression data for each gene in human tissue, human cells, and mouse/rat tissue is also provided, where available. The column “PGI II” gives the algorithm score for biological considerations (II) calculated using this additional information. The next column (“Groups I, II (Literature) and III”) provides algorithm scores computed using biological consideration information from the literature—i.e., the algorithm scores set forth in preceding Sections 8.3 and 8.4. The far right-hand column in Section 8.5 gives the “ECS algorithm score”—i.e., the sum of the original score and PGI II.

The actual number of ECS signature genes used in a particular application (i.e., for a particular “gene signature) may vary and generally depends on the need for additional information afforded by including more signature genes, balanced by considerations such as the cost and/or effort involved in addition additional signature genes, e.g., in a microarray or PCR assay platform. As an example preferred gene signatures of the invention comprise nucleic acids corresponding to five or more of the genes set forth in Sections 8.3-8.5, infra. In other embodiments, a gene signature of the invention can comprise nucleic acids corresponding to and/or capable of detecting at least 5, 10, 15, 20, 25, 30, 35, 40, 45, 50, 60, 70, 80, 90, 100, 110, 120, 130, 140, 150 or more of the signature genes set forth in Sections 8.3-8.5, infra.

Preferably, signature genes that are most informative are used. Such genes can be identified, for example, by using an algorithm such as the one set forth in Section 8.2, infra, or another objective algorithm that objectively ranks genes for use in an ECS signature. The table set forth at Section 8.5 below, which lists signature genes according to their ECS algorithm score, is therefore particularly useful for selecting useful and preferred genes in an ECS gene signature. Hence, as an example, preferred gene sgiantures of this invention comprise nucleic acids that correspond to and/or are capable of detecting at least the first 5, 10, 15, 20, 25, 30, 35, 40, 45, 50, 60, 70, 80, 90, 100, 1120, 120, 130, 140, 150 or all of the signature genes list in Sections 8.5—i.e., nucleic acids corresponding to and/or capable of detecting at least SEQ ID NOS:1-5; 1-10; 1-15; 1-20; 1-25; 1-30; 1-35; 1-40; 1-45; 1-50; 1-60; 1-70; 1-80; 1-90; 1-100; 1-110; 1-120; 1-130; 1-140; 1-150; or 1-152.

Alternatively, a gene signature of the invention may comprise nucleic acids that correspond to and/or are capable of detecting orthologs and/or homologs of these genes. As noted above, homologos and/or orthologs of the rat genes listed in the appendices, infra, can be readily identified, for example by their level of sequence identity to the recited rat nucleic acid sequences, or by the level of sequence identity and/or homology to amino acid sequences that they encode. Alternatively, homologs and orthologs (including those from other species, such as humans and mice) can be identified by hybridization under conditions of appropriate stringency, such as those described, supra, in Section 6.1. In a preferred embodiment, appropriate homologs and/or orthologs are identified by routine clustering algorithms. For example, the NCBI UniGene database (accessed via the URL <http://www.ncbi.nlm.nih.gov/UniGene>) groups genes into appropriate clusters of homologous sequences from the same and/or different species of organism. See, Schuler, J. Mol Med. 1997, 75(10):694-698; Schuler et al., Science 1996, 274:540-546; and Boyuski & Schuler, Nature Genetics 1995, 10:369-371.

As a particular example, and not by way of limitation, the gene signature of the invention can be used, e.g., in detection kits (for example in any of the applications described in Sections 7.3-7.5, below) for detecting one or more signature genes. Examples of such kits include, but are not limited to, microarrays and PCR reaction kits to name a few. Preferred kits will comprise probes and/or primers for amplifying and/or detecting one or more ECS signature genes. Hence, a typical kit of the invention will comprises nucleic acids (preferably oligonucleotides) that specifically hybridize to ECS signature genes of the invention under defined conditions (which will depend on the particular application for which the kit is used, and include hybridization conditions described in this specification supra).

Kits of the invention will typically comprise at least one probe or primer which is capable of specifically hybridizing (e.g., understringent conditions) to an ECS signature gene. Preferred kits comprises a plurality of probes or primers, each capable of specifically hybridizing to a different ECS signature gene. PCR reaction kits will typically comprise a at least one pair of primer (i.e., a primer pair) that is capable of specifically amplifying an ECS signature gene. Hence, a primer pair in such kits will typically include a “forward” primer and a “reverse” primer. Preferred PCR kits of the invention comprise a plurality of primer pairs, each of which is capable of amplifying a particular ECS signature gene by PCR. PCR kits of the invention also include reverse transcription PCR (RT-PCR) kits, that are capable of converting mRNA from ECS signature genes into cDNA, and then amplifying said cDNA in a PCR reaction. Hence, RT-PCR kits of the invention will also typically comprise at least one primer pair, and may also comprise a “reverse primer;” i.e., an oligonucleotide that is capable of priming the reverse transcription of mRNA from an ECS signature gene. Preferred RT-PCR kits comprise a plurality of primer pairs and reverse primers, which are capable of reverse transcribing and amplifying mRNAs for a plurality of different ECS signature genes.

In some embodiments, the reverse primer is a separate and distinct oligonucleotide that is different from either the forward or reverse primer. More preferably, however, either the forward or reverse primer for amplifying an ECS signature gene is also capable of reverse transcribing mRNA of that ECS signature gene, and so also functions as a reverse transcription primer in an RT-PCR kit.

Kits may contain additional reagents or other components for detecting ECS signature genes, or such additional components may be provided by a user. For example, preferred kits of the invention may contain at least a polymerase enzyme and one or more labeled nucleotides. A kit of the invention may also contain buffer or other reagents.

7.3. Evaluating Patient Treatment Using ECS Gene Signatures

The ECS gene signatures identified herein (e.g., in Sections 8.3-8.5, infra) can be used in evaluating the efficacy of a treatment of a patient suffering from depression. In this evaluation, a peripheral blood sample is taken from the patient before the treatment, and at 1 day or 1 week after initiating the treatment. Lymphocytes or other accessible peripheral cell types are isolated, and the level of expression of all genes within these cells, or a subset of genes indicated in Tables 5-7, is then determined by one or more quantitative RNA measuring techniques. For example, the sample can be treated with Trizol reagent and other chemical procedures in order to prepare RNA. Probes and microarrays for the analysis are prepared using methods established for gene expression analysis, such as by any one of the commonly available quantitative mRNA detection methods, e.g., reverse transcriptase-polymerase chain reaction (RT-PCR) or microarray analysis using commercially available chips. The alteration of gene expression is quantified in the lymphocyte sample(s) and determined to be either higher or lower than the value obtained in the pre-treatment, baseline sample(s).

Resulting data showing gene changes in the same direction as those obtained in the ECS samples sets indicates indicate that the treatment method is effective in treating depression in the patient, whereas a gene change in the opposite direction indicates that the treatment is less effective for this purpose.

7.4. Use Of ECS Gene Signatures To Identify Therapeutic Compounds

The gene signatures provided in Sections 8.3-8.5, infra, can be used in screening for antidepressive agents. In this screening method, a set of genes is selected as those which change significantly following ECS treatments (see Tables 5-7), and the corresponding probes are selected by having sequence identity with a significant region of the selected genes. Methods and procedures that are common to those skilled in the art of quantifying RNA levels in cells, using, e.g., reverse transcriptase-polymerase chain reaction (RT-PCR), microarray analysis with commercially, high-density cDNA or oligonucleotide-based chips, or miniarrays that incorporate relatively small (e.g., 10-300) gene sequences corresponding to the genes listed in Sections 8.3-8.5, are used. Microplates wherein each well contains a fixed number of cultured neuronal cells from a neuronal, neuroblastoma, astrocytic, or glial cell line, or a primary neuronal or lymphoblast culture, or another suitable cell culture, are prepared and used as described in the co-pending U.S. patent application Ser. No. 10/175,523, filed Jun. 18, 2002.

The assay is conducted by contacting neuronal cells with a number of test agents, exposing the cells to the test agents for 1-4 days, and the effect on the expression of the select set of genes is evaluated. Resulting data showing gene changes that are in the same direction as those identified following ECS exposure to rats, particularly those summarized in Tables 5-7, for a test agent shows that the agent can be effective in treating or preventing depression.

7.5. Diagnostic Uses of ECS Gene Signatures

The gene signatures identified in Section 8.3-8.5, infra, can also be used for the prognosis or diagnosis of a patient suspected to be suffering from, or at risk for developing, a depression.

In this evaluation, samples of blood or other accessible tissues are obtained before, and at 1, 2, 4 and additional weeks after, treatment. Circulating lymphocytes (white blood cells) or are obtained from the samples and subjected to gene expression analysis using any one of the commonly available quantititaive mRNA detection methods, such as reverse transcriptase-polymerase chain reaction (RT-PCR), microarray analysis using commercially available chips, or other methods. The alteration of gene expression is quantified in post-treatment sample(s) and determined to be either higher or lower than the value obtained in the pre-treatment, baseline sample(s).

Resulting data showing changes in gene expression that are in the opposite direction as gene changes associated with ECS as listed in Tables 5-7 indicates that the patient is suffering from depression or is at risk for developing a depression, whereas the change in the same direction, or absence of a change, indicates a lower risk for depression. In response to an putative antidepressant agent (to determine prognosis of disease following initial baseline assessments), changes in gene expression will have prognostic value as well. In this example of the embodiment of the invention, resulting data that showed changes in gene expression that are in the same direction as gene changes associated with ECS as listed in Tables 5-7 indicate that the therapeutic agent is improving the patients state of depression, whereas the change in the opposite direction, or absence of a change indicates a lower effectiveness of the antidepressant potential of the test agent.

8. Appendix

8.1. Electroconvulsive Shock (ECS) Therapy and Gene Regulation (Literature Review)

| | | Post final | |
|---|---|---|---|
| | ECS | shock tissue | |
| Author, YR | level/dose | collection | Altered Genes and Brain region |
| Kim, et al., Mol Cells 2001, | Single shock | 3-24 hours | Decreased Inositol 1,4,5- |
| 12: 173-177 | | (hrs) | triphosphate receptor 1 (InsP3R1) |
| | | | mRNA in dentate gyrus and CA1 |
| Jensen et al., J | 14 days | | Decreased POMC mRNA in the |
| Neuroendocrinol 2001, | | | Hypothalamic-pitutitary-adrenal |
| 13: 887-893. | | | (HPA) axis |
| Jang et al., Prog | Chronic | | Adenylate cyclase (AC) isoforms |
| Neuropsychopharmacol Biol | | | in hippocampus and cerebellum |
| Psychiatry 2001, 25: 1571-81 | | | |
| Kondratyev et al., Brain Res | Low- | | ECS treatment prevented elevation |
| Mol Brain Res 2001, 91: 1-13. | sensitivity | | of Bcl-XS mRNA after prolonged |
| | (minimal) for | | seizure activity in hippocampus |
| | 7 days | | and rhinal cortex |
| Koubi et al., Brain Res | Acute shock | 72 hrs | Decreased TH and TpOH protein |
| 2001, 905: 161-170 | | | levels in Locus Cereuleus (LC), |
| | | | Central tegmental area and Raphe |
| | | | Centralis (TpOH only) mRNA |
| | | | changes only in LC; increase |
| | | | TpOH protein levels in Frontal |
| | | | cortex and increase TH protein in |
| | | | hippocampus |
| Shen et al., Psychiatry Clin | Acute/ | 9-24 hrs/ | Decreased Serotonin transporter |
| Neurosci 2001, 55: 75-77 | chronic | 1 hr-2 | (5-HTT) mRNA in raphe nucleus |
| | | weeks | |
| Burnet et al., Eur J | Single shock/ | | Increased Substance P (tachykinin |
| Pharmacol 2001, 413: 213-219 | 5 shocks on | | NK(1)) receptor binding site |
| | alternate days | | density, but no mRNA changes in |
| | | | cerebral cortex |
| Chen et al., Synapse 2001, | Acute | 2 hrs | Increased GRFalpha-1 and |
| 39: 42-50. | Chronic | | GFRalpha-2 mRNA in dentate |
| | | | gyrus |
| Cho et al., Exp Mol Med | Chronic | | Differential changes in PDE |
| 2000, 32: 110-114 | | | isoform mRNA in hippocampus |
| | | | and striatum |
| Lammers et al., Mol | 10 days | | Increased D3 receptor mRNA |
| Psychiatry 2000, 5: 348-388 | | | Shell of the nucleus accumbens |
| Madsen et al., Neuroscience | 14 days | 24 hrs | Increased Neuropeptide Y receptor |
| 2000, 98: 33-39 | | | Y1 and Y2 mRNA in CA1 and |
| | | | CA3 |
| Husum et al., | 10 days | | Increased preproNPY mRNA in |
| Neuropharmacology 2000, | | | the CA1 and dentate gyrus |
| 39: 1463-73 | | | |
| Valentine et al., Brain Res | | 6 hrs | Increase Fragile X (fmr1) mRNA |
| Mol Brain Res 2000, | | | in dentate gyrus granule cell layer |
| 75: 337-341 | | | |
| Burnet et al., Neurosci Lett | Single, | Immediately | Decreased 5-HT1A receptor |
| 1999, 277: 79-82 | repeated, | and 3 weeks | mRNA in CA4 Hippocampus and |
| | | after last | increased in dentate gyrus by |
| | | shock | single or repeated ECS. Repeated |
| | | | ECS increased cortical 5-HT2A |
| | | | receptor mRNA |
| Pei et al., Neuroscience | Single shock/ | | Single shock = Transient reduction |
| 1999, 90: 621-627 | 5 shocks over | | of GIRK1 mRNA in dentate gyrus |
| | 10 days | | Repeated shock = increase in |
| | | | GIRK2 and GIRK1 mRNA in |
| | | | dentate gyrus |
| Takahashi et al., J Neurosci | | | Increased Phosphodiesterase (PDE) |
| 1999, 19: 610-618 | | | 4 subtypes PDE4A, PDE4B |
| | | | mRNA in prefrontal cortex |
| Watkins et al., Brain Res | Single and | Levels | Dentate gyrus: increased mRNA |
| Mol Brain Res 1998, 61: 108-113 | repeated | return to | for NMDA subunits NR2A and |
| | | control | NR2B; reduced mRNA for |
| | | values after | metabotropic glutamate receptor |
| | | 48 hrs | mGLU5b |
| Suda et al., J Neurochem | Acute and | | Increase in Phosphodiesterase type |
| 1998, 71: 1554-1563 | repeated | | IV (PDE IV) mRNA in various |
| | | | brain regions after acute shock; |
| | | | repeated shock increased PDE IV |
| | | | in frontal cortex |
| Zetterstrom et al., Brain Res | Acute and | 6-48 hrs | BDNF increased with both acute |
| Mol Brain Res 1998, 57: 106-110 | chronic | | and chronic, both evident at 6 |
| | | | hours, chronic shock effete evident |
| | | | at 48 hours, but acute shock |
| | | | changes gone at 24 hr |
| Garcia-Garcia et al. | Acute and | | Corticotropin releasing factor |
| Neuroreport 1998, 9: 73-77 | chronic | | (CRF) increased in paraventricular |
| | | | nucleus; propiomelanocortin |
| | | | (POMC) increased in arcuate |
| | | | nucleus and ventromedial nucleus |
| | | | and Proenkephalin (PENK) in |
| | | | nucleus accumbens |
| Pei et al., Neuroreport | 5 times over | 6-24 hrs | Increased mRNA MAP2 in dentate |
| 1998, 9: 391-394 | 10 days | | gyrus |
| O'Donovan et al., J | | .5-1 hr and | Increased Erg-1 mRNA in dentate |
| Neurochem 1998, 70: 1241-1248 | | 4 hrs | granule cells returned to normal by |
| | | | 4 hr Increased Erg-3 peak at 4 hr |
| Xing et al., Brain Res Mol | Single | 15 min-4 hrs | Immediate increase in |
| Brain Res 1997, 47: 251-261 | | | Nurr1mRNA in dentate gyrus |
| | | | granule cells, peak at 1 hr and |
| | | | return to normal by 4 hr |
| Wang et al., Neurosci Lett | | | Increased Preprotachykinin-A |
| 1996, 226: 151-154 | | | (PPT-A) mRNA in CSF and |
| | | | aqueous humor of eye in RABBIT |
| Pei et al., Neuroscience | Acute and | 6 hrs, 24 hrs | Acute: reduced voltage-dependent |
| 1997, 78: 343-350 | chronic | and 3 weeks | potassium channel subunits Kv1.2 |
| | | | and Kv4.2 at 6 hrs and normal at |
| | | | 24 hrs in dentate gyrus |
| | | | Chronic: Kv1.2 at 6 hrs and |
| | | | normal at 24 hrs in dentate gyrus |
| | | | Kv4.2 no change at 6 hr elevated |
| | | | at 24 hr |
| Zachrisson et al., Eur J | | | Decrease in preprotachykinin-A |
| Pharmacol 1997, 319: 191-195 | | | mRNA in caudate -putamen and |
| | | | decrease in tachykinin NK1 |
| | | | receptor mRNA positive neurons |
| | | | in Nucleus Accumbens |
| Zachrisson et al., Brain Res | 6 shocks in 2 | | Decrease in CCK mRNA in |
| Mol Brain Res 1997, 43: 347-350 | weeks | | caudate-putamen |
| Jung et al., Biol Psychiatry | Single and 20 | 30 min-90 min | Varying effects in tetradecanoyl |
| 1996, 40: 503-507 | days | | phorbol acetate-inducible |
| | | | sequences (TIS) 1, 7, 8, 1, 21 in |
| | | | varying brain regions |
| McGowan et al., Eur J | 5 shocks over | | G protein subunits Gs alpha |
| Pharmacol 1996, 306: 249-55 | 5 days | | mRNA decreased in CA3 and |
| | | | CA1, G9(o) alpha mRNA |
| | | | increased in dentate gyrus and Gi2 |
| | | | alpha mRNA reduced in dentate |
| | | | gyrus an CA3 |
| Woldbye et al., Brain Res | Single shock | Various | Prominent induction of c-fos |
| 1996, 720: 111-119 | | times up to | mRNA and FOS in dentate gyrus |
| | | 24 hrs | |
| Porter et al., Brain Res | Single and 5 | | Acute: reduced KA1 mRNA in |
| 1996, 710: 97-102 | shocks over | | CA3a increased KA2 MRNA in |
| | 10 days | | dentate gyrus. |
| | | | Repeated shock: reduced KA1 |
| | | | mRNA in CA3c and CA3c and |
| | | | CA3a-b; KA2 mRNA increased in |
| | | | dentate gyrus All changes back to |
| | | | normal at 3 weeks post shock |
| Naylor et al., Brain Res Mol | Repeated | 24 hrs | Increased mRNA for GluR1 |
| Brain Res 1996, 35: 349-353 | shock | | subunit of AMPA receptor |
| Fitzgerald et al., J | Acute | | Increased ICER and CREM |
| Neurochem 1996, 66: 429-432 | | | isoforms in hippocampus, frontal |
| | | | cortex and cerebellum |
| Chen, et al., Mol | Acute and | | FosB and FosB-like proteins |
| Pharmacol 1995, 48: 880-889 | Chronic | | |
| Nibuya et al, J Neurosci | Acute (1 d) | | Acute: increased BDNF mRNA |
| 1995, 15: 7539-7547 | and Chronic | | recovered by 18 hr Chronic: |
| | (21 d) | | decreased induction of BDNF and |
| | | | trkB mRNA, but prolonged their |
| | | | expression in dentate gyrus; in |
| | | | CA1 and CA3, elevated induction |
| | | | of BDNF and trkB Mrna |
| Smith et al., | Acute & | 4 hrs and 24 hrs | Acute and repeated: increased D1 |
| Psychopharmacology 1995, | repeated (5 | | and D2 receptor mRNA at 4 hours |
| 120: 333-340 | shocks in 10 d) | | but NOT 24 hours in Nucleus |
| | | | Accumbens |
| Burnet et al., Neuroreport | 5 shocks in 10 | | Increased 5-HT1A and 5-HT2A |
| 1995, 6: 901-904 | days | | receptor mRNA in dentate gyrus |
| | | | and neocortex and decreaed 5- |
| | | | HT1A in CA3c |
| Lindefors et al., | | 1, 3, 9, 24 hrs | Brain derived neurotrophic factor |
| Neuroscience 1995, 65: 661-670 | | | mRNA and trkB mRNA increased |
| | | | in hippocampus |
| Dziedzicka-Wasylewska & | | | Increased proenkephalin mRNA in |
| Rogoz, J Neural Transm | | | nucleus accumbens |
| Gen Sect 1995, 102: 221-228 | | | |
| Kim et al., J Neurochem | Single | 6 hrs & 12 hrs | 6 hrs: decreased Inositol 1,4,5- |
| 1994, 63: 1991-1994 | | | triphosphate (InsP3) 3-kinase |
| | | | mRNA in dentate gyrus returned to |
| | | | normal at 12 hours |
| Brady et al., J Clin Invest | Single and | 24 hrs-8 wk | Single: Increased Corticotropin- |
| 1994, 94: 1263-1268 | repeated | | releasing hormone (CRH) mRNA |
| | | | in the paraventricular nucleus and |
| | | | TH mRNA in the locus coeruleus |
| | | | at 24 hrs |
| | | | Repeated: some increase that |
| | | | persisted for up to 3 weeks (CRV) |
| | | | and up to 8 weeks (TH) |
| Passarelli et al., Neurosci | Single and | 2 hrs | Increased expression of hcs73 in |
| Lett 1994, 177: 147-150 | repeated (7 d) | | dentate gyrus, CA3 and median |
| | | | habenular nucleus; induced hsp70 |
| | | | mRNA in dentate gyrus (same in |
| | | | single and repeated shock) |
| Mikkelsen et al., Brain Res | 14 days | 24 hrs | 10 × increase in PreproNPY |
| Mol Brain Res 1994, 23: 317-322 | | | mRNA in dentate gyrus and |
| | | | piriform cortex |
| Follesa et al., Exp Neurol | Corneal shock | | Min: bFGF mRNA increased in |
| 1994, 127: 37-44 | Minimal | | entorhinal cortex and hippocampus |
| | (0.2 s, 50-70 mA | | Max: increased in bFGF mRNA |
| | 3x in | | in entorhinal cortex, hippocampus, |
| | 1 h) and | | olfactory bulb, striatum and |
| | Maximal | | cerebellum, increased NGF mRNA |
| | (0.2 s, 150 mA | | in entorhinal cortex and |
| | 3x in 1 h) | | hippocampus |
| Kragh et al., Exp Brain Res | 10 shocks, 20 | 1 d, 2 d, 30 d | Increased SS-like and NYP-like |
| 1994, 98: 305-13 | shocks, or 36 | | immunoreactivity 1 and 2 d after |
| | shocks given | | last shock in outer part of dentate |
| | on weekdays | | molecular layer Most pronounced |
| | only | | in 36 shock group |
| Butler et al., J Neurochem | Acute (1 d or | | Chronic: increased 5-HT2 |
| 1993, 61: 1270-1276 | 3 d) Chronic | | receptor mRNA |
| | (10 d or 14 d) | | |
| Passarelli and Orzi Neurosci | Repeated (7 d) | | Repeated: Increased expression of |
| Lett 1993, 153: 197-201 | and single | | somatostantin mRNA in |
| | | | hippocampus |
| Hosoda and Duman, J | 10 d | | Down-regulation of beta 1AR |
| Neurochem 1993, 60: 1335-1343 | | | mRNA in frontal cortex |
| Kapu et al., Brain Res Mol | | | Increased TH mRNA and NPY |
| Brain Res 1993, 18: 121-126 | | | mRNA in Locus Coeruleus |
| Pratt et al., Brain Res Bull | | 4-24 hrs | Increased GABAa receptor |
| 1993, 30: 691-693 | | | subunits alpha 1 and beta 2 mRNA |
| | | | in cerebellum at 4-8 hours, |
| | | | returned to normal at 24 hrs |
| Wong et al., Brain Res Mol | | | Induction of the constitutive hsp72 |
| Brain Res 1992, 13: 19-25 | | | gene in dentate gyrus |
| Lindefors et al., | 5 shocks in 10 | | Increased preprocholecystokinin |
| Neuroscience 1991, 45: 73-80 | days | | and preprotachykinin-A mRNA in |
| | | | Edinger-Westphal nucleus |
| Kang et al., | Single | 2-8 hrs | Increased Alpha 1 and gamma2 |
| Psychopharmacol Bull 1991, | | | GABA receptor subunit mRNA in |
| 27: 359-363 | | | cerebellum and hippocampus not at |
| | | | 2 hrs, increased at 4 hours |
| | | | returned to normal at 8 hours in |
| | | | hippocampus |
| Cole et al., . J Neurochem | Single and | 15 min. and | 15 min: increased zif/268, c-fos, |
| 1990, 55: 1920-1927 | repeated | 4 hrs | c-jun and jun-B in hippocampus |
| | | | and neocortex and pyriform cortex; |
| | | | all except c-jun returned to normal |
| | | | by 4 hours |
| Herman, et al., Brain Res | 7 days | | Increased CRF mRNA in medial |
| 1989, 501: 235-246 | | | parvocellular paraventricular |
| | | | nucleus (PVN) |
| Xei et al., Brain Res Mol | Single and | 0.5, 2, 6, 12 hrs | Single: decrease in DYN mRNA |
| Brain Res 1989, 6: 11-19 | repeated (1, | (single); | at 0.5 hour followed by increase at |
| | 3, 6 days) | 24 hrs | 6 hrs in hippocampus; EK mRNA |
| | | (repeated) | increased at 0.5 hour and still high |
| | | | at 12 hrs in entorhinal cortex and |
| | | | hippocampus |
| | | | Repeated: decreased DYN mRNA |
| | | | in hippocampus at 24 hrs elevated |
| | | | DYN mRNA in striatum and |
| | | | hypothalamus. Increased EK |
| | | | mRNA in entorhinal cortex at 6 hrs |
| Yoshikawa et al., ProcNatl | 10 d | | Increased preproenkephalin mRNA |
| Adad Sci USA 1985, 82: 589-593 | | | in hypothalamus and striatum |

8.2. ECS Gene Selection Algorithm

Three considerations were quantified and summed to prioritize the significance of gene “hit” as defined in the statistical analysis portion of Section 7.1. 1, infra.

I. Statistical considerations:

(High score = 27). Score = p value Score + (0.5) Ratio Score + (0.1) Abundance Score
The significance (p value), fold change (ratio), and gene abundance measured in optical
intensity units were assigned an integer value as follows:
p value Score = −log₁₀(p value)
Ratio Score = Ratio if the ratio > 1.5 or 1/Ratio if ratio ≦ 1/1.5
Abundance Score = log₁₀ (Abundance/700)

II. Biological considerations:

```
Score = A (10) + B (8) + C (6) + D (5) + E (2) + F (4) (Max. score = 35)
A. Gene change is in the opposite direction of mRNA or protein change in human
  depression or bipolar disorder, or animal model reported in the literature.
B. Same as A, but gene change is in the same direction.
C. Gene is known to be changed by effective treatments for human depression or bipolar
  disorder
D. Gene is part of a genetic or biochemical pathway known to be associated with human
  depression, bipolar disorder, or their treatment
E. Gene has been reported to change following seizures in human or animal studies.
F. Gene's human homologue is in a chromosomal hot spot for depression or bipolar disorder
  as identified by linkage analysis.
```

III. Experimental considerations:

The experimental results score is based on changes in either or both tissues and after acute,
chronic, or both ECS exposures (Max. score = 14)

| | |
|---|---|
| 14 | Both tissues after acute and chronic |
| 10 | Two tissues, chronic and one tissue acute |
| 8 | Both tissues, chronic |
| 6 | Two tissues acute, one tissue chronic |
| 4 | One tissue chronic, one tissue acute |
| 2 | Two tissues, acute |
| 1 | One tisue chronic |

ECS Algorithm Score = 2.2(I) + II + III
Maximum score possible = 2.2(27) + 35 + 14 = 108

8.3. Fold Change (RATIO), Significance (p VALUE). and Ranking of Genes Identified as Altered in Hippocampus (H) and Frontal Cortex (F) after Either Actue (A) or Chronic (C) ECS Exposure

| | | | | | Other | |
|---|---|---|---|---|---|---|
| | | | | Region | regions | |
| | | | | with | with | |
| | Accession | | p | biggest | significant | Algorithm |
| Gene Name | No. | RATIO | VALUE | change | change | Score |
| *cyclooxygenase isoform | S67722 | 7.33 | 9E−08 | HA | All | 73.9 |
| (COX-2) | | | | | | |
| putative potassium channel TWIK | AF022819 | 1.83 | 9E−04 | HC | HA | 14.5 |
| Vascular endothelial growth factor | AA850734 | 1.73 | 8E−05 | HA | FA | 11.9 |
| (VEGF) | | | | | | |
| *brain-derived neurotrophic factor | D10938 | 4.78 | 1E−03 | HC | All | 74.8 |
| (BDNF) (exon IV) | | | | | | |
| metallothionein 1 A | AI102562 | 2.04 | 2E−05 | HA | FA, HC | 62.1 |
| Phosphodiesterase 4B, cAMP- | M25350 | 1.54 | 3E−05 | FA | FC | 29.8 |
| Specific | | | | | | |
| fos-related antigen (fra-2) | AA875032 | 3.16 | 8E−05 | HA | FA, HC | 59.9 |
| neuropeptide Y (NPY) | M15880 | 2.14 | 1E−02 | HC | FC | 54.0 |
| HMG-CoA reductase | X55286 | 1.48 | 3E−05 | HC | HA, HC | 34.0 |
| TGFB inducible early growth | AI172476 | 4.68 | 1E−03 | HC | All | 49.0 |
| response (TIEG1) | | | | | | |
| nuclear orphan receptor HZF-3 | U01146 | 1.70 | 1E−05 | HA | HC | 39.9 |
| (NURR1) | | | | | | |
| Jun B | X54686 | 2.21 | 1E−07 | FA | HA | 59.3 |
| Jun | AI175959 | 1.84 | 9E−05 | HA | FA, FC | 58.8 |
| *NMDAR1 glutamate receptor | U11418 | 1.59 | 2E−02 | HA | | 42.0 |
| subunit | | | | | | |
| *Tissue inhibitor of | AI169327 | 11.39 | 2E−06 | HA | All | 56.9 |
| metalloproteinase 1 (TIMP1) | | | | | | |
| *Narp | S82649 | 5.89 | 7E−09 | HC | All | 46.4 |
| cytosolic phospholipase A2, group | U38376 | 1.79 | 6E−07 | HA | HC | 48.6 |
| IVA | | | | | | |
| silencer factor B (SF-B) | X60769 | 1.76 | 2E−04 | HA | All | 37.5 |
| Minoxidil sulfotransferase | L19998 | 0.504 | 4E−03 | HC | HA, FA | 25.6 |
| VGF nerve growth factor inducible | M74223 | 2.35 | 8E−09 | HA | All | 46.5 |
| phosphatidylinositol 3-kinase p85 | D64045 | 2.02 | 4E−04 | FA | | 19.5 |
| alpha subunit | | | | | | |
| c-fos | X06769 | 2.22 | 1E−05 | FC | HA, HC | 44.9 |
| Catalase | AA926149 | 0.60 | 8E−04 | HA | | 44.1 |
| ceruloplasmin (ferroxidase) | L33869 | 1.81 | 2E−05 | FA | HA, HC | 33.4 |
| *nerve growth factor-induced | M92433 | 2.41 | 4E−07 | HA | HC, FA | 42.5 |
| clone C | | | | | | |
| thyrotropin-releasing hormone | M23643 | 2.01 | 1E−02 | HC | | 42.4 |
| glial fibrillary acidic protein delta; | AF028784 | 1.769 | 2E−04 | FA | HA, HC | 42.0 |
| glial fibrillary acidic protein alpha | | | | | | |
| HES-1 factor | D13417 | 2.09 | 5E−03 | HC | All | 26.7 |
| activity and neurotransmitter- | AF030089 | 2.58 | 6E−07 | HA | FC, HC | 23.8 |
| induced early gene protein 4 (ania- | | | | | | |
| 4) | | | | | | |
| Cytochrome P450 1b1 | AI176856 | 2.247 | 9E−04 | FA | HA, HC | 25.3 |
| arginine vasopressin (Diabetes | M25646 | 1.52 | 4E−03 | HA | FA | 39.0 |
| insipidus) | | | | | | |
| enhancer-of-split and hairy-related | AF009329 | 2.06 | 2E−03 | HC | All | 32.5 |
| protein 1 (SHARP 1) | | | | | | |
| *Protein-tyrosine phosphatase | AI180145 | 1.66 | 2E−02 | HA | | 26.5 |
| (Ptpn1, non-receptor type 1) | | | | | | |
| Glutathione S-transferase, theta 2 | AI138143 | 0.63 | 1E−02 | HA | FA | 26.5 |
| protein phosphatase 1, regulatory | J05592 | 1.62 | 6E−03 | HC | FA, HA | 22.7 |
| (inhibitor) subunit 1A | | | | | | |
| krox20; (early growth response 2) | U78102 | 3.13 | 7E−04 | FA | HA, HC | 36.2 |
| interleukin 6 receptor | M58587 | 1.74 | 3E−05 | FA | | 22.2 |
| S-adenosylmethionine | AI008131 | 1.55 | 1E−02 | HA | FA | 24.8 |
| decarboxylase | | | | | | |
| ets variant gene 3 | AA859750 | 1.62 | 1E−05 | HA | All | 28.5 |
| *neural receptor protein-tyrosine | M55291 | 1.52 | 2E−02 | HA | HC, FA | 20.4 |
| kinase (TrkB) | | | | | | |
| *Vesl | AB003726 | 6.50 | 2E−07 | HA | FA, HC | 33.9 |
| pyruvate dehydrogenase | AF062740 | 1.83 | 2E−02 | HC | HA, FA | 17.6 |
| phosphatase isoenzyme 1 | | | | | | |
| EST195957 (Mad4 homolog | AA892154 | 0.67 | 1E−04 | HA | All | 27.2 |
| (MAD4)) | | | | | | |
| MHC class 1b RT1.S3 | AI235890 | 1.808 | 3E−03 | HA | HC | 32.2 |
| Growth factor receptor bound | AI170776 | 1.68 | 6E−06 | HA | HC | 32.0 |
| protein 2 (GRB2) | | | | | | |
| prostaglandin D synthase | J04488 | 0.35 | 6E−03 | HC | FC | 25.5 |
| *metabotropic glutamate receptor | M61099 | 0.60 | 3E−05 | HA | | 25.5 |
| (GluR1) | | | | | | |
| Decorin | AI639233 | 0.66 | 2E−02 | HA | | 31.5 |
| aldehyde dehydrogenase | M73714 | 0.45 | 2E−05 | HA | | 20.6 |
| rx00909s Rattus norvegicus cDNA | AI638960 | 0.62 | 3E−06 | HA | HC, FA | 17.3 |
| clone | | | | | | |
| activin type I receptor | L19341 | 1.63 | 3E−05 | HC | | 7.2 |
| growth factor (Arc) | U19866 | 4.82 | 6E−09 | HA | FA, HC | 29.0 |
| Interferon-related developmental | AI014163 | 2.03 | 6E−03 | HC | HA, FA | 21.7 |
| regulator 1 | | | | | | |
| neuritin | U88958 | 1.56 | 1E−08 | HA | FA, HC | 27.7 |
| myr 6 myosin heavy chain | U60416 | 0.65 | 3E−05 | HA | HC | 26.7 |
| interferon gamma receptor (Ifngr) | U68272 | 0.65 | 5E−04 | FC | | 20.1 |
| lost on transformation 1 (LOT1) | U72620 | 1.58 | 8E−05 | FA | | 6.0 |
| DNAJ (Hsp40) homolog, subfamily | AA891542 | 2.32 | 1E−07 | HA | HC, FA | 25.7 |
| B, member 5 | | | | | | |
| JE product (small inducibe | X17053 | 1.85 | 3E−03 | FA | HC, HA | 25.4 |
| cytokine A2) | | | | | | |
| Activity and Neurotransmitter | AF030088 | 4.787 | 4E−05 | HC | HA | 24.9 |
| induced early gene 3; (ania-3) | | | | | | |
| Roaz | U92564 | 0.65 | 5E−05 | HA | HC | 14.3 |
| 3CH134/CL100 PTPase (protein | S81478 | 1.67 | 8E−03 | HA | FA | 24.3 |
| tyrosine phosphatase, non-receptor | | | | | | |
| 16) | | | | | | |
| matrilysin (Matrix | L24374 | 1.62 | 9E−03 | HA | | 4.3 |
| metalloproteinase 7) | | | | | | |
| EST194857 | AA891054 | 1.82 | 5E−03 | FC | All | 24.0 |
| tachykinin 2 (Neurokinin B | M16410 | 3.36 | 2E−03 | HC | | 22.1 |
| precursor (NKB)) | | | | | | |
| rx04104s (Neurod1) | AI639109 | 0.42 | 2E−03 | HA | FA, HC | 21.8 |
| EST190175 (atrophin-1 related | AA800678 | 0.64 | 9E−06 | HA | HC, FA | 21.5 |
| protein) | | | | | | |
| solute carrier family 3, member 1 | M80804 | 0.48 | 3E−02 | FC | FC, HA | 11.4 |
| flavin-containing monooxygenase 1 | M84719 | 0.67 | 2E−03 | FA | HA | 10.3 |
| SPA-1 like protein | AI237576 | 1.57 | 1E−02 | HA | | 4.2 |
| PP1M M110 | S74907 | 1.76 | 3E−03 | HC | | 19.9 |
| leucine-rich acidic nuclear protein | D32209 | 1.60 | 4E−02 | FC | | 5.0 |
| BHF-1 | D82074 | 0.44 | 6E−04 | HA | FA, HC | 19.0 |
| rx01019s Rattus norvegicus cDNA | AI639256 | 0.62 | 4E−04 | HA | HC | 12.8 |
| clone | | | | | | |
| Nuclear receptor subfamily 4, | AI176710 | 1.62 | 8E−04 | FA | HA | 10.8 |
| group A, member 3 | | | | | | |
| Isovaleryl Coenzyme A | AI102838 | 0.64 | 3E−03 | HC | | 18.4 |
| dehydrogenase | | | | | | |
| insulin-like growth factor II | X17012 | 0.44 | 5E−02 | HC | FC | 17.8 |
| (somatomedin A) | | | | | | |
| GADD153 | U30186 | 3.16 | 8E−05 | HA | | 17.0 |
| UI-R-E0-bs-h-03-0-UI.s1 UI-R-E0 | AA859627 | 1.56 | 2E−02 | HA | | 5.0 |
| Rattus norvegicus cDNA clone | | | | | | |
| Calvasculin; S100 calcium-binding | X06916 | 1.58 | 3E−02 | HC | | 16.8 |
| protein A4 | | | | | | |
| EST189184 | AA799687 | 0.65 | 2E−03 | HA | | 4.8 |
| developmentally regulated protein | AI012275 | 0.61 | 4E−04 | HA | HC | 10.6 |
| TPO1 | | | | | | |
| UI-R-A0-bd-e-03-0-UI.s1 UI-R-A0 | AA866485 | 1.53 | 1E−03 | FA | HA | 10.0 |
| Rattus norvegicus cDNA clone | | | | | | |
| cyclic AMP-regulated | S65091 | 2.09 | 7E−07 | HA | HC | 15.8 |
| phosphoprotein | | | | | | |
| di-N-acetylchitobiase | M95768 | 0.66 | 3E−05 | HA | | 5.8 |
| mothers against dpp 3 homolog | U66479 | 1.78 | 3E−03 | HA | | 15.7 |
| EST225243 (S100 calcium binding | AI228548 | 0.65 | 3E−02 | HC | | 15.7 |
| protein A1) | | | | | | |
| CCAAT/enhancerbinding, protein | M65149 | 2.95 | 2E−05 | FA | FC | 15.4 |
| (C/EBP) delta | | | | | | |
| EST196314 (tescalcin) | AA892511 | 1.53 | 1E−02 | HC | HA | 9.1 |
| EST220045 | AI176460 | 7.81 | 3E−01 | FC | | 15.0 |
| Potassium voltage gated channel, | AI230211 | 2.437 | 5E−02 | HA | FC | 14.3 |
| Shal-related family member 2 | | | | | | |
| transducin-like enhancer of split | AA875084 | 0.60 | 1E−06 | HA | FA | 14.0 |
| 1(Tle1) | | | | | | |
| *Fibroblast growth factor receptor 1 | D12498 | 1.51 | 2E−03 | HA | | 7.7 |
| chemokine CX3C | AF030358 | 1.59 | 1E−05 | HA | HC | 12.9 |
| neuropilin | AF016296 | 1.67 | 2E−03 | HA | HC | 12.8 |
| EST197009 | AA893206 | 1.63 | 4E−05 | HA | HC | 12.8 |
| EST190350 | AA800853 | 1.55 | 2E−05 | FA | HA | 12.4 |
| EST213163 (FK506 binding | AI103874 | 0.72 | 3E−04 | HC | HA | 12.0 |
| protein 3) | | | | | | |
| DNA-damage-inducible transcript 1 | L32591 | 1.68 | 2E−03 | HC | | 5.8 |
| thymosin beta-10 | M58404 | 1.51 | 3E−04 | HC | | 3.8 |
| Y box protein 1 | AI230572 | 1.53 | 7E−03 | HA | | 3.6 |
| phosducin-like protein | L15354 | 0.56 | 6E−06 | HA | FA | 11.3 |
| Ash-s | D49847 | 1.56 | 1E−03 | HC | HA | 11.2 |
| connexin protein Cx26 (gap | X51615 | 0.66 | 1E−02 | FA | HC | 11.2 |
| junction beta-2 protein) | | | | | | |
| Early growth response 1 (EGR1) | AI176662 | 1.63 | 1E−03 | HA | FA | 11.1 |
| EST215655 (similar to G33) | AI169756 | 1.78 | 3E−03 | FA | HA | 10.7 |
| rx04422s Rattus norvegicus cDNA | AI639169 | 0.66 | 5E−04 | HA | | 4.6 |
| clone | | | | | | |
| protein tyrosine phosphatase | AJ007016 | 1.51 | 3E−03 | HA | FA | 10.4 |
| (PtP4a2) | | | | | | |
| EST195538 | AA891735 | 1.58 | 3E−04 | HA | HC | 10.3 |
| rx04826s Rattus norvegicus cDNA | AI639058 | 1.62 | 2E−02 | HC | HA | 10.0 |
| clone (similar to Nedd4) | | | | | | |
| UI-R-E0-ci-e-02-0-UI.s1 UI-R-E0 | AA874943 | 0.64 | 3E−04 | HA | HC | 9.2 |
| Rattus norvegicus cDNA clone | | | | | | |
| Transcription factor UBF | M61725 | 1.58 | 8E−03 | HA | HC | 8.8 |
| EST203592 (chromobox homolog | AI009141 | 0.63 | 3E−02 | HC | HA | 8.7 |
| 1) | | | | | | |
| Lysyl oxidase | S77494 | 2.407 | 6E−03 | HC | | 8.5 |
| Small proline-rich protein gene | AA891911 | 1.52 | 2E−02 | HC | | 6.9 |
| (cornifin alpha) | | | | | | |
| EST196165 | AA892362 | 0.65 | 3E−04 | HA | | 6.7 |
| EST106597 | H31990 | 0.64 | 1E−03 | HC | | 6.6 |
| heme oxygenase-3 | AF058787 | 1.61 | 9E−03 | HC | | 6.5 |
| EST197625 (RAD52 homolog) | AA893822 | 0.64 | 9E−04 | HA | | 6.3 |
| protein kinase MNK2 | Z21935 | 1.81 | 2E−02 | HA | | 6.2 |
| SHB (Src homology 2 domain | AA859468 | 1.77 | 7E−03 | HC | HA | 5.2 |
| containing) adaptor protein B | | | | | | |
| rx00382s Rattus norvegicus cDNA | AI639155 | 0.56 | 3E−05 | HA | | 5.2 |
| clone | | | | | | |
| UI-R-E0-bx-e-11-0-UI.s1 UI-R-E0 | AA859690 | 1.94 | 3E−08 | HC | | 4.7 |
| Rattus norvegicus cDNA clone | | | | | | |
| EST190198 (coagulation factor III) | AA800701 | 1.58 | 2E−04 | HA | | 4.4 |
| EST190228 | AA800731 | 0.61 | 7E−03 | FC | | 4.1 |
| EST197395 (reticulocalbin 2) | AA893592 | 1.67 | 5E−02 | HC | | 4.1 |
| nuclear pore complex protein | U41845 | 1.52 | 2E−04 | HA | | 3.9 |
| Nup50 | | | | | | |
| *clathrin, light polypeptide (LCB) | AA874955 | 1.62 | 4E−03 | HA | | 3.8 |
| NAC-1 protein | AF015911 | 1.63 | 1E−02 | HA | | 3.8 |
| beta defensin-1 | AF068860 | 1.24 | 2E−02 | HA | | 3.7 |
| voltage-dependent potassium | X12589 | 1.83 | 4E−02 | HA | | 3.5 |
| channel; (shaker-related subfamily) | | | | | | |
| rx00967s Rattus norvegicus cDNA | AI639015 | 0.61 | 4E−04 | HA | | 3.1 |
| clone | | | | | | |
| Beta-carotene 15, 15′-dioxygenase | AI014135 | 0.59 | 2E−02 | FA | | 2.9 |
| BRL-3A binding protein | A09811 | 0.32 | 1E−02 | HC | | 2.9 |
| potassium voltage gated channel, | M32867 | 1.61 | 1E−02 | HC | | 2.7 |
| shaker related subfamily, member 4 | | | | | | |

8.4. Supplemental List of Highly Specific Genes that Change Less than 1.5-Fold (Columns are as Defined in Section 8.3, supra)

| | | | | | Other | |
|---|---|---|---|---|---|---|
| | | | | Region | regions | |
| | | | | with | with | |
| | Accession | | p | lowest p | significant | Algorithm |
| Gene | No. | RATIO | VALUE | value | change | Score |
| Bcl-2 associated death agonist | AA818072 | 0.8 | 3E−05 | HA | HC | 32 |
| (BAD) | | | | | | |
| beta-tubulin T beta 15 (aa 1-445) | X03369 | 1.27 | 1E−05 | HC | | 8 |
| diacylglycerol kinase | S49760 | 1.37 | 4E−04 | HC | | 7 |
| EST 189694 Normalized rat | AA800197 | 0.69 | 4E−07 | HA | HC | 11 |
| heart | | | | | | |
| EST 195383 Normalized rat | AA891580 | 0.67 | 2E−05 | HC | HA | 9 |
| kidney | | | | | | |
| EST 197783 Normalized rat | AA893980 | 0.76 | 6E−06 | HA | HC | 10 |
| placenta | | | | | | |
| GABA-A receptor | X51992 | 0.79 | 2E−04 | HC | HA | 19 |
| Glypican | L02896 | 1.28 | 3E−03 | HC | FA, FC | 13 |
| Growth factor receptor bound | AA801130 | 1.23 | 5E−04 | HC | HA, FC | 16 |
| protein 2 | | | | | | |
| Light molecular weight | AF031880 | 1.4 | 1E−05 | HA | HC | 24 |
| neurofilament | | | | | | |
| Neurofilament protein middle | Z12152 | 1.26 | 4E−08 | HA | | 23 |
| (NF-M) | | | | | | |
| Neurofilament protein; rat | M25638 | 1.49 | 2E−07 | HA | HC | 22 |
| smallest (NF-L) | | | | | | |
| Neuron glucose transporter | D13962 | 1.4 | 7E−05 | FA | | 5 |
| Phospholipase C-beta 1 | L14323 | 0.79 | 3E−05 | HA | | 24 |
| Phospholipase D gene 2 | NM_033299 | 0.72 | 1E−04 | HA | HC | 23 |
| Ras homolog enriched in brain | A1009605 | 1.42 | 3E−03 | HA | HC | 12 |
| SH3-domain GRB2-like 1 | A1010110 | 1.26 | 1E−05 | HA | | 12 |
| Sodium-dependent | S56141 | 0.78 | 1E−05 | HA | | 5 |
| neurotransmitter transporter, | | | | | | |
| ventral midbrain | | | | | | |
| Synapsin 2 | M27925 | 1.34 | 4E−04 | FC | | 6 |

8.5. Confirmation of Select Gene Changes by Quantitative RT-PCR

All samples for ECS-treated groups and sham control groups used for microarray studies were analyzed for each gene (n=8-10 per group).

| | | QRT-PCR | | Microarray | |
|---|---|---|---|---|---|
| UniGene | Name | Ratio | p value | Ratio | p value |

Frontal Cortex, Acute ECS

| | | | | | |
|---|---|---|---|---|---|
| Rn.32777 | Ceruloplasmin (ferroxidase) | 1.35 | 2.8E−02 | 1.81 | 2.0E−05 |
| Rn.10599 | Phosphoinositide 3-kinase, regulatory | 1.59 | 5.8E−04 | 2.02 | 4.0E−03 |
| | subunit, | | | | |
| | polypeptide 1 | | | | |
| Rn.6977 | Lost on transformation 1 | 1.90 | 2.1E−04 | 1.58 | 8.0E−05 |
| Rn.1716 | Interleukin 6 receptor | 2.24 | 3.1E−08 | 1.74 | 3.0E−05 |

Frontal Cortex, Chronic ECS

| Rn.11400 | Prostaglandin D synthase | 0.61 | 1.0E−02 | 0.71 | 0.03 |
|---|---|---|---|---|---|
| Rn.7730 | Prostaglandin E synthase | 2.18 | 1.5E−02 | not on U34A chip | |
| Rn.506 | Synapsin 2 | 1.29 | 1.0E−03 | 1.34 | 0.00 |

Hippocampus, Acute ECS

| | | | | | |
|---|---|---|---|---|---|
| Rn.87787 | glutamate receptor, | 0.63 | 2.5E−06 | 0.60 | 3.0E−05 |
| | metabotropic 1 | | | | |
| Rn.19727 | Hairy and enhancer of split 1 | 0.71 | 3.3E−02 | 1.55 | 4.0E−05 |
| | (Drosophila) | | | | |
| Rn.3812 | Normalized rat heart, Bento | 0.72 | 1.4E−05 | 0.65 | 2.0E−03 |
| | Soares Rattus sp. cDNA | | | | |
| | RHEAE07 | | | | |
| Rn.9113 | Aldehyde dehydrogenase | 0.86 | 5.6E−03 | 0.44 | 2.0E−05 |
| | family 3, subfamily A2 | | | | |
| Rn.3360 | Growth factor receptor | 1.36 | 3.1E−02 | 1.68 | 6.0E−06 |
| | bound protein 2 | | | | |
| Rn.10815 | Neuropilin | 1.36 | 1.2E−04 | 1.67 | 2.0E−03 |
| Rn.9797 | Fibroblast growth factor | 1.44 | 1.6E−03 | 1.51 | 2.0E−03 |
| | receptor 1 | | | | |
| Rn.2045 | Protein tyrosine | 1.46 | 2.0E−03 | 1.51 | 3.0E−03 |
| | phosphatase type IVA, | | | | |
| | member 2 | | | | |
| Rn.3546 | Neuritin | 1.59 | 4.9E−06 | 1.56 | 1.0E−08 |
| Rn.9839 | Nuclear receptor subfamily | 1.76 | 6.8E−05 | 1.70 | 1.0E−05 |
| | 4, group A, member 2 | | | | |
| Rn.44320 | Avian sarcoma virus 17 (v- | 1.80 | 1.7E−07 | 1.84 | 9.0E−05 |
| | jun) oncogene homolog | | | | |
| Rn.859 | Ras homolog enriched in | 1.83 | 1.0E−07 | 1.42 | 1.0E−05 |
| | brain | | | | |
| Rn.11183 | DNA-damage inducible | 1.89 | 1.6E−08 | 1.78 | 4.0E−03 |
| | transcript 3 | | | | |
| Rn.3723 | Interferon-related | 1.93 | 1.3E−07 | 2.01 | 3.0E−05 |
| | developmental regulator 1 | | | | |
| D82074 | Rattus sp. mRNA for BHF-1 | 1.97 | 7.0E−04 | 0.44 | 6.0E−04 |
| Z21935 | R. norvegicus protein kinase | 2.28 | 1.6E−02 | 1.81 | 2.0E−02 |
| | rMNK2 | | | | |
| S65091 | cyclic AMP-regulated | 2.52 | 6.7E−05 | 2.09 | 7.0E−07 |
| | phosphoprotein | | | | |
| Rn.2398 | TGFB inducible early growth | 2.79 | 1.4E−08 | 3.62 | 1.0E−06 |
| | response | | | | |
| Rn.40517 | Activity and | 3.64 | 2.9E−10 | 2.58 | 6.0E−07 |
| | neurotransmitter-induced | | | | |
| | early gene protein 4 (ania-4) | | | | |
| Rn.44369 | Prostaglandin-endoperoxide | 4.03 | 1.5E−07 | 7.33 | 9.0E−08 |
| | synthase 2 | | | | |
| Rn.25754 | Tissue inhibitor of | 4.71 | 8.7E−08 | 11.39 | 2.0E−06 |
| | metalloproteinase 1 | | | | |
| Rn.11266 | Brain derived neurotrophic | 5.73 | 2.9E−08 | 3.85 | 2.0E−04 |
| | factor | | | | |
| S82649 | neuronal activity-regulated | 7.24 | 2.0E−10 | 5.89 | 7.0E−09 |
| | pentraxin | | | | |
| Rn.37500 | Homer, neuronal immediate | 7.42 | 2.5E−07 | 6.50 | 2.0E−07 |
| | early gene, 1 | | | | |

Hippocampus, Chronic ECS

| | | | | | |
|---|---|---|---|---|---|
| Rn.964 | Insulin-like growth factor II | 0.33 | 4.4E−02 | 0.44 | 5.0E−02 |
| | (somatomedin A) | | | | |
| Rn.11400 | Prostaglandin D synthase | 0.34 | 3.9E−02 | 0.35 | 6.0E−03 |
| Rn.19727 | Hairy and enhancer of split 1 | 1.27 | 1.8E−02 | 2.09 | 5.0E−03 |
| | (Drosophila) | | | | |
| M25638 | Rat smallest neurofilament | 1.31 | 2.1E−03 | | |
| | protein (NF-L) mRNA | | | | |
| Rn.7044 | Glypican 1 | 1.33 | 1.6E−02 | | |
| Rn.78188 | heme oxygenase-3 (HO-3) | 1.42 | 2.3E−02 | 1.61 | 9.0E−03 |
| | mRNA | | | | |
| Rn.859 | Ras homolog enriched in | 1.54 | 6.7E−04 | | |
| | brain | | | | |
| Rn.9756 | Protein phosphatase 1, | 1.58 | 4.8E−06 | 1.62 | 6.0E−03 |
| | regulatory (inhibitor) subunit | | | | |
| | 1A | | | | |
| Rn.3546 | Neuritin | 1.58 | 8.6E−05 | 1.44 | 8.0E−04 |
| Rn.31799 | Pyruvate dehydrogenase | 1.72 | 1.3E−03 | 1.83 | 2.0E−02 |
| | phosphatase isoenzyme 1 | | | | |
| Rn.9714 | Neuropeptide Y | 2.40 | 5.2E−06 | 2.14 | 1.0E−02 |
| Rn.22 | Thyrotropin releasing | 21.42 | 1.8E−03 | 2.01 | 1.0E−02 |
| | hormone | | | | |

8.6. Electroconvulsive Seizure (ECS) Signature Genes

| | | | | | PGI Data, Group II: Scores | | | Algorithm Scores | | |
|---|---|---|---|---|---|---|---|---|---|---|
| | | Unigene number | | | for gene overlaps with: | | | | Groups I, II | ECS |
| Gene Name | Accession | Rat | Human | Mouse | Human | Human | Mouse + | | (Literature) | Algorithm |
| (SEQ ID NO.) | No. | unigene | Unigene | Unigene | tissue | cells | Rat tissue | PGI II | and III | Score |
| cyclooxygenase isoform | S67722 | Rn.44369 | Hs.196384 | Mm.3137 | 8 | 6 | 0 | 14 | 73.9 | 87.9 |
| (COX-2) | | | | | | | | | | |
| (SEQ ID NO: 1) | | | | | | | | | | |
| putative potassium | AF022819 | Rn.15693 | Hs.79351 | Mm.10800 | 50 | 12 | 0 | 62 | 14.5 | 76.5 |
| channel TWIK | | | | | | | | | | |
| (SEQ ID NO: 2) | | | | | | | | | | |
| Vascular endothelial | AA850734 | Rn.1923 | Hs.73793 | Mm.31540 | 46 | 6 | 12 | 64 | 11.9 | 75.9 |
| growth factor (VEGF) | | | | | | | | | | |
| (SEQ ID NO: 3) | | | | | | | | | | |
| brain-derived neurotrophic | AI030286 | Rn.11266 | Hs.56023 | Mm.1442 | 0 | 0 | 0 | 0 | 74.8 | 74.8 |
| factor (BDNF) (exon IV) | | | | | | | | | | |
| (SEQ ID NO: 4) | | | | | | | | | | |
| metallothionein 1 A | AI102562 | Rn.54397 | Mm.192991 | Mm.192991 | 0 | 0 | 6 | 6 | 62.1 | 68.1 |
| (SEQ ID NO: 5) | | | | | | | | | | |
| Phosphodiesterase 4B, | M25350 | Rn.2485 | Hs.188 | Mm.20181 | 38 | 0 | 0 | 38 | 29.8 | 67.8 |
| cAMP-Specific | | | | | | | | | | |
| (SEQ ID NO: 6) | | | | | | | | | | |
| fos-related antigen (fra-2) | AA875032 | Rn.3212 | NA | NA | 0 | 0 | 6 | 6 | 59.9 | 65.9 |
| (SEQ ID NO: 7) | | | | | | | | | | |
| HMG-CoA reductase | X55286 | Rn.9437 | Hs.11899 | NA | 24 | 6 | 0 | 30 | 34 | 64 |
| (SEQ ID NO: 8) | | | | | | | | | | |
| neuropeptide Y (NPY) | M15880 | Rn.9714 | Hs.1832 | Mm.154796 | 10 | 0 | 0 | 10 | 54 | 64 |
| (SEQ ID NO: 9) | | | | | | | | | | |
| TGFB inducible early | AI071299 | Rn.2398 | Hs.82173 | Mm.4292 | 0 | 12 | 0 | 12 | 49 | 61 |
| growth response (TIEG1) | | | | | | | | | | |
| (SEQ ID NO: 10) | | | | | | | | | | |
| nuclear orphan receptor | U01146 | Rn.88129 | Hs.82120 | Mm.3507 | 8 | 0 | 12 | 20 | 39.9 | 59.9 |
| HZF-3 (NURR1) | | | | | | | | | | |
| (SEQ ID NO: 11) | | | | | | | | | | |
| FGF receptor-1 | D12498 | Rn.9797 | Hs.748 | Mm.3157 | 40 | 12 | 0 | 52 | 7.7 | 59.7 |
| (SEQ ID NO: 12) | | | | | | | | | | |
| Jun B | X54686 | NA | NA | NA | 0 | 0 | 0 | 0 | 59.3 | 59.3 |
| (SEQ ID NO: 13) | | | | | | | | | | |
| Jun | AI175959 | Rn.44320 | NA | NA | 0 | 0 | 0 | 0 | 58.8 | 58.8 |
| (SEQ ID NO: 14) | | | | | | | | | | |
| NMDAR1 glutamate | U11418 | Rn.9840 | Hs.105 | Mm.3292 | 16 | 0 | 0 | 16 | 42 | 58 |
| receptor subunit | | | | | | | | | | |
| (SEQ ID NO: 15) | | | | | | | | | | |
| Tissue inhibitor of | AI169327 | Rn.25754 | NA | NA | 0 | 0 | 0 | 0 | 56.9 | 56.9 |
| metalloproteinase 1 | | | | | | | | | | |
| (TIMP1) | | | | | | | | | | |
| (SEQ ID NO: 16) | | | | | | | | | | |
| Narp | S82649 | NA | NA | NA | 10 | 0 | 0 | 10 | 46.4 | 56.4 |
| (SEQ ID NO: 17) | | | | | | | | | | |
| cytosolic phospholipase | U38376 | Rn.10162 | Hs.211587 | Mm.4186 | 0 | 6 | 0 | 6 | 48.6 | 54.6 |
| A2, group IVA | | | | | | | | | | |
| (SEQ ID NO: 18) | | | | | | | | | | |
| silencer factor B (SF-B) | X60769 | Rn.6479 | Hs.99029 | Mm.4863 | 0 | 6 | 6 | 12 | 37.5 | 49.5 |
| (SEQ ID NO: 19) | | | | | | | | | | |
| Minoxidil sulfotransferase | L19998 | Rn.1507 | Hs.142 | Mm.17339 | 10 | 0 | 12 | 22 | 25.6 | 47.6 |
| (SEQ ID NO: 20) | | | | | | | | | | |
| VGF nerve growth factor | M74223 | Rn.9704 | Hs.171014 | NA | 0 | 0 | 0 | 0 | 46.5 | 46.5 |
| inducible | | | | | | | | | | |
| (SEQ ID NO: 21) | | | | | | | | | | |
| phosphatidylinositol 3- | D64045 | Rn.10599 | Hs.6241 | Mm.3058 | 26 | 0 | 0 | 26 | 19.5 | 45.5 |
| kinase p85 alpha subunit | | | | | | | | | | |
| (SEQ ID NO: 22) | | | | | | | | | | |
| c-fos | X06769 | NA | NA | NA | 0 | 0 | 0 | 0 | 44.9 | 44.9 |
| (SEQ ID NO: 23) | | | | | | | | | | |
| Catalase | AA891848 | NA | Hs.395771 | Mm.4215 | 0 | 0 | 0 | 0 | 44.1 | 44.1 |
| (SEQ ID NO: 24) | | | | | | | | | | |
| phospholipase C-beta 1 | L14323 | Rn.45523 | Hs.41143 | Mm.42083 | 10 | 0 | 0 | 10 | 34 | 44 |
| (SEQ ID NO: 25) | | | | | | | | | | |
| ceruloplasmin | L33869 | Rn.32777 | Hs.296634 | Mm.13787 | 10 | 0 | 0 | 10 | 33.4 | 43.4 |
| (ferroxidase) | | | | | | | | | | |
| (SEQ ID NO: 26) | | | | | | | | | | |
| nerve growth factor- | M92433 | NA | NA | NA | 0 | 0 | 0 | 0 | 42.5 | 42.5 |
| induced clone C | | | | | | | | | | |
| (SEQ ID NO: 27) | | | | | | | | | | |
| thyrotropin-releasing | M23643 | NA | NA | NA | 0 | 0 | 0 | 0 | 42.4 | 42.4 |
| hormone (& precursor) | | | | | | | | | | |
| (SEQ ID NO: 28) | | | | | | | | | | |
| glial fibrillary acidic protein | AF028784 | NA | NA | NA | 0 | 0 | 0 | 0 | 42 | 42 |
| delta; glial fibrillary acidic | | | | | | | | | | |
| protein alpha | | | | | | | | | | |
| (SEQ ID NO: 29) | | | | | | | | | | |
| HES-1 factor | D13417 | Rn.19727 | Hs.250666 | Mm.4451 | 8 | 6 | 0 | 14 | 26.7 | 40.7 |
| (SEQ ID NO: 30) | | | | | | | | | | |
| activity and | AF030089 | Rn.40517 | Hs.21355 | Mm.44752 | 10 | 0 | 6 | 16 | 23.8 | 39.8 |
| neurotransmitter-induced | | | | | | | | | | |
| early gene protein 4 (ania- | | | | | | | | | | |
| 4) | | | | | | | | | | |
| (SEQ ID NO: 31) | | | | | | | | | | |
| Cytochrome P450 1b1 | AI176856 | Rn.10125 | Hs.154654 | Mm.4443 | 8 | 6 | 0 | 14 | 25.3 | 39.3 |
| (SEQ ID NO: 32) | | | | | | | | | | |
| arginine vasopressin | M25646 | Rn.9976 | Hs.89648 | Mm.6190 | 0 | 0 | 0 | 0 | 39 | 39 |
| (Diabetes insipidus) | | | | | | | | | | |
| (SEQ ID NO: 33) | | | | | | | | | | |
| enhancer-of-split and | AF009329 | Rn.10784 | Hs.33829 | Mm.89873 | 0 | 0 | 6 | 6 | 32.5 | 38.5 |
| hairy-related protein 1 | | | | | | | | | | |
| (SHARP 1) | | | | | | | | | | |
| (SEQ ID NO: 34) | | | | | | | | | | |
| Glutathione S-transferase, | AI138143 | Rn.87212 | NA | NA | 0 | 12 | 0 | 12 | 26.5 | 38.5 |
| theta 2 | | | | | | | | | | |
| (SEQ ID NO: 35) | | | | | | | | | | |
| protein phosphatase 1, | J05592 | Rn.9756 | Hs.76780 | Mm.143788 | 8 | 0 | 6 | 14 | 22.7 | 36.7 |
| regulatory (inhibitor) | | | | | | | | | | |
| subunit 1A | | | | | | | | | | |
| (SEQ ID NO: 36) | | | | | | | | | | |
| interleukin 6 receptor | M58587 | Rn.1716 | Hs.193400 | Mm.2856 | 8 | 6 | 0 | 14 | 22.2 | 36.2 |
| (SEQ ID NO: 37) | | | | | | | | | | |
| krox20; (early growth | U78102 | Rn.89235 | Hs.1395 | Mm.1353 | 0 | 0 | 0 | 0 | 36.2 | 36.2 |
| response 2) | | | | | | | | | | |
| (SEQ ID NO: 38) | | | | | | | | | | |
| light molecular weight | AF031880 | Rn.18568 | Hs.211584 | Mm.1956 | 0 | 6 | 0 | 6 | 30 | 36 |
| neurofilament | | | | | | | | | | |
| (SEQ ID NO: 39) | | | | | | | | | | |
| neurofilament protein | Z12152 | Rn.10971 | Hs.71346 | Mm.142140 | 0 | 6 | 0 | 6 | 29 | 35 |
| middle (NF-M) | | | | | | | | | | |
| (SEQ ID NO: 40) | | | | | | | | | | |
| S-adenosylmethionine | AI008131 | Rn.29949 | Hs.262476 | Mm.7880 | 10 | 0 | 0 | 10 | 24.8 | 34.8 |
| decarboxylase | | | | | | | | | | |
| (SEQ ID NO: 41) | | | | | | | | | | |
| ets variant gene 3 | AA859750 | Rn.7937 | NA | NA | 0 | 0 | 6 | 6 | 28.5 | 34.5 |
| (SEQ ID NO: 42) | | | | | | | | | | |
| neural receptor protein- | M55291 | Rn.11246 | Hs.47860 | Mm.3993 | 8 | 6 | 0 | 14 | 20.4 | 34.4 |
| tyrosine kinase (TrkB) | | | | | | | | | | |
| (SEQ ID NO: 43) | | | | | | | | | | |
| neurofilament protein; rat | M25638 | Rn.18568 | Hs.211584 | Mm.1956 | 0 | 6 | 0 | 6 | 28 | 34 |
| smallest (NF-L) | | | | | | | | | | |
| (SEQ ID NO: 44) | | | | | | | | | | |
| Vesl | AB003726 | Rn.37500 | NA | NA | | 0 | 0 | 0 | 33.9 | 33.9 |
| (SEQ ID NO: 45) | | | | | | | | | | |
| pyruvate dehydrogenase | AF062740 | Rn.31799 | Hs.22265 | NA | 16 | 0 | 0 | 16 | 17.6 | 33.6 |
| phosphatase isoenzyme 1 | | | | | | | | | | |
| (SEQ ID NO: 46) | | | | | | | | | | |
| EST195957 (Mad4 | AA892154 | Rn.3279 | NA | NA | 0 | 0 | 6 | 6 | 27.2 | 33.2 |
| homolog) | | | | | | | | | | |
| (SEQ ID NO: 47) | | | | | | | | | | |
| MHC class 1b RT1.S3 | AI235890 | Rn.40130 | NA | NA | 0 | 0 | 0 | 0 | 32.2 | 32.2 |
| (SEQ ID NO: 48) | | | | | | | | | | |
| Bad: bcl-2 associated | AA818072 | Rn.36696 | Hs.76366 | Mm.4387 | 0 | 0 | 0 | 0 | 32 | 32 |
| death agonist | | | | | | | | | | |
| (SEQ ID NO: 49) | | | | | | | | | | |
| Growth factor receptor | AI170776 | Rn.3360 | Hs.381152 | Mm.6900 | 0 | 0 | 0 | 0 | 32 | 32 |
| bound protein 2 (GRB2) | | | | | | | | | | |
| (SEQ ID NO: 50) | | | | | | | | | | |
| Growth factor receptor | AA801130 | Rn.3360 | Hs.381152 | Mm.6900 | 0 | 0 | 0 | 0 | 32 | 32 |
| bound protein 2 (GRB2) | | | | | | | | | | |
| (SEQ ID NO: 51) | | | | | | | | | | |
| Decorin | AI639233 | Rn.3819 | Hs.433989 | Mm.56769 | 0 | 0 | 0 | 0 | 31.5 | 31.5 |
| (SEQ ID NO: 52) | | | | | | | | | | |
| metabotropic glutamate | M61099 | Rn.87787 | Hs.32945 | Mm.157764 | 0 | 6 | 0 | 6 | 25.5 | 31.5 |
| receptor (GluR1) | | | | | | | | | | |
| (SEQ ID NO: 53) | | | | | | | | | | |
| prostaglandin D synthase | J04488 | Rn.11400 | Hs.430637 | Mm.1008 | 0 | 0 | 6 | 6 | 25.5 | 31.5 |
| (SEQ ID NO: 54) | | | | | | | | | | |
| gamma-aminobutyric acid | X51992 | Rn.10368 | Hs.24969 | Mm.261561 | 0 | 6 | 0 | 6 | 25 | 31 |
| A receptor, alpha 5 | | | | | | | | | | |
| (SEQ ID NO: 55) | | | | | | | | | | |
| aldehyde dehydrogenase | M73714 | Rn.9113 | Hs.159608 | Mm.4210 | 10 | 0 | 0 | 10 | 20.6 | 30.6 |
| (SEQ ID NO: 56) | | | | | | | | | | |
| rx00909s Rattus | AI638960 | Rn.16596 | NA | NA | 0 | 0 | 12 | 12 | 17.3 | 29.3 |
| norvegicus cDNA clone | | | | | | | | | | |
| (SEQ ID NO: 57) | | | | | | | | | | |
| activin type I receptor | L19341 | Rn.87899 | Hs.150402 | Mm.689 | 16 | 0 | 6 | 22 | 7.2 | 29.2 |
| (SEQ ID NO: 58) | | | | | | | | | | |
| growth factor (Arc) | U19866 | Rn.10086 | NA | Mm.25405 | 0 | 0 | 0 | 0 | 29 | 29 |
| (SEQ ID NO: 59) | | | | | | | | | | |
| Interferon-related | AI014163 | Rn.3723 | Hs.7879 | Mm.168 | 0 | 0 | 6 | 6 | 21.7 | 27.7 |
| developmental regulator 1 | | | | | | | | | | |
| (SEQ ID NO: 60) | | | | | | | | | | |
| neuritin | U88958 | Rn.3546 | NA | NA | 0 | 0 | 0 | 0 | 27.7 | 27.7 |
| (SEQ ID NO: 61) | | | | | | | | | | |
| myr 6 myosin heavy chain | U60416 | Rn.10640 | NA | Mm.3536 | 0 | 0 | 0 | 0 | 26.7 | 26.7 |
| (SEQ ID NO: 62) | | | | | | | | | | |
| interferon gamma receptor | U68272 | Rn.19927 | Hs.180866 | Mm.549 | 0 | 6 | 0 | 6 | 20.1 | 26.1 |
| (Ifngr) | | | | | | | | | | |
| (SEQ ID NO: 63) | | | | | | | | | | |
| lost on transformation 1 | U72620 | Rn.6977 | Hs.75825 | Mm.220978 | 8 | 12 | 0 | 20 | 6 | 26 |
| (LOT1) | | | | | | | | | | |
| (SEQ ID NO: 64) | | | | | | | | | | |
| Synapsin 2 | M27925 | Rn.506 | Hs.6439 | Mm.20892 | 10 | 0 | 0 | 10 | 16 | 26 |
| (SEQ ID NO: 65) | | | | | | | | | | |
| DNAJ (Hsp40) homolog, | AA891542 | Rn.4189 | NA | NA | 0 | 0 | 0 | 0 | 25.7 | 25.7 |
| subfamily B, member 5 | | | | | | | | | | |
| (SEQ ID NO: 66) | | | | | | | | | | |
| JE product (small inducibe | X17053 | NA | NA | NA | 0 | 0 | 0 | 0 | 25.4 | 25.4 |
| cytokine A2) | | | | | | | | | | |
| (SEQ ID NO: 67) | | | | | | | | | | |
| Activity and | AF030088 | Rn.37500 | NA | NA | 0 | 0 | 0 | 0 | 24.9 | 24.9 |
| Neurotransmitter induced | | | | | | | | | | |
| early gene 3; (ania-3) | | | | | | | | | | |
| (SEQ ID NO: 68) | | | | | | | | | | |
| matrilysin (Matrix | L24374 | Rn.10282 | Hs.2256 | Mm.4825 | 8 | 12 | 0 | 20 | 4.3 | 24.3 |
| metalloproteinase 7) | | | | | | | | | | |
| SEQ ID NO: 69) | | | | | | | | | | |
| 3CH134/CL100 PTPase | S81478 | NA | NA | NA | 0 | 0 | 0 | 0 | 24.3 | 24.3 |
| (SEQ ID NO: 70) | | | | | | | | | | |
| Roaz | U92564 | Rn.9981 | Hs.137168 | Mm.23452 | 10 | 0 | 0 | 10 | 14.3 | 24.3 |
| (SEQ ID NO: 71) | | | | | | | | | | |
| EST194857 | AA891054 | Rn.4287 | NA | NA | 0 | 0 | 0 | 0 | 24 | 24 |
| (SEQ ID NO: 72) | | | | | | | | | | |
| EST 189694 Normalized | AA800197 | Rn.3866 | NA | NA | 0 | 0 | 6 | 6 | 17 | 23 |
| rat heart | | | | | | | | | | |
| (SEQ ID NO: 73) | | | | | | | | | | |
| phospholipase D gene 2 | NM_033299 | Rn.9798 | Hs.104519 | Mm.121970 | 0 | 0 | 0 | 0 | 23 | 23 |
| (SEQ ID NO: 74) | | | | | | | | | | |
| tachykinin 2 (Neurokinin B | M16410 | Rn.9708 | Hs.9730 | Mm.2374 | 0 | 0 | 0 | 0 | 22.1 | 22.1 |
| precursor (NKB)) | | | | | | | | | | |
| (SEQ ID NO: 75) | | | | | | | | | | |
| rx04104s (Neurd1) | AI639109 | Rn.43903 | NA | NA | 0 | 0 | 0 | 0 | 21.8 | 21.8 |
| (SEQ ID NO: 76) | | | | | | | | | | |
| EST190175 (atrophin-1 | AA800678 | Rn.98495 | NA | NA | 0 | 0 | 0 | 0 | 21.5 | 21.5 |
| related protein) | | | | | | | | | | |
| (SEQ ID NO: 77) | | | | | | | | | | |
| solute carrier family 3, | M80804 | Rn.11196 | Hs.239106 | Mm.227176 | 10 | 0 | 0 | 10 | 11.4 | 21.4 |
| member 1 | | | | | | | | | | |
| (SEQ ID NO: 78) | | | | | | | | | | |
| Neuron glucose | D13962 | Rn.95055 | Hs.7594 | Mm.3726 | 8 | 0 | 0 | 8 | 13 | 21 |
| transporter | | | | | | | | | | |
| (SEQ ID NO: 79) | | | | | | | | | | |
| flavin-containing | M84719 | Rn.867 | Hs.1424 | Mm.976 | 10 | 0 | 0 | 10 | 10.3 | 20.3 |
| monooxygenase 1 | | | | | | | | | | |
| (SEQ ID NO: 80) | | | | | | | | | | |
| SPA-1 like protein | AI237576 | Rn.10835 | Hs.172180 | Mm.203907 | 16 | 0 | 0 | 16 | 4.2 | 20.2 |
| (SEQ ID NO: 81) | | | | | | | | | | |
| PP1MM110 | S74907 | NA | NA | NA | 0 | 0 | 0 | 0 | 19.9 | 19.9 |
| (SEQ ID NO: 82) | | | | | | | | | | |
| BHF-1 | D82074 | NA | NA | NA | 0 | 0 | 0 | 0 | 19 | 19 |
| (SEQ ID NO: 83) | | | | | | | | | | |
| leucine-rich acidic nuclear | D32209 | Rn.10123 | Hs.285013 | Mm.613 | 8 | 6 | 0 | 14 | 5 | 19 |
| protein | | | | | | | | | | |
| (SEQ ID NO: 84) | | | | | | | | | | |
| Nuclear receptor | AI176710 | Rn.62694 | Hs.80561 | Mm.101224 | 8 | 0 | 0 | 8 | 10.8 | 18.8 |
| subfamily 4, group A, | | | | | | | | | | |
| member 3 (NOR1) | | | | | | | | | | |
| (SEQ ID NO: 85) | | | | | | | | | | |
| rx01019s Rattus | AI639256 | Rn.40672 | NA | NA | 0 | 0 | 6 | 6 | 12.8 | 18.8 |
| norvegicus cDNA clone | | | | | | | | | | |
| (SEQ ID NO: 86) | | | | | | | | | | |
| Isovaleryl Coenzyme A | AI102838 | Rn.147 | Hs.374536 | Mm.6635 | 0 | 0 | 0 | 0 | 18.4 | 18.4 |
| dehydrogenase | | | | | | | | | | |
| (SEQ ID NO: 87) | | | | | | | | | | |
| insulin-like growth factor II | X17012 | NA | NA | NA | 0 | 0 | 0 | 0 | 17.8 | 17.8 |
| (somatomedin A) | | | | | | | | | | |
| (SEQ ID NO: 88) | | | | | | | | | | |
| GADD153 | U30186 | Rn.11183 | Hs.400353 | Mm.7549 | 0 | 0 | 0 | 0 | 17 | 17 |
| (SEQ ID NO: 89) | | | | | | | | | | |
| UI-R-E0-bs-h-03-0-UI.s1 | AA859627 | Rn.25 | NA | NA | 0 | 0 | 12 | 12 | 5 | 17 |
| UI-R-E0 Rattus | | | | | | | | | | |
| norvegicus cDNA clone | | | | | | | | | | |
| (SEQ ID NO: 90) | | | | | | | | | | |
| Calvasculin; S100 | X06916 | NA | NA | NA | 0 | 0 | 0 | 0 | 16.8 | 16.8 |
| calcium-binding protein A4 | | | | | | | | | | |
| (SEQ ID NO: 91) | | | | | | | | | | |
| UI-R-A0-bd-e-03-0-UI.s1 | AA866485 | Rn.3018 | NA | NA | 0 | 0 | 6 | 6 | 10 | 16 |
| UI-R-A0 Rattus | | | | | | | | | | |
| norvegicus cDNA clone | | | | | | | | | | |
| (SEQ ID NO: 92) | | | | | | | | | | |
| cyclic AMP-regulated | S65091 | NA | NA | NA | 0 | 0 | 0 | 0 | 15.8 | 15.8 |
| phosphoprotein | | | | | | | | | | |
| (SEQ ID NO: 93) | | | | | | | | | | |
| di-N-acetylchitobiase | M95768 | Rn.11199 | Hs.135578 | Mm.45396 | 10 | 0 | 0 | 10 | 5.8 | 15.8 |
| (SEQ ID NO: 94 | | | | | | | | | | |
| EST225243 (S100 | AI228548 | Rn.11091 | Hs.433503 | Mm.24662 | 0 | 0 | 0 | 0 | 15.7 | 15.7 |
| calcium binding protein | | | | | | | | | | |
| A1) | | | | | | | | | | |
| (SEQ ID NO: 95) | | | | | | | | | | |
| mothers against dpp 3 | U66479 | Rn.10636 | Hs.288261 | Mm.7320 | 0 | 0 | 0 | 0 | 15.7 | 15.7 |
| homolog | | | | | | | | | | |
| (SEQ ID NO: 96) | | | | | | | | | | |
| CCAAT/enhancerbinding, | M65149 | Rn.6975 | NA | NA | 0 | 0 | 0 | 0 | 15.4 | 15.4 |
| protein (C/EBP) delta | | | | | | | | | | |
| (SEQ ID NO: 97) | | | | | | | | | | |
| EST196314 (tescalcin) | AA892511 | Rn.14758 | Hs.18791 | Mm.26378 | 0 | 6 | 0 | 6 | 9.1 | 15.1 |
| (SEQ ID NO: 98) | | | | | | | | | | |
| EST220045 | AI176460 | NA | NA | NA | 0 | 0 | 0 | 0 | 15 | 15 |
| (SEQ ID NO: 99) | | | | | | | | | | |
| Potassium voltage gated | AI230211 | Rn.10540 | Hs.184889 | Mm.56904 | 0 | 0 | 0 | 0 | 14.3 | 14.3 |
| channel, Shal-related | | | | | | | | | | |
| family member 2 | | | | | | | | | | |
| (SEQ ID NO: 100) | | | | | | | | | | |
| transducin-like enhancer | AA875084 | Rn.6875 | NA | NA | 0 | 0 | 0 | 0 | 14 | 14 |
| of split 1(Tle1) | | | | | | | | | | |
| (SEQ ID NO: 101) | | | | | | | | | | |
| Glypican 1 | L02896 | Rn.7044 | Hs.2699 | Mm.24193 | 0 | 0 | 0 | 0 | 13 | 13 |
| (SEQ ID NO: 102) | | | | | | | | | | |
| chemokine CX3C | AF030358 | Rn.4106 | Hs.80420 | Mm.103711 | 0 | 0 | 0 | 0 | 12.9 | 12.9 |
| (SEQ ID NO: 103) | | | | | | | | | | |
| EST197009 | AA893206 | Rn.3665 | NA | NA | 0 | 0 | 0 | 0 | 12.8 | 12.8 |
| (SEQ ID NO: 104) | | | | | | | | | | |
| neuropilin | AF016296 | Rn.10815 | Hs.69285 | Mm.27448 | 0 | 0 | 0 | 0 | 12.8 | 12.8 |
| (SEQ ID NO: 105) | | | | | | | | | | |
| EST190350 | AA800853 | Rn.36357 | NA | NA | 0 | 0 | 0 | 0 | 12.4 | 12.4 |
| (SEQ ID NO: 106) | | | | | | | | | | |
| EST213163 (FK506 | AI103874 | Rn.1464 | NA | NA | 0 | 0 | 0 | 0 | 12 | 12 |
| binding protein 3) | | | | | | | | | | |
| (SEQ ID NO: 107) | | | | | | | | | | |
| Protein-tyrosine | AI180145 | Rn.11317 | Hs.155894 | Mm.259235 | 0 | 6 | 6 | 12 | 0 | 12 |
| phosphatase (Ptpn1, non- | | | | | | | | | | |
| receptor type 1) | | | | | | | | | | |
| (SEQ ID NO: 108) | | | | | | | | | | |
| Ras homolog enriched in | A1009605 | NA | NA | NA | 0 | 0 | 0 | 0 | 12 | 12 |
| brain | | | | | | | | | | |
| (SEQ ID NO: 109) | | | | | | | | | | |
| SH3-domain GRB2-like 1 | A1010110 | NA | NA | NA | 0 | 0 | 0 | 0 | 12 | 12 |
| (SEQ ID NO: 110) | | | | | | | | | | |
| DNA-damage-inducible | L32591 | Rn.10250 | Hs.80409 | Mm.1236 | 0 | 6 | 0 | 6 | 5.8 | 11.8 |
| transcript 1 | | | | | | | | | | |
| (SEQ ID NO: 111) | | | | | | | | | | |
| Early growth response 1 | AI176662 | Rn.9096 | Hs.326035 | Mm.181959 | 0 | 0 | 0 | 0 | 11.8 | 11.8 |
| (EGR1) | | | | | | | | | | |
| (SEQ ID NO: 112) | | | | | | | | | | |
| thymosin beta-10 | M58404 | Rn.5983 | Hs.76293 | NA | 8 | 0 | 0 | 8 | 3.8 | 11.8 |
| (SEQ ID NO: 113) | | | | | | | | | | |
| Y box protein 1 | AI230572 | Rn.3181 | Hs.74497 | Mm.21054 | 8 | 0 | 0 | 8 | 3.6 | 11.6 |
| (SEQ ID NO: 114) | | | | | | | | | | |
| phosducin-like protein | L15354 | Rn.51153 | Hs.9302 | Mm.30709 | 0 | 0 | 0 | 0 | 11.3 | 11.3 |
| (SEQ ID NO: 115) | | | | | | | | | | |
| Ash-s | D49847 | Rn.3360 | Hs.381152 | Mm.6900 | 0 | 0 | 0 | 0 | 11.2 | 11.2 |
| (SEQ ID NO: 116) | | | | | | | | | | |
| connexin protein Cx26 | X51615 | NA | NA | NA | 0 | 0 | 0 | 0 | 11.2 | 11.2 |
| (gap junction beta-2 | | | | | | | | | | |
| protein) | | | | | | | | | | |
| (SEQ ID NO: 117) | | | | | | | | | | |
| EST215655 (similar to | AI169756 | Rn.10581 | NA | NA | 0 | 0 | 0 | 0 | 10.7 | 10.7 |
| G33) | | | | | | | | | | |
| (SEQ ID NO: 118) | | | | | | | | | | |
| rx04422s Rattus | AI639169 | Rn.43195 | Hs.296420 | Mm.41508 | 0 | 0 | 6 | 6 | 4.6 | 10.6 |
| norvegicus cDNA clone | | | | | | | | | | |
| (SEQ ID NO: 119) | | | | | | | | | | |
| protein tyrosine | AJ007016 | Rn.1072 | NA | NA | 0 | 0 | 0 | 0 | 10.4 | 10.4 |
| phosphatase (PtP4a2) | | | | | | | | | | |
| (SEQ ID NO: 120) | | | | | | | | | | |
| EST195538 | AA891735 | Rn.22703 | NA | NA | 0 | 0 | 0 | 0 | 10.3 | 10.3 |
| (SEQ ID NO: 121) | | | | | | | | | | |
| EST 197783 Normalized | AA893980 | Rn.7498 | NA | NA | 0 | 0 | 0 | 0 | 10 | 10 |
| rat placenta | | | | | | | | | | |
| (SEQ ID NO: 122) | | | | | | | | | | |
| rx04826s Rattus | AI639058 | Rn.20963 | NA | NA | 0 | 0 | 0 | 0 | 10 | 10 |
| norvegicus cDNA clone | | | | | | | | | | |
| (similar to Nedd4) | | | | | | | | | | |
| (SEQ ID NO: 123) | | | | | | | | | | |
| UI-R-E0-ci-e-02-0-UI.s1 | AA874943 | Rn.4040 | NA | NA | 0 | 0 | 0 | 0 | 9.2 | 9.2 |
| UI-R-E0 Rattus | | | | | | | | | | |
| norvegicus cDNA clone | | | | | | | | | | |
| (SEQ ID NO: 124) | | | | | | | | | | |
| EST 195383 Normalized | AA891580 | Rn.22698 | NA | NA | 0 | 0 | 0 | 0 | 9 | 9 |
| rat kidney | | | | | | | | | | |
| (SEQ ID NO: 125) | | | | | | | | | | |
| Transcription factor | M61725 | Rn.22469 | NA | NA | 0 | 0 | 0 | 0 | 8.8 | 8.8 |
| UBF(SEQ ID NO: 126) | | | | | | | | | | |
| EST203592 (chromobox | AI009141 | Rn.29900 | NA | NA | 0 | 0 | 0 | 0 | 8.7 | 8.7 |
| homolog 1) | | | | | | | | | | |
| (SEQ ID NO: 127) | | | | | | | | | | |
| Lysyl oxidase | S77494 | NA | NA | NA | 0 | 0 | 0 | 0 | 8.5 | 8.5 |
| (SEQ ID NO: 128) | | | | | | | | | | |
| beta-tubulin T beta15 (aa | X03369 | Rn.37849 | NA | NA | 0 | 0 | 0 | 0 | 8 | 8 |
| 1-445) | | | | | | | | | | |
| (SEQ ID NO: 129) | | | | | | | | | | |
| diacylglycerol kinase | S49760 | NA | NA | NA | 0 | 0 | 0 | 0 | 7 | 7 |
| (SEQ ID NO: 130) | | | | | | | | | | |
| Small proline-rich protein | AA891911 | Rn.14720 | NA | NA | 0 | 0 | 0 | 0 | 6.9 | 6.9 |
| gene (cornilin alpha) | | | | | | | | | | |
| (SEQ ID NO: 131) | | | | | | | | | | |
| EST196165 | AA892362 | Rn.14752 | NA | NA | 0 | 0 | 0 | 0 | 6.7 | 6.7 |
| (SEQ ID NO: 132) | | | | | | | | | | |
| EST106597 | H31990 | Rn.22664 | NA | NA | 0 | 0 | 0 | 0 | 6.6 | 6.6 |
| (SEQ ID NO: 133) | | | | | | | | | | |
| heme oxygenase-3 | AF058787 | NA | NA | NA | 0 | 0 | 0 | 0 | 6.5 | 6.5 |
| (SEQ ID NO: 134) | | | | | | | | | | |
| EST197625 (RAD52 | AA893822 | Rn.8154 | NA | NA | 0 | 0 | 0 | 0 | 6.3 | 6.3 |
| homolog) | | | | | | | | | | |
| (SEQ ID NO: 135) | | | | | | | | | | |
| protein kinase MNK2 | Z21935 | Rn.92317 | NA | NA | 0 | 0 | 0 | 0 | 6.2 | 6.2 |
| (SEQ ID NO: 136) | | | | | | | | | | |
| rx00382s Rattus | AI639155 | Rn.96446 | NA | NA | 0 | 0 | 0 | 0 | 5.2 | 5.2 |
| norvegicus cDNA clone | | | | | | | | | | |
| (SEQ ID NO: 137) | | | | | | | | | | |
| SHB (Src homology 2 | AA859468 | Rn.226 | NA | NA | 0 | 0 | 0 | 0 | 5.2 | 5.2 |
| domain containing) | | | | | | | | | | |
| adaptor protein B | | | | | | | | | | |
| (SEQ ID NO: 138) | | | | | | | | | | |
| Sodium-dependant | S56141 | NA | NA | NA | 0 | 0 | 0 | 0 | 5 | 5 |
| neurotransmitter | | | | | | | | | | |
| transporter | | | | | | | | | | |
| (SEQ ID NO: 139) | | | | | | | | | | |
| UI-R-E0-bx-e-11-0-UI.s1 | AA859690 | Rn.51 | NA | NA | 0 | 0 | 0 | 0 | 4.7 | 4.7 |
| UI-R-E0 Rattus | | | | | | | | | | |
| norvegicus cDNA clone | | | | | | | | | | |
| (SEQ ID NO: 140) | | | | | | | | | | |
| EST190198 (coagulation | AA800701 | Rn.97659 | NA | NA | 0 | 0 | 0 | 0 | 4.4 | 4.4 |
| factor III) | | | | | | | | | | |
| (SEQ ID NO: 141) | | | | | | | | | | |
| EST190228 | AA800731 | Rn.6626 | NA | NA | 0 | 0 | 0 | 0 | 4.1 | 4.1 |
| (SEQ ID NO: 142) | | | | | | | | | | |
| EST197395 (reticulocalbin | AA893592 | Rn.98490 | NA | NA | 0 | 0 | 0 | 0 | 4.1 | 4.1 |
| 2) | | | | | | | | | | |
| (SEQ ID NO: 143) | | | | | | | | | | |
| nuclear pore complex | U41845 | Rn.3242 | Hs.367697 | Mm.28379 | 0 | 0 | 0 | 0 | 3.9 | 3.9 |
| protein Nup50 | | | | | | | | | | |
| (SEQ ID NO: 144) | | | | | | | | | | |
| clathrin, light polypeptide | AA874955 | Rn.3440 | Hs.380749 | Mm.36879 | 0 | 0 | 0 | 0 | 3.8 | 3.8 |
| (LCB) | | | | | | | | | | |
| (SEQ ID NO: 145) | | | | | | | | | | |
| NAC-1 protein | AF015911 | Rn.94891 | NA | NA | 0 | 0 | 0 | 0 | 3.8 | 3.8 |
| (SEQ ID NO: 146) | | | | | | | | | | |
| beta defensin-1 | AF068860 | Rn.31800 | NA | Mm.5341 | 0 | 0 | 0 | 0 | 3.7 | 3.7 |
| (SEQ ID NO: 147) | | | | | | | | | | |
| voltage-dependent | X12589 | NA | NA | NA | 0 | 0 | 0 | 0 | 3.5 | 3.5 |
| potassium channel; | | | | | | | | | | |
| (shaker-related subfamily) | | | | | | | | | | |
| (SEQ ID NO: 148) | | | | | | | | | | |
| rx00967s Rattus | AI639015 | Rn.28835 | NA | NA | 0 | 0 | 0 | 0 | 3.1 | 3.1 |
| norvegicus cDNA clone | | | | | | | | | | |
| (SEQ ID NO: 149) | | | | | | | | | | |
| Beta-carotene 15,15′- | AI014135 | NA | NA | NA | 0 | 0 | 0 | 0 | 2.9 | 2.9 |
| dioxygenase | | | | | | | | | | |
| (SEQ ID NO: 150) | | | | | | | | | | |
| BRL-3A binding protein | A09811 | NA | NA | NA | 0 | 0 | 0 | 0 | 2.9 | 2.9 |
| (SEQ ID NO: 151) | | | | | | | | | | |
| potassium voltage gated | M32867 | Rn.9884 | Hs.1854 | Mm.142718 | 0 | 0 | 0 | 0 | 2.7 | 2.7 |
| channel, shaker related | | | | | | | | | | |
| subfamily, member 4 | | | | | | | | | | |
| (SEQ ID NO: 152) | | | | | | | | | | |

9. References Cited

Numerous references, including patents, patent applications and various publications, are cited and discussed in the description of this invention. The citation and/or discussion of such references is provided merely to clarify the description of the present invention and is not an admission that any such reference is “prior art” to the invention described here. All references cited and/or discussed in this specification (including references, e.g., to biological sequences or structures in the GenBank, PDB or other public databases) are incorporated herein by reference in their entirety and to the same extent as if each reference was individually incorporated by reference.

What is claimed is:

1. A method for identifying a compound to treat a neuropsychiatric disorder, which method comprises:
(a) contacting a cell with a test compound;
(b) determining expression by the cell of one or more signature genes, each said signature gene comprising a nucleic acid that hybridizes to a nucleic acid selected from the group consisting of SEQ ID NOS:1-154 and the complements thereof; and
(c) comparing the determined expression of the one or more signature genes to expression in a cell not contacted with the test compound,
wherein changes in expression of the one or more signature genes consistent with a therapeutic effect indicate that the test compound is useful for treating the neuropsychiatric disorder.

2. The method according to claim 1 in which the neuropsychiatric disorder is selected from the group consisting of, schizophrenia, autism, major depressive disorder (MDD), bipolar affective disorder (BAD), schizophrenia and psychotic depression.

3. The method according to claim 1 in which the cell is a neuronal cell.

4. The method according to claim 1, wherein changes in expression of signature genes which are similar to changes observed in ECS indicate that the test compound is useful for treating the disease or disorder.

5. The method according to claim 1, wherein changes in expression of signature genes which are similar to changes observed in ECT indicate that the test compound is useful for treating the disease or disorder.

6. The method according to claim 1, in which changes in the expression of signature genes are evaluated from a value (V) comprising the sum of each signature gene's change in expression.

7. The method according to claim 6, in which said value (V) is determined from the normalized change (Eᵢ) in expression of each efficacy gene (i) weighted by the score value (ωᵢ) according to the relation:
$$V = {\sum }_{i} {ω}_{i} {E}_{i} .$$

8. A method for selecting one or more signature genes that are indicative of an effective therapy for treating a neuropsychiatric disorder, which method comprises identifying nucleic acids that are differentially expressed in an individual subjected to electroconvulsive seizure (ECS) compared to an individual not subjected to ECS.

9. The method according to claim 8 in which the neuropsychiatric disorder is selected from the group consisting of, schizophrenia, autism, major depressive disorder (MDD), bipolar affective disorder (BAD), schizophrenia and psychotic depression.

10. The method according to claim 8 wherein the individual is subjected to acute ECS.

11. The method according to claim 8 wherein the individual is subject to chronic ECS.

12. The method according to claim 8, wherein nucleic acids are identified that are differentially expressed in the hippocampus of an individual subjected to ECS compared to expression in the hippocampus of an individual not subjected to ECS.

13. The method according to claim 8, wherein nucleic acids are identified that are differentially expressed in the frontal cortex of an individual subjected to ECS compared to expression in the frontal cortex of an individual not subjected to ECS.

14. The method according to claim 8, wherein:
(a) a score value is obtained for each of the identified nucleic acids, the score value for each gene being a function of each gene's differential expression in individuals subjected to ECS, and
(b) signature genes are selected which have the highest score value.

15. The method according to claim 8, wherein nucleic acids that are differentially expressed comprise one or more nucleic acids that hybridize to a nucleic acid selected from the group consisting of SEQ ID NOS:1-54 or to a complement thereof.

16. A kit for detecting an ECS gene signature, wherein said kit comprises a plurality of oligonucleotides, each of which is capable of specifically hybridizing to a different ECS signature gene.

17. A kit according to claim 16, wherein each ECS signature gene is a nucleic acid having a nucleotide sequence selected from the group consisting of SEQ ID NOS:1-152, a complement thereof or a homolog thereof.

18. A kit according to claim 17, wherein the ECS signature genes comprise at least one homolog of a sequence selected from the group consisting of SEQ ID NOS:1-152, or a complement thereof.

19. A kit according to claim 18, wherein said homolog specifically hybridizes from a nucleic acid comprising a nucleotide sequence selected from the group consisting of SEQ ID NOS:1-152 and their complementary sequences.

20. A kit according to claim 18, wherein said homolog comprises a nucleotide sequence that is at least 80% identical to a nucleotide sequence selected from the group consisting of SEQ ID NOS:1-52 and their complementary sequences.

21. A kit according to claim 16, wherein each of the plurality of oligonucleotides is immobilized on a solid surface or support.

22. A kit according to claim 21, wherein each oligonucleotide is immobilized at a known position on the solid surface or support.

23. A kit according to claim 16, wherein at least some oligonucleotides in the plurality of oligonucleotides are capable of priming reverse transcription of an ECS signature gene.

24. A kit according to claim 23, further comprising a polymerase and nucleotide bases.

25. A kit according to claim 24 wherein the nucleotide bases are detectably labeled.

26. A kit according to claim 16 comprising oligonucleotides capable of specifically hybridizing to at least 10 different ECS signature genes.

27. A kit according to claim 16 comprising oligonucleotides capable of specifically hybridizing to at least 50 different ECS signature genes.

28. A kit according to claim 16 comprising oligonucleotides capable of specifically hybridizing to at least 100 different ECS signature genes.

29. A kit according to claim 16 comprising oligonucleotides capable of specifically hybridizing to at least 150 different ECS signature genes.
